SEQUENCE LISTING (193 sequences)

SEQ ID NO: 1 - protein, 7 aa, synthetic construct
ASSLNIA

SEQ ID NO: 2 - protein, 7 aa, synthetic construct
PYDQLRH

SEQ ID NO: 3 - protein, 7 aa, synthetic construct
KAMHQMQ

SEQ ID NO: 4 - protein, 13 aa, synthetic construct
CGFECVRQCPERC

SEQ ID NO: 5 - protein, 11 aa, synthetic construct
ACDCRGDCFCG

SEQ ID NO: 6 - protein, 15 aa, synthetic construct
  MOD_RES 1: note = Acp
  MOD_RES 3: note = Acp
  MOD_RES 9: note = Acp
  MOD_RES 14: note = Acp
  MOD_RES 6: note = bAla
  MOD_RES 12: note = bAla
  MOD_RES 15: note = bAla
XRXRRXRRXRRXRXX

SEQ ID NO: 7 - DNA, 1464 bp, synthetic construct
  source 1..1464: mol_type = other DNA
atgaaccagagcattccagtggctcctacacctcctagaagagtgagactgaaaccttggctggtggctcaggtgaactcttgtcagtatccaggactccagtgggtgaacggagaaaagaagctgttttgcatcccttggagacacgctacaagacacggaccttctcaggacggagataacaccatcttcaaggcttgggctaaggagacaggaaagtacacagagggagtggacgaagcagatccagctaagtggaaggctaatctcaggtgcgccctgaataagagcagagatttcaggctgatctacgacggacctagagatatgcctcctcagccttacaagatctacgaagtgtgctctaacggaccagctcctacagattctcagccaccagaagattatagctttggagccggagaagaagaggaggaagaagaagaactgcagagaatgctgcctagtctgtctctgacagaagatgtcaagtggcctcctacactgcagcctcctacactgcagcctccagtggtgctgggacctccagctccagatccttctcctctggctcctcctccaggaaatccagcaggatttagagagctgctgtcagaagtgctggaaccaggacctctgccagcttctctgcctccagcaggagaacagctgctgccagatctgctgatttctcctcatatgctgcctctgacagatctggagatcaagttccagtacaggggaagacctcctagagctctgacaatctctaaccctcacggctgtagactgttctactctcagctggaagctacacaggaacaggtggaactgttcggacctatctctctggaacaggtgagattcccttctccagaggatatccctagcgataagcagaggttctacacaaatcagctgctggacgtgctggatagaggactgattctgcagctgcagggacaggatctgtacgctattaggctctgccagtgtaaggtgttttggtcaggaccttgcgctagcgctcacgattcttgtcctaatcctatccagagggaggtgaagacaaagctgttctctctggagcacttcctgaacgaactgatcctgttccagaaaggccagacaaatacccctcctcctttcgagatcttcttttgcttcggcgaagagtggccagatagaaagcctagagagaagaagctgattacagtgcaggtggtgccagtggcagctagactgctcctggaaatgtttagcggagaactggattgggacgcagacgatatcagactgcagatcgacaacccagatctgaaggacagaatggtggagcagttcaaggagctgcatcacatttggcagagtcagcagagactgcagccagtggctcaggctcctccaggagcaggactgggagtgggacagggaccttggcctatgcatccagcaggaatgcag

SEQ ID NO: 8 - protein, 488 aa, synthetic construct
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELDWDADDIRLQIDNPDLKDRMVEQFKELHHIWQSQQRLQPVAQAPPGAGLGVGQGPWPMHPAGMQ

SEQ ID NO: 9 - protein, 39 aa, synthetic construct
HGEGTFTSDLSKQMEEEAVRLFIEWLKNGGPSSGAPPPS

SEQ ID NO: 10 - protein, 5 aa, synthetic construct
KKEEE

SEQ ID NO: 11 - protein, 32 aa, synthetic construct
YTIWMPENPRPGTPCDIFTNSRGKRASNGGGG

SEQ ID NO: 12 - protein, 30 aa, synthetic construct
KSVRTWNEIIPSKGCLRVGGRCHPHVNGGG

SEQ ID NO: 13 - protein, 12 aa, synthetic construct
APWHLSSQYSRT

SEQ ID NO: 14 - protein, 9 aa, synthetic construct
CARSKNKDC

SEQ ID NO: 15 - protein, 7 aa, synthetic construct
PKRGFQD

SEQ ID NO: 16 - protein, 7 aa, synthetic construct
SNTRVAP

SEQ ID NO: 17 - protein, 16 aa, synthetic construct
KKEEEKKEEEKKEEEK

SEQ ID NO: 18 - protein, 4 aa, synthetic construct
EEEE

SEQ ID NO: 19 - protein, 11 aa, synthetic construct
RHLKRHLKGSC

SEQ ID NO: 20 - protein, 12 aa, synthetic construct
HLKRFKRFKGSC

SEQ ID NO: 21 - protein, 9 aa, synthetic construct
LRKRLKRHA

SEQ ID NO: 22 - protein, 12 aa, synthetic construct
LRKRLKRLKGSC

SEQ ID NO: 23 - protein, 9 aa, synthetic construct
LRKRLKRHC

SEQ ID NO: 24 - protein, 23 aa, synthetic construct
  MOD_RES 11: note = Acp
  MOD_RES 14: note = bAla
  MOD_RES 17: note = Acp
  MOD_RES 20: note = bAla
  MOD_RES 22: note = Acp
  MOD_RES 23: note = bAla
LRKRLKRHCRXRRXRRXRRXRXX

SEQ ID NO: 25 - protein, 19 aa, synthetic construct
  MOD_RES 19: note = Acp
LRKRLKRHCGAASSLNIAX

SEQ ID NO: 26 - protein, 33 aa, synthetic construct
  MOD_RES 19: note = Acp
  MOD_RES 21: note = Acp
  MOD_RES 24: note = bAla
  MOD_RES 27: note = Acp
  MOD_RES 30: note = bAla
  MOD_RES 32: note = Acp
  MOD_RES 33: note = bAla
LRKRLKRHCGAASSLNIAXRXRRXRRXRRXRXX

SEQ ID NO: 27 - protein, 26 aa, synthetic construct
  MOD_RES 14: note = Acp
  MOD_RES 17: note = bAla
  MOD_RES 20: note = Acp
  MOD_RES 23: note = bAla
  MOD_RES 25: note = Acp
  MOD_RES 26: note = bAla
LRKRLKRLKRHCRXRRXRRXRRXRXX

SEQ ID NO: 28 - protein, 8 aa, synthetic construct
LRLRKGSC

SEQ ID NO: 29 - protein, 4 aa, synthetic construct
HRKK

SEQ ID NO: 30 - protein, 9 aa, synthetic construct
RFIKKEKRG

SEQ ID NO: 31 - protein, 10 aa, synthetic construct
SYRKLRFKER

SEQ ID NO: 32 - protein, 9 aa, synthetic construct
RLKHGDKLR

SEQ ID NO: 33 - protein, 10 aa, synthetic construct
HVRKHVRKVR

SEQ ID NO: 34 - protein, 11 aa, synthetic construct
LHRKRHLKGSC

SEQ ID NO: 35 - protein, 8 aa, synthetic construct
RLRLKLRL

SEQ ID NO: 36 - protein, 22 aa, synthetic construct
  MOD_RES 22: note = Acp
LRKRLKRLKGSCGAASSLNIAX

SEQ ID NO: 37 - protein, 28 aa, synthetic construct
  MOD_RES 28: note = Acp
LRKRLKRLKGSCHHHHHHGAASSLNIAX

SEQ ID NO: 38 - protein, 18 aa, synthetic construct
  CARBOHYD 18: note = (S-S)-alpha-D-Thiomannose
LRKRLKGSGGSGGSGGSC

SEQ ID NO: 39 - protein, 14 aa, synthetic construct
  CARBOHYD 14: note = (S-S)-alpha-D-Thiomannose
EEEEKGSGGSGGSC

SEQ ID NO: 40 - protein, 4 aa, synthetic construct
LRLR

SEQ ID NO: 41 - protein, 12 aa, synthetic construct
KLKKLKKLKGSC

SEQ ID NO: 42 - protein, 9 aa, synthetic construct
KLKKLKGSC

SEQ ID NO: 43 - protein, 6 aa, synthetic construct
KLKGSC

SEQ ID NO: 44 - protein, 12 aa, synthetic construct
LRKLRKLRKGSC

SEQ ID NO: 45 - protein, 12 aa, synthetic construct
RLKRLKLRKGSC

SEQ ID NO: 46 - protein, 12 aa, synthetic construct
RLKRLKRLKGSC

SEQ ID NO: 47 - protein, 12 aa, synthetic construct
RLKLRKLRKGSC

SEQ ID NO: 48 - protein, 9 aa, synthetic construct
RLKRLKGSC

SEQ ID NO: 49 - protein, 6 aa, synthetic construct
RLKGSC

SEQ ID NO: 50 - protein, 12 aa, synthetic construct
KLKLKKLKKGSC

SEQ ID NO: 51 - protein, 12 aa, synthetic construct
LKKLKKLKKGSC

SEQ ID NO: 52 - protein, 12 aa, synthetic construct
KLKKLKKLKGSA

SEQ ID NO: 53 - protein, 9 aa, synthetic construct
KKKKKKGSC

SEQ ID NO: 54 - protein, 12 aa, synthetic construct
KLKKLKKLKKLC

SEQ ID NO: 55 - protein, 5 aa, synthetic construct
RKRHC

SEQ ID NO: 56 - protein, 7 aa, synthetic construct
RKRKRHC

SEQ ID NO: 57 - protein, 9 aa, synthetic construct
RKRKRKRHC

SEQ ID NO: 58 - protein, 6 aa, synthetic construct
LRKRHC

SEQ ID NO: 59 - protein, 12 aa, synthetic construct
LRKRLKRLKRHC

SEQ ID NO: 60 - protein, 10 aa, synthetic construct
LRKRLKRLKA

SEQ ID NO: 61 - protein, 12 aa, synthetic construct
LRKRLKRLKRFW

SEQ ID NO: 62 - protein, 12 aa, synthetic construct
LRKRLKRLKRYM

SEQ ID NO: 63 - protein, 11 aa, synthetic construct
LRKRLKRLKYM

SEQ ID NO: 64 - protein, 21 aa, synthetic construct
LRKRLKRLKGSCRHCRFWRYM

SEQ ID NO: 65 - protein, 11 aa, synthetic construct
RLRKRLRKRHC

SEQ ID NO: 66 - protein, 15 aa, synthetic construct
RLRKRLRKRLRKRHC

SEQ ID NO: 67 - protein, 13 aa, synthetic construct
LRLRKLRLRKGSC

SEQ ID NO: 68 - protein, 18 aa, synthetic construct
LRLRKLRLRKLRLRKGSC

SEQ ID NO: 69 - protein, 12 aa, synthetic construct
HKKKHKKLKGSC

SEQ ID NO: 70 - protein, 12 aa, synthetic construct
LRKRLKREKGSC

SEQ ID NO: 71 - protein, 12 aa, synthetic construct
WRKRMKRFKGSC

SEQ ID NO: 72 - protein, 12 aa, synthetic construct
  MOD_RES 1: note = bAla
  MOD_RES 5: note = bAla
  MOD_RES 8: note = Acp
XRKRXKRXKGSC

SEQ ID NO: 73 - protein, 12 aa, synthetic construct
YRKRTKRQKGSC

SEQ ID NO: 74 - protein, 12 aa, synthetic construct
FRKRQKRWKGSC

SEQ ID NO: 75 - protein, 12 aa, synthetic construct
QRKRTKRYKGSC

SEQ ID NO: 76 - protein, 12 aa, synthetic construct
MRKRCKRCKGSC

SEQ ID NO: 77 - protein, 9 aa, synthetic construct
RRKLLKGSC

SEQ ID NO: 78 - protein, 12 aa, synthetic construct
  MOD_RES 3: note = Dbu
  MOD_RES 6: note = Dbu
  MOD_RES 9: note = Dbu
LRXRLXRLXGSC

SEQ ID NO: 79 - protein, 36 aa, synthetic construct
  MOD_RES 22: note = Acp
  MOD_RES 24: note = Acp
  MOD_RES 27: note = bAla
  MOD_RES 30: note = Acp
  MOD_RES 33: note = bAla
  MOD_RES 35: note = Acp
  MOD_RES 36: note = bAla
LRKRLKRLKGSCGAASSLNIAXRXRRXRRXRRXRXX

SEQ ID NO: 80 - protein, 12 aa, synthetic construct
HLKRLKRLKGSC

SEQ ID NO: 81 - protein, 12 aa, synthetic construct
HLKHLKHLKGSC

SEQ ID NO: 82 - protein, 12 aa, synthetic construct
RLKRWKRWKGSC

SEQ ID NO: 83 - protein, 25 aa, synthetic construct
  MOD_RES 2: note = Acp
  MOD_RES 5: note = bAla
  MOD_RES 8: note = Acp
  MOD_RES 11: note = bAla
  MOD_RES 13: note = Acp
  MOD_RES 14: note = bAla
  MOD_RES 25: note = Acp
RXRRXRRXRRXRXXKGAASSLNIAX

SEQ ID NO: 84 - protein, 11 aa, synthetic construct
RHLKRHLKRHC

SEQ ID NO: 85 - protein, 26 aa, synthetic construct
LRKRLKRLKGSCGARRQPPRSISSHP

SEQ ID NO: 86 - protein, 18 aa, synthetic construct
  MOD_RES 18: note = Acp
EEEEKGSCGAASSLNIAX

SEQ ID NO: 87 - protein, 24 aa, synthetic construct
  DISULFID 15..23: note = Disulfide bridge
  DISULFID 17..21: note = Disulfide bridge
EEEEKGSGGSGGSACDCRGDCFCG

SEQ ID NO: 88 - protein, 20 aa, synthetic construct
ESGESGESGKGSEGSEGSEC

SEQ ID NO: 89 - protein, 5 aa, synthetic construct
RKGSC

SEQ ID NO: 90 - protein, 5 aa, synthetic construct
RKRKR

SEQ ID NO: 91 - protein, 5 aa, synthetic construct
  SITE 1: note = D-Lysine
  SITE 3..4: note = D-Lysine
  SITE 2: note = D-Leucine
  SITE 5: note = D-Leucine
KLKKL

SEQ ID NO: 92 - protein, 5 aa, synthetic construct
  SITE 1: note = D-Leucine
  SITE 5: note = D-Leucine
  SITE 2: note = D-Arginine
  SITE 4: note = D-Arginine
LRKRL

SEQ ID NO: 93 - protein, 17 aa, synthetic construct
EEEEEEEEGSGGSGGSC

SEQ ID NO: 94 - protein, 12 aa, synthetic construct
RFIKLRLRKRFW

SEQ ID NO: 95 - protein, 9 aa, synthetic construct
RWKRWKRHA

SEQ ID NO: 96 - protein, 10 aa, synthetic construct
RFIKHKKRFW

SEQ ID NO: 97 - protein, 7 aa, synthetic construct
HGDKRHC

SEQ ID NO: 98 - protein, 12 aa, synthetic construct
HVRKRLRLKRYM

SEQ ID NO: 99 - protein, 10 aa, synthetic construct
RHLKRFIKLR

SEQ ID NO: 100 - protein, 18 aa, synthetic construct
LEKELKELKGSGGSGGSC

SEQ ID NO: 101 - protein, 14 aa, synthetic construct
LELEKGSGGSGGSC

SEQ ID NO: 102 - protein, 16 aa, synthetic construct
LEKELKEHCGGSGGSC

SEQ ID NO: 103 - protein, 17 aa, synthetic construct
EHLKEHLKGSGGSGGSC

SEQ ID NO: 104 - protein, 11 aa, synthetic construct
RHLKSYRKRHA

SEQ ID NO: 105 - protein, 10 aa, synthetic construct
LRFKLLKRFW

SEQ ID NO: 106 - protein, 5 aa, synthetic construct
  SITE 1: note = D-Leucine
  SITE 4: note = D-Arginine
LRKRL

SEQ ID NO: 107 - protein, 5 aa, synthetic construct
  SITE 1: note = D-Arginine
  SITE 4: note = D-Arginine
  SITE 2: note = D-Leucine
  SITE 5: note = D-Leucine
  SITE 3: note = D-Lysine
RLKRL

SEQ ID NO: 108 - protein, 7 aa, synthetic construct
RLRKRHC

SEQ ID NO: 109 - protein, 12 aa, synthetic construct
RKRLKRLRKRHC

SEQ ID NO: 110 - protein, 12 aa, synthetic construct
LRKRKRLRKRHC

SEQ ID NO: 111 - protein, 13 aa, synthetic construct
LRKRKRLRLKRHC

SEQ ID NO: 112 - protein, 100 aa, synthetic construct
EEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEEE

SEQ ID NO: 113 - protein, 12 aa, synthetic construct
  SITE 1: note = D-Leucine
  SITE 5: note = D-Leucine
  SITE 8: note = D-Leucine
  SITE 2: note = D-Arginine
  SITE 4: note = D-Arginine
  SITE 7: note = D-Arginine
  SITE 3: note = D-Lysine
  SITE 6: note = D-Lysine
  SITE 9: note = D-Lysine
  SITE 11: note = D-Serine
  SITE 12: note = D-Cysteine
LRKRLKRLKGSC

SEQ ID NO: 114 - protein, 12 aa, synthetic construct
  SITE 1: note = D-Leucine
  SITE 5: note = D-Leucine
  SITE 8: note = D-Leucine
  SITE 2: note = D-Arginine
  SITE 4: note = D-Arginine
  SITE 7: note = D-Arginine
  SITE 11: note = D-Serine
  SITE 12: note = D-Cysteine
LRKRLKRLKGSC

SEQ ID NO: 115 - protein, 12 aa, synthetic construct
  SITE 1: note = D-Leucine
  SITE 8: note = D-Leucine
  SITE 6: note = D-Lysine
  SITE 4: note = D-Arginine
LRKRLKRLKGSC

SEQ ID NO: 116 - protein, 12 aa, synthetic construct
  SITE 1: note = D-Arginine
  SITE 4: note = D-Arginine
  SITE 7: note = D-Arginine
  SITE 2: note = D-Leucine
  SITE 5: note = D-Leucine
  SITE 8: note = D-Leucine
  SITE 3: note = D-Lysine
  SITE 6: note = D-Lysine
  SITE 9: note = D-Lysine
  SITE 11: note = D-Serine
  SITE 12: note = D-Cysteine
RLKRLKRLKGSC

SEQ ID NO: 117 - protein, 12 aa, synthetic construct
  SITE 1: note = D-Lysine
  SITE 3..4: note = D-Lysine
  SITE 6..7: note = D-Lysine
  SITE 9: note = D-Lysine
  SITE 2: note = D-Leucine
  SITE 5: note = D-Leucine
  SITE 8: note = D-Leucine
  SITE 11: note = D-Serine
  SITE 12: note = D-Cysteine
KLKKLKKLKGSC

SEQ ID NO: 118 - protein, 7 aa, synthetic construct
RHLKRHL

SEQ ID NO: 119 - protein, 8 aa, synthetic construct
HLKRFKRF

SEQ ID NO: 120 - protein, 5 aa, synthetic construct
LRKRL

SEQ ID NO: 121 - protein, 8 aa, synthetic construct
LRKRLKRL

SEQ ID NO: 122 - protein, 6 aa, synthetic construct
RFIKKE

SEQ ID NO: 123 - protein, 7 aa, synthetic construct
SYRKLRF

SEQ ID NO: 124 - protein, 6 aa, synthetic construct
RLKHGD

SEQ ID NO: 125 - protein, 7 aa, synthetic construct
HVRKHVR

SEQ ID NO: 126 - protein, 7 aa, synthetic construct
LHRKRHL

SEQ ID NO: 127 - protein, 4 aa, synthetic construct
RLRL

SEQ ID NO: 128 - protein, 8 aa, synthetic construct
KLKKLKKL

SEQ ID NO: 129 - protein, 5 aa, synthetic construct
KLKKL

SEQ ID NO: 130 - protein, 8 aa, synthetic construct
LRKLRKLR

SEQ ID NO: 131 - protein, 8 aa, synthetic construct
RLKRLKLR

SEQ ID NO: 132 - protein, 8 aa, synthetic construct
RLKRLKRL

SEQ ID NO: 133 - protein, 8 aa, synthetic construct
RLKLRKLR

SEQ ID NO: 134 - protein, 5 aa, synthetic construct
RLKRL

SEQ ID NO: 135 - protein, 8 aa, synthetic construct
KLKLKKLK

SEQ ID NO: 136 - protein, 8 aa, synthetic construct
LKKLKKLK

SEQ ID NO: 137 - protein, 5 aa, synthetic construct
KKKKK

SEQ ID NO: 138 - protein, 7 aa, synthetic construct
RLRKRLR

SEQ ID NO: 139 - protein, 11 aa, synthetic construct
RLRKRLRKRLR

SEQ ID NO: 140 - protein, 9 aa, synthetic construct
LRLRKLRLR

SEQ ID NO: 141 - protein, 14 aa, synthetic construct
LRLRKLRLRKLRLR

SEQ ID NO: 142 - protein, 8 aa, synthetic construct
HKKKHKKL

SEQ ID NO: 143 - protein, 8 aa, synthetic construct
LRKRLKRE

SEQ ID NO: 144 - protein, 8 aa, synthetic construct
WRKRMKRF

SEQ ID NO: 145 - protein, 8 aa, synthetic construct
  MOD_RES 1: note = bAla
  MOD_RES 5: note = bAla
  MOD_RES 8: note = Acp
XRKRXKRX

SEQ ID NO: 146 - protein, 8 aa, synthetic construct
YRKRTKRQ

SEQ ID NO: 147 - protein, 8 aa, synthetic construct
FRKRQKRW

SEQ ID NO: 148 - protein, 8 aa, synthetic construct
QRKRTKRY

SEQ ID NO: 149 - protein, 8 aa, synthetic construct
MRKRCKRC

SEQ ID NO: 150 - protein, 5 aa, synthetic construct
RRKLL

SEQ ID NO: 151 - protein, 5 aa, synthetic construct
  MOD_RES 3: note = Dbu
LRXRL

SEQ ID NO: 152 - protein, 8 aa, synthetic construct
HLKRLKRL

SEQ ID NO: 153 - protein, 8 aa, synthetic construct
HLKHLKHL

SEQ ID NO: 154 - protein, 8 aa, synthetic construct
RLKRWKRW

SEQ ID NO: 155 - protein, 9 aa, synthetic construct
ESGESGESG

SEQ ID NO: 156 - protein, 8 aa, synthetic construct
RFIKLRLR

SEQ ID NO: 157 - protein, 5 aa, synthetic construct
RWKRW

SEQ ID NO: 158 - protein, 6 aa, synthetic construct
RFIKHK

SEQ ID NO: 159 - protein, 8 aa, synthetic construct
HVRKRLRL

SEQ ID NO: 160 - protein, 7 aa, synthetic construct
RHLKRFI

SEQ ID NO: 161 - protein, 8 aa, synthetic construct
LEKELKEL

SEQ ID NO: 162 - protein, 5 aa, synthetic construct
LEKEL

SEQ ID NO: 163 - protein, 7 aa, synthetic construct
EHLKEHL

SEQ ID NO: 164 - protein, 7 aa, synthetic construct
RHLKSYR

SEQ ID NO: 165 - protein, 6 aa, synthetic construct
LRFKLL

SEQ ID NO: 166 - protein, 8 aa, synthetic construct
RKRLKRLR

SEQ ID NO: 167 - protein, 8 aa, synthetic construct
LRKRKRLR

SEQ ID NO: 168 - protein, 9 aa, synthetic construct
LRKRKRLRL

SEQ ID NO: 169 - protein, 8 aa, synthetic construct
  SITE 1: note = D-Leucine
  SITE 5: note = D-Leucine
  SITE 8: note = D-Leucine
  SITE 2: note = D-Arginine
  SITE 4: note = D-Arginine
  SITE 7: note = D-Arginine
  SITE 3: note = D-Lysine
  SITE 6: note = D-Lysine
LRKRLKRL

SEQ ID NO: 170 - protein, 8 aa, synthetic construct
  SITE 1: note = D-Leucine
  SITE 5: note = D-Leucine
  SITE 8: note = D-Leucine
  SITE 2: note = D-Arginine
  SITE 4: note = D-Arginine
  SITE 7: note = D-Arginine
LRKRLKRL

SEQ ID NO: 171 - protein, 8 aa, synthetic construct
  SITE 1: note = D-Leucine
  SITE 8: note = D-Leucine
  SITE 6: note = D-Lysine
  SITE 4: note = D-Arginine
LRKRLKRL

SEQ ID NO: 172 - protein, 8 aa, synthetic construct
  SITE 1: note = D-Arginine
  SITE 4: note = D-Arginine
  SITE 7: note = D-Arginine
  SITE 2: note = D-Leucine
  SITE 5: note = D-Leucine
  SITE 8: note = D-Leucine
  SITE 3: note = D-Lysine
  SITE 6: note = D-Lysine
RLKRLKRL

SEQ ID NO: 173 - protein, 8 aa, synthetic construct
  SITE 1: note = D-Lysine
  SITE 3..4: note = D-Lysine
  SITE 6..7: note = D-Lysine
  SITE 2: note = D-Leucine
  SITE 5: note = D-Leucine
  SITE 8: note = D-Leucine
KLKKLKKL

SEQ ID NO: 174 - protein, 5 aa, synthetic construct
HLKRF

SEQ ID NO: 175 - protein, 5 aa, synthetic construct
KLKKL

SEQ ID NO: 176 - protein, 5 aa, synthetic construct
LRKLR

SEQ ID NO: 177 - protein, 5 aa, synthetic construct
RLKLR

SEQ ID NO: 178 - protein, 5 aa, synthetic construct
KLKLK

SEQ ID NO: 179 - protein, 5 aa, synthetic construct
LKKLK

SEQ ID NO: 180 - protein, 4 aa, synthetic construct
KKKK

SEQ ID NO: 181 - protein, 5 aa, synthetic construct
HKKKH

SEQ ID NO: 182 - protein, 5 aa, synthetic construct
WRKRM

SEQ ID NO: 183 - protein, 5 aa, synthetic construct
YRKRT

SEQ ID NO: 184 - protein, 5 aa, synthetic construct
FRKRQ

SEQ ID NO: 185 - protein, 5 aa, synthetic construct
QRKRT

SEQ ID NO: 186 - protein, 5 aa, synthetic construct
MRKRC

SEQ ID NO: 187 - protein, 4 aa, synthetic construct
LRRL

SEQ ID NO: 188 - protein, 5 aa, synthetic construct
HLKRL

SEQ ID NO: 189 - protein, 5 aa, synthetic construct
HLKHL

SEQ ID NO: 190 - protein, 5 aa, synthetic construct
RLKRW

SEQ ID NO: 191 - protein, 4 aa, synthetic construct
RKRL

SEQ ID NO: 192 - protein, 4 aa, synthetic construct
LRKR

SEQ ID NO: 193 - protein, 5 aa, synthetic construct
  SITE 1: note = D-Leucine
  SITE 5: note = D-Leucine
  SITE 2: note = D-Arginine
  SITE 4: note = D-Arginine
  SITE 3: note = D-Lysine
LRKRL

(19) United States
(12) Patent Application Publication
(10) Pub. No.: US 2026/0014278 A1
(43) Pub. Date: Jan. 15, 2026

(54) HYBRID PEPTIDE DENDRIMER SYSTEMS AND EXTRAHEPATIC DELIVERY

(71) Applicant: NUNTIUS THERAPEUTICS LIMITED, London (GB)

(72) Inventors: Albert Kwok, London (GB); Liam Thomas MARTIN, London (GB); Lawrence James BILLING, London (GB); Parisa SAMANGOUEI, London (GB); Benita NAGEL, London (GB); Marina APOSTOLIDOU, London (GB)

(21) Appl. No.: 19/100,421

(22) PCT Filed: Aug. 3, 2023
(86) PCT No.: PCT/EP2023/071607

§ 371 Date: Jan. 31, 2025

(30) Foreign Application Priority Data
Aug. 3, 2022 (GB) 2211331.0
Aug. 3, 2022 (GB) 2211332.8
Oct. 31, 2022 (GB) 2216131.9
Nov. 7, 2022 (GB) 2216550.0

(51) Int. Cl. A61K 48/00; C12N 15/88

(52) U.S. Cl. CPC A61K 48/0058; A61K 48/0041; A61K 48/0091; C12N 15/88; C12N 2800/22

(57) ABSTRACT

The present invention relates to compositions that can deliver therapeutic molecules such as nucleic acids to mammalian cells, and to the human and animal body and methods of preparing and using the same. The compositions comprise nanoparticles comprising a peptide dendrimer, a nucleic acid and a lipid. The compositions of the invention find utility in the field of medicine, such as for treating cancer and autoimmune diseases.

### FIELD OF THE INVENTION

The present invention relates to compositions that can deliver therapeutic molecules such as nucleic acids to mammalian cells, and to the human and animal body.

### BACKGROUND

Nucleic acid therapeutics have the potential to be the next generation of precision medicines which will transform healthcare. However, key challenges remain. One such challenge is the efficient and safe delivery of the nucleic acid therapies to patients. Current viral and non-viral vector platforms often fall at the clinical translation stage due to off-target effects, immune activation and difficulty manufacturing said vectors at scale.

In the context of in vivo delivery of nucleic acids, viral-derived vectors have been studied extensively and some are very advanced in the clinic, including adeno-associated viruses (AAVs) (Sheridan et al, 2011 and Wang et al, 2019). The use of these systems are however limited to delivering DNA of <5 kb and cannot transport RNA or larger DNA. Despite the advances in these delivery systems, there is also still the potential for random insertions and immunotoxicity resulting from the use of these systems. In particular, when targeting non-liver tissues, which requires the use of higher doses, the AAV systems can be highly immunogenic. This tendency to generate an immune response to the AAV system also limits the usefulness of the system for repeat dosing as patients typically develop immunity to the AAV delivery system. Finally, AAV delivery systems are known to be expensive and difficult to manufacture at scales required for therapeutic use and at Good Manufacturing Practice (GMP) grade.

Non-viral vector systems for nucleic acid delivery to cells and tissues in vivo are also being investigated. These systems include those designed for delivery of smaller nucleic acids such as siRNA and antisense oligonucleotides (ASOs). One approach which has been explored is bioconjugated oligonucleotide delivery systems, wherein an siRNA or ASO is conjugated to an antibody or ligand (Benizri et al, 2019). However, these approaches are limited to gene silencing or exon skipping and cannot be used to express genes in a target tissue. Bioconjugate-based delivery systems are challenging to apply to large genetic payloads such as plasmid DNA and mRNA as each are large, negatively charged molecules that cannot easily pass through the negatively charged plasma membrane of cells.

To address the difficulty of getting plasmid DNA and mRNA to traverse the plasma membrane it can be useful to encapsulate the nucleic acids and neutralise the charge for effective delivery. Lipid nanoparticles (LNPs), as encompassing non-viral delivery vehicles, have been used to encapsulate and delivery mRNA, for example in COVID-19 vaccines (Qui et al, 2021). These systems can be useful for vaccine delivery as it is only necessary to transfect a small number of muscle and immune cells local to the site of delivery to train the immune systems to fight the infection. Therefore, the RNA-based COVID-19 vaccines are administered intramuscularly. However, for a wide range of diseases it will be necessary to transfect a large number of target cells in order to effectively treat the disease. While the LNPs currently used in the clinic might make them a good fit for targeting the liver when administered intravenously, they are less suitable for targeting other tissues and thus make them less suitable for targeting diseases associated with diseases other than the liver.

Liposome based systems have also been investigated using the uncharged lipids such as dioleoylphosphatidylethanolamine (DOPE) and/or cationic lipids such as 1,2-dioleoyl-3-trimethylammonium-propane chloride (DOTAP) and N-[1-(2,3-dioleyloxy)propyl]-N,N,N-trimethylammonium chloride (DOTMA) (Braum, 2019; Ren et al, 2000).

To overcome the various drawbacks of the above delivery systems, the addition of peptides to lipid based vectors has been investigated. In these hybrid systems, the peptides and lipids associated with the nucleic acid to form nanoparticles that can be internalised by cells. The peptide element may be linear (Kwok et al, 2016) or branched, such as a peptide dendrimer (Kwok et al, 2013). These peptide/lipid systems were initially used to investigate delivery of plasmid DNA and siRNA in serum-free in vitro conditions, differing substantially from in vivo conditions. However, it was not clear if these systems could be used to deliver long nucleic acids in vivo. The only reports demonstrating in vivo liver delivery of ASOs using peptide dendrimer/lipid nanoparticles were only able to achieve a low 20-30% increase in delivery compared to ASO alone (Saher et al, 2018; Saher et al, 2019). Given the low delivery rate of ASOs using these systems, one may conclude that these peptide dendrimer/lipid systems are not suitable or able to deliver larger nucleic acids, such as mRNA, to tissues in vivo.

In PCT application WO2022162200, it was recently demonstrated by the inventors that delivery of long nucleic acids, such as mRNA, can be achieved in vivo using peptide dendrimer/lipid hybrid systems. The inventors developed a framework for nucleic acid delivery, using peptide dendrimers as disclosed in WO2022162200, which is incorporated by reference in its entirety. The dendrimers disclosed in WO2022162200 are branched peptides displaying one, two, three or four amino acid residues between ‘branching residues’ which act as branching units within the dendrimer molecule. It was show in WO2022162200 that these dendrimer/lipid compositions are surprisingly effective at delivering larger nucleic acids (e.g. larger than antisense oligonucleotides, ASOs) to, for example, extrahepatic tissues. While some smaller nucleic acids such as ASOs are readily taken up into cells even without vector systems, the compositions of WO2022162200 allow larger nucleic acids such as mRNA to be effectively delivered.

Despite the advances provided by the compositions disclosed in WO2022162200, there is still a need to develop nucleic acid delivery systems with improved transfection efficiency and cell- and tissue-specificity to maximise the therapeutic potential of nucleic acid therapies.

The present invention has been devised in light of the above considerations.

### SUMMARY OF THE INVENTION

The development of nucleic acid therapies is dependent on efficient and targeted nucleic acid delivery. The inventors have found that peptide dendrimer/lipid-based nucleic acid delivery systems can be modified to improve delivery of nucleic acids to specific cells and tissues. One way in which the inventors have found cell and/or tissues specific nucleic acid delivery can be achieved is by modifying the ratio of peptide dendrimer (also referred to herein as “dendrimers”) to nucleic acid in a nanoparticle and/or modifying the ratio of lipid to nucleic acid. Alternatively, targeted delivery can be achieved by including a cell or tissue specific targeting motif in a nanoparticle. The location of the targeting motif is not particularly limited and may be, for example, covalently bound to the peptide dendrimer, covalently bound to a second peptide (that may be additionally included in the nanoparticle), or covalently bound to a polymer (that may be additionally included in the nanoparticle).

The inventors have also found that peptide dendrimer/lipid nanoparticles comprising a combination of two different dendrimers can be even more effective at achieving in vitro and in vivo nucleic acid delivery compared to nanoparticles comprising a single peptide dendrimer. For instance, combining a ‘first generation’ peptide dendrimer with a ‘second’ or ‘third generation’ peptide dendrimer in a single composition can allow for more efficient delivery of nucleic acids in vitro and in vivo compared to compositions comprising a single first-, second-, or third-generation peptide dendrimer alone. This may be achieved, for example, by varying the number of charged and hydrophobic residues in each peptide dendrimer capable of forming non-covalent bonds with a nucleic acid. Alternatively, or in addition to, a mixture of dendrimers may be chosen based on the stability of each dendrimer/nucleic acid complex and the ability to form a monodisperse population of nanoparticles. For instance, a nanoparticle may comprise one dendrimer incapable of forming a monodisperse population of dendrimer/lipid nanoparticles when used alone (indicative of a relatively high rate of dendrimer-nucleic acid dissociation) with a second dendrimer which is capable of forming a monodisperse population of dendrimer/lipid nanoparticles alone (indicative of a relatively low rate of dendrimer-nucleic acid dissociation). The peptide dendrimers may therefore be selected based on the polydispersity index of the respective single dendrimer/lipid nanoparticles. Moreover, the nanoparticles may comprise an advantageous combination of lipids, described herein, which can improve transfection efficiency.

These new findings further open up the field of nucleic acid therapies and provide promising solutions to the outstanding challenge of providing efficient, safe and targeted vectors for nucleic acid delivery in vivo.

The present invention may also be useful in the ex vivo transfection of cells/tissue derived from a patient. For example, the nanoparticles of the invention may be adapted for more efficient nucleic acid delivery to ex vivo myeloid or lymphoid cells. This discovery will be particularly useful in the field of chimeric antigen receptor (CAR)-based therapeutics, such as CAR-T and CAR-M, for the treatment of various cancers. Embodiments in which e.g. advantageous lipid combinations exhibit enhanced transfection efficiencies find use in in vitro applications.

Based on the surprising findings in WO2022162200 that peptide dendrimer/lipid nanoparticles comprising a single peptide dendrimer can effectively deliver nucleic acid cargos to cells in vivo, it is expected that the current mixed peptide dendrimer/lipid nanoparticles will also effectively deliver nucleic acid cargos to cells in vivo. In addition, based on the increased efficiency of cargo delivery achieved in vitro with mixed dendrimer nanoparticles compared to single dendrimer nanoparticles, it is also expected that the efficiency of delivery of nucleic acid cargos to cells in vivo will also be greater than that displayed by single dendrimer/lipid nanoparticles. Thus, taking account of the inventors findings in WO2022162200, the mixed dendrimer nanoparticles can effectively deliver nucleic acids, for example DNA and RNA, to various tissues, especially to the lungs and immune cell-rich tissues, including spleen, lymph nodes and bone marrow. This allows the development of improved DNA- and RNA-based therapies directed to extrahepatic tissues.

The present invention can effectively and specifically deliver nucleic acids, including DNA and mRNA, to various cells and tissues, especially to myeloid and lymphoid cells and to muscle and lung tissues as well as cancer cells. The nanoparticles can also be adapted to target other organs and tissues. The ability to deliver nucleic acids, and in particular mRNA, to immune cells, and to a greater degree the myeloid cells including monocytes, macrophages, neutrophils and dendritic cells, enables this technology to be developed to treat all cancer types (including solid tumours and blood cancer such as Myelodysplastic Syndromes (MDS) and Chronic Myelomonocytic Leukemia (CMML), autoimmune diseases (e.g. Diabetes, including Type I Diabetes, Rheumatoid Arthritis, Crohn's diseases, Uveitis, Inflammatory bowel disease) and other immune cell related disorders such as a graft versus host disease, allograft rejection, acute rejection after transplant, chronic rejection after transplant, primary graft dysfunction, chronic granulamatous disease (CGD) or Gaucher's diseases. This invention also allows nucleic acids, including DNA and mRNA, to be delivered to the lungs and/or immune cells, making treatments for lung-related diseases such as cystic fibrosis and chronic obstructive pulmonary diseases possible. In addition, this invention allows delivery of nucleic acids, including DNA and mRNA to muscle tissue and muscle cells to treat myopathies, including muscular dystrophies (e.g. as Duchenne muscular dystrophy, myotonic dystrophy, facioscapulohumeral muscular dystrophy, Becker muscular dystrophy, limb-girdle muscular dystrophy, oculopharyngeal muscular dystrophy, Emery-Dreifuss muscular dystrophy, inheriting muscular dystrophy, congenital muscular dystrophy, and distal muscular dystrophy) and muscle wasting diseases.

Thus, in one aspect, this invention provides a nanoparticle comprising a peptide dendrimer, a nucleic acid and a lipid. The skilled person will understand that more than one peptide dendrimer and/or more than one lipid can be included. The peptide dendrimer comprises at least: a core peptide sequence, a first branching residue and two first peptide motifs. The branching residue may be lysine, 2,4-diaminobutyric acid, ornithine, or diaminopropionic acid. The nanoparticle may be targeted to a specific cell type or tissue, e.g. a myeloid cell, a lymphoid cell, a muscle cell, a lung cell, and/or a cancer cell and/or a myeloid tissue, lymphoid tissue, muscle tissue, a lung tissue and/or a tumor tissue. The nanoparticle may be targeted to a bone marrow, pancreas, neuronal tissue, kidney tissue, heart tissue, liver tissue, eye, joint or prostate, or to a stem cell, pancreatic cell, neuronal cell, a kidney cell, a cardiac cell, liver cell, ocular cell, synovial cell or prostate cell. As used herein, myeloid tissue includes tissue of the bone marrow and cells of the bone marrow cell lineage. As used herein, lymphoid tissue includes any organ of the lymphatic system, including the spleen, thymus, lymph nodes and bone marrow. The nanoparticle finds uses in vivo, ex vivo and in vitro. The nanoparticle is capable of transfecting the target cell or tissue, e.g. a myeloid, lymphoid, muscle, lung, or cancer cells in vitro at an efficiency of at least 10%, at least 12.5%, at least 15%, at least 17.5%, at least 20%, at least 25%, at least 30%, at least 40%, at least 45%, at least 50%, at least 55%, at least 60%, at least 65%, at least 70%, at least 75%, at least 80%, at least 85%. Transfection efficiency may be determined by any standard method in the art, for example by fluorescence microscopy, real time PCR (qPCR), plasmid reporting system, reporter gene assay, flow cytometry, western blot or immunofluorescent staining. Such methods are described in Chong, Z., et al (2021) or as described in Example 1. Preferably, flow cytometry is used to determine the percentage transfection efficiency of the cell population following treatment with nanoparticles comprising a fluorescently labelled nucleic acid. Flow cytometry is used to determine uptake of the nanoparticles. Preferably, the transfection efficiency is measured following transfection of a 600 μL volume of the cell population for four hours in a 24 well plate in serum containing medium at 37 C. Preferably the nanoparticle population is added to the well at a dose comprising 1.5 μg of nucleic acid. Alternatively, flow cytometry is used to determine the percentage transfection efficiency of the cell population following treatment with nanoparticles comprising a nucleic acid expressing a reporter gene. Flow cytometry is used to determine reporter gene expression. In this instance, reporter gene expression is measured following transfection of a 600 μL volume of the cell population for 24 hours in a 24 well plate in serum containing medium at 37 C. Preferably the nanoparticle population is added to the well at a dose comprising 1.5 μg of nucleic acid. Some modifications of the testing protocol can be made, such as using higher or lower cell volumes, e.g. within the 300 to 1,200 μL range. In some embodiments, a 12 well plate, a 48 well plate or a 96 well plate can be used instead of a 24 well plate. For instance, 120,000 macrophages can be used in a 24 well plate, or 240,000 macrophages can be used in a 12 well plate. 60,000 C2c12 cells can be used in a 24 well plate. The transfection time could be varied, e.g. at a 1 or 2 hour incubation time with the nanoparticle population. Higher incubation time, e.g. 16 or 24 hours could be used, particularly when flow cytometry is used to determine reporter gene expression. Slightly higher amounts of nanoparticle can also be used, e.g. 3 μg.

In another aspect, the invention provides a nanoparticle comprising a peptide dendrimer, a nucleic acid and a lipid for use in medicine. The peptide dendrimer comprises at least: a core peptide sequence, a first branching residue and two first peptide motifs. The branching residue may be lysine, 2,4-diaminobutyric acid, ornithine, or diaminopropionic acid. The nanoparticle is targeted to a myeloid, lymphoid, muscle, a lung and/or cancer cell and/or a myeloid, lymphoid, muscle, a lung tissue and/or a tumor. The nanoparticle is capable of transfecting the target cell or tissue, e.g. a myeloid, lymphoid, muscle, lung and/or cancer cells in vitro at an efficiency of at least 10%, at least 12.5%, at least 15%, at least 17.5%, at least 20%, at least 25%, at least 30%, at least 40%, at least 45%, at least 50%, at least 55%, at least 60%, at least 65%, at least 70%, at least 75%, at least 80%, at least 85%.

In a further aspect, the invention provides a nanoparticle comprising a peptide dendrimer, a nucleic acid and a lipid for use in treating cancer, an autoimmune disease, a lung disease and/or a myopathy. The peptide dendrimer comprises at least: a core peptide sequence, a first branching residue and two first peptide motifs. The branching residue may be lysine, 2,4-diaminobutyric acid, ornithine, or diaminopropionic acid. The nanoparticle may be targeted to a myeloid, lymphoid, muscle and/or a lung cell and/or a myeloid, lymphoid, muscle, a lung tissue, and/or a tumor. The nanoparticle may be targeted to bone marrow, pancreas, neuronal tissue, kidney tissue, heart tissue, liver tissue, eye, joint or prostate, or to a stem cell, pancreatic cell, neuronal cell, a kidney cell, a cardiac cell, liver cell, ocular cell, synovial cell or prostate cell. The nanoparticle is capable of transfecting the target cell or tissue, e.g. a myeloid, lymphoid, muscle, lung and/or cancer cells in vitro at an efficiency of at least 10%, at least 12.5%, at least 15%, at least 17.5%, at least 20%, at least 25%, at least 30%, at least 40%, at least 45%, at least 50%, at least 55%, at least 60%, at least 65%, at least 70%, at least 75%, at least 80%, at least 85%.

In a further aspect, the invention provides a method of treating cancer, an autoimmune disease, a lung disease and/or a myopathy, the method comprising administering a nanoparticle comprising a peptide dendrimer, a nucleic acid and a lipid to a patient or subject. The peptide dendrimer comprises at least: a core peptide sequence, a first branching residue and two first peptide motifs. The branching residue may be lysine, 2,4-diaminobutyric acid, ornithine, or diaminopropionic acid. The nanoparticle may be targeted to a myeloid, lymphoid, muscle, a lung and/or cancer cell and/or a myeloid, lymphoid, muscle, lung tissue and/or a tumor. The nanoparticle may be targeted to bone marrow, pancreas, neuronal tissue, kidney tissue, heart tissue, liver tissue, eye, joint or prostate, or to a stem cell, pancreatic cell, neuronal cell, a kidney cell, a cardiac cell, liver cell, ocular cell, synovial cell or prostate cell. The nanoparticle is capable of transfecting the target cell or tissue, e.g. a myeloid, lymphoid, muscle, lung and/or cancer cells in vitro at an efficiency of at least 10%, at least 12.5%, at least 15%, at least 17.5%, at least 20%, at least 25%, at least 30%, at least 40%, at least 45%, at least 50%, at least 55%, at least 60%, at least 65%, at least 70%, at least 75%, at least 80%, at least 85%.

In a further aspect, the invention provides a nanoparticle comprising a peptide dendrimer selected from Table 1, a nucleic acid, and a lipid. For example, the nanoparticle of the invention may comprise a dendrimer having the structure (RHL)4(KRHL)2KGSC-NH2, (HL)8(KRF)4(KRF)2KGSC-NH2, (LR)4(KRL)2KRHA-NH2, (LR)₈(KRL)₄(KRL)₂KGSC-NH₂, (LR)4(KRL)2KRHC-NH₂, (LR)4(KRL)2KR HCR-(Acp)-RR-(β-A)-RR-(Acp)-RR-(β-A)-R-(Acp)-(β-A)-NH2, (LR)4(KRL)2KRHCGAASSLNIA-(Acp)-NH2, (LR)4(KRL)2KRHCGAASSLNIA-(Acp)-R-(Acp)-RR-(β-A)-RR-(Acp)-RR-(β-A)-R-(Acp)-(β-A)-NH2, (LR)8(KRL)4(KRL)2KRHCR-(Acp)-RR-(β-A)-RR-(Acp)-RR-(β-A)-R-(Acp)-(β-A)-NH2, (LRLR)2KGSC-NH2, (HR)2KK-NH2, (RFI)4(KKE)2KRG-NH2, (SYR)4(KLRF)2KER-NH2, (RL)4(KHGD)2KLR-NH2, (HVR)4(KHVR)2KVR-NH2, (LHR)4(KRHL)2KGSC-NH2, (RLRL)2KLRL-NH2, (LR)8(KRL)4(KRL)2KGSCGAASSLNIA(Acp)-NH2, (LR)8(KRL)4(KRL)2KGSCHHHHHHGAASSLNIA(Acp)-NH2, (LR)4(KRL)2KGSGGSGGSGGSC[(S-S)-α-D-Thiomannose], (Ac-EEEE)2KGSGGSGGSC[(S-S)-α-D-Thiomannose]. In some embodiments, the nanoparticle may comprise a dendrimer with the structure (RHL)4(KRHL)2KGSC-NH2, (HL)8(KRF)4(KRF)2KGSC-NH2, (LR)4(KRL)2KRHA-NH2, (LR)4(KRL)2KRHCR-(Acp)-RR-(β-A)-RR-(Acp)-RR-(β-A)-R-(Acp)-(β-A)-NH2, (LR)8(KRL)4(KRL)2KRHCR-(Acp)-RR-(β-A)-RR-(Acp)-RR-(β-A)-R-(Acp)-(β-A)-NH2, (HR)2KK-NH2, (RFI)4(KKE)2KRG-NH2, (SYR)4(KLRF)2KER-NH2, (RL)4(KHGD)2KLR-NH2, or (HVR)4(KHVR)2KVR-NH2, (LHR)4(KRHL)2KGSC-NH2, (RLRL)2KLRL-NH2, (LR)4(KRL)2KRHC-NH2, (LRLR)2KGSC.

In a further aspect, the invention provides a composition comprising a first peptide dendrimer, a second peptide dendrimer, a nucleic acid and a lipid. The first and second peptide dendrimer comprises at least: a core peptide sequence, a first branching residue and two first peptide motifs. The branching residue may be lysine, 2,4-diaminobutyric acid, ornithine, or diaminopropionic acid.

In another aspect, the invention provides a composition for use in medicine, wherein the composition comprises a first peptide dendrimer, a second peptide dendrimer, a nucleic acid and a lipid. The first and second peptide dendrimer comprises as least: a core peptide sequence, a first branching residue and two first peptide motifs. The branching residue may be lysine, 2,4-diaminobutyric acid, ornithine, or diaminopropionic acid.

In another aspect, the invention provides a composition comprising a first peptide dendrimer, a second peptide dendrimer, a nucleic acid and a lipid for use in a method of treating cancer and/or an autoimmune disease. The first and second peptide dendrimer comprises as least: a core peptide sequence, a first branching residue and two first peptide motifs. The branching residue may be lysine, 2,4-diaminobutyric acid, ornithine, or diaminopropionic acid.

In another aspect, the invention provides a method of treating a patient with a composition comprising a first peptide dendrimer, a second peptide dendrimer, a nucleic acid and a lipid. The first and second peptide dendrimer comprises as least: a core peptide sequence, a first branching residue and two first peptide motifs. The branching residue may be lysine, 2,4-diaminobutyric acid, ornithine, or diaminopropionic acid. The method may comprise treating a patient with cancer and/or an autoimmune disease.

The skilled person will understand that these compositions comprising a first peptide dendrimer, a second peptide dendrimer can include more than two dendrimers, such as three or four or more dendrimers.

Relatedly, the invention provides a composition comprising the nanoparticle of the invention. The invention further provides a pharmaceutical composition comprising the nanoparticle of the invention and a pharmaceutically acceptable excipient. The pharmaceutical composition may be used in medicine. The pharmaceutical composition may be for use in the treatment of a cancer, an autoimmune disease, a lung disease and/or a myopathy. Also provided is a method of treating a cancer, an autoimmune disease, a lung disease and/or a myopathy, wherein the method comprises administering the pharmaceutical composition to a patient or subject. In some embodiments, the composition or pharmaceutical composition is comprised within a liquid. In other embodiments, the composition or pharmaceutical composition is provided as a dry composition, e.g. a dry powder. The dry composition may be prepared using lyophilisation and/or freeze-drying techniques.

The nanoparticle of the invention may also comprise a myeloid, lymphoid, muscle, lung or cancer cell targeting motif. For example, the muscle cell targeting motif may comprise the peptide motif ASSLNIA (SEQ ID NO:1), PYDQLRH (SEQ ID NO:2), or KAMHQMQ (SEQ ID NO:3). The muscle cell targeting motif may comprise a variant of the peptide motif ASSLNIA (SEQ ID NO:1), PYDQLRH (SEQ ID NO:2), or KAMHQMQ (SEQ ID NO:3). For example, the variant muscle cell targeting motif may comprise one, two or three amino acid substitutions, deletions or additions relative to the peptide motifs ASSLNIA (SEQ ID NO:1) PYDQLRH (SEQ ID NO:2), or KAMHQMQ (SEQ ID NO:3), provided that the variant retains the ability to target the nanoparticle to a muscle cell.

In some embodiments, the targeting motif is a lung cell targeting motif. In some embodiments, the lung cell targeting motif may comprise the peptide sequence CGFECVRQCPERC (SEQ ID NO:4). The lung cell targeting motif may comprise a variant of the peptide sequence CGFECVRQCPERC (SEQ ID NO:4). For example, the variant lung cell targeting motif may comprise one, two or three amino acid substitutions, deletions or additions relative to the peptide sequence CGFECVRQCPERC (SEQ ID NO:4), provided that the variant retains the ability to target the nanoparticle to a lung cell.

In another example, the targeting motif is a cancer cell targeting motif. In some embodiments, the cancer cell targeting motif is a peptide comprising an RGD integrin targeting peptide motif. In some embodiments, the cancer cell targeting motif is a peptide comprising the ACDCRGDCFCG (SEQ ID NO:5) integrin targeting peptide motif. The cancer cell targeting motif may comprise a variant of the peptide sequence CGFECVRQCPERC (SEQ ID NO:5). For example, the variant cancer cell targeting motif may comprise one, two or three amino acid substitutions, deletions or additions relative to the peptide sequence CGFECVRQCPERC (SEQ ID NO:5), provided that the variant retains the ability to target the nanoparticle to a cancer cell. In another example, the cancer cell targeting motif may be a maltotriose sugar. A maltotriose sugar may be used as maltriose can bind to GLUT receptors highly expressed on a cancer cell (Sakamaki, Y., et al (2021)).

A myeloid cell targeting motif may comprise a sugar. A myeloid cell targeting motif may comprise a mannose sugar for targeted delivery to macrophages. In another example, the myeloid cell targeting motif may be a maltotriose. Maltotriose is a trisaccharide consisting of three glucose molecules linked with α-1,4 glycosidic bonds. A mannose sugar, mannose glycosylation and/or a maltotriose may be used to target the nanoparticle to macrophages with an M2 phenotype. For example, the targeting motif may comprise a mannose sugar or maltotriose each of which can bind to the CD206 receptor which is expressed on M2 phenotype macrophages. Other sugars may be selected for conjugation to peptide dendrimers to target other tissues and cell types. For example, N-Acetylgalactosamine (GalNAc) may be chosen for hepatocyte targeting (Holland et al (2021)).

In some embodiments, the cell targeting motif targets lymphocytes, e.g. T cells. The cell targeting motif may be a CD3, CD4 or CD8 binder, e.g. an antibody that specifically binds one of these markers. In some embodiments, an anti-CD3 antibody is used. This may be conjugated to a negatively charged polymer such as PGA or a glutamic acid containing peptide.

The nanoparticles may be targeted for delivery to lung tissue. Cells of the lung which may be targeted include alveolar macrophages, ciliated cells, epithelial cells, basal cells, secretory cells, club cells, alveolar cells, fibroblasts, and/or a endothelial cells. In some embodiments, targeting to tissues such as the lung may be achieved without additional ligands or peptide motifs. For instance, adjusting the lipid:nucleic acid ratio can dramatically enhance lung targeting, as described herein. Additionally, adjusting the surface charge of the nanoparticle can also affect tissue targeting specificity, as described herein.

The nanoparticle of the invention may also comprise a bone marrow, pancreas, neuronal tissue, kidney tissue, heart tissue, liver tissue, eye, joint or prostate, or to a stem cell, pancreatic cell, neuronal cell, a kidney cell, a cardiac cell, liver cell, ocular cell, synovial cell or prostate cell targeting motif. For example, the targeting motif may comprise a motif that targets a bone marrow or stem cell, such as an antibody that specifically binds CD34, and/or a lipid comprising a bisphosphonate (BP) group (Xue et al, 2022, which is hereby incorporated by reference in its entirety). The skilled person will understand that such BP comprising lipids can be conjugated to any component of the nanoparticle, e.g. the peptide and/or dendrimer component.

In some embodiments, the targeting motif is a pancreas targeting peptide such as a glucagon-like peptide-1 (GLP-1) homolog. Jones et al, 2018, which is hereby incorporated by reference in its entirety, discloses ‘Exendin-4’ and related peptides. Exendin-4 has amino acid sequence HGEGTFTSDLSKQMEEEAVRLFIEWLKNGGPSSGAPPPS (SEQ ID NO:9). Such peptides, and variants and binding fragments thereof, can be used as pancreas targeting peptides of the present invention. Preferably, a variant has at least 60%, 65%, 70%, 75%, 80%, 85%, 90% or 95% amino acid identity to SEQ ID NO:9, e.g. over at least 20 consecutive amino acids. A binding fragment preferably has at least 8, at least 10, at least 12, at least 14, at least 16, at least 18 or at least 20 contiguous amino acids of SEQ ID NO:9.

In some embodiments, the targeting motif is a kidney targeting peptide such as (KKEEE)₃K (Wischnjow et al, 2016, which is hereby incorporated by reference in its entirety). KKEEE is SEQ ID NO:10.

In some embodiments, the targeting motif is a neuronal tissue/cell targeting peptide such as YTIWMPENPRPGTPCDIFTNSRGKRASNGGGG (SEQ ID NO:11) or KSVRTWNEIIPSKGCLRVGGRCHPHVNGGG (SEQ ID NO:12) (Wang et al, 2021, which is hereby incorporated by reference in its entirety). In some embodiments, the neuronal targeting peptide comprises a Phe-Arg-Trp (FRW) motif (Tang et al, 2019, which is hereby incorporated by reference in its entirety).

In some embodiments, the targeting motif is a heart targeting or cardiac cell targeting peptide such as APWHLSSQYSRT (SEQ ID NO:13) (Zahid et al, 2018, which is hereby incorporated by reference in its entirety).

In some embodiments, the targeting motif is a liver targeting or hepatocyte targeting moiety such as an asialoglycoprotein receptor binding sugar moiety. An exemplary asialoglycoprotein receptor binder are N-Acetylgalactosamine (Gal-NAc) sugar moieties (Cui et al, 2021, which is hereby incorporated by reference in its entirety). The skilled person will understand that such sugars moieties can be conjugated to any component of the nanoparticle, e.g. the peptide and/or dendrimer component. In other embodiments, the liver targeting or hepatocyte targeting moiety is vitamin A (Senoo et al, 2010, which is hereby incorporated by reference in its entirety).

In some embodiments, the targeting motif is an eye targeting or ocular cell targeting moiety such as CARSKNKDC (SEQ ID NO:14) (Vähätupa et al, 2021, which is hereby incorporated by reference in its entirety).

In some embodiments, the targeting motif is a joint targeting or synovial cell targeting moiety such as an antibody that specifically binds CD44, and/or a hyaluronan sugar (Gorantla et al, 2021, which is hereby incorporated by reference in its entirety).

In some embodiments, the targeting motif is a prostate targeting moiety such as PKRGFQD (SEQ ID NO:15) or SNTRVAP (SEQ ID NO:16) (Mandelin et al, 2015, which is hereby incorporated by reference in its entirety).

In some embodiments, the nanoparticle further comprises a cell penetrating peptide. The cell penetrating peptide may comprises a TAT derived sequence. The cell penetrating peptide may comprise the peptide sequence XRXRRBRRXRRBRXB (SEQ ID NO:6), where X is 6-aminohexanoic acid and B is beta-alanine. The cell penetrating peptide may comprise a variant of the peptide sequence XRXRRBRRXRRBRXB (SEQ ID NO:6). For example, the variant may comprise one, two or three amino acid substitutions, deletions or additions relative to the peptide sequence XRXRRBRRXRRBRXB (SEQ ID NO:6) provided that the variant retains biological activity.

In some embodiments, the myeloid, lymphoid, muscle or lung cell targeting motif may be an antibody. By “antibody” we include a fragment or derivative thereof, or a synthetic antibody or synthetic antibody fragment. Fragments of antibodies, such as Fab and Fab₂ fragments may also be used as can genetically engineered antibodies and antibody fragments. Single chain Fv (scFv) antibodies may also be used. The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855). By “ScFv molecules” we mean molecules wherein the V_{H} and V_{L} partner domains are covalently linked, e.g. by a flexible oligopeptide. In some embodiments, the antibody may be a T-cell specific antibody, for example, an anti-CD3 antibody (e.g. clone OKT3, BioXCell; Cat #BE0001-2). In some embodiments, the antibody may be an anti-CD4 antibody (e.g. clone OKT4, BioXCell; Cat #BE0003-2), anti-CD8 antibody (clone OKT8, BioXCell; Cat #BE0004-2), or an anti-CD28 antibody (e.g. clone 9.3, BioXCell; Cat #BE0248).

In some embodiments, the cell and/or tissue targeting motif may be covalently bound to a polymer or lipid. In some embodiments, the cell and/or tissue targeting motif may be covalently bound to a polymer or lipid that is included in the nanoparticle. The polymer or lipid may be positively charged. Alternatively, the polymer or lipid may be a negatively charged or neutrally charged polymer. The polymer or lipid may be selected from polyglutamic acid (PGA), poly(acrylic acid), alginic acid, polyethylene glycol (PEG), cholesteryl hemisuccinate/1,2-dioleoyl-sn-glycero-3-phosphoethanolamine, a neutrally-charged zwitterionic polymer or lipid, or a glutamic acid containing peptide. The PGA, poly(acrylic acid), alginic acid, PEG, cholesteryl hemisuccinate/1,2-dioleoyl-sn-glycero-3-phosphoethanolamine, neutrally-charged zwitterionic polymer or lipid, or glutamic acid containing peptide may comprise a further targeting domain as described herein, e.g. an antibody or target-binding fragment thereof. The cell and/or tissue targeting motif, e.g. an antibody, may be conjugated to one or more glutamic acid residues. PGA is a peptide comprising a region that is predominantly comprised of glutamic acid residues and preferably comprises at least two consecutive glutamic acid residues. (Other amino acid residues may be present in the PGA molecule.) In some embodiments, the PGA is a peptide that comprises a region that has a glutamic acid residue at every third position (EXXEXX . . . ) and comprises 2 to 50, 2 to 40, 2 to 30, 2 to 20, or 2 to 10 amino acid residues in total. The PGA may be a linear or a dendritic (branched) PGA. The PGA can be derivatised at its N- and/or C-terminal end(s). For instance, the C-terminal end can be derivatized with an amine group (NH₂). The linear PGA may be a short PGA. A short PGA is any PGA molecule with fewer than 100, fewer than 75, fewer than 50 or fewer than 20 glutamic acid residues. In embodiments wherein the PGA is linear, the nanoparticle preferably does not include a PBAE polymer. For example, the PGA may comprise 2 to 100, 2 to 75, 2 to 50, 2 to 40, 2 to 30, 2 to 20, or 2 to 10 glutamic acid residues. In another embodiment, the PGA may be dendritic PGA. Dendritic PGA is a peptide dendrimer comprising two or more branches that each have at least two consecutive glutamic acid residues. The dendritic PGA may be a first generation, a second generation or a third generation peptide dendrimer. In some embodiments, the dendritic PGA is a short dendritic PGA. A short dendritic PGA may comprise 2 to 100, 2 to 75, 2 to 50, 2 to 40, 2 to 30, 2 to 20, or 2 to 10 glutamic acid residues. Short dendritic PGA may prove beneficial as it may provide better binding/coating of the nanoparticle compared to linear PGA because short dendritic PGA can provide better flexibility to “wrap” through the nanocarriers. PGA is a peptide polymer. Therefore, in embodiments that comprise a PGA molecule, the PGA may be termed a ‘polymer’ or a ‘second peptide’. In some embodiments, the nanoparticle comprises PGA as the only peptide. In these embodiments, the nanoparticle is initially formed with lipid and nucleic acid only and then coated with PGA.

In some embodiments, the polymer is a glutamic acid containing peptide. Glutamic acid containing peptides have a domain that comprises at least two glutamic acid (E) residues, preferably at least three, at least four, at least five, at least six, at least seven, at least eight, or at least nine E residues. Preferably, the E residues comprise at least 10%, at least 15%, at least 20%, at least 25% or at least 30% of the amino acid residues of this glutamic acid rich domain. Overall, the glutamic acid rich domain may comprise at least 4 amino acids (for instance split across two dipeptide motifs within a given generation of a dendrimer), or at least 6, at least 8, at least 10, or at least 12 amino acid residues. Preferably, the E residues are present at a frequency of every two, every three, or every four amino acid residues (EXEX . . . ), (EXXEXX . . . ), or (EXXXEXXX . . . ) in the glutamic acid containing domain. Most preferably, the E residues are present at a frequency of every three amino acid residues in a glutamic acid rich domain that spans at least 10 amino acid residues. Preferably, the glutamic acid containing peptide comprises 2 to 50, 2 to 40, 2 to 30, 2 to 20, or 2 to 10 amino acid residues in total. The glutamic acid containing peptide may be a first generation, a second generation or a third generation peptide dendrimer, or it may be a linear sequence. The cell and/or tissue targeting motif, e.g. an antibody, may be conjugated to one or more glutamic acid residues. In some examples of this invention, such glutamic acid containing peptides that comprise several glutamic acid residues are termed “PGA”. In embodiments that comprise a glutamic acid containing peptide, the glutamic acid containing peptide may be termed a ‘polymer’ or a ‘second peptide’. In other embodiments, the nanoparticle comprises the glutamic acid containing peptide is the only peptide. In these embodiments, the nanoparticle may be initially formed with lipid and nucleic acid only and then coated with a glutamic acid containing peptide.

In embodiments where the lipid that the targeting motif is attached to is PEG, this may be used as a ‘linker’. In these embodiments, the PEG lipid may be covalently bound to the peptide dendrimer or the lipid (in addition to being bound to the cell and/or tissue targeting motif).

In some embodiments, the cell and/or tissue targeting motif is a mannose sugar covalently linked to a polymer. In embodiments where the polymer is a peptide, the mannose may be linked at the C- and/or N-terminus. In other embodiments where the polymer is a peptide, the mannose may be linked at other positions along the peptide. In some embodiments, the mannose sugar is covalently linked to a PGA molecule. In some embodiments, the mannose sugar is covalently linked to a PGA molecule comprising 2 to 100, 2 to 75, 2 to 50, 2 to 40, 2 to 30, 2 to 20, or 2 to 10 glutamic acid residues. PGA molecules derivatized with mannose are particularly preferred for targeting CD206 expressing cells such as specific lymphatic or endothelial cells, dendritic cells, neutrophils and macrophages, particularly N2 neutrophils and M2 macrophages respectively.

In some embodiments, the cell and/or tissue targeting motif is an antibody covalently linked to a polymer or lipid. In some embodiments, the antibody is covalently linked to a PGA molecule. In some embodiments, the antibody is covalently linked to a PGA molecule comprising 2 to 100, 2 to 75, 2 to 50, 2 to 40, 2 to 30, 2 to 20, or 2 to 10 glutamic acid residues. The antibody may be, for example, an anti-CD3 antibody.

In some embodiments, the cell and/or tissue targeting motif may be covalently bound to a linear peptide. The linear peptide is not, for example, part of the peptide dendrimer. Where the targeting motif comprises a peptide sequence, the peptide sequence may be continuous with the linear peptide amino acid sequence. In some embodiments the linear peptide comprises the ASSLNIA (SEQ ID NO:1), PYDQLRH (SEQ ID NO:2), or KAMHQMQ (SEQ ID NO:3) muscle targeting motif. The muscle cell targeting motif may comprise a variant of the peptide motif ASSLNIA (SEQ ID NO:1), PYDQLRH (SEQ ID NO:2), or KAMHQMQ (SEQ ID NO:3). For example, the variant muscle cell targeting motif may comprise one, two or three amino acid substitutions, deletions or additions relative to the peptide motifs ASSLNIA (SEQ ID NO:1) PYDQLRH (SEQ ID NO:2), or KAMHQMQ (SEQ ID NO:3), provided that the variant retains the ability to target the nanoparticle to a muscle cell. In some embodiments, the linear peptide comprises the RGD or ACDCRGDCFCG (SEQ ID NO:5) integrin targeting peptide motif. The cancer cell targeting motif may comprise a variant of the peptide sequence CGFECVRQCPERC (SEQ ID NO:5). For example, the variant cancer cell targeting motif may comprise one, two or three amino acid substitutions, deletions or additions relative to the peptide sequence CGFECVRQCPERC (SEQ ID NO:5), provided that the variant retains the ability to target the nanoparticle to a cancer cell. In some embodiments the linear peptide comprises the CGFECVRQCPERC (SEQ ID NO:4) lung targeting motif. The lung cell targeting motif may comprise a variant of the peptide sequence CGFECVRQCPERC (SEQ ID NO:4). For example, the variant lung cell targeting motif may comprise one, two or three amino acid substitutions, deletions or additions relative to the peptide sequence CGFECVRQCPERC (SEQ ID NO:4), provided that the variant retains the ability to target the nanoparticle to a lung cell. The linear peptide may comprise one or more units of the targeting motif. For example, the linear peptide may comprise 1, 2, 3, 4, 5, 6, 7, 8, 9, 10 or more targeting motifs. In some embodiments, the linear peptide consists of the ASSLNIA (SEQ ID NO:1), PYDQLRH (SEQ ID NO:2), or KAMHQMQ (SEQ ID NO:3) muscle targeting motif, the RGD or ACDCRGDCFCG (SEQ ID NO:5) integrin targeting peptide motif, or the CGFECVRQCPERC (SEQ ID NO:4) lung targeting peptide motif. In some embodiments, the linear peptide consists of the lung targeting peptide sequence CGFECVRQCPERC (SEQ ID NO:4). Where the targeting motif is a sugar the linear peptide may comprise one or more glycosylations. For example, the linear peptide may comprise one or more mannose glycosylations.

In some embodiments, the cell and/or tissue targeting motif may be covalently bound to the peptide dendrimer of the nanoparticle. The cell and/or tissue targeting motif may be covalently bound to the C-terminus and/or the N-terminus of the peptide dendrimer. The peptide dendrimer core may comprise the cell and/or tissue targeting motif. The cell and/or tissue targeting motif may be bound to the outermost layer of peptide motifs. Where the targeting motif is a sugar, the peptide dendrimer may comprise one or more glycosylations. For example, the peptide dendrimer may comprise one or more mannose glycosylations. For example, the peptide dendrimer may have the structure Mannose-G1-RL, 2-LR. In some embodiments, the peptide dendrimer may have the structure Mannose-G1-EEEE. For example, the peptide dendrimer may have the structure Mannose-G1-RL, 2-LR. In some embodiments, the peptide dendrimer may have the structure Mannose-G1-EEEE. In some embodiments, the peptide dendrimer may have the structure (LR)4(KRL)2KGSGGSGGSGGSC[(S-S)-α-D-Thiomannose]. In some embodiments, the peptide dendrimer may have the structure (Ac-EEEE)2KGSGGSGGSC[(S-S)-α-D-Thiomannose], which may be derivatized at its C-terminal end, e.g. with an amine group (NH2). In some embodiments, the cell and/or tissue targeting motif is an antibody. In some embodiments, the antibody is an anti-CD3 antibody.

In some embodiments, the nanoparticle comprises a second peptide that comprises the cell and/or tissue targeting motif. The second peptide may be a linear peptide or a dendritic peptide. In embodiments where the second peptide is a dendritic peptide, it may be termed a “second peptide dendrimer” herein. Thus, in some embodiments, the nanoparticle comprises a second peptide dendrimer covalently bound to the cell and/or tissue targeting motif. The cell and/or tissue targeting motif may be covalently bound to the C-terminus and/or the N-terminus of the second peptide dendrimer. The second peptide dendrimer core may comprise the cell and/or tissue targeting motif. The cell and/or tissue targeting motif may be bound to the outermost layer of peptide motifs of the second peptide dendrimer. Where the targeting motif is a sugar, the second peptide dendrimer may comprise one or more glycosylations. For example, the second peptide dendrimer may comprise one or more mannose glycosylations. In some embodiments, the second peptide dendrimer may have the structure Mannose-G1-RL, 2-LR. In some embodiments, the second peptide dendrimer may have the structure Mannose-G1-EEEE. For example, the second peptide dendrimer may have the structure Mannose-G1-RL, 2-LR. In some embodiments, the peptide second dendrimer may have the structure Mannose-G1-EEEE. In some embodiments, the second peptide dendrimer has the structure (Ac-EEEE)2KGSGGSGGSC[(S-S)-α-D-Thiomannose]. In some embodiments, the second peptide dendrimer has the structure (Ac-EEEE)2KGSGGSGGSC[(S-S)-α-D-Thiomannose](“Ac” represent acetylation of the N-terminus of the peptide dendrimer). In some embodiments, the cell and/or tissue targeting motif is an antibody. In some embodiments, the antibody is an anti-CD3 antibody.

In some embodiments, the nanoparticle is targeted to particular tissues and/or cell types is achieved by selecting the NP ratio of dendrimer:nucleic acid, and/or the w/w ratio of lipid:nucleic acid. For instance, higher NP ratios favour spleen targeting, with some lung targeting also seen at lipid:nucleic acid w/w ratios of around 10:1. Lung targeting can be enhanced by increasing the lipid:nucleic acid w/w ratio, for example to around 23:1.

The dendrimers used in the invention are first, second or third generation peptide dendrimers, meaning that they have up to three ‘layers’ of peptide motifs interspersed between ‘branching’ residues, such as lysine. First generation dendrimers have the following structure, shown in the N-termini to C-terminus orientation, and taking Lys to be the branching unit:

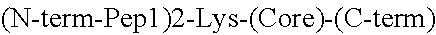

Second generation dendrimers have the following structure, shown in the N-termini to C-terminus orientation, and taking Lys to be the branching unit:

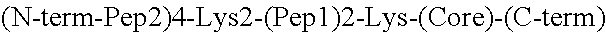

Third generation dendrimers have the following structure, shown in the N-termini to C-terminus orientation, and taking Lys to be the branching unit:

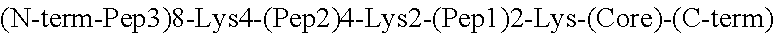

Third generation dendrimers are represented diagrammatically (with N-termini on the left and C-terminus on the right) in FIG. 1.

In FIG. 1, the circle represents the core sequence. Each triangle represents a branching residue, such as lysine. Each rectangle represents a peptide motif. There are two peptide motifs in the first layer, four peptide motifs in the second layer, and eight peptide motifs in the third layer of the third generation dendrimer. The N- and C-termini may be derivatised with further chemical motifs, as discussed herein. For instance, while in underivatized embodiments, the C-terminus is a carboxylic acid, in other embodiments the C-terminus is derivatised e.g. to comprise a primary amide group, CONH2 (instead of COOH), as a result of the chemical pathway used to synthesise the dendrimer. Functionally important derivatisations such as targeting moieties (e.g. antibodies, peptide groups, sugar groups and/or lipid chains) are also envisaged, which can be attached to the N- and/or C-termini, or at other positions along the dendrimer. The N-terminus of the peptide dendrimers disclosed herein may be derivatized, e.g. acetylated.

As described herein, the dendrimers can be first, second or third generation. This can be defined structurally as follows: First generation dendrimers comprise a core peptide sequence, a first branching residue and two first peptide motifs each attached to the first branching residue. The two first peptide motifs independently consist of a single amino acid, dipeptide, tripeptide or tetrapeptide motifs. Second generation dendrimers further comprise two second branching residues (e.g. lysine) and four second peptide motifs, wherein one of the second branching residues is covalently bound to one of the first peptide motifs and the other second branching residue is covalently bound to the other first peptide motif, and wherein each second branching residue is covalently bound to two second peptide motifs. The four second peptide motifs independently consist of a single amino acid, dipeptide, tripeptide or tetrapeptide motifs. Third generation dendrimers further comprises four third branching residues (e.g. lysine) and eight third peptide motifs, wherein each second peptide motif is respectively covalently bound to one of the third branching residues such that each third branching residue is covalently bound to one second peptide motif, and wherein each third branching residue is covalently bound to two third peptide motifs. The eight third peptide motifs independently consist of a single amino acid, dipeptide, tripeptide or tetrapeptide motifs. Each of the first, second and third peptide motifs, where present, may comprise (1) an amino acid with a basic side chain such as, but not limited to, Lysine (K) or Arginine (R) or Histidine (H), (2) an amino acid with an acidic side chain such as but not limited to Aspartic acid (D) and Glutamic acid (E), (3) an amino acid with a non-polar side chain such as, but not limited, to Glycine (G), Alanine (A), Valine (V), Isoleucine (I), Leucine (L), Methionine (M), Phenylalanine (F), Beta-alanine (B), Tryptophan (W), Proline (P), aminohexanoic acid (X) and Cysteine (C) and (4) an amino acid with a uncharged polar side chain such as, but not limited to, Asparagine (N), Glutamine (Q), Serine (S), Threonine (T) and Tyrosine (Y).

The core peptide motif of the dendrimer is a single amino acid residue or a short peptide motif such as a dipeptide or tripeptide motif. The core sequence may comprise any amino acid (L- and/or D-isomers), e.g. a glycine (G), a serine (S), a cysteine (C), an alanine (A), a lysine (K), a leucine (L), a valine (V), an isoleucine (I), a phenylalanine (F), a methionine (M), a tyrosine (Y), a tryptophan (W), a proline (P), a threonine (T), an asparagine (N), a glutamine (Q), an aspartic acid (D), a glutamic acid (E), an arginine (R), and or a histidine (H). The core sequence may also comprise non-naturally occurring amino acids (L- and/or D-isomers), e.g. s Beta-alanine (B) and/or an aminohexanoic acid (X). Where the core is a tripeptide motif, it may comprise a glycine (G), a serine (S), and either a cysteine (C) or an alanine (A). Preferably, the core comprises an ionisable residue, such as a histidine (H). The core sequence may comprise an arginine (R), a histidine (H) and a cysteine (C). The core sequence may comprise an arginine (R) or glycine (G), a histidine (H) or serine (S) and a cysteine (C) or an alanine (A). For instance, the core sequence may be GSC or RHC. The tripeptide motif may comprise an alanine (A), a lysine (K) and a leucine (L). For instance, the core sequence may be KLA. The core peptide may be covalently bound to a further moiety, such as a cell specific targeting peptide, or may be derivatized with a lipid molecule. One, some or all of the amino acids of the dendrimer, e.g. of the core peptide motif may be covalently bound to a further moiety, such as an antibody, a cell specific targeting peptide, sugar ligands such as glucose, mannose, galactose and GalNAc (or glycans comprising the same) and/or a lipid substituent. The skilled person is readily able to select further moieties that do not adversely affect solubility or nucleic acid binding characteristics.

Preferred dendrimers are presented in Table 1 below. Certain examples are discussed in particular. For instance, in dendrimers where each peptide motif is an Arg-His-Leu (RHL) tripeptide, this structure can be denoted G1-RHL, G1,2-RHL and G1,2,3-RHL. In dendrimers where the peptide motifs are not the same across all generations, e.g. in a third generation dendrimer wherein the two first peptide motifs and the four second peptide motifs are Arg-Leu (RL) dipeptides and the eight third peptide motifs are Leu-Arg (LR) dipeptides, this structure can be denoted G1, 2-RL, 3-LR. In dendrimers where the peptide motifs are not the same across a second generation dendrimer, e.g. in a dendrimer wherein the two first peptide motifs are Arg-Leu (RL) and the four second peptide motifs are Leu-Arg (LR) dipeptides, this structure can be denoted G1-RL, 2-LR. In dendrimers where each peptide motif is an Arg-Leu (RL) dipeptide, this structure can be denoted G1-RL, G1,2-RL and G1,2,3-RL. In dendrimers where each peptide motif is a Lys-Leu (KL) dipeptide, this structure is denoted G1-KL, G1,2-KL and G1,2,3-KL. In dendrimers where each peptide motif is a Leu-Arg (LR) dipeptide, this structure is denoted G1-LR, G1,2-LR and G1,2,3-LR

‘G1’. ‘G2’ and ‘G3’ refer to the ‘generation-1’, ‘generation-2’ and ‘generation-3’ peptide motifs of the first, second and third layers, respectively. Each amino acid residue can be an L-amino acid or a D-amino acid. D-amino acids may be designated using lower case letters in the single-letter code. Alternatively, dendrimers in which each amino acid is the D-isoform can be written with a preceding “D-” before the short-form denotation of the dendrimer.

Peptide dendrimers with a specified core are also discussed herein. For example, in dendrimers where a defined peptide core is intended, for example a peptide core of Arg-His-Cys, this structure can be denoted as RHCG1, 2-RL. It will be understood that per the nomenclature of the peptide dendrimers disclosed here, in the previous example, ‘G’ refers to the ‘generation’ of the peptide motif and not a Glycine residue. In contrast, a peptide dendrimer with a structure denoted as GSCG1, 2-RL, 3-LR it will be understood that the first ‘G’ in this context will refer to a Glycine residue while the second ‘G’ refers to the ‘generation’ of the peptide motifs. In other examples, where a defined core is intended, the core sequence will be underlined, leaving the “G” denoting the generation not underlined (e.g. GSCG1, 2-RL, 3-LR).

In some embodiments, the peptide dendrimer further comprises an alkyl chain, alkenyl chain, an antibody or a fragment thereof, a sugar, and/or a fatty acid. An alkyl or alkenyl chain may be conjugated to the core peptide sequence, for instance at the C terminus of the peptide dendrimer. Alternatively, or in addition to, the alkyl or alkenyl chain may be conjugated to the N terminus of the peptide dendrimer.

In some embodiments, alkyl or alkenyl chains comprise from about 5 carbons to about 50 carbons, preferably from about 12 to about 30 carbons.

In some embodiments, the peptide dendrimer comprises a fatty acid conjugated to the C terminus of the peptide dendrimer. In other embodiments, the peptide dendrimer comprises a fatty acid conjugated to the N terminus of the peptide dendrimer.

Preferably, the N/P ratio, which is the amount of peptide (measured by the number of 1+ charged nitrogen atoms on the peptide, N) to the amount of nucleic acid (measured by the number of 1− charged phosphate groups in the backbone, P) is greater than 0.05:1, for instance greater than 0.1:1. (The N/P ratio terminology can be expressed as “N/P”, “N:P”, or “NP”.) In some embodiments the N/P ratio is 0.15:1, or about 0.15:1, or at least 0.15:1. In some embodiments the N/P ratio is 0.16:1, or about 0.16:1, or at least 0.16:1. In some embodiments the N/P ratio is 0.6:1, or about 0.6:1, or at least 0.6:1. In some embodiments, the N/P ratio is at least, or greater than, 1:1, for instance about 2:1 or greater, about 2.5:1 or greater, about 3:1 or greater, about 4:1 or greater, about 5:1 or greater, about 10:1 or up to 20:1. In some embodiments, the N/P ratio is about 5:1, about 8:1, about 10:1, or about 20:1. In some embodiments, the N/P ratio is in the range of about 0.01:1 and 100:1, about 2:1 to about 20:1, or about 2.5:1 to about 10:1.

In some embodiments, the peptide dendrimer has the structure G1,2-RL, 3-LR; G1-RL, 2-LR; G1,2-RHL; G1-LRLR; G1,2-RF, 3-HL; or G1-R. In some embodiments, the peptide dendrimer has the structure GSCG1,2-RL, 3-LR; RHCG1-RL, 2-LR; GSCG1,2-RHL; GSCG1-LRLR; GSCG1,2-RF, 3-HL; or GSCG1-R.

In some embodiments, the peptide dendrimer has the structure G1,2-RL, 3-LR; G1-RL, 2-LR; G1,2-RHL; G1-LRLR; G1,2-RF, 3-HL; or G1-R wherein the N:P ratio is between 0.05:1 and 20:1, for example the N:P ratio may be 0.16:1 or 0.6:1 for delivery of the nanoparticle to myeloid cells, In some embodiments, the peptide dendrimer has the structure GSCG1,2-RL, 3-LR; RHCG1-RL, 2-LR; GSCG1,2-RHL; GSCG1-LRLR; GSCG1,2-RF, 3-HL; GSCG1-R wherein the N:P ratio is between 0.05:1 and 20:1, for example N:P ratio may be 0.16:1 or 0.6:1, for delivery of the nanoparticle to macrophages. In some embodiments, the peptide dendrimer has the structure GSCG1,2-RL, 3-LR, wherein the N:P ratio is 0.16:1 for delivery of the nanoparticle to macrophages. In some embodiments, the peptide dendrimer has the structure RHCG1-RL, 2-LR, wherein the N:P ratio is 8:1 for delivery of the nanoparticle to macrophages. In some embodiments, the peptide dendrimer has the structure GSCG1,2-RHL, wherein the N:P ratio is between 5:1 for delivery of the nanoparticle to macrophages.

Typically, compositions comprising nanoparticles including a first generation peptide dendrimer, lipid and a nucleic acid do not form a monodisperse population of nanoparticles, when the nanoparticle has a dendrimer:nucleic acid NP ratio of above about 2:1, for instance at N:P at about 8:1. However, using higher NP ratios enables certain properties of the nanoparticle to be controlled. Moreover, the ability to form a monodisperse population is important for development of pharmaceutical compositions as this will limit the batch-to-batch variability, allow for more well defined nanoparticle characterisation and consistent results to be obtained in vitro and in vivo. The distribution of the size of nanoparticles in a population can be expressed in terms of the populations polydispersity index (PDI). A PDI of 1 indicates a completely polydisperse population of nanoparticles, whereas a PDI of 0 indicates a completely monodisperse population of nanoparticles. Therefore, the PDI can be used as a measure of the uniformity of a population of nanoparticles in a composition. A PDI of 0.35 or less is considered to provide a suitably monodisperse population of nanoparticles for, e.g., development of suitable pharmaceutical compositions. However, for nanoparticles used for in vitro or ex vivo transfection of cells the PDI may be higher than 0.35, for example, the PDI may be equal to or lower than 0.65, 0.6, 0.55, 0.5, 0.45, 0.4, 0.35, or 0.3. For simplicity and clarity, as used herein, where a PDI value is given for a particular peptide dendrimer, or mix of peptide dendrimers, this PDI value is in reference to a population of nanoparticles comprising the peptide dendrimer(s), a nucleic acid, and a lipid. For example, where the peptide dendrimer RHCG1-R is referred to as having a PDI of 0.566, this PDI value is associated with the nanoparticle comprising RHCG1-R, nucleic acid and lipid. The PDI of a particular nanoparticle formulation can be measured by any method standard in the art, for example, using the Zetasizer Advance Series—Pro according to the manufacturer's instructions and the conditions disclosed in Example 3.

A non-uniform or non-monodisperse population of nanoparticles (e.g. wherein the PDI of the nanoparticle is greater than 0.35) and may indicate that the binding of a peptide dendrimer to the nucleic acid in a nanoparticle is unstable (i.e. the peptide dendrimer and nucleic acid have a low binding affinity and readily dissociate). In contrast, a uniform or monodisperse population of nanoparticles may indicate that the binding of a peptide dendrimer to a nucleic acid in a nanoparticle is stable (i.e. the peptide dendrimer and nucleic acid bind with high affinity and do not readily dissociate).

Without wishing to be bound by any particular theory, it is proposed that nanoparticles comprising a first generation peptide dendrimer tend to form a less uniform population of nanoparticles (and thus have a “high” PDI of greater than 0.35) compared to populations of nanoparticles comprising second or third generation peptide dendrimers because first generation dendrimers have fewer cationic groups per peptide dendrimer. With fewer cationic groups to interact with the anionic groups on a nucleic acid, first generation peptide dendrimer/nucleic acid may form unstable complexes that can dissociate at a relatively high rate compared to a second/third generation dendrimer/nucleic acid complex, resulting in a heterogenous mix of nanoparticles in solution. By including a second or third generation peptide dendrimer in combination with a first generation peptide dendrimer, it is thought that this can balance the relatively stable association needed to form a uniform particle (provided by the second or third generation peptide dendrimer) with the relatively unstable association needed for efficient release of the nucleic acid once delivered to a cell (provided by the first generation peptide dendrimer).

The inventors have also found that transfection efficiency can be improved by using nanoparticles that comprise two peptide dendrimers that can, when used alone in a nanoparticle comprising a nucleic acid and a lipid, form suitably uniform nanoparticle populations. That is, improved transfection efficiency can be achieved even when each of the peptide dendrimers individually form monodisperse populations of nanoparticles in a solution. For example, the first and second peptide dendrimer may be independently selected from a second or third generation peptide dendrimer which, when used individually, form monodisperse nanoparticle populations.

Thus, in some embodiments, the first peptide dendrimer may be selected based on the polydispersity index (PDI) of a reference peptide dendrimer/lipid nanoparticle comprising the first peptide dendrimer at an NP ratio of 8:1. Likewise, the second peptide dendrimer may be selected based on the PDI of a reference peptide dendrimer/lipid nanoparticle comprising the second peptide dendrimer at an NP ratio of 8:1. In some embodiments, the first peptide dendrimer, when used in a first reference nanoparticle consisting of the first peptide dendrimer, a nucleic acid and a lipid, the first reference nanoparticle has a PDI higher than the PDI of a second reference nanoparticle consisting of the second peptide dendrimer, a nucleic acid and a lipid, wherein the reference nanoparticles are prepared at an NP ratio of 8:1.

In some embodiments, the PDI of the nanoparticle comprising a first peptide dendrimer, a second peptide dendrimer, a nucleic acid and a lipid is less than or equal to 0.6, 0.55, 0.5, 0.45, 0.4, 0.39, 0.38, 0.37, 0.36, 0.35, 0.34, 0.33, 0.32, 0.31, 0.30. In some embodiments, the PDI of the nanoparticle comprising a first peptide dendrimer, a second peptide dendrimer, a nucleic acid and a lipid is less than or equal to 0.35.

In some embodiments, the PDI of the first dendrimer is greater than about 0.20, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40. In some embodiments, the PDI of the second dendrimer is less than about 0.40, about 0.39, about 0.38, about 0.37, about 0.36, about 0.35, about 0.34, about 0.33, about 0.32, about 0.31, about 0.30, about 0.29, about 0.28, about 0.27, about 0.26, about 0.25, about 0.24, about 0.23, about 0.22, about 0.21, about 0.20. In some embodiments, the PDI of the first peptide dendrimer is greater than about 0.25, about 0.26, about 0.27, about 0.28, about 0.29, or about 0.30 and the PDI of the second peptide dendrimer is lower than about 0.25, about 0.24, about 0.23, about 0.22, about 0.21, about 0.20. In some embodiments, the PDI of the first peptide dendrimer is greater than about 0.40 and the PDI of the second peptide dendrimer is lower than about 0.40. In some embodiments, the PDI of the first peptide dendrimer is greater than about 0.35 and the PDI of the second peptide dendrimer is lower than about 0.35. In some embodiments, the PDI of the first peptide dendrimer is greater than about 0.30 and the PDI of the second peptide dendrimer is lower than about 0.30. In some embodiments, the PDI of the first peptide dendrimer is greater than about 0.30 and the PDI of the second peptide dendrimer is lower than about 0.30. In some embodiments, the PDI of the first peptide dendrimer is greater than about 0.20 and the PDI of the second peptide dendrimer is lower than about 0.20. In some embodiments, the PDI of the first peptide dendrimer is greater than about 0.10 and the PDI of the second peptide dendrimer is lower than about 0.10. In some embodiments, the PDI of the first peptide dendrimer is greater than about 0.10 and the PDI of the second peptide dendrimer is lower than about 0.20.

In some embodiments, the first peptide dendrimer has a PDI at least about 0.05, about 0.06, about 0.07, about 0.08, about 0.09, about 0.10, about 0.11, about 0.12, about 0.13, about 0.14, about 0.15, about 0.2, about 0.3, about 0.4, about 0.5, about 0.6, about 0.7, about 0.8, about 0.9 greater than the PDI of the second peptide dendrimer. In some embodiments, the first peptide dendrimer has a PDI of between 0.05 and 0.9, between 0.06 and 0.8, between 0.07 and 0.6, between 0.08 and 0.5, between 0.09 and 0.4, about 0.1 and 0.3, between 0.12 and 0.2, between 0.13 and 0.15 greater than the PDI of the second peptide dendrimer.

In some embodiments, the first peptide dendrimer, when used in a first reference nanoparticle consisting of the first peptide dendrimer, a nucleic acid and a lipid, the first reference nanoparticle has a PDI of between 0.05 and 0.9, between 0.06 and 0.8, between 0.07 and 0.6, between 0.08 and 0.5, between 0.09 and 0.4, between 0.1 and 0.3, between 0.12 and 0.2, or between 0.13 and 0.15 greater than the PDI of a second reference nanoparticle consisting of the second peptide dendrimer, a nucleic acid and a lipid.

The transfection efficiency and PDI of a nanoparticle comprising two peptide dendrimers can be optimised by altering the relative levels of each peptide dendrimer in the nanoparticle. For example, the molar ratio with respect to the nitrogen contributed from each of the first peptide dendrimer and the second peptide dendrimer in a nanoparticle can be selected from between 1:4 and 4:1, 1:3 and 3:1, 1:2 and 2:1. In some examples, the molar ratio of nitrogen contributed by each of the first peptide dendrimer and the second peptide dendrimer is 1:4, 1:3, 1:2, 1:1, 2:1, 2:1, 3:1, or 4:1.

In embodiments in which the composition comprises a first peptide dendrimer and a second peptide, the first peptide dendrimer may be a first generation peptide dendrimer comprising a core peptide sequence, a first branching unit and two first peptide motifs. In some embodiments, the first peptide dendrimer may comprise a cell or tissue targeting motif.

In other such embodiments, the first peptide dendrimer is a second generation peptide dendrimer comprising a core peptide sequence, a first branching unit and two first peptide motifs, at least two second branching units and four second peptide motifs. One of the second branching residues is covalently bound to one of the first peptide motifs and the other second branching residue is covalently bound to the other first peptide motif. Each of the second branching residues is covalently bound to two second peptide motifs. Each of the first branching unit and the two second branching units may be independently selected from a lysine, 2,4-diaminobutyric acid, ornithine, or diaminopropionic acid.

In other such embodiments, the first peptide dendrimer is a third generation peptide dendrimer comprising a core peptide sequence, a first branching unit and two first peptide motifs, at least two second branching units and four second peptide motifs and at least four third branching residues and eight third peptide motifs. One of the second branching residues is covalently bound to one of the first peptide motifs and the other second branching residue is covalently bound to the other first peptide motif. Each of the second branching residues is covalently bound to two second peptide motifs. Each second peptide motif is respectively covalently bound to one of the third branching residues, such that each third branching residue is covalently bound to one second peptide motifs. Each third branching residue is covalently bound to two third peptide motifs. Each of the first branching unit, the two second branching units and the four third branching units may be independently selected from a lysine, 2,4-diaminobutyric acid, ornithine, or diaminopropionic acid.

The first peptide dendrimer may be a first generation peptide dendrimer comprising a core peptide sequence, a first branching unit and two first peptide motifs. Alternatively, the first peptide dendrimer is a second generation peptide dendrimer comprising a core peptide sequence, a first branching unit and two first peptide motifs, at least two second branching units and four second peptide motifs. One of the second branching residues is covalently bound to one of the first peptide motifs and the other second branching residue is covalently bound to the other first peptide motif. Each of the second branching residues is covalently bound to two second peptide motifs. Each of the first branching unit and the two second branching units may be independently selected from a lysine, 2,4-diaminobutyric acid, ornithine, or diaminopropionic acid.

In some embodiments, the second peptide dendrimer is a second generation peptide dendrimer comprising a core peptide sequence, a first branching unit and two first peptide motifs, at least two second branching units and four second peptide motifs. One of the second branching residues is covalently bound to one of the first peptide motifs and the other second branching residue is covalently bound to the other first peptide motif. Each of the second branching residues is covalently bound to two second peptide motifs. Each of the first branching unit and the two second branching units may be independently selected from a lysine, 2,4-diaminobutyric acid, ornithine, or diaminopropionic acid.

In some embodiments, the second peptide dendrimer is a third generation peptide dendrimer comprising a core peptide sequence, a first branching unit and two first peptide motifs, at least two second branching units and four second peptide motifs and at least four third branching residues and eight third peptide motifs. One of the second branching residues is covalently bound to one of the first peptide motifs and the other second branching residue is covalently bound to the other first peptide motif. Each of the second branching residues is covalently bound to two second peptide motifs. Each second peptide motif is respectively covalently bound to one of the third branching residues, such that each third branching residue is covalently bound to one second peptide motifs. Each third branching residue is covalently bound to two third peptide motifs. Each of the first branching unit, the two second branching units and the four third branching units may be independently selected from a lysine, 2,4-diaminobutyric acid, ornithine, or diaminopropionic acid.

In some embodiments, the second peptide dendrimer is a first generation peptide dendrimer comprising a core peptide sequence, a first branching unit and two first peptide motifs.

In some embodiments, the first peptide dendrimer is a first generation peptide dendrimer and the second peptide dendrimer is a first generation peptide dendrimer. In some embodiments, the first peptide dendrimer is a first generation peptide dendrimer and the second peptide dendrimer is a second generation peptide dendrimer. In some embodiments, the first peptide dendrimer is a first generation peptide dendrimer and the third peptide dendrimer is a third generation peptide dendrimer.

In some embodiments, the first peptide dendrimer is a second generation peptide dendrimer and the second peptide dendrimer is a first generation peptide dendrimer. In some embodiments, the first peptide dendrimer is a second generation peptide dendrimer and the second peptide dendrimer is a second generation peptide dendrimer. In some embodiments, the first peptide dendrimer is a second generation peptide dendrimer and the third peptide dendrimer is a third generation peptide dendrimer.

In some embodiments, the first peptide dendrimer is a third generation peptide dendrimer and the second peptide dendrimer is a first generation peptide dendrimer. In some embodiments, the first peptide dendrimer is a third generation peptide dendrimer and the second peptide dendrimer is a second generation peptide dendrimer. In some embodiments, the first peptide dendrimer is a third generation peptide dendrimer and the third peptide dendrimer is a third generation peptide dendrimer.

In some embodiments, the peptide motifs of the first and second peptide dendrimers are independently selected from a single amino acid, a dipeptide, tripeptide or tetrapeptide motif. For the avoidance of doubt, the two first peptide motifs of the first or second peptide dendrimer, the four second peptide motifs of the first or second peptide dendrimer, and the eight third peptide motifs of the first or second peptide dendrimer are independently selected from a single amino acid, a dipeptide, tripeptide or tetrapeptide motif.

Each peptide motif of the first and second peptide dendrimer independently comprises naturally occurring L- or D-amino acids and/or non-naturally occurring L- or D-amino acids, for example, Beta-alanine (B) or aminohexanoic acid (X or Acp). For the avoidance of doubt, the two first peptide motifs of the first or second peptide dendrimer, the four second peptide motifs of the first or second peptide dendrimer, and the eight third peptide motifs of the first or second peptide dendrimer independently comprises naturally occurring L- or D-amino acids and/or non-naturally occurring L- or D-amino acids, for example, Beta-alanine (B) or aminohexanoic acid (X or Acp).

In some embodiments, the first, second and/or third peptide motifs, where present, of the first and/or second peptide dendrimer comprise an amino acid with a basic side chain.

In some embodiments, the core sequence of the first and/or second peptide dendrimer comprises an amino acid residue with an ionisable group such as histidine.

In some embodiments, the first, second and/or third peptide motifs, where present, of the first and/or second peptide dendrimer comprise an amino acid with a non-polar side chain.

In some embodiments, the first, second and/or third peptide motifs, where present, of the first and/or second peptide dendrimer comprise an amino acid with an acidic side chain.

In some embodiments, the first, second and/or third peptide motifs, where present, of the first and/or second peptide dendrimer comprise an amino acid with an uncharged polar side chain.

In some embodiments, each of the first, second and third peptide motifs, where present, may comprise (1) an amino acid with a basic side chain such as, but not limited to, Lysine (K) or Arginine (R) or Histidine (H), (2) an amino acid with an acidic side chain such as but not limited to Aspartic acid (D) and Glutamic acid (E), (3) an amino acid with a non-polar side chain such as, but not limited, to Glycine (G), Alanine (A), Valine (V), Isoleucine (I), Leucine (L), Methionine (M), Phenylalanine (F), Beta-alanine (B), Tryptophan (W), Proline (P), aminohexanoic acid (X) and Cysteine (C) and (4) an amino acid with a uncharged polar side chain such as, but not limited to, Asparagine (N), Glutamine (Q), Serine (S), Threonine (T) and Tyrosine (Y).

Preferably at least one of the first, second and third peptide motifs (where present) of the first and/or second peptide dendrimer comprise a leucine (L), an arginine (R) and/or a histidine (H). At least two of the first, second and third peptide motifs (where present) may comprise a leucine (L), an arginine (R) and/or a histidine (H). In some embodiments, all of the first, second and third peptide motifs (where present) of the first and/or second peptide dendrimer comprise a leucine (L), an arginine (R) and/or a histidine (H).

Preferably at least one of the first, second and third peptide motifs comprise a leucine (L). At least two of the first, second and third peptide motifs may comprise a leucine (L). In some embodiments, all of the first, second and third peptide motifs comprise a leucine (L).

In some embodiments, the core peptide sequence of the first and/or second peptide dendrimer comprises the amino acid sequence RHC, GSA or GSC.

In some embodiments, the peptide dendrimer, nucleic acid and lipid form a positively charged particle.

In other embodiments, the peptide dendrimer, nucleic acid and lipid form a negatively charged particle or a particle with neutral charge.

The lipid component of the nanoparticle may comprise a mixture of lipids, including a cationic lipid. For instance, the lipid component may comprise dioleoylphosphatidylethanolamine (DOPE) and N-[1-(2,3-dioleyloxy)propyl]-N,N,N-trimethylammonium chloride (DOTMA). The DOPE:DOTMA w/w ratio can be readily determined for optimal properties for a given application, but will typically range from 1:10 to 10:1, 1:8 to 8:1, or 1:5 to 5:1. Preferably the range is 3:1 to 1:3, or 2:1 to 1:2. Most preferably the DOPE:DOTMA ratio is 1:1. In other embodiments, the lipid comprises 1,2-dioleoyl-3-trimethylammonium-propane chloride (DOTAP), e.g. as the sole lipid or in combination with DOPE.

In other embodiments, the lipid component of the composition may comprise other lipids, in addition to (or instead of) DOPE and DOTMA, for instance DODAP, DOTAP and/or DORI (N-(2-hydroxyethyl)-N,N-dimethyl-2,3-bis(oleoyloxy)propan-1-aminium bromide). Exemplary lipid components are set out below:

### Cationic Lipids:

N-[1-(2,3-dioleyloxy)propyl]-N,N,N-trimethylammonium chloride (DOTMA)
1,2-dioleoyl-3-trimethylammonium-propane chloride (DOTAP)
N-(2-hydroxyethyl)-N,N-dimethyl-2,3-bis(oleoyloxy)propan-1-aminium bromide (DORI)
2,3-dioleyloxy-N-(2[spermine-carboxamido]ethyl)-N,N-dimethyl-1-propanaminium trifluoroacetate (DOSPA)
3β-[N—(N′,N′-dimethylaminoethane)-carbamoyl]cholesterol hydrochloride (DC-chol)

### Neutral Lipids:

1,2-dioleoyl-sn-glycero-3-phosphoethanolamine (DOPE)
1,2-dioleoyl-sn-glycero-3-phosphocholine (DOPC)

### Cholesterol

### Anionic Lipids:

1,2-dioleoyl-sn-glycero-3-phospho-(1′-rac-glycerol) (DOPG)

### Ionisable Lipids:

1,2-dioleoyloxy-3-(dimethylamino)propane (DODAP)
   DLin-DMA
   DLin-KC2-DMA
   DLin-MC3-DMA
   SM-102
   ALC-0315

Other potential lipids include 4-(2-aminoethyl)-morpholino-cholesterol-hemisuccinate, (MoChol) cholesterolhemisuccinate (CHEMS), phosphatidylcholine (PC), phosphatidylethanolamine (PE), dimyristoylphosphatidylcholine (DMPC), dipalmitoylphosphatidylcholine (DPPC), distearoylphosphatidylcholine (DSPC), dimyristoylphosphatidylethanolamine (DMPE), dipalmitoylphosphatidylethanolamine (DPPE), cholesterol-(3-imidazol-1-yl propyl)carbamate (CHIM), dimethyldioctadecylammonium bromide (DDAB), dioleoylphosphatidylserine (DOPS), dioleoylphosphatidylglycerol (DOPG), cholesterol sulfate (chol-S04).

It is envisaged that any of the aforementioned lipids can be used alone or in combination with each other in the compositions of the invention. Additionally, the lipids can be derivatized via linkage to PEG group such as PEG2000.

In some embodiments, the lipid of the nanoparticle comprises a cationic lipid, a neutral lipid, an anionic lipid and/or an ionisable lipid.

In some embodiments, the lipid of the nanoparticle comprises a saturated fatty acid. Additionally, or alternatively, the lipid of the composition may comprise an unsaturated fatty acid.

In some embodiments, the lipid comprises 1, 2, 3, 4, 5 or 6 fatty acid chains. Preferably, the lipid comprises 2, 3, 4 or 6 fatty acid chains.

In some embodiments, the lipid comprises dioleoylphosphatidylethanolamine (DOPE) and/or N-[1-(2,3-dioleyloxy)propyl]-N,N,N-trimethylammonium chloride (DOTMA). In some embodiments, the lipid comprises dioleoylphosphatidylethanolamine (DOPE) and dioleoylphosphatidylglycerol (DOPG).

The lipid component of the nanoparticle may comprise DOTMA, DOPE, DOPC and/or DOPG.

The amount of lipid component can be expressed in a weight:weight ratio (“w/w”, or “w:w”), with respect to the amount of the nucleic acid in the nanoparticle may be in the range of 1:50 to 50:1. More preferably, the amount of lipid (by weight) is 1:1 to 50:1, or 2:1 to 25:1, or at least about 0.5:1, at least about 1:1, at least about 2.5:1, or at least about 5:1 with respect to the amount of nucleic acid (by weight). The lipid:nucleic acid ratio can be at least 2:1. These ratios refer to the weight of the total lipid. As described herein, the nanoparticle may comprise a lipid that includes more than one lipid component, e.g. a mixture of two, three or four lipids. The weight of the lipid component is the total (combined) weight of these lipid components. Preferably, each lipid component is mixed in approximately equal proportions.

In some embodiments, the lipid:nucleic acid w/w can be selected to target the nanoparticle to lung tissue. For example, the nanoparticle may comprise a lipid:nucleic acid w/w of between 2:1 and 40:1 to target the nanoparticle to the lung and/or spleen. In a further example, the nanoparticle may comprise a lipid:nucleic acid w/w of 10:1 or 23:1. At a lipid:nucleic acid w/w of 10:1 both spleen and lung is targeted, with spleen being targeted more strongly than lung. At a lipid:nucleic acid w/w of 23:1 both spleen and lung is targeted, with lung being targeted more strongly than spleen.

The peptide dendrimer may comprise a cell penetrating peptide, an endosomal escape peptide, a nuclear localisation motif, and/or a fatty acid. The cell penetrating peptide, endosomal escape peptide, nuclear localisation motif, and/or fatty acid may be conjugated to the C-terminus of the peptide dendrimer. Alternatively, or in addition to, the cell penetrating peptide, endosomal escape peptide, nuclear localisation motif, and/or fatty acid may be conjugated to the N-terminus of the peptide dendrimer.

Transfection efficiency may be increased following targeted delivery of the nanoparticle to a cell or tissue by any of the mechanisms described above (e.g. by including a targeting motif, selecting an appropriate lipid:nucleic acid w/w ratio, and/or selecting an appropriate dendrimer:lipid N:P ratio) by including a dendrimer with one or more charged, hydrophobic and/or ionisable amino acids such as arginine, aspartic acid, cysteine, glutamic acid, histidine, lysine, leucine and tyrosine. In some embodiments, the ionisable amino acid is histidine.

In some embodiments, the core peptide sequence comprises an amino acid such as arginine, aspartic acid, cysteine, glutamic acid, histidine, lysine and tyrosine. In some embodiments, the core peptide sequence comprises the ionisable amino acid histidine.

The inclusion of histidine, for example, in the core and/or peptide motifs of one or both peptide dendrimers may confer extracellular stability to a nanoparticle but aid in the intracellular release of the nucleic acid from the nanoparticle. For example, the five-member imidazole ring of histidine comprises two nitrogen atoms which can form hydrogen bonds to provide stability to the nanoparticle. However, when exposed to the acidic endosomal environment, protonation of histidine may result in endosomal swelling, lysis and release of the nucleic acid contained within the nanoparticle.

In some embodiments, the nucleic acid is RNA. For instance, the RNA may be selected from an mRNA, a circular RNA (circRNA), an ssRNA, a dsRNA, an sgRNA, a crRNA, a tracrRNA, a lncRNA, an siRNA, an saRNA and/or a self-amplifying RNA.

In some embodiments, the nucleic acid is DNA. For instance, the DNA may comprise a ssDNA, a dsDNA, a plasmid, and/or a cDNA.

To avoid any doubt; the nanoparticle may comprise more than one nucleic acid (for instance more than one type of RNA molecule). Similarly, the composition may comprise more than one lipid.

In some embodiments, the nanoparticle comprises an RNA nucleic acid and a DNA nucleic acid. The RNA nucleic acid and a DNA nucleic acid may be part of a single nucleic acid molecule.

In some embodiments, the nucleic acid comprises a modified nucleic acid. Exemplary nucleic acid modifications are described herein.

The nucleic acid may encode a transgene and can express the transgene in a target cell. The transgene may be a protein or peptide. Additionally or alternatively, the nucleic acid can modulate expression or activity of an endogenous gene. The modulation can be an increase in the expression of the gene and/or exogenous expression of further copies of the gene, or the modulation can be a decrease in the expression of the gene.

In some embodiments, the nucleic acid expresses a target transgene identified in Table A and the nanoparticle composition is for use in treating a corresponding disease identified in Table A.

TABLE A
Exemplary target diseases and corresponding target transgenes.

| Diseases | Target |
|---|---|
| Cystic fibrosis | CFTR |
| Pulmonary arterial | SERCA2A/BMPR2 |
| hypertension | |
| Rheumatoid arthritis | IRF3/IRF4/IL-35 |
| Idiopathic pulmonary | CXCL12/OSM/IL-6/CXCL10/plasminogen |
| fibrosis | activator urokinase/telomerase reverse |
| | transcriptase/A20 enzyme/IL-7/IL-27/IL-35/IL- |
| | 37/hPTX-2 |
| Type 1 diabetes | anti-MHC II: peptide complex (whole complex |
| | or peptide fragment)/IL-35 |
| Multiple sclerosis | PADRE-Kv1.3 peptide/IRF3/IRF4/IL-35 |
| Gaucher disease | GBA |
| Chronic granulmatous | CYBB |
| disease | |
| Organ transplantation | IL-35/PD-1 |
| rejection | |

In some embodiments the modulated endogenous gene is a gene that expresses a protein or peptide.

In some embodiments, the protein or peptide comprises an antigen, a hormone, a receptor, a chimeric antigen receptor, a transcription factor and/or a cytokine.

In some embodiments, the nucleic acid encodes a CAR which specifically binds to carcinoembryonic antigen (CEA) or CEA Cell Adhesion Molecule 7 (CEACAM7).

In some embodiments, the nucleic acid encodes one or more a transcription factors selected from interferon regulatory factor 5 (IRF5), activated IRF5, inhibitor of nuclear factor kappa B kinase subunit beta (IKK2), or CCAAT enhancer binding protein alpha (CEBPA).

In some embodiments, the nucleic acid encodes an anti-CEA or anti-CEACAM7 CAR and one or more transcription factors selected from interferon regulatory factor 5 (IRF5), activated IRF5, inhibitor of nuclear factor kappa B kinase subunit beta (IKK2), or CCAAT enhancer binding protein alpha (CEBPA).

In some embodiments, the nucleic acid encodes an activated IRF5 according to SEQ ID NO:7, or encoding an activated IRF5 according to SEQ ID NO:8. Activated IRF5 is particularly preferred in embodiments wherein the nucleic acid therapy is for use in treating a cancer.

In some embodiments, the transgene comprises a tumour antigen, a viral protein, a bacterial protein or a protein of a microorganism that is parasitic to a mammal.

In some embodiments, the nucleic acid comprises or encodes a self-amplifying RNA.

In some embodiments, the use comprises a treatment for a genetic disorder in the subject.

In some embodiments, the nucleic acid expresses a functional version of a gene that is non-functional, downregulated, inactive or impaired in the subject.

In some embodiments, the nucleic acid encodes and/or comprises one or more components of a system for editing a genome or a system for altering gene expression. For instance, the system for editing a genome or a system for altering gene expression may be a CRISPR/Cas system. The nucleic acid may encode a Cas protein or peptide, and/or comprises an sgRNA, a crRNA, and/or a tracrRNA. The nucleic acid may comprise an mRNA encoding a Cas protein or peptide, and an RNA sequence comprising sgRNA. The composition may comprise an mRNA that encodes a Cas protein or peptide, and another RNA comprising sgRNA (as separate molecules). In some embodiments, one or more of the sgRNA, crRNA, tracrRNA and nucleic acid encoding a Cas protein, where present, are part of a single nucleic acid. In some embodiments, one or more of the sgRNA, crRNA, tracrRNA and nucleic acid encoding a Cas protein, where present, are present on two or more nucleic acids.

It is envisaged that this invention can be used to deliver nucleic acid therapies to treat myopathies. It is also envisaged that this invention can be used to deliver nucleic acid therapies to treat muscular dystrophies such as Duchenne muscular dystrophy, myotonic dystrophy, facioscapulohumeral muscular dystrophy, Becker muscular dystrophy, limb-girdle muscular dystrophy, oculopharyngeal muscular dystrophy, Emery-Dreifuss muscular dystrophy, inheriting muscular dystrophy, congenital muscular dystrophy, and distal muscular dystrophy.

The nucleic acid therapy may be for treating muscle wasting conditions such as cachexia. The nucleic acid therapy may be for treating other muscular disorders, such as inherited muscular disorders, e.g. myotonia congenita, or familial periodic paralysis. The nucleic acid therapy may be for treating a motor neuron disease, such as ALS (amyotrophic lateral sclerosis), spinal-bulbar muscular atrophy (SBMA) or spinal muscular atrophy (SMA). The nucleic acid therapy may be for treating a mitochondrial disease, such as Friedreich's ataxia (FA), or a mitochondrial myopathy such as Kearns-Sayre syndrome (KSS), Leigh syndrome (subacute necrotizing encephalomyopathy), mitochondrial DNA depletion syndromes, mitochondrial encephalomyopathy, lactic acidosis and stroke-like episodes (MELAS), mitochondrial neurogastrointestinal encephalomyopathy (MNGIE), myoclonus epilepsy with ragged red fibers (MERRF), neuropathy, ataxia and retinitis pigmentosa (NARP), Pearson syndrome or progressive external opthalmoplegia (PEO). The nucleic acid therapy may be for treating a congenital myopathy, such as a cap myopathy, a centronuclear myopathy, a congenital myopathies with fiber type disproportion, a core myopathy, a central core disease, a multiminicore myopathies, a myosin storage myopathies, a myotubular myopathy, or a nemaline myopathy. The nucleic acid therapy may be for treating a distal myopathy, such as GNE myopathy/Nonaka myopathy/hereditary inclusion-body myopathy (HIBM), Laing distal myopathy, Markesbery-Griggs late-onset distal myopathy, Miyoshi myopathy, Udd myopathy/tibial muscular dystrophy, VCP Myopathy/IBMPFD, vocal cord and pharyngeal distal myopathy, or Welander distal myopathy. The nucleic acid therapy may be for treating an endocrine myopathy, such as hyperthyroid myopathy or hypothyroid myopathy. The nucleic acid therapy may be for treating an inflammatory myopathy such as dermatomyositis, inclusion body myositis, or polymyositis. The nucleic acid therapy may be for treating a metabolic myopathy, such as Acid maltase deficiency (AMD, Pompe disease), carnitine deficiency, carnitine palmitoyltransferase deficiency, debrancher enzyme deficiency (Cori disease, Forbes disease), lactate dehydrogenase deficiency, myoadenylate deaminase deficiency, phosphofructokinase deficiency (Tarui disease), phosphoglycerate kinase deficiency, phosphoglycerate mutase deficiency, or phosphorylase deficiency (McArdle disease). The nucleic acid therapy may be for treating a myofibrillar myopathy, or a scapuloperoneal myopathy. The nucleic acid therapy may be for treating a neuromuscular junction disease, such as congenital myasthenic syndromes (CMS), Lambert-Eaton myasthenic syndrome (LEMS), or myasthenia gravis (MG). The nucleic acid therapy may be for treating a peripheral nerve disease, such as Charcot-Marie-Tooth disease (CMT), or giant axonal neuropathy (GAN). The nucleic acid therapy may be for treating a cardiovascular disease such as Thromboangiitis obliterans/Buerger disease, diabetic peripheral neuropathy (also tested in ALS, critical limb ischemia and foot ulcers), peripheral artery disease, limb ischemia, critical limb ischemia (also known as chronic limb threatening ischemia and diabetic limb ischemia), severe peripheral artery occlusive disease (PAOD), or intermittent claudication/arteriosclerosis. The nucleic acid therapy may be for treating a cancer, such as a sarcoma, melanoma, breast cancer, lung cancer, pancreatic cancer, prostate cancer, liver cancer, acute myeloid leukaemia or B-cell lymphoma, prostate cancer or anal cancer. The nucleic acid therapy may be for treating an allergy, such as peanut allergy. The nucleic acid therapy may be for treating multiple sclerosis (MS). The nucleic acid therapy may be for treating myelodysplastic syndrome (MDS).

Pompe disease results from a defect in human acid α-glucosidase (GAA), a lysosomal enzyme that cleaves terminal α1-4 and α1-6 glucose from glycogen. The composition of the invention may be used to treat Pompe disease. The composition of the invention, comprising a nucleic acid that encodes GAA, may be administered to a subject that suffers from Pompe disease in order to deliver the nucleic acid to target tissue of the subject, to express the GAA in a target tissue described herein, particularly the liver and skeletal muscle. The enzyme may be secreted from tissues into the circulation.

Follistatin is an inhibitor of TGF-β superfamily ligands that repress skeletal muscle growth and promote muscle wasting. The composition of the invention, comprising a nucleic acid that encodes follistatin, may be administered to a subject that suffers from a muscle wasting disorder in order to deliver the nucleic acid to target tissue of the subject, to express the follistatin in a target tissue described herein, particularly the skeletal muscle. The protein may be secreted from tissues into the circulation.

Thus, this invention provides methods for treating such disorders, and compositions for use in such methods of treatment.

In some embodiments, the first peptide dendrimer comprises a structure set forth in Table 7. In some embodiments, the first peptide dendrimer comprises the structure G1-LRLR. In some embodiments, the first peptide dendrimer comprises the structure GSCG1-LRLR. In some embodiments, the first peptide dendrimer comprises the structure G1-R. In some embodiments, the first peptide dendrimer comprises the structure RHCG1-R. In some embodiments, the first peptide dendrimer comprises the structure G1-RLR. In some embodiments, the first peptide dendrimer comprises the structure RHCG1-RLR. In some embodiments, the first peptide dendrimer comprises the structure G1,2-R. In some embodiments, the first peptide dendrimer comprises the structure RHCG1,2-R. In some embodiments, the first peptide dendrimer comprises the structure G1,2-LR. In some embodiments, the first peptide dendrimer comprises the structure RHCG1,2-LR.

In some embodiments, the second peptide dendrimer comprises a structure set forth in Table 8. In some embodiments, the second peptide dendrimer comprises the structure G1,2-RL, G3-LR. In some embodiments, the second peptide dendrimer comprises the structure GSCG1,2-RL, G3-LR. In some embodiments, the second peptide dendrimer comprises the structure G1-RL, G2-LR. In some embodiments, the second peptide dendrimer comprises the structure RHCG1-RL, G2-LR. In some embodiments, the second peptide dendrimer comprises the structure G1,2-KL. In some embodiments, the second peptide dendrimer comprises the structure GSCG1,2-KL. In some embodiments, the second peptide dendrimer comprises the structure G1,2,-RL. In some embodiments, the second peptide dendrimer comprises the structure G1,2-R. In some embodiments, the second peptide dendrimer comprises the structure RHCG1,2-R. In some embodiments, the second peptide dendrimer comprises the structure G1,2-RLR. In some embodiments, the second peptide dendrimer comprises the structure RHCG1,2-RLR. In some embodiments, the second peptide dendrimer comprises the structure G1,2-LRLR. In some embodiments, the second peptide dendrimer comprises the structure GSCG1,2-LRLR. In some embodiments, the second peptide dendrimer comprises the structure G1,2-RHL. In some embodiments, the second peptide dendrimer comprises the structure GSCG1,2-RHL. In some embodiments, the second peptide dendrimer comprises the structure G1-RHL, 2-LHR. In some embodiments, the second peptide dendrimer comprises the structure GSCG1-RHL, 2-LHR.

In some embodiments, the first peptide dendrimer comprises the structure G1-LRLR and the second peptide dendrimer comprises the structure G1,2-RL, 3-LR. In some embodiments, the first peptide dendrimer comprises the structure GSCG1-LRLR and the second peptide dendrimer comprises the structure GSCG1,2-RL, 3-LR. In some embodiments, the first peptide dendrimer comprises the structure G1-R and the second peptide dendrimer comprises the structure G1-RL, 2-LR. In some embodiments, the first peptide dendrimer comprises the structure G1-R and the second peptide dendrimer comprises the structure G1-RL, 2-LR. In some embodiments, the first peptide dendrimer comprises the structure RHCG1-R and the second peptide dendrimer comprises the structure RHCG1-RL, 2-LR. In some embodiments, the first peptide dendrimer comprises the structure G1,2-R and the second peptide dendrimer comprises the structure G1-RL, 2-LR. In some embodiments, the first peptide dendrimer comprises the structure RHCG1,2-R and the second peptide dendrimer comprises the structure RHCG1-RL, 2-LR. In some embodiments, the first peptide dendrimer comprises the structure G1-RLR and the second peptide dendrimer comprises the structure G1-RL, 2-LR. In some embodiments, the first peptide dendrimer comprises the structure RHCG1-RLR and the second peptide dendrimer comprises the structure RHCG1-RL, 2-LR. In some embodiments, the first peptide dendrimer comprises the structure G1-LRLR and the second peptide dendrimer comprises the structure G1-RL, 2-LR. In some embodiments, the first peptide dendrimer comprises the structure GSCG1-LRLR and the second peptide dendrimer comprises the structure RHCG1-RL, 2-LR.

In some embodiments, the molar ratio of nitrogen contributed by each of the first peptide dendrimer and the second peptide dendrimer is about 0.1:10, 1:10, 1:2, 1:1, 2:1, 10:1, or 10:0.1. In some embodiments, the molar ratio of nitrogen contributed by each of the first peptide dendrimer and the second peptide dendrimer is 1:2, 1:1, or 2:1.

In some embodiments, delivery of the nucleic acid to a tissue or cell using a composition comprising a first and second peptide dendrimer is increased by at least 10%, 15%, 20%, 25%, 30%, 35%, 40%, 45%, 50%, 55%, 60%, 65%, 75%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, compared to delivery of the same nucleic acid to the same tissue or cell type using a composition comprising a lipid, nucleic acid and only either the first or second peptide dendrimer. The cell or tissue is a cell type or organ/tissue defined herein. For instance, the tissue may be spleen, lymphoid organs, skeletal muscle, brain and adipose tissues, as well as to lungs, tumour tissue, heart, skeletal muscle, adipose tissue, brain, liver and kidney.

In some embodiments, the first and/or second peptide dendrimer further comprises a cell penetrating peptide. The cell penetrating peptide may comprises a TAT derived sequence. The cell penetrating peptide may comprise the peptide sequence XRXRRBRRXRRBRXB (SEQ ID NO:1), where X is 6-aminohexanoic acid and B is beta-alanine.

In some embodiments, the first and/or second peptide dendrimer further comprises an alkyl chain, alkenyl chain, an antibody or a fragment thereof, a sugar, and/or a fatty acid. An alkyl or alkenyl chain may be conjugated to the core peptide sequence, for instance at the C terminus of the peptide dendrimer. Alternatively, the alkyl or alkenyl chain may be conjugated to the N terminus of the peptide dendrimer.

In some embodiments, alkyl or alkenyl chains comprise from about 5 carbons to about 50 carbons, preferably from about 12 to about 30 carbons.

In some embodiments, the first and/or second peptide dendrimer comprises a fatty acid conjugated to the C terminus of the peptide dendrimer. In other embodiments, the peptide dendrimer comprises a fatty acid conjugated to the N terminus of the peptide dendrimer.

In some embodiments, the lipid of the composition comprises a cationic lipid, a neutral lipid, an anionic lipid and/or an ionisable lipid.

In some embodiments, the lipid of the composition comprises a saturated fatty acid. Additionally or alternatively, the lipid of the composition may comprise an unsaturated fatty acid.

In some embodiments, the lipid comprises 1, 2, 3, 4, 5 or 6 fatty acid chains. Preferably, the lipid comprises 2, 3, 4 or 6 fatty acid chains.

In some embodiments, the lipid comprises dioleoylphosphatidylethanolamine (DOPE) and/or N-[1-(2,3-dioleyloxy)propyl]-N,N,N-trimethylammonium chloride (DOTMA). In some embodiments, the lipid comprises dioleoylphosphatidylethanolamine (DOPE), dioleoylphosphatidylglycerol (DOPG), DMG-PEG, and/or N-(2-hydroxyethyl)-N,N-dimethyl-2,3-bis(oleoyloxy)propan-1-aminium bromide (DORI).

In some embodiments, the peptide dendrimer, nucleic acid and lipid form a positively charged particle.

In other embodiments, the peptide dendrimer, nucleic acid and lipid form a negatively charged particle or a particle with neutral charge.

The lipid component of the composition may comprise DOTMA, DOPE, DOPC and/or DOPG.

The lipid based nucleic acid delivery system may be DOTMA/DOPE.

The amount of lipid component can be expressed in a weight:weight ratio (“w/w”, or “w:w”), with respect to the amount of the nucleic acid in the composition may be in the range of 1:50 to 50:1. More preferably, the amount of lipid (by weight) is 1:1 to 50:1, or 2:1 to 25:1 with respect to the amount of nucleic acid (by weight). The lipid:nucleic acid ratio can be at least 2:1. The weight:weight ratio of lipid:nucleic acid is about 10:1 to 25:1. These ratios refer to the weight of the total lipid. As described herein, the composition may comprise a lipid that includes more than one lipid component, e.g. a mixture of two, three or four lipids. The weight of the lipid component is the total (combined) weight of these lipid components. Preferably, each lipid component is mixed in approximately equal proportions.

Preferably, the N/P ratio, which is the amount of peptide (measured by the number of 1+ charged nitrogen atoms on the peptide, N) to the amount of nucleic acid (measured by the number of 1− charged phosphate groups in the backbone, P) is greater than 0.05:1, for instance greater than 0.1:1. (The N/P ratio terminology can be expressed as “N/P”, “N:P”, or “NP”.) In some embodiments the N/P ratio is 0.15:1, or about 0.15:1, or at least 0.15:1. In some embodiments the N/P ratio is 0.16:1, or about 0.16:1, or at least 0.16:1. In some embodiments, the N/P ratio is at least, or greater than, 1:1, for instance about 2:1 or greater, about 2.5:1 or greater, about 3:1 or greater, about 4:1 or greater, about 5:1 or greater, about 10:1 or up to 20:1. In some embodiments, the N/P ratio is about 5:1, about 8:1, about 10:1, or about 20:1. In some embodiments, the N/P ratio is in the range of about 0.01:1 and 100:1, about 2:1 to about 20:1, or about 2.5:1 to about 10:1. The first and/or second peptide dendrimer may comprise a cell penetrating peptide, an endosomal escape peptide, a nuclear localisation motif, and/or a fatty acid. The cell penetrating peptide, endosomal escape peptide, nuclear localisation motif, and/or fatty acid may be conjugated to the C-terminus of the first and/or second peptide dendrimer. Alternatively, or in addition to, the cell penetrating peptide, endosomal escape peptide, nuclear localisation motif, and/or fatty acid may be conjugated to the N-terminus of the first and/or second peptide dendrimer.

Relatedly, the invention provides a composition comprising the nanoparticle of the invention. The invention further provides a pharmaceutical composition comprising the nanoparticle of the invention and a pharmaceutically acceptable excipient. The pharmaceutical composition may be used in medicine. The pharmaceutical composition may be for use in the treatment of a cancer, an autoimmune disease, a lung disease and/or a myopathy. Also provided is a method of treating a cancer, an autoimmune disease, a lung disease and/or a myopathy, wherein the method comprises administering the pharmaceutical composition to a patient or subject. In some embodiments, the composition or pharmaceutical composition is comprised within a liquid. In other embodiments, the composition or pharmaceutical composition is provided as a dry composition, e.g. a dry powder. The dry composition may be prepared using lyophilisation and/or freeze-drying techniques.

In a further aspect, the invention provides methods of producing coated nanoparticles that can transfect target cells, by mixing a solution of peptide with a solution of preformed nanoparticles, to form the coated nanoparticle. In some instances, the preformed nanoparticles have a positive surface charge and the peptides have a net charge that is negative. In some embodiments, the peptide is a dendrimer, a PGA or a glutamic acid containing peptide. The dendrimer can be first, second or third generation and may have one, two, three, four or more amino acids in each generation and in its core sequence, as defined herein. Without being bound by theory, the surface charge and hydrophobicity may change the binding of serum components to the nanocarrier (the corona) which can influence tissue distribution. This can be achieved, for example by coating a positive nanoparticle with linear or branched PGA (as defined herein) because PGA is a negatively charged peptide. In some embodiments, the glutamic acid containing peptide comprises a glutamic acid rich domain comprising at least 4, at least 6, or at least 8 amino acid residues in total, of which at least 2 are glutamic acid, and wherein at least 20% of the amino acid residues of the glutamic acid rich domain are glutamic acid. In some embodiments, the peptide dendrimer may comprise a glutamic acid containing peptide. In some instances, the preformed nanoparticles have a negative surface charge and the dendrimers have a net charge that is positive. In some instances, the surface of the preformed nanoparticles is uncharged and the dendrimers comprise a hydrophobic region.

In some embodiments, the peptide dendrimer, PGA or glutamic acid containing peptide comprises a cell targeting motif, e.g. a myeloid, lymphoid, muscle, lung, CD206+ cell or tumor cell targeting motif. In some embodiments, the muscle cell targeting motif comprises ASSLNIA (SEQ ID NO:1), PYDQLRH (SEQ ID NO:2), or KAMHQMQ (SEQ ID NO:3) peptide motif. In some embodiments, the cell targeting motif comprises an integrin targeting motif, optionally comprising an RGD or ACDCRGDCFCG (SEQ ID NO:5) peptide motif. In some embodiments, the lung targeting motif comprises the peptide sequence CGFECVRQCPERC (SEQ ID NO:4). In still further embodiments, the cell targeting motif is a mannose receptor targeting motif, for example a mannose sugar or maltotriose. In some embodiments, the cell targeting motif targets lymphocytes, e.g. T cells. In further embodiments, the cell targeting motif may be a CD3, CD4 or CD8 binder, e.g. an antibody that specifically binds one of these markers. In some embodiments, an anti-CD3 antibody is used.

In a related aspect, the invention provides a coated nanoparticle that is able to transfect a target cell, wherein the coated nanoparticle comprises a peptide at its surface. In some embodiments, the nanoparticle is a nanoparticle according to any one of the aspects described herein. In some embodiments, the peptide at the surface of the coated nanoparticle may be a linear PGA, for example a short PGA comprising 2 to 100 glutamic acid residues, or the peptide may be a branched PGA. In some embodiments, the peptide is a glutamic acid containing peptide comprising a glutamic acid rich domain comprising at least 4, at least 6, or at least 8 amino acid residues in total, of which at least 2 are glutamic acid, and wherein at least 20% of the amino acid residues of the glutamic acid rich domain are glutamic acid. In some embodiments, the nanoparticle is coated with a dendrimer selected from Table 1 or Table 1B, or a peptide selected from Table 1A, wherein the peptide is not E100. In some embodiments, the nanoparticle is a nanoparticle according to any one of the aspects described herein that is coated with a linear PGA, a branched PGA, a glutamic acid containing peptide defined herein, a dendrimer selected from Table 1 or Table 1B, or a peptide selected from Table 1A.

In some embodiments, the linear or branched PGA, or the glutamic acid containing peptide, may further comprise a cell targeting motif. For example, the cell targeting motif may comprise a myeloid, lymphoid, muscle, lung, CD206+ cell or tumor cell targeting motif. In some embodiments, the muscle cell targeting motif comprises ASSLNIA (SEQ ID NO:1), PYDQLRH (SEQ ID NO:2), or KAMHQMQ (SEQ ID NO:3) peptide motif. In some embodiments, the cell targeting motif comprises an integrin targeting motif, optionally comprising an RGD or ACDCRGDCFCG (SEQ ID NO:5) peptide motif. In some embodiments, the lung targeting motif comprises the peptide sequence CGFECVRQCPERC (SEQ ID NO:4). In still further embodiments, the cell targeting motif is a mannose receptor targeting motif, for example a mannose sugar or maltotriose. In some embodiments, the cell targeting motif targets lymphocytes, e.g. T cells. The cell targeting motif may be a CD3, CD4 or CD8 binder, e.g. an antibody that specifically binds one of these markers. In some embodiments, an anti-CD3 antibody is used. This may be conjugated to a negatively charged polymer such as PGA or a glutamic acid containing peptide, or conjugated to a negatively charged lipid.

In related aspects, the invention provides coated nanoparticles that are able to transfect a target cell, wherein the coated nanoparticles have peptides at their surface. The nanoparticles of this aspect may be as defined according to the preceding aspects of the invention. The peptides preferably comprise a peptide dendrimer. In some instances, the peptide is PGA as defined herein.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. Third generation dendrimers are represented diagrammatically (with N-termini on the left and C-terminus on the right). The circle represents the core sequence. Each triangle represents a branching residue, such as lysine. Each rectangle represents a peptide motif. There are two peptide motifs in the first layer, four peptide motifs in the second layer, and eight peptide motifs in the third layer of the third generation dendrimer. A corresponding second generation dendrimer would lack the third layer of eight peptide motifs. A first generation dendrimer would lack the third layer of eight peptide motifs and the second layer of four peptide motifs as well as the layer of branching residues between the third and second layer.

FIG. 2. eGFP expression in mouse macrophages following transfection with peptide dendrimer/lipid nanoparticles, naked mRNA or DOTMA/DOPE-mRNA nanoparticles (LPX-mRNA). Cells were transfected with 1) eGFP mRNA alone; 2) DOTMA/DOPE and eGFP mRNA (w/w=3.2:1 lipid:mRNA); 3) GSCG1,2-RHL, DOTMA/DOPE and eGFP mRNA (N:P=0.6:1); 4) GSCG1,2-RL, 3-LR, DOTMA/DOPE and eGFP mRNA (N:P=0.16:1); or 5) RHCG1,RL, 2-LR, DOTMA/DOPE and eGFP mRNA (N:P=8:1). D/D denotes DOTMA:DOPE (w/w=10:1 to mRNA).

FIG. 3. eGFP expression in human macrophages following transfection with peptide dendrimer/lipid nanoparticles, naked mRNA or DOTMA/DOPE-mRNA nanoparticles (LPX-mRNA). Cells were either 1) untransfected or transfected with 2) eGFP mRNA alone; 3) DOTMA/DOPE and eGFP mRNA (w/w=3.2:1 lipid:mRNA); 4) GSCG1,2-RL, 3-LR, DOTMA/DOPE and eGFP mRNA (N:P=0.16:1); 5) GSCG1,2-RHL, DOTMA/DOPE and eGFP mRNA (N:P=0.6:1); 6) GSCG1-LRLR, DOTMA/DOPE and eGFP mRNA (N:P=0.6:1); 7) GSCG1,2-RHL, DOTMA/DOPE and eGFP mRNA (N:P=5:1); 8) GSCG1,2-RF, 3HL, DOTMA/DOPE and eGFP mRNA (N:P=0.6:1); or 9) RHCG1-R, DOTMA/DOPE and eGFP mRNA (N:P=0.6:1). D/D denotes DOTMA:DOPE (w/w=10:1 to mRNA). The LPX-RNA formulation comprised DOTMA:DOPE at w/w=3.2:1 lipid:mRNA.

FIG. 4. eGFP expression in mouse macrophages following transfection with peptide dendrimer/lipid nanoparticles, naked mRNA or DOTMA/DOPE-mRNA nanoparticles (LPX-mRNA). Cells were either 1) untransfected or transfected with 2) eGFP mRNA alone; 3) GSCG1,2-RHL, DOTMA/DOPE and eGFP mRNA (N:P=8:1); or 4) GSCG1,2-RHL, DOTMA/DOPE and eGFP mRNA (N:P=8:1) nanoparticles comprising a Mannose-G1-EEEE coat. The mannose-G1-EEEE dendrimer was used at 1× the mass of mRNA in the nanoparticles (1 eq). D/D denotes DOTMA:DOPE (w/w=2.5:1 to mRNA).

FIG. 5. eGFP expression in mouse macrophages following transfection with peptide dendrimer/lipid nanoparticles, naked mRNA or DOTMA/DOPE-mRNA nanoparticles (LPX-mRNA). Cells were either 1) untransfected or transfected with 2) eGFP mRNA alone; 3) DOTMA/DOPE and eGFP mRNA (w/w=3.2:1 lipid:mRNA); 4) GSCG1,2-RHL, DOTMA/DOPE and eGFP mRNA (N:P=8:1); or 5) GSCG1,2-RHL, DOTMA/DOPE and eGFP mRNA (N:P=8:1) nanoparticles comprising a Mannose-G1-EEEE coat. The mannose-G1-EEEE dendrimer was used at 0.5× the mass of mRNA in the nanoparticles (0.5 eq). D/D denotes DOTMA:DOPE (w/w=5:1 to mRNA). The LPX-RNA formulation comprised DOTMA:DOPE w/w=3.2:1 lipid:mRNA.

FIG. 6. eGFP expression in mouse macrophages following transfection with peptide dendrimer/lipid nanoparticles, naked mRNA or DOTMA/DOPE-mRNA nanoparticles (LPX-mRNA). Cells were either 1) untransfected or transfected with 2) eGFP mRNA alone; 3) DOTMA/DOPE and eGFP mRNA (w/w=3.2:1 lipid:mRNA); 4) GSCG1,2-RHL, DOTMA/DOPE and eGFP mRNA (N:P=8:1); or 5) GSCG1,2-RHL, DOTMA/DOPE and eGFP mRNA (N:P=8:1) nanoparticles comprising a Mannose-G1-EEEE coat. The mannose-G1-EEEE dendrimer was used at 1× the mass of mRNA in the nanoparticles (1 eq). D/D denotes DOTMA:DOPE (w/w=10:1 to mRNA). The LPX-RNA formulation comprised DOTMA:DOPE at w/w=3.2:1 lipid:mRNA.

FIG. 7. eGFP expression in mouse macrophages following transfection with peptide dendrimer/lipid nanoparticles, naked mRNA or DOTMA/DOPE-mRNA nanoparticles (LPX-mRNA). Cells were either 1) untransfected or transfected with 2) eGFP mRNA alone; 3) DOTMA/DOPE and eGFP mRNA (w/w=3.2:1 lipid:mRNA); 4) GSCG1,2-RHL, DOTMA/DOPE and eGFP mRNA (N:P=0.6:1); or 5) GSCG1,2-RHL, DOTMA/DOPE and eGFP mRNA (N:P=0.16:1) nanoparticles comprising a Mannose-G1-EEEE coat. The mannose-G1-EEEE dendrimer was used at 3× the mass of mRNA in the nanoparticles (3 eq). D/D denotes DOTMA:DOPE (w/w=10:1 to mRNA). The LPX-RNA formulation comprised DOTMA:DOPE at w/w=3.2:1 lipid:mRNA.

FIG. 8. eGFP expression in mouse macrophages following transfection with peptide dendrimer/lipid nanoparticles, naked mRNA or DOTMA/DOPE-mRNA nanoparticles (mRNA-LPX). Cells were either 1) untransfected (HEPES) or transfected with 2) eGFP mRNA alone; 3) DOTMA/DOPE and eGFP mRNA (w/w=3.2:1 lipid:mRNA); 4) GSCG1,2-RL, 3-LR, DOTMA/DOPE and eGFP mRNA (N:P=8:1); or 5) GSCG1,2-RL, 3-LR, DOTMA/DOPE and eGFP mRNA (N:P=8:1) nanoparticles comprising a Mannose-G1-EEEE coat. The mannose-G1-EEEE dendrimer was used at 1× the mass of mRNA in the nanoparticles (1 eq). D/D denotes DOTMA:DOPE (w/w=10:1 to mRNA). The LPX-RNA formulation comprised DOTMA:DOPE at w/w=3.2:1 lipid:mRNA.

FIG. 9. mRNA delivery to muscle cells. Mouse muscle cells (C2c12) transfected with mRNA expressing eGFP. The cells were transfected with mRNA alone, mRNA with GSCG1,2-RL, 3-LR and DOTMA:DOPE (N:P=0.6:1), mRNA with NTX2 and DOTMA:DOPE (N:P=0.6:1) or mRNA with NTX3 and DOTMA:DOPE (N:P=0.6:1). The cells were harvested 24 hours post-transfection. The relative fluorescence unit (RFU) was measured and normalised with the protein content of cells to yield RFU/mg. NTX2 is GSCG1,2-RL, 3-LR linked with a muscle cell targeting peptide; while NTX3 is GSCG1,2-RL, 3-LR linked with a muscle cell targeting peptide with 6 histidines. D/D denotes DOTMA:DOPE (w/w=10:1 to mRNA).

FIG. 10. mRNA delivery to muscle cells. Mouse muscle cells (C2c12) expressing eGFP following transfection with eGFP mRNA. The cells were transfected with mRNA alone, mRNA with RHCG1-RL, 2-RL and DOTMA:DOPE (N:P=0.6:1), mRNA with NTX5 and DOTMA:DOPE (N:P=0.6:1). The cells were harvested 24 hours post-transfection. The relative fluorescence unit (RFU) was measured and normalised with the protein content of cells to yield RFU/mg. NTX5 is RHCG1-RL, 2-LR linked with a muscle cell targeting peptide. D/D denotes DOTMA:DOPE (w/w=10:1 to mRNA).

FIG. 11. A: Luciferase expression in mice tissue following intravenous administration of compositions comprising GSCG1-LRLR with DOTMA/DOPE and mRNA (w/w=23:1, lipid to mRNA), DOTMA/DOPE and mRNA (w/w=23:1, lipid to mRNA), or mRNA alone encoding luciferase. The dendrimer composition was injected at an N:P ratio of 0.6:1. Mice were injected with the compositions and 6 hours later the tissues were harvested to measure luciferase signal in lung, spleen, liver, heart, kidney, muscle (gastrocnemius) and brain. B: Luciferase expression in the lung of mice tissue following intravenous administration of compositions comprising GSCG1,2-RHL (N:P=0.6) with DOTMA/DOPE and mRNA (w/w=23:1, lipid to mRNA), DOTMA/DOPE and mRNA (w/w=23:1, lipid to mRNA), or mRNA alone encoding luciferase. The dendrimer composition was injected at an N:P ratio of 0.6:1. Mice were injected with the compositions and 6 hours later and the luciferase signal in the lung area was measured by in vivo IVIS imaging. C: Luciferase expression in mice tissue following intravenous administration of compositions comprising GSCG1,2-RHL with DOTMA/DOPE and mRNA (w/w=23:1, lipid to mRNA) and mRNA alone encoding luciferase. The dendrimer composition was injected at an N:P ratio of 0.6:1. Mice were injected with the compositions and 6 hours later the tissues were harvested to measure luciferase signal in lung, spleen, liver, heart, kidney, muscle (gastrocnemius) and brain.

FIG. 12. A: Luciferase expression in mice tissue following intravenous administration of compositions comprising GSCG1,2-RL, 3-LR (N:P 0.16:1 to mRNA), GSCG1,2-RHL (N:P=0.6:1 to mRNA), GSCG1-LRLR (N:P=0.6:1 to mRNA) with DOTMA/DOPE (w/w=10:1 to mRNA) and mRNA expressing luciferase. mRNA alone treatment were used as controls. Mice were injected with the compositions and 6 hours later the tissues were harvested to measure luciferase signal in muscle (gastrocnemius), liver, lung, heart, spleen, kidney, adipose tissues and brain. B. Luciferase expression in mice tissue following intravenous administration of compositions comprising LPX with mRNA expressing luciferase. The LPX-RNA represents DOTMA:DOPE at w/w=3.2:1 to mRNA. BALB/c mice were injected with the compositions and 6 hours later the tissues were harvested to measure luciferase signal in muscle (gastrocnemius), liver, lung, heart, spleen, kidney, adipose tissues (adipose) and brain. C. Luciferase expression in mice tissue following intravenous administration of compositions comprising GSCG1,2-RL, 3-LR (N:P 0.16:1 to mRNA), GSCG1,2-RHL (N:P=0.6:1 to mRNA), GSCG1-LRLR (N:P=0.6:1 to mRNA) with DOTMA/DOPE (w/w=10:1 to mRNA) and mRNA expressing luciferase. mRNA alone treatment used as a control. The LPX-RNA represents DOTMA:DOPE at w/w=3.2:1 to mRNA. CD-1 mice were injected with the compositions and 6 hours later the tissues were harvested to measure luciferase signal in muscle (gastrocnemius), liver, lung, heart, spleen, kidney, adipose tissues (adipose) and brain. D. Luciferase expression in mice tissue following intravenous administration of compositions comprising LPX with mRNA expressing luciferase. The LPX-RNA represents DOTMA:DOPE at w/w=3.2:1 to mRNA. CD-1 mice were injected with the compositions and 6 hours later the tissues were harvested to measure luciferase signal in muscle (gastrocnemius), liver, lung, heart, spleen, kidney, adipose tissues (adipose) and brain.

FIG. 13. In vivo mRNA delivery to lung cells and myeloid cells in cancer model. The MC38 bearing mice were injected with either Alexafluor488 tagged mRNA with LPX (at a dose of 2.25 mg/kg, mRNA to body weight), Alexafluor488 tagged mRNA with GSCG1,2-RL, 3-LR and DOTMA:DOPE (N:P=0.16:1) (at a dose of 2.25 mg/kg, mRNA to body weight), Alexafluor488 tagged mRNA with GSCG1,2-RL, 3-LR, DOTMA:DOPE (N:P=0.16:1) and a Mannose-G1-EEEE ((Ac-EEEE)2KGSGGSGGSC[(S-S)-α-D-Thiomannose]) coat (at a dose of 2.25 mg/kg, mRNA to body weight), and Alexafluor488 tagged mRNA with GSCG1,2-RHL and DOTMA:DOPE (N:P=0.6:1) (at a dose of 2.25 mg/kg, mRNA to body weight). The mannose-G1-EEEE dendrimer was used at 3× the mass of mRNA in the nanoparticles (3 eq). D/D denotes DOTMA:DOPE (w/w=10:1 to mRNA). The LPX-RNA represents DOTMA:DOPE at w/w=3.2:1 to mRNA. Cells were isolated for flow cytometry analysis 4 hours post-injection of the formulations. (A) % of all the live cells uptake of the Alexafluor488 tagged mRNA in the lung, (B) % of all the live CD206+(mannose receptor+) cells uptake of the Alexafluor488 tagged mRNA in the lung, (C) % of all the live myeloid cells uptake of the Alexafluor488 tagged mRNA in the lung, and (D) % of all the live M2 macrophages (CD206+, mannose receptor+, expressing cells) uptake of the Alexafluor488 tagged mRNA in the lung.

FIG. 14. Luciferase and eGFP expression in HeLa or C2c12 cells following transfection with dendrimer compositions or commercially available transfection reagents. HeLa cells were transfected with 1) luciferase mRNA alone, a composition comprising GSCG1,2-RL, 3-LR (NP=0.16:1), DOTMA/DOPE (w/w=10:1 to mRNA) and luciferase mRNA or a composition comprising Lipofectamine 2000™ and Luciferase mRNA (top left panel); or 2) eGFP mRNA alone, a composition comprising GSCG1,2-RL, 3-LR (NP=0.16:1), DOTMA/DOPE (w/w=10:1 to mRNA) and eGFP mRNA or a composition comprising DLin-MC3-DMA: Cholesterol: DSPC: DMG-PEG lipid nanoparticle and eGFP mRNA (top right panel). C2c12 cells were transfected with luciferase mRNA alone, a composition comprising GSCG1,2-RL, 3-LR (NP=8:1), DOTMA/DOPE (w/w=10:1 to mRNA) and luciferase mRNA, a composition comprising Lipofectamine 2000™ and luciferase mRNA or polyethylenimine and luciferase mRNA (bottom panel). Luciferase and eGFP expression was measured 24 hours after transfection. Peptide dendrimers are more efficient at transfecting HeLa cells and C2c12 cells in vitro compared to commercially available transfection reagents, such as Lipofectamine 2000 and LNP.

FIG. 15. Comparison of the transfection efficiency of G1,2,3-RL with other commercial transfection reagents including DOTMA/DOPE, Polyethylenimine and Lipofectamine 2000 for DNA delivery. (A) Transfection in HeLa cells without the presence of serum, (B) transfection in HeLa cells with the presence of serum, (C) transfection in Neuro2A cells without the presence of serum and (D) transfection in Neuro2A cells with the presence of serum. Cells transfected with a luciferase-expressing plasmid (pCl-Luc) in the presence of GSCG1,2,3-RL with D/D (measured 24 h after transfection). The luminescence values were normalized by dividing them by the analogous values for cells treated with a D/D DNA complex to generate the % of transfection. Conditions: 1×10⁴ cells were transfected with 0.25 μg pCl-Luc for 4 hours without serum or in the presence of 10% serum. Error bars refer to the mean±SEM for experiments carried out in triplicate. GSCG1,2,3-RL mediated transfection significantly more potent than other reagents in HeLa and Neuro2A cells. * denotes p<0.05, *** denotes p<0.001 and **** p<0.0001. D/D is DOTMA/DOPE at w/w 1:1.

FIG. 16. Comparison of the transfection efficiency of dendrimers with different generations (G1, G2 and G3) with different N/P ratios and cationic residues (e.g. KL vs RL) for DNA delivery. (A) Transfection efficiency in HeLa cells, (B) transfection efficiency in Neuro2A cells. Cells transfected with a luciferase-expressing plasmid (pCl-Luc) in the presence of peptide dendrimers with D/D (measured 24 h after transfection). The luminescence values were normalized by dividing them by the analogous values for cells treated with a D/D DNA complex (w/w 1:1, 0.25 μg) to generate the % of transfection. Conditions: 1×10⁴ cells were transfected with 0.25 μg pCl-Luc. Error bars refer to the mean±SEM for experiments carried out in triplicate. * denotes p<0.05. D/D is DOTMA/DOPE at w/w 1:1. Generation 2 and generation 3 dendrimers perform better at transfecting both HeLa and Neuro2A cells compared to generation 1 dendrimers.

FIG. 17. Comparison of transfection efficacy between single dendrimer compositions and hybrid dendrimer compositions. HeLa cells were transfected with A) GSCG1,2-RL, 3-LR or a 1:2 mixture of GSCG1,2-RL, 3-LR and GSCG1-LRLR; B) RHCG1-RL, 2-LR, a 2:1 mixture of RHCG1-RL, 2-LR and RHCG1-R, or a 1:2 mixture of RHCG1-RL, 2-LR and RHCG1-R; C) RHCG1-RL, 2-LR or a 1:1 mixture of RHCG1-RL, 2-LR and RHCG1, 2-R; D) RHCG1-RL, 2-LR or a 1:1 mixture of RHCG1-RL, 2-LR and RHCG1-RLR or a 1:2 mixture of RHCG1-RL, 2-LR and RHCG1-RLR; E) RHCG1-RL, 2-LR or a 1:1 mixture of RHCG1-RL, 2-LR and GSCG1-LRLR or a 1:2 mixture of RHCG1-RL, 2-LR and GSCG1-LRLR. The final N:P ratio of all compositions was 8:1. DOTMA:DOPE was added to the mRNA complexes at a w/w=10:1. The ratio between the dendrimers is the molar ratio of the N contributed from each dendrimer. The % of transfection was calculated by normalising the transfection value of the mRNA complexes formed with 1 dendrimer to transfection value of the mRNA complexes formed with 2 dendrimers times 100%. Hybrid dendrimer systems outperform single dendrimer systems for in vitro transfection.

FIG. 18. Demonstration of transfection efficiency in C2c12 cells. Labelled mRNA delivery to cells. Mouse muscle cells (C2c12) took up mRNA labelled with an Alexa488 fluorophore following transfection. The cells were transfected with labelled mRNA alone, labelled mRNA with NTX3 (N:P=0.6:1) and DOTMA:DOPE for 4 hours, and the uptake of the mRNA was measured by flow cytometry. NTX3 is GSCG1,2-RL, 3-LR linked with a muscle cell targeting peptide with 6 histidines. DOTMA:DOPE was used at w/w=10:1 to mRNA.

FIG. 19. Demonstration of transfection efficiency in macrophages. Labelled mRNA delivery to cells. Macrophage (J774 cells) took up mRNA labelled with an Alexa488 fluorophore following transfection. The cells were transfected with labelled mRNA with GSCG1,2-RHL (N:P=0.6:1) and DOTMA:DOPE for 4 hours, and the uptake of the mRNA was measured by flow cytometry. The cells were transfected at different amount of mRNA, from 0.0015 ug to 1.5 ug. DOTMA:DOPE was used at w/w=10:1 to mRNA.

FIG. 20. Demonstration of transfection efficiency in T cells. Labelled mRNA delivery to cells. Human T cells (Jurkat cells) took up mRNA labelled with an Alexa488 fluorophore following transfection. The cells were transfected with labelled mRNA with GSCG1,2-RHL (N:P=0.6:1) and DOTMA:DOPE for 4 hours, and the uptake of the mRNA was measured by flow cytometry. The cells were transfected at different amount of mRNA, from 0.0015 ug to 1.5 ug. DOTMA:DOPE was used at w/w=10:1 to mRNA.

FIG. 21. Further demonstration of transfection efficiency in T cells. Labelled mRNA delivery to cells. HeLa cells took up mRNA labelled with an Alexa488 fluorophore following transfection. The cells were transfected with labelled mRNA with GSCG1,2-RHL (N:P=0.6:1 to mRNA) and DOTMA:DOPE for 2 hours, and the uptake of the mRNA was measured by flow cytometry. The cells were transfected at different amount of mRNA, from 0.1875 ug to 1.5 ug. DOTMA:DOPE was used at w/w=10:1 to mRNA.

FIG. 22. Transfection of HeLa cells with nanocarriers formulated with different lipid components. Nanocarriers comprising three lipid systems exhibit a higher transfection efficacy than nanocarriers comprising DOTMA:DOPE.

FIG. 23. Transfection of HeLa cells with nanocarriers formulated with different lipid components. Nanocarriers comprising three lipid systems exhibit a higher transfection efficacy than nanocarriers comprising DOTMA:DOPE.

FIG. 24. Transfection of A549 cells with nanocarriers formulated with different lipid components. Nanocarriers comprising three lipid systems exhibit a higher transfection efficacy than nanocarriers comprising DOTMA:DOPE.

FIG. 25. Dendritic PGA coating enhances delivery to T cells. Human T cells were transfected with formulations comprising mRNA expressing eGFP (alone) or with GSCG1,2-RHL and coated with either linear PGA or dendritic PGA. eGFP expression by the cells was quantified by flow cytometry and normalised to the level achieved by the formulation coated with linear PGA. Panel A (above): equivalent molar quantity of linear and dendritic PGA. Panel B (below): equivalent molar charge of linear and dendritic PGA.

FIG. 26. Enhanced targeting of undifferentiated muscle cells with particles comprising mRNA expressing eGFP. The nanocarriers were coated with molar equivalent of dendritic PGA with or without a muscle targeting domain. The particles coated with dendrimer comprising the muscle targeting domain achieve a dramatic increase in eGFP expression compared with particles coated with dendrimer without a muscle targeting domain.

FIG. 27. Enhanced targeting of differentiated muscle cells with particles comprising mRNA expressing eGFP. The nanocarriers were coated with molar equivalent of dendritic PGA with or without a muscle targeting domain. The particles coated with dendrimer comprising the muscle targeting domain achieve a dramatic increase in eGFP expression compared with particles coated with dendrimer without a muscle targeting domain.

FIG. 28. Enhanced targeting of tumour cells with particles with particles comprising mRNA expressing eGFP. The nanocarriers were coated with molar equivalent of dendritic PGA with or without an integrin targeting domain. The particles coated with dendrimer comprising the integrin targeting domain achieve a dramatic increase in eGFP expression compared with particles coated with dendrimer without an integrin targeting domain.

FIG. 29. Enhanced targeting of human T cells with particles with particles comprising mRNA expressing eGFP. The nanocarriers were coated with 1× or 3× equivalent of dendritic PGA with or without an anti-CD3 antibody (the T cell targeting domain). The particles coated with dendrimer comprising the anti-CD3 antibody achieve a dramatic increase in eGFP expression compared with particles coated with dendrimer without an anti-CD3 antibody. D/D denotes DOTMA:DOPE (w/w=10:1 to mRNA). eq refers to equivalent; hCD3 refers to human CD3; Ab refers to antibody and ctr refers to control; ITC refers to isotype control. eGFP expressing mRNA was used.

FIG. 30. Functional delivery of two mRNA molecules using the nanocarriers of the invention into cancer cells, in vitro. HeLa cells were transfected with nanocarrier mixed with two mRNAs, or with formulations of two mRNAs without the nanocarrier of the invention. Approximately 100% of cells exhibit functional expression of eGFP and/or mCherry when transfected with a nanocarrier comprising eGFP and/or mCherry respectively.

FIG. 31. Functional delivery of mRNA molecules using nanocarriers of the invention comprising two nucleic acids, into subjects in vivo. High luciferase levels were observed in lung and spleen six hours after mice were administered intravenously with nanocarriers comprising luciferase mRNA and a CpG containing nucleic acid.

FIG. 32. Transfection of primary leucocytes with nanocarriers formulated with multiple nucleic acids. Primary murine monocyte-derived dendritic cells (moDCs) were transfected with nanocarriers comprising CpG molecules and mRNA expressing eGFP. Functional expression of eGFP is observed after 2 hours transfection (panel A), 4 hours transfection (panel b) and 22 hours transfection (panel c). D/D denotes DOTMA:DOPE (w/w=10:1 to mRNA).

FIG. 33. Inducing M1 macrophages using nanocarriers of the invention. Nanocarriers were used to deliver activated IRF5 to primary M2 macrophages. Panel A is a volcano plot showing significant upregulation M1 genes and downregulation M2 genes. Panel B plots the results of a gene set enrichment analysis (GSEA) showing the number of differentially expressed genes in cells that were delivered modified IRF5 via the nanocarriers of the invention.

FIG. 34. Cytokine secretion by M1 macrophages induced using nanocarriers of the invention. Modified IRF5 was delivered to M2 macrophages using nanocarriers of the invention, to polarize them to M1 macrophages. Controls: M2 macrophages that were delivered luciferase via nanocarriers of the invention. Cytokine secretion was measured 24 hours later. Panels A and B show IL12 p70 and p40 secretion, respectively, following repolarization of mouse macrophages. Panels C and D show IL12 p70 and TNFα secretion, respectively, following repolarization of human macrophages.

FIG. 35. Delivery of therapeutic mRNA with nanocarriers of the invention significantly suppresses tumour growth. The therapeutic mRNA expresses a modified IRF5 that induces M1 macrophages. MC38 carcinoma growth was significantly suppressed.

FIG. 36. Jurkat cells were transfected with nanocarriers formulated with different lipid components. The ‘mRNA alone’ control, and the control nanocarrier comprising DOTMA:DOPE contained a 1.5 ug ‘dose’ of mRNA expressing eGFP (in a well of a 12 well plate). In contrast, the nanocarriers with test lipids contained 20% of the mRNA used in the control transfections, e.g. 0.3 ug for each test nanocarrier formulation.

FIG. 37. HeLa cells were transfected with nanocarriers formulated with different lipid components. The ‘mRNA alone’ control, and the control nanocarrier comprising DOTMA:DOPE contained a 0.25 ug ‘dose’ of mRNA expressing eGFP (in a well of a 96 well plate). In contrast, the nanocarriers with test lipids contained 67% of the mRNA used in the control transfections, e.g. 0.17 ug for each test nanocarrier formulation.

FIG. 38. A549 cells were transfected with nanocarriers formulated with different lipid components. The ‘mRNA alone’ control, and the control nanocarrier comprising DOTMA:DOPE contained a 0.25 ug ‘dose’ of mRNA expressing eGFP (in a well of a 96 well plate). In contrast, the nanocarriers with test lipids contained 67% of the mRNA used in the control transfections, e.g. 0.17 ug for each test nanocarrier formulation.

FIG. 39. In vivo mRNA delivery to myeloid cells in tumour. The MC38 tumour bearing mice were injected with either Alexafluor488 tagged mRNA with LPX (at a dose of 2.25 mg/kg, mRNA to body weight), Alexafluor488 tagged mRNA with GSCG1,2-RL, 3-LR and DOTMA:DOPE (N:P=0.16:1) (at a dose of 2.25 mg/kg, mRNA to body weight), Alexafluor488 tagged mRNA with GSCG1,2-RL, 3-LR, DOTMA:DOPE (N:P=0.16:1) and a Mannose-G1-EEEE ((Ac-EEEE)2KGSGGSGGSC[(S-S)-α-D-Thiomannose]) coat (at a dose of 2.25 mg/kg, mRNA to body weight), and Alexafluor488 tagged mRNA with GSCG1,2-RHL and DOTMA:DOPE (N:P=0.6:1) (at a dose of 2.25 mg/kg, mRNA to body weight). The mannose-G1-EEEE dendrimer was used at 3× the mass of mRNA in the nanoparticles (3 eq). DID denotes DOTMA:DOPE (w/w=10:1 to mRNA). The LPX-RNA represents DOTMA:DOPE at w/w=3.2:1 to mRNA. Cells were isolated for flow cytometry analysis 4 hours post-injection of the formulations. (A) % of all the live myeloid cells uptake of the Alexafluor488 tagged mRNA, (B) % of all the live M2 macrophages (CD206+, mannose receptor+, expressing cells) uptake of the Alexafluor488 tagged mRNA in the tumour, and (C) % of all the live myeloid derived suppressor cells (MDSCs) uptake of the Alexafluor488 tagged mRNA in the tumour.

FIG. 40. Luciferase expression in tumour of MC38 tumour bearing mice following intravenous administration of compositions comprising GSCG1,2-RHL (N:P=0.6:1 to mRNA) with DOTMA/DOPE (w/w=10:1 to mRNA) and mRNA expressing luciferase. mRNA alone treatment and LPX-RNA were used as controls. The LPX-RNA represents DOTMA:DOPE at w/w=3.2:1 to mRNA. Mice were injected with the compositions and 6 hours later and the luciferase signal in the tumour area was measured by in vivo IVIS imaging.

FIG. 41. A: Primary murine bone marrow derived macrophages were polarized to the M2 phenotypes. Cells were then transfected for 24 hours with an mRNA expressing a modified version of IRF5 protein which can polarise M2 cells to M1 cells. Control: Cells were transfected with an mRNA expressing luciferase (Control mRNA). Modified IRF5 is a protein with mutations which functions as the activated version of the WT IRF5. The formulation used was comprising GSCG1,2-RHL (N:P=0.6:1 to mRNA) with DOTMA/DOPE (w/w=10:1 to mRNA) and mRNA. The WT IRF5 and modified IRF5 protein expression level was detected by flow cytometry, and expressed as % of IRF5 expressing cells. B: Primary murine bone marrow derived macrophages were polarized to the M2 phenotypes. Cells were then transfected for 24 hours with an mRNA expressing a modified version of IRF5 protein which can polarise M2 cells to M1 cells. Control: Cells were transfected with an mRNA expressing luciferase (Control mRNA). Modified IRF5 is a protein with mutations which functions as the activated version of the WT IRF5. The formulation used was comprising GSCG1,2-RHL (N:P=0.6:1 to mRNA) with DOTMA/DOPE (w/w=10:1 to mRNA) and mRNA. The M2 cells transfected with the modified IRF5 mRNA resulted in increased expression the M1 marker (CD80), indicating M1 polarisation.

FIG. 42. Nanocarriers coated with human CD3 targeting antibody improve human T cells targeting mRNA delivery. Jurkat cells were transfected with (1) GSCG1,2-RHL and DOTMA/DOPE (N:P=0.6 to mRNA), with mRNA, (2) GSCG1,2-RHL and DOTMA/DOPE (N:P=0.6 to mRNA) with mRNA, coated with 3 equivalent of anti-CD3 antibody conjugated dendrimer, (3) GSCG1,2-RHL and DOTMA/DOPE (N:P=0.6 to mRNA) with mRNA, coated with dendrimer alone (control for (2)), (4) GSCG1,2-RHL and DOTMA/DOPE (N:P=0.6 to mRNA) with mRNA, coated with 1 equivalent of anti-CD3 antibody conjugated dendrimer and (5) GSCG1,2-RHL and DOTMA/DOPE (N:P=0.6 to mRNA) with mRNA, coated with dendrimer alone (control for (4)). DID denotes DOTMA:DOPE (w/w=10:1 to mRNA). Transfection was analysed by flow cytometry 24 hours post-transfection of the formulations. eq refers to equivalent; hCD3 refers to human CD3; Ab refers to antibody and ctr refers to control. eGFP expressing mRNA was used.

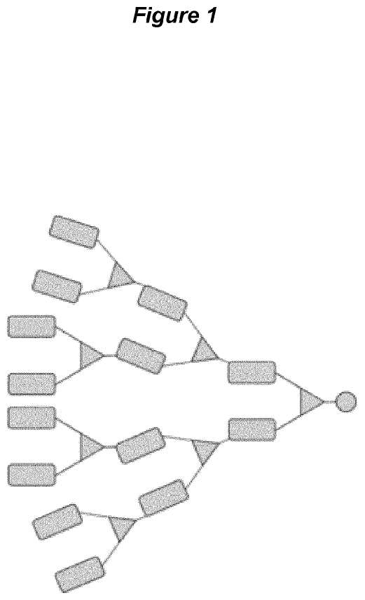

### DETAILED DESCRIPTION OF THE INVENTION

Aspects and embodiments of the present invention will now be discussed with reference to the accompanying figures and the technical definitions that follow below. Further aspects and embodiments will be apparent to those skilled in the art. All documents mentioned in this text are incorporated herein by reference.

Current immunotherapy response rates are around 15-20%, there is an urgent need to improve the treatment outcome. One strategy is to deliver mRNA to express proteins (such as CEBPA, IRF5, IRF8, cGAS-STING, SOCS1 and/or SOCS3) to revert the immunosuppressive phenotypes of myeloid cells in the tumour microenvironment, which would provide a more favourable environment for immunotherapy to be responsive. Another strategy can be to transfer mRNA into the immune cells in the tumour to express cytokines (such as IL-2, IL-7, IL-12, IL-15, IL-21 and/or interferon) to activate the immune cells to fight against cancer cells. It is also possible to deliver mRNA to the macrophage to express a chimeric antigen receptor so that the macrophage can be activated to kill tumour cells. Delivering mRNA to express tumour antigens in antigen presenting cells would help activate the immune system to attack cancer cells. These strategies can be applied to treat all tumours, especially Non-small cell lung cancers or small cell lung cancer (SCLC), Advanced melanoma, Prostate cancer, Ovarian cancer, Breast cancer, Lung cancer, Bile duct cancer (Cholangiocarcinoma), Gallbladder cancer, Neuroendocrine tumours, Hepatocellular carcinoma, Colorectal cancer, Pancreatic cancer, liver cancer, prostate cancer, thyroid cancer, a pancreatic cancer such as pancreatic ductal adenocarcinoma (PDAC), acute myeloid lymphoma (AML), myelodysplastic syndromes (MDS), colorectal cancer such as WT KRAS CRC, a KRAS mutated metastatic tumour, a haematological tumour, an oesophageal cancer, breast cancer, prostate cancer, bladder cancer, a tumour of the GI tract, head and neck squamous cell carcinomas (HNSCC), renal cancer, myelofibrosis, a CD206+ cancer, melanoma, prostate cancer or anal cancer and Solid tumours.

The compositions of the invention can be used to treat lung diseases such as cystic fibrosis, asthma, tuberculosis (TB), acute lung injury (ALI), pulmonary fibrosis, such as idiopathic pulmonary fibrosis, allergic airway disease, chronic obstructive lung disease (COPD), a lpha-1 antitrypsin deficiency (AATD), pulmonary arterial hypertension, fibrotic lung disease, chronic lung disease or a respiratory tract infection. The compositions of the invention can be used to treat muscle diseases such as a muscular dystrophy or a muscle wasting disease. The compositions of the invention can be used to treat kidney disease. The compositions of the invention can be used to treat rare genetic diseases (in haematology, neurology, amyloidosis, pulmonology, endocrinology and nephrology). The compositions of the invention can be used to deliver anti-infectives (for instance, to target macrophages to kill bacteria/deliver antibiotic payload).

The nanoparticles and compositions of the invention can be used to deliver the nucleic acids described herein to certain tissues of the human or animal body. For instance, delivery to skeletal muscle, liver, lung, heart, white and/or brown adipose tissue, brain, spleen, bone marrow, joints, kidney, gastrointestinal tract, eyes, thymus, skin, lymph nodes, pancreas, adrenal gland, testis, prostate, ovary, uterus, bladder, diaphragm, and tumours is possible. In some embodiments, the invention can be used to deliver the nucleic acids to skeletal muscle, lung, spleen, bone marrow, thymus, lymph nodes and tumours. In particular, the nanoparticles of the invention are particularly effective at targeting delivery of nucleic acid cargos to myeloid, lymphoid, muscle and lung cells.

With regards to myeloid cells, the nanoparticles of the invention can efficiently delivery nucleic acid cargo to CD206 expressing cells, such as macrophages, neutrophils, dendritic cells, endothelial cells and lymphatic cells, including specifically to M2 phenotype macrophages.

With regards to lung cells, the nanoparticles of the invention can target delivery of cargo to alveolar macrophages, ciliated cells, epithelial cells, basal cells, secretory cells, club cells, alveolar cells, fibroblasts, and/or a endothelial cells. Improved delivery of nucleic acids to lung tissue using the nanoparticles of the invention will lead to new and improved treatment options for diseases of the lung, such as cystic fibrosis.

### Cell and Tissue Targeting

The inventors have found that the nanoparticles of the invention can be specifically targeted to cells within a specific tissue with greater efficiency by altering the physical characteristics of the nanoparticles. This can be achieved in a number of ways.

For example, the nanoparticle may comprise a tissue or cell specific targeting motif. In this context, a tissue or cell specific targeting motif may be a cell-surface receptor ligand. The receptor ligand may be a protein or a fragment of a protein ligand, e.g. a peptide. The protein or peptide ligand may form part of the peptide dendrimer. For example, the protein or peptide ligand may be present in the peptide dendrimer core or one or more the peptide motifs. Alternatively, or in addition to, the protein or peptide ligand may also be present in a linear peptide included in the nanoparticle.

The receptor ligand may also be a sugar. For example, the sugar may be selected from a mannose, galactose or glucose sugar. Preferably, the sugar is a mannose. The sugar may be covalently bound to a component of the nanoparticle, for example, it may be covalently bound to the peptide dendrimer, or it may be covalently bound to a linear peptide in the nanoparticle. The sugar may also be covalently bound to a polymer (e.g. PGA) or lipid.

Regardless of the type of ligand which forms the cell- and/or tissue-targeting motif, the ligand will preferably bind to a receptor with a restricted tissue distribution. For example, the receptor may only be expressed on the target cell or within the target tissue. Alternatively, the receptor may be predominantly expressed on in a target cell type or tissue. For example, CD206 on a CD206+ cell. In an example, an M2 phenotype macrophage can be targeted for nucleic acid delivery by including a mannose ligand in the nanoparticle. The mannose ligand can target nucleic acid delivery to M2 macrophages as M2 macrophages display high levels of mannose receptor expression.

The inventors have also found that the delivery of a nucleic acid to specific cell and tissue types can be achieved by altering the lipid:nucleic acid weight/weight ratio. In particular, using a lipid:nucleic acid w/w ratio of between 2:1 and 40:1, preferably 23:1 or 10:1, specific delivery of a nucleic acid to the lungs can be achieved.

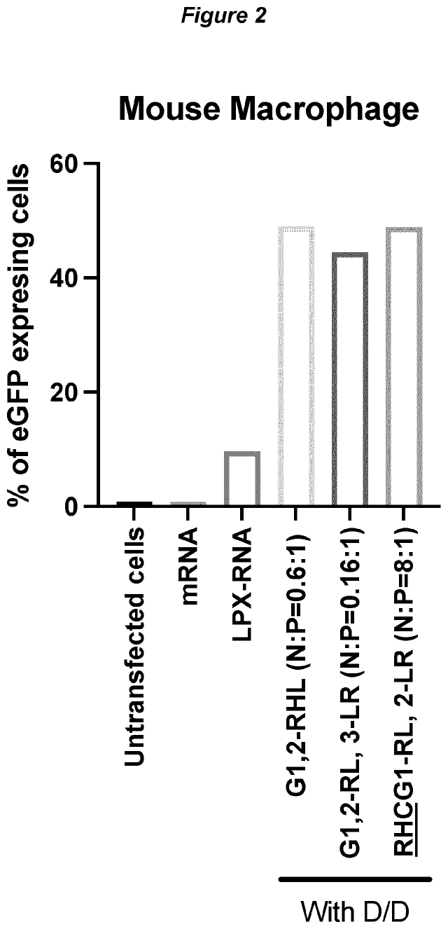

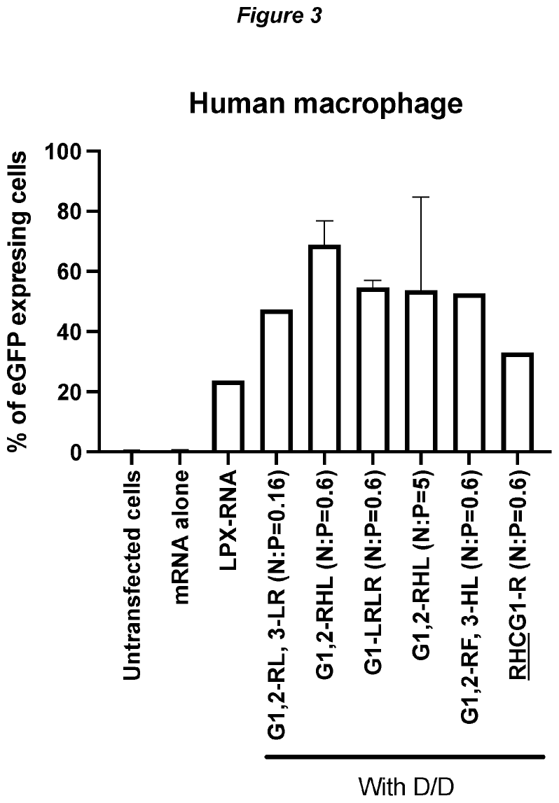

The efficiency of macrophage transfection using nanoparticles comprising one of three peptide dendrimers at varying N:P ratios was studied by transfecting human and mouse macrophages in vitro in full growth medium conditions. It was shown that each peptide dendrimer is capable of transfecting human and mouse macrophages with high efficiency (FIGS. 2 and 3). In particular, it was shown that for mouse macrophages, the peptide dendrimer/lipid nanoparticles outperformed the lipid-based delivery vector comprising only DOTMA/DOPE (FIG. 2).

Certain macrophage populations, namely M2 phenotype macrophages, have enriched expression of the mannose receptor CD206. Accordingly, in some examples the dendrimers used in the nanoparticles of the invention comprise a mannose sugar for binding to the CD206 receptor. Certain dendrimers of the invention that are particularly preferred for mRNA delivery to myeloid cells include G1,2-RHL, G1,2-RL, 3-LR, G1-RL, 2LR, G1-RL, 2-LR, and in particular GSCG1,2-RL, 3-LR, RHCG1-RL, 2-LR, GSCG1,2-RHL, GSCG1-LRLR, GSCG1,2-RF, 3-HL, or GSCG1-R. These dendrimers may be derivatized to comprise a mannose glycosylation.

Certain dendrimers of the invention that are particularly preferred for mRNA delivery to lymphoid cells include dendrimers conjugated or covalently bound to an anti-CD3 antibody, or an anti-CD3 antibody fragment.

Certain dendrimers of the invention that are particularly preferred for mRNA delivery to muscle cells include G1,2-RL,3-LR and G1-RL,2-LR, and in particular GSCG1,2-RL,3-LR and GSCG1-RL,2-LR comprising a ASSLNIA (SEQ ID NO:1) peptide motif (e.g. NTX2, NTX3, and NTX5). The NP ratio may be 0.6:1.

Certain dendrimers of the invention that are particularly preferred for mRNA delivery to lung cells include G1-LRLR, and in particular GSCG1-LRLR. The NP ratio may be 0.6:1.

### Coated Nanoparticles

In aspects that provide a nanoparticle coated with a peptide and/or dendrimer, this means that the peptide/dendrimer is present at the surface of the coated nanoparticle. This can be achieved simply by first preparing the nanoparticle (which preferably comprises one or more peptide dendrimers, together with a nucleic acid and a lipid), and then mixing the nanoparticle formulation with the peptide and/or dendrimer that is to be coated on the surface of the nanoparticle. Successful coating can be readily determined by monitoring the zeta potential of the nanoparticle, for nanoparticles with a positive or negative surface charge. The zeta potential of a positively charged nanoparticle becomes substantially less positive after it has been coated with a negatively charged peptide/dendrimer. Conversely, the zeta potential of a negatively charged nanoparticle becomes substantially less negative after it has been coated with a negatively charged peptide/dendrimer. In some embodiments, the zeta potential of the nanoparticle changes from positive to negative, or from negative to positive, following coating with a peptide/dendrimer. (In contrast, inclusion of such peptide/dendrimers throughout the nanoparticle does not substantially affect its zeta potential, which measures charge at the nanoparticle's surface.) Other analytical methods can be used, such as determining particle size before and after coating. Such methods can be used to confirm successful coating of uncharged nanoparticles with e.g. hydrophobic peptides/dendrimers.

mRNA Transfection

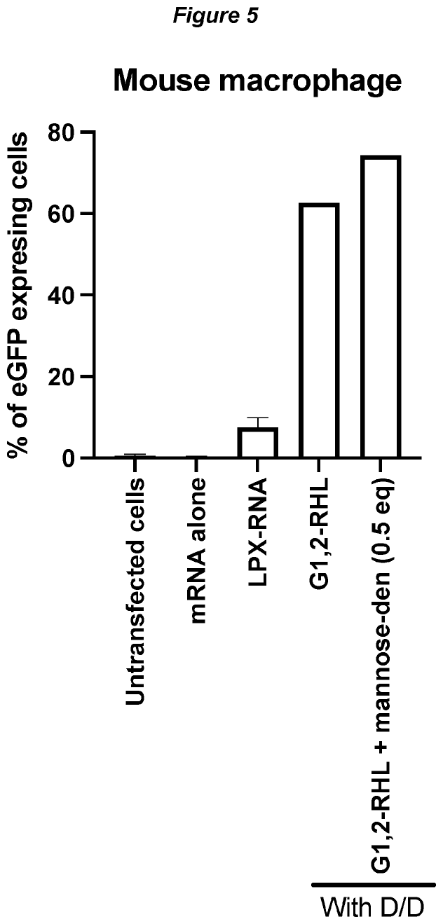

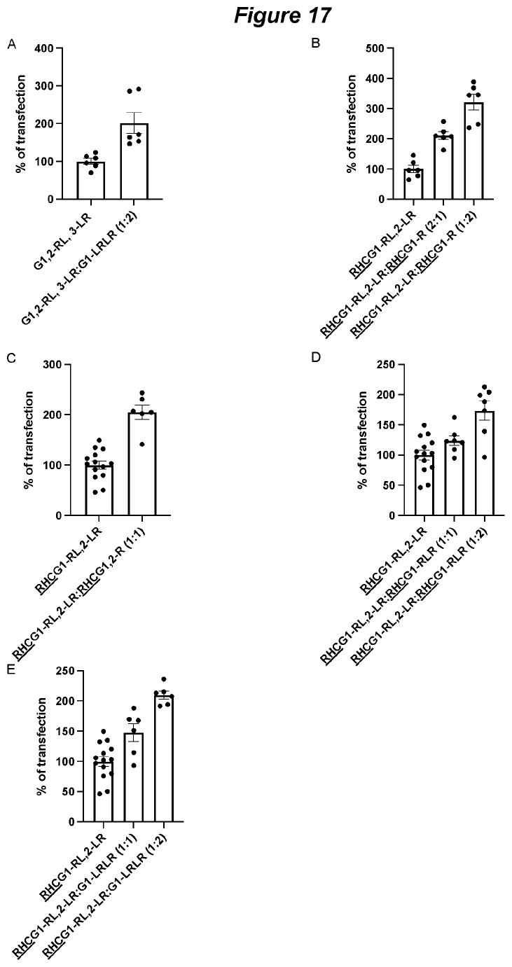

The effect of using compositions comprising two distinct peptide dendrimers for delivery of mRNA was studied by transfecting HeLa cells in full growth medium conditions. It was shown that by including two distinct peptide dendrimers in a transfection composition, transfection efficiency can be significantly increased (FIG. 17). Specifically, it was shown that by including a generation 1 (‘G1’) dendrimer in combination with a generation 2 (‘G2’) dendrimer this can result in a transfection efficiency of up to 3 times that seen when using a G2 dendrimer alone (FIG. 5B). Transfection efficiency can also be modulated in compositions comprising two distinct peptide dendrimers by altering the ratio of G2:G1 peptide dendrimer in a composition. For example, in compositions comprising the G2 dendrimer RHCG1-RL, 2-LR and the G1 dendrimer RHCG1-R, a G2:G1 ratio of 2:1 has a transfection efficiency of approximately 66% compared to a G2:G1 ratio of 1:2 (FIG. 17B).

The inventors have also studied the effects of using two distinct G2 dendrimers on transfection efficiency and found that compositions comprising, for example, the peptide dendrimers RHCG1-RL, 2-LR and RHCG1,2-R have improved transfection efficiency compared to RHCG1-RL, 2-LR alone (FIG. 17C).

Messenger RNA (mRNA)

Messenger RNA (mRNA) is a single-stranded molecule of RNA that takes the coding sequence of a gene to be translated into the corresponding amino acid sequence by a ribosome. mRNA is created during the process of transcription, where an enzyme (RNA polymerase) converts the gene into primary transcript mRNA (also known as pre-mRNA). This pre-mRNA usually still contains introns, regions that will not go on to code for the final amino acid sequence. These are removed in the process of RNA splicing, leaving only exons, regions that will encode the protein. This exon sequence constitutes mature mRNA. Mature mRNA is then read by the ribosome, thereby producing the encoded protein. The invention can be used to deliver mRNA molecules to target cells and tissues as a means of inducing expression of a desired protein or peptide. Inducing peptide/protein expression via mRNA delivery is particularly useful when transient expression is desired. To improve mRNA expression, synonymous codons within mRNA may be changed based on an organism's codon bias—i.e. the mRNA may be codon optimized. For example, the mRNA may be codon optimized for expression in the organism type of the subject to be administered the composition of the invention. For example, the mRNA may be codon optimised for expression in a mammal, for example a human.

In particular, delivery of mRNA encoding chimeric antigen receptors (CARs) and transcription factors are envisaged.

mRNAs and lncRNAs (discussed below) are typically large molecules with a negatively charged side and a hydrophobic side. mRNAs and lncRNAs will therefore require a balance between hydrophobic and hydrophilic interactions to be encapsulated and delivered to target tissues and cells. This balance between hydrophobic and hydrophilic interactions will be different from, for example, double stranded nucleic acid such as pDNA and siRNA which has charge on both sides. As mRNAs and lncRNAs are significantly larger than, for example, ASOs the requirement for encapsulation and delivery will also likely be different. As such the optimal NP ratio of dendrimer and w/w ratio of DOTMA/DOPE for mRNA and lncRNA delivery will differ compared to ASO delivery.

### Modified Nucleic Acids

Modified nucleotide bases can be used in addition to the naturally occurring bases, and may confer advantageous properties on nucleic acids containing them.

For example, modified bases may increase the stability of the nucleic acid molecule, thereby reducing the amount required. The provision of modified bases may also provide nucleic acid molecules which are more, or less, stable than unmodified nucleic acids.

The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2′-O-alkyl; 2′-O-allyl; 2′-S-alkyl; 2′-S-allyl; 2′-fluoro-; 2′-halo or azido-ribose, carbocyclic sugar analogues, a-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine,5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5-methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5-ethylcytosine, 5-butyluracil, 5-pentyluracil, 5-pentylcytosine, and 2,6,diaminopurine, methylpsuedouracil, 1-methylguanine, 1-methylcytosine.

### RNA Interference

The present invention facilitates the therapeutic down regulation of target gene expression via delivery of nucleic acids. These include RNA interference (RNAi). Small RNA molecules may be employed to regulate gene expression.

These include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 21-nt siRNA is generally long enough to induce gene-specific silencing, but short enough to evade host response. The decrease in expression of targeted gene products can be extensive with 90% silencing induced by a few molecules of siRNA.

In the art, these RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

Accordingly, the present invention provides the use of these sequences in a composition of the invention for down-regulating the expression of a target gene. For example, it is envisaged that the nanoparticles of the present invention can be used to deliver RNAi-based therapy for use in treating diabetes, for example type I or type II diabetes.

The siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed on John et al, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design suitable siRNA and miRNA sequences, for example using resources such as Ambion's online siRNA finder. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example Myers et al (2003)). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific promoter. In a further embodiment, the siRNA, longer dsRNA or miRNA is produced exogenously (in vitro) by transcription from a vector.

Alternatively, siRNA molecules may be synthesized using standard solid or solution phase synthesis techniques which are known in the art. Linkages between nucleotides may be phosphodiester bonds or alternatives, for example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—.

### Long Non-Coding RNA

Mammalian genomes are pervasively transcribed, producing a vast array of transcripts including many thousands of long non-coding RNA molecules (lncRNAs). It has been shown that lncRNAs can regulate the chromatin state, transcription, RNA stability, and the translation of certain genes. lncRNAs may be delivered to a target cell or tissue using the nanoparticles of the invention.

### RNA Activation (RNAa)

RNA activation (RNAa) is a process mediated by RNAs to enhance gene expression via a highly regulated and evolutionarily conserved pathway. RNAa can be induced by small activating RNA (saRNA), which is a class of noncoding RNA consisting of a 21-nucleotide dsRNA with 2-nucleotide overhangs at both ends. saRNA has an identical structure and chemical components to siRNA despite the fact that saRNA mediates gene activation in a sequence specific manner. To activate gene expression, the guide strand of the saRNA is loaded to AGO2, and the complex is then transported to the nucleus. Once in the nucleus, the guide strand-AGO2 complex binds directly to gene promoters or associated transcripts, recruiting key components including RNA polymerase II to initiate gene activation (Kwok et al. 2019).

### Antisense Oligonucleotides (ASOs)

Antisense oligonucleotides (ASOs) are single strands of DNA or RNA that are complementary to a target sequence. The ASO hybridises with the target nucleic acid. For instance, an ASO can be used to target a coding or non-coding RNA molecule in the cell. Following target binding, the ASO/target complex may be enzymatically degraded, e.g. by RNase H.

### Circular RNA

The present invention contemplates the use of circular RNA (circRNA) as a nucleic acid component. circRNA is a type of single-stranded RNA which forms a continuous closed loop due to a covalent bond being formed between the 5′ and 3′ ends of the RNA molecule. The closed loop structure of circRNA, and the lack of a Poly-A tail, are predicted to confer exonuclease resistance and thereby increases circRNA stability. Consequently, circRNA have an increased half-life compared to comparable non-circular RNA. For example, circRNAs which arise from a protein coding gene as an alternative splice form are more stable than the corresponding linear mRNA of the same protein coding gene. A number of functions have been ascribed to circRNAs including protein complex scaffolding, parental gene modulation, RNA-protein interactions, and microRNA sponges. Recently, it has been realised that circRNA may be useful in a range of therapeutic approaches. For example, circRNAs may be used as microRNA “sponges” to sequester microRNAs. circRNAs may also be used as sources of protein translation which can persist in cells longer than standard linear mRNAs. circRNAs may also be used to control protein activity by acting as aptamers.

### Modified Nucleic Acids

Modified nucleotide bases can be used in addition to the naturally occurring bases, and may confer advantageous properties on nucleic acids containing them.

For example, modified bases may increase the stability of the nucleic acid molecule, thereby reducing the amount required. The provision of modified bases may also provide nucleic acid molecules which are more, or less, stable than unmodified nucleic acids.

The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2′-O-alkyl; 2′-O-allyl; 2′-S-alkyl; 2′-S-allyl; 2′-fluoro-; 2′-halo or azido-ribose, carbocyclic sugar analogues, a-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine,5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5-methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5-ethylcytosine, 5-butyluracil, 5-pentyluracil, 5-pentylcytosine, and 2,6,diaminopurine, methylpsuedouracil, 1-methylguanine, 1-methylcytosine.

### Medical Therapies

The present invention contemplates use in gene therapy regimens. The use of gene therapy regimens employing either DNA or RNA, and in particular mRNA, are contemplated for use in the present invention. The nucleic acid can be present in a composition which, when introduced into target cells, results in expression of a therapeutic gene product, e.g. a transgene. Target cells include myocytes, plenocytes, hepatocytes, stellate cells, brain cells (neurons, astrocytes), splenocytes, lung cells, cardiomyocytes, kidney cells, adipose cells, stem cells, monocytes, macrophages, dendritic cells, neutrophils, B cell, T cell, myeloid derived suppressor cells, tumour associated macrophages, tumour associated neutrophils or tumour cells. In some embodiments, target cells include myocytes, plenocytes, lung cells, cardiomyocytes, stem cells, monocytes, macrophages, dendritic cells, neutrophils, B cell, T cell, myeloid derived suppressor cells, tumour associated macrophages, tumour associated neutrophils or tumour cells.

For gene therapy to be practical, it is desirable to employ a DNA/RNA transfer system that: (1) directs the therapeutic sequence into the target cell, (2) mediates uptake of the therapeutic nucleic acid into a proportion of the target cell population, and (3) is suited for use in vivo and/or ex vivo for therapeutic application.

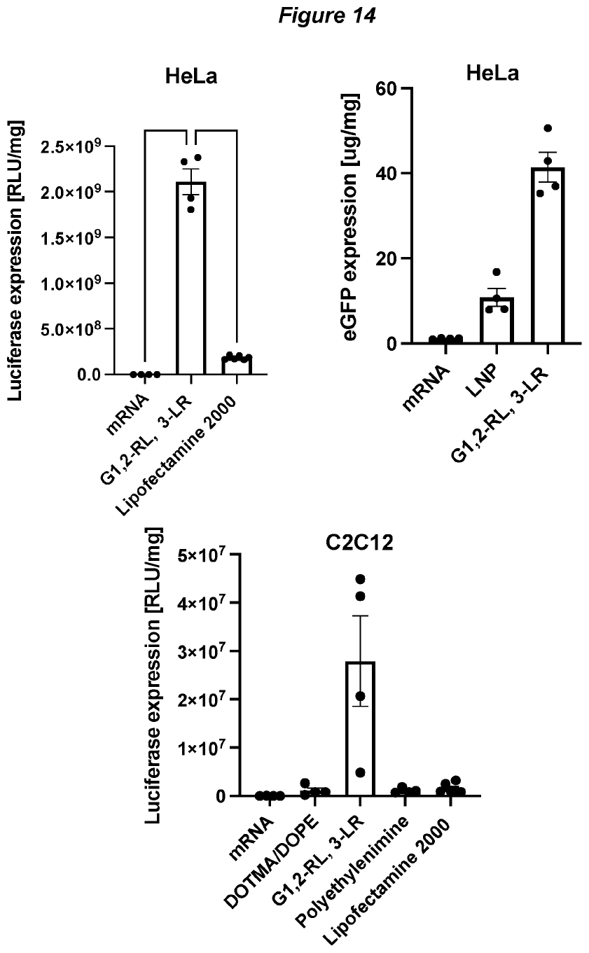

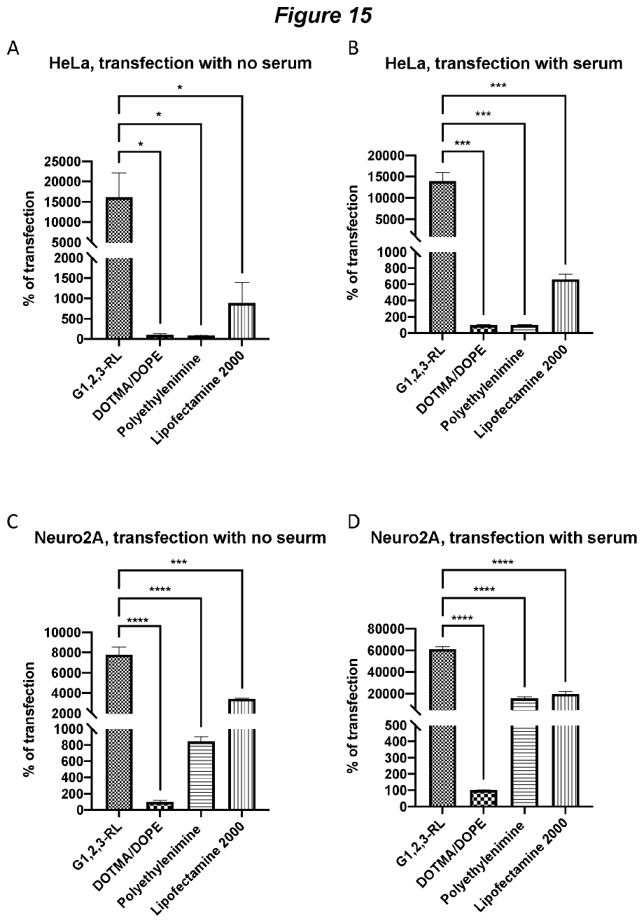

The compositions of the present invention are particularly well suited to mediating the uptake of the therapeutic nucleic acid into a relatively high proportion of a target cell population, as demonstrated in e.g., Example 9 and FIGS. 14 and 15.

Nucleic acids encoding a transgene can express the transgene in a target cell. The transgene may be a protein or peptide. Additionally, or alternatively, the nucleic acid can modulate expression or activity of an endogenous gene. The modulation can be an increase in the expression of the gene and/or exogenous expression of further copies of the gene, or the modulation can be a decrease in the expression of the gene.

The transgene may be a viral protein, a bacterial protein or a protein of a microorganism that is parasitic to a mammal. The composition expressing a viral protein, a bacterial protein or a parasitic microbial protein may be used as a vaccine. For instance, an effective amount of the composition may be delivered systemically to a subject (e.g. intravenously) to achieve expression of the viral protein, bacterial protein or parasitic microbial protein in the skeletal muscle of the subject in order to prime an immune response to that viral, bacterial or parasitic protein. Thus, this invention provides methods of vaccinating a subject, and compositions for use in the vaccination of a subject. In such examples, the transgene may be expressed in an immune cell described herein, e.g., a leucocyte, such as a B lymphocyte, a T lymphocyte, a monocyte, a neutrophil, a dendritic cell, a macrophage, or a monocyte; a lymph node tissue cell.

The transgene may be an immune molecule, for example, a T cell receptor, chimeric antigen receptor, a cytokine, a decoy receptor, an antibody, a costimulatory receptor, a costimulatory ligand, a checkpoint inhibitor, an immunoconjugate, or a tumour antigen.

The transgene may express a therapeutic protein for use in a gene therapy. The gene therapy may be for treating an autoimmune disorder, such as type I diabetes (also known as juvenile diabetes), cancers and/or a genetic disorder in a patient. The transgenes may be a function version of a gene that is non-functional, downregulated, inactive or impaired in a subject.

The genetic disorder may be monogenic disorder, e.g. muscular dystrophy in the patient. In embodiments where the monogenic disorder is a muscular dystrophy, the transgene may be dystrophin. In embodiments where the disorder is ischemia, the transgene may be hepatocyte growth factor (HGF), vascular endothelial growth factor (VEGF) and Fibroblast growth factors (FGF). In embodiments where the disorder is muscle wasting, the transgene may be follistatin. In embodiments where the disorder is a neuromuscular disease, the transgene may be acid α-glucosidase (GAA). While the transgene may be expressed in one or more of the tissues disclosed herein, the expressed protein may be secreted from the tissue(s) into the circulation.

It is envisaged that this invention can be used to deliver nucleic acid therapies to treat myopathies, It is also envisaged that this invention can be used to deliver nucleic acid therapies to treat muscular dystrophies such as Duchenne muscular dystrophy, myotonic dystrophy, facioscapulohumeral muscular dystrophy, Becker muscular dystrophy, limb-girdle muscular dystrophy, oculopharyngeal muscular dystrophy, Emery-Dreifuss muscular dystrophy, inheriting muscular dystrophy, congenital muscular dystrophy, and distal muscular dystrophy.

The nucleic acid therapy may be for treating muscle wasting conditions such as cachexia. The nucleic acid therapy may be for treating other muscular disorders, such as inherited muscular disorders, e.g. myotonia congenita, or familial periodic paralysis. The nucleic acid therapy may be for treating a motor neuron disease, such as ALS (amyotrophic lateral sclerosis), spinal-bulbar muscular atrophy (SBMA) or spinal muscular atrophy (SMA). The nucleic acid therapy may be for treating a mitochondrial disease, such as Friedreich's ataxia (FA), or a mitochondrial myopathy such as Kearns-Sayre syndrome (KSS), Leigh syndrome (subacute necrotizing encephalomyopathy), mitochondrial DNA depletion syndromes, mitochondrial encephalomyopathy, lactic acidosis and stroke-like episodes (MELAS), mitochondrial neurogastrointestinal encephalomyopathy (MNGIE), myoclonus epilepsy with ragged red fibres (MERRF), neuropathy, ataxia and retinitis pigmentosa (NARP), Pearson syndrome or progressive external opthalmoplegia (PEO). The nucleic acid therapy may be for treating a congenital myopathy, such as a cap myopathy, a centronuclear myopathy, a congenital myopathies with fiber type disproportion, a core myopathy, a central core disease, a multiminicore myopathies, a myosin storage myopathies, a myotubular myopathy, or a nemaline myopathy. The nucleic acid therapy may be for treating a distal myopathy, such as GNE myopathy/Nonaka myopathy/hereditary inclusion-body myopathy (HIBM), Laing distal myopathy, Markesbery-Griggs late-onset distal myopathy, Miyoshi myopathy, Udd myopathy/tibial muscular dystrophy, VCP Myopathy/IBMPFD, vocal cord and pharyngeal distal myopathy, or Welander distal myopathy. The nucleic acid therapy may be for treating an endocrine myopathy, such as hyperthyroid myopathy or hypothyroid myopathy. The nucleic acid therapy may be for treating an inflammatory myopathy such as dermatomyositis, inclusion body myositis, or polymyositis. The nucleic acid therapy may be for treating a metabolic myopathy, such as Acid maltase deficiency (AMD, Pompe disease), carnitine deficiency, carnitine palmitoyltransferase deficiency, debrancher enzyme deficiency (Cori disease, Forbes disease), lactate dehydrogenase deficiency, myoadenylate deaminase deficiency, phosphofructokinase deficiency (Tarui disease), phosphoglycerate kinase deficiency, phosphoglycerate mutase deficiency, or phosphorylase deficiency (McArdle disease). The nucleic acid therapy may be for treating a myofibrillar myopathy, or a scapuloperoneal myopathy. The nucleic acid therapy may be for treating a neuromuscular junction disease, such as congenital myasthenic syndromes (CMS), Lambert-Eaton myasthenic syndrome (LEMS), or myasthenia gravis (MG). The nucleic acid therapy may be for treating a peripheral nerve disease, such as Charcot-Marie-Tooth disease (CMT), or giant axonal neuropathy (GAN).

The nucleic acid therapy may be for treating a cardiovascular disease such as Thromboangiitis obliterans/Buerger disease, diabetic peripheral neuropathy (also tested in ALS, critical limb ischemia and foot ulcers), peripheral artery disease, limb ischemia, critical limb ischemia (also known as chronic limb threatening ischemia and diabetic limb ischemia), severe peripheral artery occlusive disease (PAOD), or intermittent claudication/arteriosclerosis. For example, the nucleic acid may encode one or more of the transgenes hepatocyte growth factor (HGF), vascular endothelial growth factor (VEGF) and/or Fibroblast growth factor (FGF). In particular, the transgenes hepatocyte growth factor (HGF), vascular endothelial growth factor (VEGF) and/or Fibroblast growth factor (FGF) may be useful in the treatment of a limb ischemia, such as diabetic limb ischemia, in a subject.

The nucleic acid therapy may be for treating an infectious disease, such as COVID-19, HIV, HBV, HCV, Ebola and Marburg virus, West Nile fever, SARS, avian flu, HPV, cytomegalovirus, or malaria. The nucleic acid therapy may be for treating a cancer, such as a sarcoma, melanoma, breast cancer, lung cancer, pancreatic cancer, prostate cancer, liver cancer, acute myeloid leukaemia or B-cell lymphoma. The nucleic acid therapy may be for treating an allergy, such as peanut allergy. The nucleic acid therapy may be for treating multiple sclerosis (MS). The nucleic acid therapy may be for treating myelodysplastic syndrome (MDS).

Pompe disease results from a defect in human acid α-glucosidase (GAA), a lysosomal enzyme that cleaves terminal α1-4 and α1-6 glucose from glycogen. The composition of the invention may be used to treat Pompe disease. The composition of the invention, comprising a nucleic acid that encodes GAA, may be administered to a subject that suffers from Pompe disease in order to deliver the nucleic acid to target tissue of the subject, to express the GAA in a target tissue described herein, particularly the liver and skeletal muscle. The enzyme may be secreted from tissues into the circulation.

Follistatin is an inhibitor of TGF-β superfamily ligands that repress skeletal muscle growth and promote muscle wasting. The composition of the invention, comprising a nucleic acid that encodes follistatin, may be administered to a subject that suffers from a muscle wasting disorder in order to deliver the nucleic acid to target tissue of the subject, to express the follistatin in a target tissue described herein, particularly the liver and skeletal muscle. The protein may be secreted from tissues into the circulation.

Thus, this invention provides methods for treating such disorders, and compositions for use in such treatments.

The nucleic acid-containing nanoparticles of the invention can be stored and administered in a sterile pharmaceutically acceptable carrier. Various sterile solutions may be used for administration of the composition, including water, PBS, TRIS buffers, HEPES buffers, ethanol, lipids, etc. The concentration of the DNA/RNA will be sufficient to provide a therapeutic dose, which will depend on the efficiency of transport into the cells.

Actual delivery of the composition of the invention to a patient can be carried out by a variety of techniques including direct injection, instillation of lung and other epithelial surfaces, or by intravenous injection. Administration may be by syringe needle, trocar, cannula, catheter, etc, as a bolus, a plurality of doses or extended infusion, etc. It is also envisaged that the nucleic acid cargo may be delivered to patient cells or donor cells ex vivo prior to perfusion of the patient or donor cells into a subject.

### Chimeric Antigen Receptors (CARs)

The present invention contemplates transfecting target cells with DNA/RNA encoding Chimeric antigen receptors (CARs). Target cells for CAR transfection include, but is not limited to, T cells, including γδ-T cells, Macrophages and Natural killer (NK) cells. Examples of CAR constructs envisaged by the invention include an anti-carcinoembryonic antigen (CEA) CAR, an anti-CEA Cell Adhesion Molecule 7 (CEACAM7) CAR and an anti-CEACAM5 CAR.

CARs are a class of recombinant proteins which typically comprise an antigen recognition domain, typically a single chain variable fragment (scFv), a hinge region or ectodomain, transmembrane domain and an intracellular signalling/activation domain. An scFv domain is a chimeric peptide comprising a variable light chain (VL) domain and a variable heavy chain (VH) domain of an immunoglobulin linked together such that the scFv can interact with the target antigen. Other antigen recognition domains may be used in place of scFv domains, for example, TNF receptors, innate immune receptors, cytokines, structure protein, and growth factors.

The hinge region, also known as an ectodomain or spacer, is present between the antigen recognition domain and the transmembrane domain. Ideally, the hinge region will lack FcγR binding activity. The hinge may be derived from an IgG, for example the hinge may comprise the CH2 and CH3 of an IgG. Alternatively, the hinge region may be derived from CD28, CD8α which naturally lack FcγR binding activity.

The transmembrane domain is present between the hinge region and the intracellular signalling domain. Any suitable transmembrane domain may be used in a CAR. Typically, the transmembrane domain is derived from CD3-ξ, CD4, CD8 or CD28.

The intracellular signalling domain of a CAR typically comprises a CD3-zeta cytoplasmic domain as the main intracellular signalling domain. In addition, a CAR will normally also comprise one or more co-stimulatory domains. The co-stimulatory domains may be derived from CD27, CD28, CD134 and CD137.

Binding of the antigen recognition domain of a CAR to its target antigen causes the clustering CARs. This clustering results in the transmission of an activation signal for the intracellular T-cell signalling domain, which in turn activates intracellular signalling pathways to stimulate the desired biological response.

The CAR platform was first described in T-cell based immunotherapies (CAR-T) and have been shown to successfully treat a number of haematological cancers. More recently, the CAR platform has been extended to other leukocytes such as CAR-expressing NK (CAR-NK) and γδ-T cells (CAR-γδT). The CAR platform has also been extended to myeloid cells including CAR-expressing macrophages (CAR-M) which are particularly useful at targeting and treating solid tumors.

### CAR-Macrophages: Polarising Macrophages

Macrophages in cancers often adopt an anti-inflammatory or “alternatively activated” phenotype—referred to herein as an M2 phenotype. M2 macrophages can mediate tissue repair and secrete immunoregulatory cytokines such as IL-4, IL-10, IL-13 and TGF-β. In the tumor microenvironment, chemoattractant such as CCL2 are often secreted which recruits monocytes that then go on to differentiate into M2-like macrophages which secrete the immunoregulatory cytokines IL-4, IL-10, IL-13 and TGF-ρ. M2-like macrophages in the tumor microenvironment will favour regulatory T cell function over effector T cell functions, promote vascularisation and tumorigenesis. Thus, enrichment of the tumor microenvironment with M2-like macrophages is often correlated with a poor prognosis (Sloas, C., et al 2021). It is therefore an aim of the invention to reprogramme M2 phenotype macrophages, which are enriched in the tumor microenvironment and have pro-tumor properties, to M1 phenotype macrophages which have anti-tumor properties.

Therefore, the present invention envisages transfecting M2 phenotype macrophages in situ or ex vivo, which are often enriched in the solid tumor microenvironment, and which have pro-tumor properties, with one or more transgenes suitable for reprogramming M2 phenotype macrophages to M1 phenotype macrophages. Examples of suitable transgenes include, but are not limited to, interferon regulatory factor 5 (IRF5), activated IRF5, inhibitor of nuclear factor kappa B kinase subunit beta (IKK2), or CCAAT enhancer binding protein alpha (CEBPA).

In a further example, the present invention envisages co-transfecting M2 phenotype macrophages in situ or ex vivo with one or more transgenes suitable for reprogramming M2 phenotype macrophages to M1 phenotype macrophages and a CAR construct. Suitable transgenes for reprogramming M2 phenotype macrophages include, for example, interferon regulatory factor 5 (IRF5), activated IRF5, inhibitor of nuclear factor kappa B kinase subunit beta (IKK2), or CCAAT enhancer binding protein alpha (CEBPA). Suitable CAR constructs include, for example, anti-CEA CAR, anti-CEACAM7 CAR, and anti-CEACAM5 CAR. In some examples, M2 phenotype macrophages are transfected with an anti-CEA CAR and activated IRF5. In some examples, M2 phenotype macrophages are transfected with an anti-CEACAM7 CAR and activated IRF5. In some examples, M2 phenotype macrophages are transfected with an anti-CEACAM5 CAR and activated IRF5.

It is also envisaged that the nanoparticles of the invention may be used to transfect M2 phenotype macrophages and/or M1 phenotype macrophages in vivo or ex vivo with the nanoparticles of the invention comprising a nucleic acid encoding a CAR. Examples CARs include anti-CEA CAR, anti-CEACAM7 CAR, and anti-CEACAM5 CAR. The macrophages may be patient derived or derived from a donor blood sample.

### Gene Editing

The present invention contemplates use in gene editing therapies, including gene editing therapies using technologies those well known in the art such as CRISPR/Cas (e.g. CRISPR/Cas9 systems), TALENS and Zinc finger nucleases.

In some embodiments, the CRISPR/Cas system comprises a Cas nuclease, a crispr RNA (crRNA) and a trans-activating crRNA (trRNA or tracrRNA). In this system, the crRNA comprises a sequence complementary to the target DNA and serves to direct the Cas nuclease to the target site in the genome and the tracrRNA serves as a binding scaffold for the Cas nuclease which is required for Cas activity. In some embodiments, the CRISPR/Cas system comprises a Cas nuclease and a single-guide RNA (sgRNA) to direct the Cas nuclease to the target site in the target gene. An sgRNA comprises a target-specific crRNA fused to a scaffold tracrRNA in a single nucleic acid.

In some embodiments, the nucleic acid comprises a DNA or an mRNA encoding a Cas protein or peptide, for example a Cas9 protein or peptide. In some embodiments, the nucleic acid comprises an sgRNA. In some embodiments, the nucleic acid comprises a crRNA and/or a tracrRNA. In some embodiments, the nucleic acid comprises a DNA or mRNA encoding a Cas protein or peptide, a crRNA and a tracrRNA. In some embodiments, the nucleic acid comprises a DNA or mRNA encoding a Cas protein or peptide and a sgRNA.

The CRISPR/Cas system can also be used to direct repair or modification of a target gene. For example, the CRISPR/Cas system can include a nucleic acid template to promote DNA repair or to introduce an exogenous nucleic acid sequence into the target gene by, for example, promoting homology directed repair. The CRISPR/Cas system may also be used to introduce a targeted modification to the target genomic DNA, for example using base editing technology. This can be achieved using Cas proteins fused to a base editor, such as a cytidine deaminase, as disclosed in, for example, WO2017070633A2 which is incorporated by reference. In another example, the CRISPR/Cas system may be used to “rewrite” a nucleic acid sequence in a genome. For example, the CRISPR/Cas system may be a Prime editing system. In such a prime editing system, a fusion protein may be used. For example, the fusion protein may comprise a catalytically impaired Cas domain (e.g. a “nickase”) and a reverse transcriptase. The catalytically impaired Cas domain may be capable of cutting a single strand of DNA to produce a nicked DNA duplex. A Prime editing system may include a prime editing guide RNA (pegRNA) which includes an extended sgRNA comprising a primer binding site and a reverse transcriptase template sequence. Upon nicking of the DNA duplex by the catalytically impaired Cas, the primer binding site allows the 3′ end of the nicked DNA strand to hybridize to the pegRNA, while the RT template serves as a template for the synthesis of edited genetic information.

In some embodiments, the CRISPR/Cas gene editing system may include a nucleic acid template to direct repair of the target gene of interest. In other embodiments, the Cas protein or peptide may include a base editor. In still further embodiments, the CRISPR/Cas system may be a prime editing system.

### CRISPR/Cas Gene Silencing and Gene Activation

CRISPR/Cas systems have been adapted for use in gene silencing and activation. Such systems are envisaged for use with the current invention. For example, in some embodiments, the nucleic acid may encode a fusion protein comprising a Cas protein or peptide fused to a transcriptional repressor or activator. In some embodiments, the Cas protein is catalytically dead. The fusion protein may be directed to a site of interest in the genome by either an sgRNA or a crRNA. On binding of the fusion protein to the site of interest, the transcriptional repressor or activator can regulate the expression of a gene of interest.

### Nucleic Acid-Based Vaccines

DNA vaccines, as defined by the World Health Organisation (WHO), and RNA vaccines involve the direct introduction into appropriate tissues (of the subject to be vaccinated) a plasmid containing the DNA sequence or RNA encoding the antigen(s) against which an immune response is sought and relies on the in situ production of the target antigen. These approaches offer a number of potential advantages over traditional approaches, including the stimulation of both B- and T-cell responses, improved vaccine stability, the absence of any infectious agent and the relative ease of large-scale manufacture. As proof of the principle of DNA vaccination, immune responses in animals have been obtained using genes from a variety of infectious agents, including influenza virus, hepatitis B virus, human immunodeficiency virus, rabies virus, lymphocytic chorio-meningitis virus, malarial parasites and mycoplasmas. In some cases, protection from disease in animals has also been obtained. However, the value and advantages of DNA vaccines must be assessed on a case-by-case basis and their applicability will depend on the nature of the agent being immunized against, the nature of the antigen and the type of immune response required for protection.

The field of DNA and RNA vaccination is developing rapidly. Vaccines currently being developed use not only DNA, but also include adjuncts that assist DNA to enter cells, target it towards specific cells, or that may act as adjuvants in stimulating or directing the immune response. As of 2020, the WHO noted that the first nucleic acid vaccines licensed for marketing were likely to use plasmid DNA derived from bacterial cells, but that, in future, others may use RNA or may use complexes of nucleic acid molecules and other entities. However, with the onset of the COVID-19 pandemic in 2020, a concerted effort was made to bring the first RNA-based, COVID-19 vaccines to market and these were approved for use in mid- to late-2020. Since approval, these RNA-based vaccines have been successfully rolled out worldwide to immunise the population against COVID-19.

Intramuscular delivery of DNA vaccines, in common with other vaccine technologies, is a common approach (Lim et al, 2020). The low replication rate of myocytes (muscle cells) in the skeletal muscle makes this an attractive target for DNA vaccination, because stable expression does not rely on genomic integration.

The RNA vaccines on the market currently use mRNA encoding the antigen as a payload. An area now being explored to increase the effectiveness of RNA vaccines is the use of self-amplifying RNA. self-amplifying RNA shares many of the structural features of mRNA and may include a 5′ cap, 3′ polyA tail and 5′ and 3′ untranslated regions (UTRs). In addition to encoding the antigen of interest a self-amplifying RNA will also comprise a system for self-amplification. For example, a self-amplifying RNA may also encode an RNA-dependent RNA polymerase (RDRA), a promoter and the antigen of interest. Upon translation of an RDRA by the subjects translation machinery, the RDRA can engage the self-amplifying RNA and replicate the RNA. Including a system for self-amplification reduces the minimal RNA required in a vaccine and as a result will reduce the likelihood of a subject experiencing side effects.

### Combination Therapies

Compounds of the present invention or identified by methods of the present invention may be used in the treatment of tumours and cancer in subjects in need of treatment thereof. The compounds may be administered alone or in combination with other anticancer agents.

An “anticancer agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository. Some chemotherapeutic agents are cytotoxic.

Cytotoxic chemotherapeutic agents trigger cell death via mechanisms or means that are not receptor mediated. Cytotoxic chemotherapeutic agents trigger cell death by interfering with functions that are necessary for cell division, metabolism, or cell survival. Because of this mechanism of action, cells that are growing rapidly (which means proliferating or dividing) or are active metabolically will be killed preferentially over cells that are not. The status of the different cells in the body as dividing or as using energy (which is metabolic activity to support function of the cell) determines the dose of the chemotherapeutic agent that triggers cell death. Cytotoxic chemotherapeutic agents non-exclusively relates to alkylating agents, anti-metabolites, plant alkaloids, topoisomerase inhibitors, antineoplastics and arsenic trioxide, carmustine, fludarabine, IDA ara-C, myalotang, GO, mustargen, cyclophosphamide, gemcitabine, bendamustine, total body irradiation, cytarabine, etoposide, melphalan, pentostatin and radiation. Ibrutinib (BTK inhibitor) is another anticancer agent that can be used in combination with medical applications of this invention. BTK inhibitors enhance TAM repolarisation to M1 phenotype. This combination therapy may be particularly useful for treating solid tumours, particularly ‘cold’ tumours e.g. PDAC.

Anticancer agents also include protein kinase inhibitors which can be used in the treatment of a diverse range of cancers, including blood and lung cancers. Protein kinases typically promote cell proliferation, survival and migration and are often constitutively overexpressed or active in cancer. Inhibitors of protein kinases are therefore a common drug target in the treatment of cancers.

Examples of kinase inhibitors for use in the clinic include Crizotinib, Ceritinib, Alectinib, Brigatinib, Bosutinib, Dasatinib, Imatinib, Nilotinib, Ponatinib, Vemurafenib, Dabrafenib, Ibrutinib, Palbociclib, Sorafenib, and Ribociclib.

Anticancer agents also include agents for use in immunotherapy, including antibodies. Immunotherapies can elicit, amplify, reduce or suppress an immune response depending on the specific disease context. For example, tumour cells expressing the PDL1 ligand suppress the normal immune response in a subject by binding to PD-1 receptor expressed on T cells. In this way, tumour cells resist immunity-induced apoptosis and promote tumour progression. Anti-PD-1 and anti-PDL1 antibodies have been employed successfully in the clinic to inhibit this immune checkpoint and promote immune cell-mediated killing of tumour cells. Other examples of immunotherapy include oncolytic viral therapies, T-cell therapies, and cancer vaccines.

### Pharmaceutical Compositions

The nanoparticles of the invention may be formulated as a pharmaceutical composition or formulation. The pharmaceutical compositions provided herein may include one or more pharmaceutically acceptable excipients or carriers, e.g., solvents, solubility enhancers, suspending agents, buffering agents, isotonicity agents, antioxidant, antimicrobial preservatives, diluents, binders, lubricants and disintegrants. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

When used, the excipients of the compositions will not adversely affect the stability, bioavailability, safety, and/or efficacy of the active ingredients. Thus, the skilled person will appreciate that compositions are provided wherein there is no incompatibility between any of the components of the dosage form. Excipients may be selected from the group consisting of buffering agents, tonicity agents, chelating agents, antioxidants, antimicrobial agents, and preservatives.

The nucleic acid-containing compositions of the invention can be stored and administered in a sterile pharmaceutically acceptable carrier. Various sterile solutions may be used for administration of the composition, including water, PBS, ethanol, lipids, etc. The concentration of the DNA/RNA will be sufficient to provide a therapeutic dose, which will depend on the efficiency of transport into the cells.

### Expression of Therapeutic Products

The nucleic acid that is delivered by the compositions of the invention may exhibit a therapeutic action (e.g. by acting directly to down or up regulate a target gene) or it may express a gene product (which could be a therapeutic protein or therapeutic nucleic acid) via an expression cassette comprising a coding sequence operably linked to a promoter. In this specification the term “operably linked” may include the situation where a selected nucleotide sequence and regulatory nucleotide sequence are covalently linked in such a way as to place the expression of a coding sequence under the influence or control of the regulatory sequence. Thus, a regulatory sequence is operably linked to a selected nucleotide sequence if the regulatory sequence is capable of effecting transcription of a coding sequence which forms part or all of the selected nucleotide sequence. Where appropriate, the resulting transcript may then be translated into a desired protein or polypeptide.

### Route of Administration

The compositions according to aspects of the present invention may be formulated for administration by a number of routes, including but not limited to, intravenous, parenteral, intra-arterial, intramuscular, intratumoural, subcutaneous, oral and nasal.

Actual delivery of the composition of the invention to a subject (human or animal) can be carried out by a variety of techniques including direct injection, inhalation, instillation of lung and other epithelial surfaces, or by intravenous parenteral, intra-arterial, intramuscular, intratumoural, or subcutaneous injection. Administration may be by syringe needle, trocar, cannula, catheter, etc, as a bolus, a plurality of doses or extended infusion, etc.

### Subject

The subject to be treated may be any animal or human. The subject is preferably mammalian, more preferably human. The subject may be a non-human mammal but is more preferably human. The subject may be male or female. The subject may be a patient. Therapeutic uses may be in human or animals (veterinary use).

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value.

When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1—Methods and Material for In Vitro Transfection of Cells and Tissues

In vitro macrophage transfection efficiency of eGFP mRNA can be evaluated using certain dendrimers from the table below and discussed in the following examples. Tables 1, 1A and 1B below present dendrimers and peptides that can be used in various aspects and embodiments of the invention.

TABLE 1

| Name | Dendrimer structure | MW |
|---|---|---|
| GSCG1, 2, 3-KL | (KL)₈(KKL)₄(KKL)₂KGSC-NH₂ | 4540.13 |
| GSCG1, 2-KL | (KL)₄(KKL)₂KGSC-NH₂ | 2096.80 |
| GSCG1-KL | (KL)₂KGSC-NH₂ | 875.13 |
| GSCG1, 2, 3-LR | (LR)₈(KLR)₄(KLR)₂KGSC-NH₂ | 4932.31 |
| GSCG1-LR, G2, 3-RL | (RL)₈(KRL)₄(KLR)₂KGSC-NH₂ | 4932.31 |
| GSCG1, 2, 3-rl | (rl)₈(krl)₄(krl)₂kgsc-NH₂ | 4932.31 |
| GSCG1, 2-RL, G3-LR | (LR)₈(KRL)₄(KRL)₂KGSC-NH₂ | 4932.31 |
| GSCG1, 2-LR, G3-RL | (RL)₈(KLR)₄(KLR)₂KGSC-NH₂ | 4932.31 |
| GSCG1, 2, 3-RL | (RL)₈(KRL)₄(KRL)₂KGSC-NH₂ | 4932.31 |
| GSCG1, 2, -RL | (RL)₄(KRL)₂KGSC-NH₂ | 2264.88 |
| GSCG1-RL | (RL)₂KGSC-NH₂ | 931.16 |
| GSCG1, 2-LK, G3-KL | (KL)₈(KLK)₄(KLK)₂KGSC-NH₂ | 4540.13 |
| GSCG1, 2, 3-LK | (LK)₈(KLK)₄(KLK)₂KGSC-NH₂ | 4540.13 |
| GSA-G1, 2, 3-KL | (KL)₈(KKL)₄(KKL)₂KGSA-NH₂ | 4508 |
| GSCG1, 2, 3-K | (K)₈(KK)₄(KK)₂KGSC-NH₂ | 2955.92 |
| KLCG1, 2, 3-KL | (KL)₈(KKL)₄(KKL)₂KKLC-NH₂ | 4637.33 |
| gscG1, 2, 3kl | (kl)₈(kkl)₄(kkl)₂kgsc-NH₂ | 4540.13 |
| RHCG1-R | (R)2KRHC-NH2 | 854.06 |
| RHCG1, 2-R | (R)4(KR)2KRHC-NH2 | 1735.16 |
| RHCG1, 2, 3-R | (R)8(KR)4(KR)2KRHC-NH2 | 3497.36 |
| RHCG1-LR | (LR)2KRHC-NH2 | 1080.38 |
| RHCG1-RL, 2-LR | (LR)4(KRL)2KRHC-NH2 | 2414.12 |
| RHCG1, 2-RL, 3-LR | (LR)8(KRL)4(KRL)2KRHC-NH2 | 5081.59 |
| AG1, 2-RL, 3-LR | (LR)8(KRL)4(KRL)2KA-NH2 | 4756.19 |
| RFWG1, 2-RL, 3-LR | (LR)8(KRL)4(KRL)2KRFW-NH2 | 5174.69 |
| RYMG1, 2-RL, 3-LR | (LR)8(KRL)4(KRL)2KRYM-NH2 | 5135.68 |
| YMG1, 2-RL, 3-LR | (LR)8(KRL)4(KRL)2KYM-NH2 | 4979.49 |
| LinearG1, 2-RL, 3-LR | (LR)8(KRL)4(KRL)2KGSCRHCRFWRYM-NH2 | 6269.00 |
| RHCG1-RLR | (RLR)2KRHC-NH2 | 1392.76 |
| RHCG1, 2-RLR | (RLR)4(KRLR)2KRHC-NH2 | 3351.24 |
| RHCG1, 2, 3-RLR | (RLR)8(KRLR)4(KRLR)2KRHC-NH2 | 7268.21 |
| GSCG1-LRLR | (LRLR)2KGSC-NH2 | 1469.88 |
| GSCG1, 2-LRLR | (LRLR)4(KLRLR)2KGSC-NH2 | 3881.00 |
| GSCG1, 2, 3-LRLR | (LRLR)8(KLRLR)4(KLRLR)2KGSC-NH2 | 8703.24 |
| RHCG1-RLR, 2-RL, 3-R | (R)8(KRL)4(KRLR)2KRHC-NH2 | 4488.69 |
| RHCG1-RLR, 2-R, 3-LR | (LR)8(KR)4(KRLR)2KRHC-NH2 | 4941.33 |
| RHCG1-RLRL, 2-R, 3-LR | (LR)8(KR)4(KRLRL)2KRHC-NH2 | 5167.64 |
| GSCG1-KL, 2-KH, 3-HK | (HK)8(KKH)4(KKL)2KGSC-NH2 | 4827.99 |
| GSCG1-RE, 2-RL, 3-LR | (LR)8(KRL)4(KRE)2KGSC-NH2 | 4964.30 |
| GSCG1-RF, 2-RM, 3-WR | (WR)8(KRM)4(KRF)2KGSC-NH2 | 5657.01 |
| GSCG1-RX, 2-RB, 3-BR | (BR)8(KRB)4(KRX)2KGSC-NH2 | 4427.33 |
| GSCG1-RQ, 2-RT, 3-YR | (YR)8(KRT)4(KRQ)2KGSC-NH2 | 5314.25 |
| GSCG1-RW, 2-RQ, 3-FR | (FR)8(KRQ)4(KRW)2KGSC-NH2 | 5410.52 |
| GSCG1-RY, 2-RT, 3-QR | (QR)8(KRT)4(KRY)2KGSC-NH2 | 5103.98 |
| GSCG1-RC, 2-RC, 3-MR | (MR)8(KRC)4(KRC)2KGSC-NH2 | 5016.62 |
| GSCG1-LL, 2-RR | (RR)4(KLL)2KGSC-NH2 | 2350.97 |
| gscG1, 2-rl, 3-lr (D-form | (lr)8(krl)4(krl)2kgsc-NH2 | 4932.39 |
| branching lysines) | | |
| gscG1, 2-rl, 3-lr | (lr)8(Krl)4(Krl)2Kgsc-NH2 | 4932.39 |
| GSCG1-Rl, 2-rL, 3-lR | (lR)8(KrL)4(KRL)2KGSC-NH2 | 4932.39 |
| GSCG1, 2-RL, 3-LR (Dab | (LR)8(DabRL)4(DabRL)2DabGSC-NH2 | 4736.36 |
| branching residues) | | |
| GSCG1-LR, 2, 3-RL | (RL)8(KRL)4(KLR)2KGSC-NH2 | 4932.39 |
| GSCG1, 2-RL, 3-LR | (LR)8(KRL)4(KRL)2KGSC-NH2 | 4932.39 |
| GSCG1, 2-LR, 3-RL | (RL)8(KLR)4(KLR)2KGSC-NH2 | 4932.39 |
| GSCG1, 2, 3-LR | (LR)8(KLR)4(KLR)2KGSC-NH2 | 4932.39 |
| NTX1 | (LR)8(KRL)4(KRL)2KGSCGAASSLNIAXRXRR | 7632.43 |
| | BRRXRRBRXB-NH2 | |
| NTX2 | (LR)8(KRL)4(KRL)2KGSCGAASSLNIA(Acp)-NH2 | 5830.37 |
| GSCG1, 2-RL, 3-HL | (HL)8(KRL)4(KRL)2KGSC-NH2 | 4780 |
| GSCG1, 2, 3-HL | (HL)8(KHL)4(KHL)2KGSC-NH2 | 4666 |
| GSCG1, 2-RW, 3-RL | (RL)8(KRW)4(KRW)2KGSC-NH2 | 5371 |
| GSCG1, 2-RHL | (RHL)4(KRHL)2KGSC-NH2 | 3088 |
| GSCG1, 2-RF, 3-HL | (HL)8(KRF)4(KRF)2KGSC-NH2 | 4984 |
| RHAG1-RL, 2-LR | (LR)4(KRL)2KRHA-NH2 | 2383 |
| CPP-RHCG1-RL, 2-LR | (LR)4(KRL)2KRHCR-(Acp)-RR-(ß-A)-RR-(Acp)- | 4216 |
| | RR-(ß-A)-R-(Acp)-(ß-A)-NH2 | |
| NTX5 | (LR)4(KRL)2KRHCGAASSLNIA-(Acp)-NH2 | 3312 |
| SMT1-RHCG1-RL,2-LR | (LR)4(KRL)2KRHCGAASSLNIA-(Acp)-R-(Acp)- | 5114 |
| | RR-(ß-A)-RR-(Acp)-RR-(ß-A)-R-(Acp)-(ß-A)- | |
| | NH2 | |
| SMT1-G1-CPP | R-(Acp)-RR-(ß-A)-RR-(Acp)-RR-(ß-A)-R-(Acp)- | 4647 |
| | (ß-A)2KGAASSLNIA-(Acp)-NH2 | |
| CPP-RHCG1,2-RL,3-LR | (LR)8(KRL)4(KRL)2KRHCR-(Acp)-RR-(ß-A)- | 6883 |
| | RR-(Acp)-RR-(ß-A)-R-(Acp)-(ß-A)-NH2 | |
| KG1-HR | (HR)2KK-NH2 | 860 |
| RGG1-KE, 2-RFI | (RFl)4(KKE)2KRG-NH2 | 2795 |
| ERG1-LRF, 2-SYR | (SYR)4(KLRF)2KER-NH2 | 3146 |
| LRG1-HGD, 2-RL | (RL)4(KHGD)2KLR-NH2 | 2367 |
| VRG1, 2-HVR | (HVR)4(KHVR)2KVR-NH2 | 3012 |
| GSCG1-RHL, 2-LHR | (LHR)4(KRHL)2KGSC-NH2 | 3088 |
| LRLG1-RLRL | (RLRL)2KLRL-NH2 | 1605 |
| RHCG1,2-RHL | (RHL)4(KRHL)2KRHC-NH2 | 3228 |
| NTX3 | (LR)8(KRL)4(KRL)2KGSCHHHHHHGAASSLNI | 6653 |
| | A(Acp)-NH2 | |
| GSCGARRQPPRSISSHP | (LR)8(KRL)4(KRL)2KGSCGARRQPPRSISSHP-NH2 | 6460 |
| G1, 2-RL, 3-LR | | |
| Mannose-G1-EEEE | (Ac-EEEE)2KGSGGSGGSC[(S-S)-α-D- | 2106.03 |
| | Thiomannose] | |
| Mannose-G1-RL, 2-LR | (LR)4(KRL)2KGSGGSGGSGGSC[(S-S)-a-D- | 3063 |
| | Thiomannose] | |
| Muscle-G1-EEEE | (Ac-EEEE)2KGSCGAASSLNIA-(Acp)-NH2) | 2407 |
| GSGGSGGSCG1-EEEE | (Ac-EEEE)2KGSGGSGGSC-NH2 | 1912 |
| Int-GSGGSGGSCG1- | (Ac-EEEE)2KGSGGSGGSACDCRGDCFCG- | 2936.9 |
| EEEE | NH2 (Disulfide bridges: C1-C4, C2-C3); | |
| GSEGSEGSEC(OH)G1- | (Ac-ESGESGESG)2KGSEGSEGSEC-OH | 2793 |
| (Ac)ESGESGESG | | |

Exemplary dendrimers. The NH2 groups present at the C terminus of the core sequences are
a result of the peptide synthesis method. X or Acp =6-aminohexanoic acid, B or ß-A =
beta-alanine, Dab = 2,4-Diaminobutyric acid. Lowercase letters refer to D-form amino
acids, whereas uppercase letters refers to L-form amino acids. I is the D-form leucine.
There is no D or L form for glycine. Italics represent the branching residues. Ac denotes
acetylation at the N-terminus.

TABLE 1A

| Name | Peptide structure | MW |
|---|---|---|
| PGA-100 | (E)100 | 15000 |
| Linear ESG | Ac-ESGESGESGGSEGSEGSEC | 1803 |
| Linear E8 | Ac-EEEEEEEEGSGGSGGSC | 1742 |

Exemplary peptides. Ac denotes acetylation at
the N-terminus.

TABLE 1B

| Name | Dendrimer structure | MW |
|---|---|---|
| RFWG1-LRLR, 2-RFl | (RFl)4(KLRLR)2KRFW-NH2 | 3635 |
| RHAG1, 2-RW | (RW)4(KRW)2KRHA-NH2 | 2820 |
| RFWG1-HK, 2-RFl | (RFl)4(KHK)2KRFW-NH2 | 3088 |
| RHCG1-HGD | (HGD)2KRHC-NH2 | 1160 |
| RYMG1-RLRL, 2-HVR | (HVR)4(KRLRL)2KRYM-NH2 | 3499 |
| LRG1-RFI, 2-RHL | (RHL)4(KRFl)2KLR-NH2 | 3130 |
| GSGGSGGSCG1, 2-EL, 3-LE | (Ac-LE)8(KEL)4(KEL)2KGSGGSGGSC-NH2 | 5292 |
| GSGGSGGSCG1-LELE | (Ac-LELE)2KGSGGSGGSC-NH2 | 1848 |
| EHCGGSGGSCG1-EK, 2-LE | (Ac-LE)4(KEL)2KEHCGGSGGSC-NH2 | 2898 |
| GSGGSGGSCG1, 2, -EHL | (Ac-EHL)4(KEHL)2KGSGGSGGSC-NH2 | 3496 |
| RHAG1-SYR, 2-RHL | (RHL)4(KSYR)2KRHA-NH2 | 3205 |
| RFWG1-LL, 2-LRF | (LRF)4(KLL)2KRFW-NH2 | 3010 |

Exemplary dendrimers. The NH2 groups present at the C terminus of the
core sequences are a result of the peptide synthesis method. Lowercase
letters refer to D-form amino acids, whereas uppercase letters refers
to L-form amino acids. I is the D-form leucine. There is no D or L
form for glycine. Italics represent the branching residues. Ac denotes
acetylation at the N-terminus.

### Methods

### Mouse BMDM (Bone Marrow Derived Monocytes) Polarisation

BMDM cells were isolated using the StemCell Tech EasySep Mouse Monocyte Isolation Kit from mouse bone marrow cell suspension. The cells were resuspended in complete medium (DMEM+1% glutamax+10% heat inactivated FBS+1% Pen/Strep) enriched with 50 ng/ml murine M-CSF (1:1000 of 50 μg/ml stock) at 1E+6 cells/mi. 2 million cells (i.e. 2 mL) was seeded in each well of 6-well plate(s). The plate was incubated at 37° C. for 4 days. Half media exchange was performed using complete media enriched with 50 μg/ml murine M-CSF and 40 ng/ml murine IL-4. Cells were then harvested the next day (day 5) by washing cells with PBS and a 10 minute incubation with 5 mM EDTA (diluted in PBS) at 37° C. followed by plate tapping and resuspension. The cells were resuspended in complete media containing 50 ug/ml murine M-CSF and 20 ng/ml murine IL-4 (activation media) and seeded into each well of a 24-well plate. The cells were stained with F4/80 & CD11b to confirm the macrophage phenotype.

### Mouse BMDM Transfection

The medium overlaying the polarised BMDM cells was aspirated and exchanged with 480 ul/well activation medium. 120 ul/well of each mRNA formulation was added to the cells. The cells were transfected for 24 hrs at 37° C.

### J774.2 Subculture and Macrophage Polarisation

J774.2 cells were subcultured in DMEM supplemented with 2 mM glutamax and 10% heat-inactivated FBS (complete media). Twice a week (at about 70-80% confluency; every 3-4 days), cells were scrapped and collected into Falcon tubes, and pelleted at 400 g for 5 minutes. The cells were resuspended in complete media, counted and seeded into 10 cm dishes at a density of 1.7-2.5E+4 cells/cm\hat{ }2. For M2 polarisation, the cells were treated with complete media supplemented with 20 ng/ml recombinant murine IL-4 and 20 ng/ml recombinant murine IL-13 (activation media) over a 48 hr period. After 24 hrs in IL-4 and IL-13 containing medium, the cells scrapped and collected as described above. The cells were seeded into 24-well plates at a density of 1.2E+5 cells/well in 0.5mls/well complete media containing 20 ng/ml IL-4 and IL-13.

### Transfection

The media overlaying the polarised J774.2 cells was aspirated and exchanged with 480 ul/well activation media. 120 ul/well of each test condition (total volume was 600 ul/well) was added (test conditions were prepared in 25 mM HEPES solution). Control conditions were prepared consisting of neat 25 mM HEPES and an mRNA control. Between addition of each test condition the plate was shaken back and forth to ensure mixing. The plate was placed into the incubator overnight.

### Human THP-1 Subculture and Macrophage Polarisation

THP-1 cells (a human leukaemia monocytic cell line) were sub-cultured in RPMI (with glutamax)+10% heat inactivated FBS (complete media) at 3E+5 cells/ml and passaged every 3-4 days. For macrophage polarisation 7.5E+6 cells were seeded in T75 flasks in a total of 20 ml complete media containing 5 ng/ml PMA (1:1000 of 5 ug/ml stock). The cells were transferred to the 37° C. incubator for overnight. After 24 hrs, THP-1 cells were washed with complete medium and a fresh flask complete medium (not containing PMA) was overlaid on the cells. After a further 72 hrs, the complete medium was exchanged for fresh complete media containing 20 ng/ml rhlL-4 (1:500 of 10 μg/ml stock). 24 hrs later the cells were harvested by firstly washing the cells with PBS followed by a 5 minute treatment at 37° C. with 5 mM EDTA (diluted in PBS). Cells were spun down at 300 g for 5 minutes and resuspended in a complete medium containing 20 ng/ml rhlL-4 at 2.4E+5 cells/mi. 500 ul of cells were seeded into each well on a 24-well plate and left overnight in the 37° C. incubator.

### THP-1 Transfection

The medium overlaying the polarised THP-1 cells was aspirated and exchanged with 480 ul/well complete media containing 20 ng/ml rhlL-4. 120 ul/well of each mRNA formulation was added (the formulations were prepared in 25 mM HEPES solution). Control conditions were prepared consisting of neat 25 mM HEPES and an mRNA control. The cells were transfected for 24 hrs at 37° C.

### Staining and Fixation of Polarised Macrophages

Staining and fixation for both human and mouse-derived macrophages was carried out as follows. After 24 hrs the media overlaying each test condition was transferred to corresponding 1.5 ml Eppendorf tubes (in order to conserve cells that may have died during transfection). Cells were then washed with 300 ul/well PBS which was transferred to the corresponding tubes. 500 ul/well 5 mM EDTA was added to each well and the plate(s) transferred to the 37° C. incubator for 5-10 minutes until the cells started to detach. Cells were then transferred from the plate to the corresponding 1.5 ml tubes. Cells were pelleted down at 300 g for 5 minutes and resuspended in 100 ul/sample fixable Live/Dead Aqua stain. ArC beads were stained with 3 ul neat Live/Dead Aqua for compensation. These cells were stained on ice for 30 minutes. Following this, the tubes were spun down at 300 g for 5 minutes. The supernatant was discarded, and the cells were resuspended in 500 ul/sample PBS. Negative ArC beads were added to the compensation control. GFP compensation beads were dispensed into separate tube as the GFP compensation control. The samples were spun down at 300 g for 5 minutes. Samples were then resuspended in 100 ul/sample of Reagent A from the Invitrogen Fix/Perm kit and left at room temperature for 15 minutes. The samples were spun down at 400 g for 5 minutes and the supernatant was disposed. The samples were once again washed with PBS and spun down at 300 g for 5 minutes. The cells were finally resuspended in 400 ul/sample FACS buffer (PBS+2 mM EDTA+0.5% w/v BSA) for flow analysis. Samples were stored in the fridge prior to flow analysis and run within 24 hrs of fixation.

Surface Marker Staining for BMDM and J774.2 Cells (after Live/Dead Staining and Prior to Fixation)

Cells were resuspended in 50 ul/sample 2× FcX block diluted in FACS buffer (1:50; end concentration 1:100), and incubated in fridge for 5 minutes. 50 ul/sample 2× surface antigen antibody mixes (final concentration: F4/80-PE 1:100, CD206-BV605 1:25, CD86-PECy7 1:25 & CD11b 1:200) were added on top (equal volume to block) to the FACS buffer. For each antibody used, 1 drop of UltraComp beads and 3 ul of each antibody were added on top (antibody compensation control). The cells were incubated in the fridge for 20 minutes, and spun down at 400 g for 5 minutes. Following a PBS wash, the cells were fixed and the subsequent steps were proceeded as detailed in above section.

### C2c12 Muscle Cell Culture and Transfection

Cell lines, transfection reagents and mRNA. C2c12 cells were maintained in DMEM medium with 10% (v/v) FCS and 1% (v/v) L-glutamine in a humidified atmosphere in 5% CO, and 37° C. The Alexa Fluor 488 tagged mRNA, expressing an eGFP, was purchased from RiboPro. The mRNA either expressing an eGFP or firefly luciferase was purchased from Trilink. DOTMA:DOPE, 1:1 (w/w) were obtained from Invitrogen or Encapsula NanoSciences LLC.

Transfection procedure. 24 hours before transfection, C2c12 cells were seeded in 96well plates in order to reach 70% confluence. mRNA transfection complexes were formed by mixing mRNA with the dendrimers in 25 mM HEPES buffer, then with DOTMA:DOPE in 25 mM HEPES buffer at 25° C. The transfection complexes were then overlaid to the cells in full growth medium. The cells were harvested 4 hours after transfection for FACS analysis or 24 hours post transfection for reporter genes assays.

### Cell Harvest and Staining.

Cells medium, and PBS used to wash each well, was collected from each treatment before the addition of 5 uM EDTA in PBS. After 10 minutes of incubation at 37° C., the cells were gently detached from the plates and pelleted down at 400 g for 8 minutes, 4° C. Cells were stained with LIVE/DEAD fixable aqua fluorescent reactive dye (Invitrogen) according to manufacture's instructions. ArC beads were stained with 3 ul neat Live/Dead Aqua for compensation. Following staining, the cells were pelleted down at 400 g for 8 minutes, 4° C., and resuspended in 500 ul/sample PBS wash before repeat of centrifugation. Negative ArC beads were added to the compensation control. GFP compensation beads were dispensed into separate tube as the GFP compensation control. All samples, including beads, were fixed for 15 minutes at room temperature with 4% paraformaldehyde in PBS. Following this, the samples were once again spun down at 400 g for 8 minutes, 4° C. and resuspended in FACS buffer (PBS+2 mM EDTA+0.5% w/v BSA). Samples were stored at 4° C. and flow analysis was carried out within 24 hours of fixation.

### Flow Cytometry.

Data were collected using a BD LSRFortessa I analyser running FACSDIVA software (Beckton Dickinson). All collected data were analysed using FlowJo 10.0 software.

### Nanoparticle Formulation Procedure

Tube A: Peptide dendrimer stock solution was added to a sterile 0.5 ml polypropylene tube containing 100 mM HEPES buffer (1.00 μl), sterile water (to give a final volume of 4.0 μl). The tube was shaken gently then spun down for 10-15 seconds using a mini centrifuge to ensure all liquid is at the bottom of the tube. The concentration and volume of dendrimer stock solution and water varied depending on the molecular weight and charge of the dendrimer and N/P ratio targeted. The final concentration of HEPES buffer in Tube A is 25 mM.

Tube B (800.0 μg/ml mRNA in 25 mM HEPES buffer) (Excess volume): 1 mg/ml mRNA stock solution (42.00 μl) was added to a sterile 0.5 ml polypropylene tube containing sterile water (3.94 μl) and 200 mM HEPES buffer (6.56 μl). The tube was mixed with gentle shaking then spun down for 10-15 seconds using a mini centrifuge to ensure all liquid is at the bottom of the tube.

Tube C (3076.9 μg/ml DOTMA/DOPE liposome in 25 mM HEPES buffer) (excess volume): 25 mM (17.68 mg/ml) DOTMA/DOPE liposome solution (11.88 μl) was added to a sterile 0.5 ml polypropylene falcon tube, sterile water (47.84 μl) and 200 mM HEPES buffer (8.53 μl). The tube was mixed with gentle shaking then spun down for 3-5 seconds using a mini centrifuge to ensure all liquid is at the bottom of the tube.

Mixing: 800 μg/ml mRNA solution (10 μl, 8.0 μg, 2.42×10-5 mmoles Phosphate) was mixed into tube A by rapidly pipetting up and down 10-15 times. This was allowed to sit for 2-5 minutes. 3076.9 μg/ml DOTMA/DOPE liposome solution (26.0 μl, 80.0 μg) was mixed into tube A by rapidly pipetting up and down 10-15 times.

Formulation example 1: (RHL)4(KRHL)2KGSC-NH2, N/P ratio 0.6 (4912 g/mol, 10 charges per dendrimer) for a 40 μl formulation.

Tube A (Dendrimer): to a sterile 0.5 ml polypropylene tube, 5 mg/ml dendrimer stock solution (1.430 μl, 7.14 μg, 1.45×10-6 mmoles dendrimer 1.45×10-5 mmoles N), sterile water (1.570 μl) and 100 mM HEPES buffer (1.00 μl) was added.

Formulation example 2: (RHL)4(KRHL)2KGSC-NH2, N/P ratio 8 (4912 g/mol, 10 charges per dendrimer) for a 30 ul formulation Tube A (Dendrimer): to a sterile 0.5 ml polypropylene tube, 50 mg/ml dendrimer stock solution (1.430 μl, 71.4 μg, 1.45×10-5 mmoles dendrimer 1.45×10-4 mmoles N), sterile water (0.821 μl) and 100 mM HEPES buffer (0.75 μl) was added.

### Varying Liposome/mRNA Ratio

To vary the mass ratio of liposome to mRNA in the formulation, the concentration of liposome in Tube C should be altered, such that the same volume of liposome solution is still added (i.e. 26 μl in the case of formulation with final volume of 40 μl.)

### Applyinq Mannose-Dendrimer Coating

The amount of coating applied is defined as a mass ratio of coating dendrimer with respect to mRNA in the sample. The mRNA:coating mass ratios applied are typically 1:0.5, 1:1 or 1:3.

12 μl of a nanoparticle formulation was aliquoted into a sterile 0.5 ml polypropylene tube. 4 μl of a 0.3 mg/ml, 0.6 mg/ml or 1.8 mg/ml solution of (Ac-EEEE)2KGSGGSGGSC[(S-S)-α-D-Thiomannose] (for a mRNA:coating mass ratio of 1:0.5, 1:1 or 1:3, respectively) was mixed by rapidly pipetting up and down 10-15 times, and allowed to incubate for 5 minutes.

### In Vivo Delivery of Peptide Dendrimer/Nanoparticles

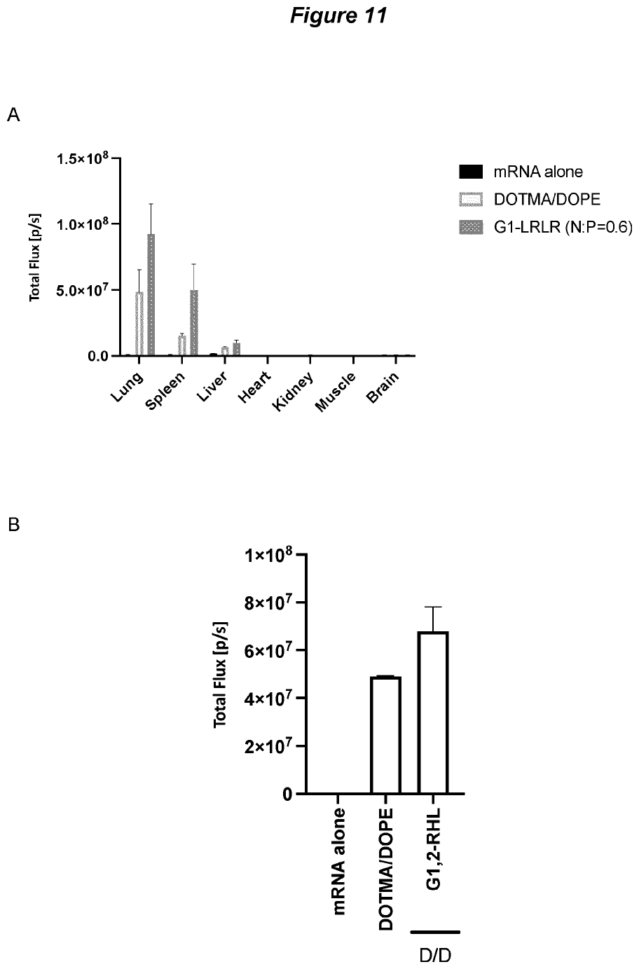

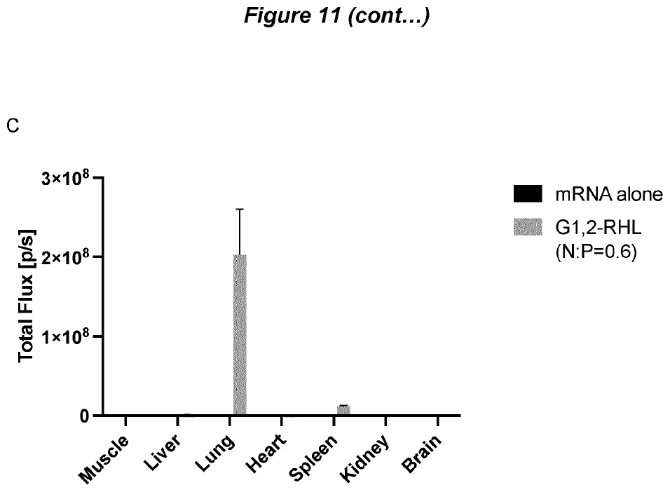

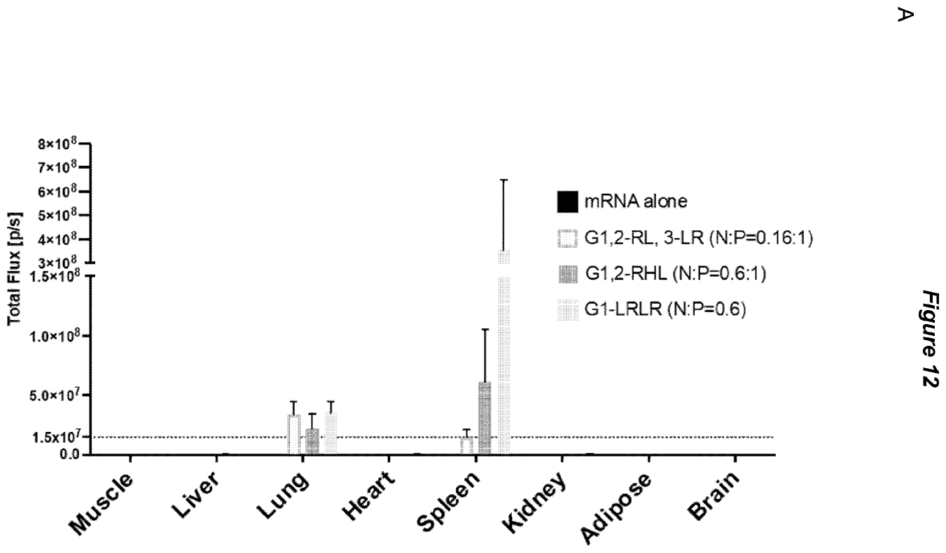

All animal procedures were conducted following the Animal Research Reporting of In Vivo Experiments and approved by the UK Home Office for the conduct of regulated procedures under license. For the data underlying FIG. 11, Balb/c mice aged 4 weeks were purchased from Jackon's Laboratory and left to acclimate in the new housing environment for 1-week upon their arrival. All mice were weighed and warmed for 15 min at 37 C in a heating chamber. The formulations were delivered intravenously (5 mL/kg) via the tail vein using 30 G insulin syringes (BD biosciences).

### Bioluminescence Imaging (BLI)

All mice were imaged 6 hours post injection. BLI was performed using an IVIS Lumina II (Perkin Elmer) imaging system. Mice were administered D-luciferin (GoldBio) at a dose of 10 uL/g. Mice were anaesthetised in a chamber with 2.5% isoflurane and then placed on a heated imaging platform while being maintained on 2.5% isoflurane. For the data underlying FIG. 11, mice were imaged 10 min post administration of D-luciferin with an exposure time set at 45 sec to ensure the signal acquired was within an effective detection range (binning medium). Bioluminescence signal was quantified by measuring photon flux (photons/s) using the Living Image software (Perkin Elmer). Following in vivo whole-body imaging, the mice were euthanised, cardiac blood taken, and the tissues extracted for ex vivo imaging. Here each tissue was placed on a dish containing D-luciferin in PBS during collection. The organs were placed in the centre of imaging platform (Lumina II system) and signal was measured using the acquisition settings detailed above. Finally, the tissues were placed in storage vials and flash frozen in liquid nitrogen. Blood was collected in EDTA-K2 tubes, spun at 10,000 rpm for 15 minutes. Plasma was transferred to fresh tube and frozen.

### Example 2—Mouse and Human Macrophage Transfection Using Peptide Dendrimers

### Mouse Macrophage Transfection

Transfection efficiency of macrophage-polarised primary bone marrow derived monocytes using various peptide dendrimer/lipid nanoparticles was assessed and compared to lipid-only nanoparticles (LPX-RNA). Each nanoparticle, with or without dendrimer, included eGFP mRNA to allow analysis of absolute numbers of cells transfected to be assessed by flow cytometry. As can be seen in FIG. 2, transfection efficiency with all peptide dendrimer/lipid based nucleic acid delivery systems tested outperforms either naked mRNA or mRNA comprised in lipid-only nanoparticles (DOTMA/DOPE lipid based nanoparticles). Lipid based nanoparticles are only able to achieve an approximate transfection efficiency of 10-15%, while transfection efficiency with GSCG1,2-RHL (NP=0.6:1), GSCG1,2-RL, 3-LR (NP=0.16:1) or GSCG1,RL, 2-LR (NP=8:1) display transfection efficiencies of approximately 35%, 40% and 50%, respectively (FIG. 2).

### Human Macrophage Transfection

Next, to test whether peptide dendrimer/lipid nanoparticles were generally useful for transfecting human cells, and in particular human macrophages, a panel of peptide dendrimer/lipid eGFP mRNA-containing nanoparticles were tested for transfection efficiency of human THP-1 cells. As was seen with primary mouse macrophage cells, each of the tested dendrimers is able to transfect THP-1 cells with high efficiency, and in each case exceeding the efficiency demonstrated by lipid-only nanoparticles (FIG. 3). GSCG1,2-RL, 3-LR (NP=0.16:1), GSCG1-LRLR (NP=0.6), GSCG1,2-RHL (NP=5:1) and GSCG1,2-RF, 3HL (NP=0.6:1) can each achieve a transfection efficiency of between 50% and 60%, while using lipid-only nanoparticles only approximately 20% of cells express eGFP following transfection. Interestingly, G1,2-RHL nanoparticles having a lower NP ratio (NP=0.6:1 compared to NP=5:1) can further increase the transfection efficiency of these nanoparticles by approximately 10%. RHCG1-R (NP=0.6:1) also outperforms lipid-only nanoparticles achieving a transfection efficiency of ˜30-35%.

In view of the primary macrophage mouse transfection experiments and the human macrophage transfection experiments it is clear that peptide dendrimer/lipid nanoparticles offer an improved mechanism to achieve transfection of macrophages.

### Example 3—Mannose-Conjugated Peptide Dendrimers Increases Transfection Efficiency

M2-phenotype macrophages are highly enriched in many solid tumors and are known to be anti-inflammatory, promote regulatory T cell functions, and promote vascularisation and tumorigenesis. Typically, patients with a high M2-phenotype macrophage content in a tumor have poor prognosis.

With the above in mind, the inventors next investigated whether macrophage transfection efficiencies could be further improved by targeting the peptide dendrimer/lipid nanoparticles to bind a specific receptor on macrophages. Macrophages, and in particular M2-phenotype macrophages, are known to have enriched surface expression of the receptor CD206, the ligand for which is mannose. Therefore, various peptide dendrimer/lipid nanoparticles were coated with a second peptide dendrimer conjugated to a mannose sugar to investigate whether this could improve delivery of mRNA cargos to macrophages.

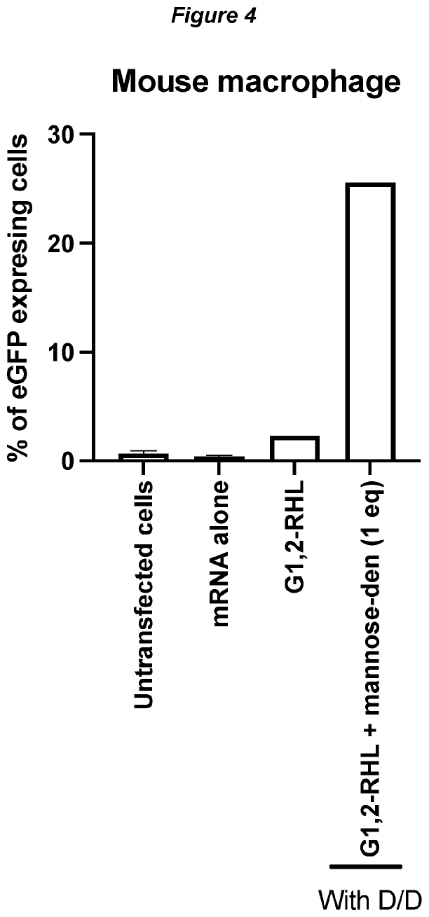

GSCG1,2-RHL/lipid nanoparticles (NP=8:1, with a lipid:mRNA w/w ratio of 2.5:1) were seen to perform poorly and transfect macrophages at a very low level of ˜2-3% (FIG. 4). This poor performance can be attributed to the lipid:mRNA w/w ratio of 2.5:1 in this formulation as, generally, GSCG1,2-RHL/lipid nanoparticles transfect macrophages with a high efficiency when the lipid:mRNA ratio is increased (see, for example, FIGS. 2, 3, and 5-7). However, even when a suboptimal lipid:mRNA ratio is used, coating the nanoparticles with (Ac-EEEE)2KGSGGSGGSC[(S-S)-α-D-Thiomannose], at a w/w ratio of 1:1 (1 eq) with respect to coating:mRNA, an increase of transfection efficiency from ˜2-3% to ˜25% can be achieved (FIG. 4). Thus, it appears that even “poorly performing” peptide dendrimer/lipid nanoparticles can show improved mRNA delivery to macrophages by coating the nanoparticle with a mannose sugar.

Using a further optimised GSCG1,2-RHL/lipid nanoparticle composition (NP=8:1, lipid:mRNA w/w ratio of 5:1), GSCG1,2-RHL containing nanoparticles can transfect macrophages with higher efficiency than when using lipid-only nanoparticles (FIG. 5; ˜60% vs ˜10%). Transfection efficiency can be further improved by applying the same dendrimer-mannose coat to the nanoparticles, at a w/w ratio of 0.5:1 (0.5 eq) with respect to coating:mRNA. Using this dendrimer-mannose coat, transfection efficiencies are further increased from ˜60% to ˜75% (FIG. 5). This further demonstrates that coating a dendrimer/lipid nanoparticle with a ligand for a receptor expressed on a target cell can increase transfection efficiencies.

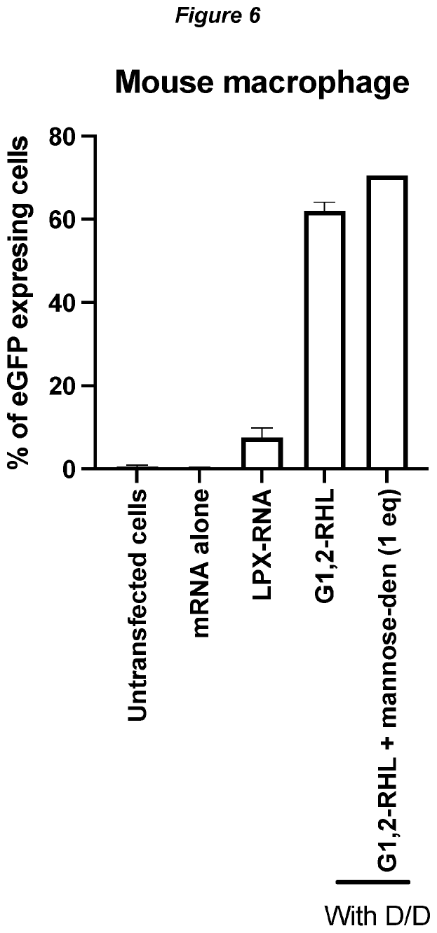

Next, it was investigated whether further increasing the DOTMA/DOPE:mRNA w/w ratio from 5:1 (FIG. 5) to 10:1 (FIG. 6) could further improve transfection efficiencies of GSCG1,2-RHL/lipid nanoparticles. Using a DOTMA/DOPE:mRNA w/w ratio of 10:1 does not appear to further increase transfection efficiency of nanoparticles comprising GSCG1,2-RHL which achieve a transfection rate of ˜60% (FIG. 6), as is seen when a DOTMA/DOPE:mRNA w/w ratio of 5:1 is used (FIG. 5). Importantly, however, it is noted that increasing the lipid:mRNA ratio does not negatively impact the transfection efficiency, further demonstrating the versatility of the peptide dendrimer/lipid nanoparticle nucleic acid delivery system.

Again, it was also observed that applying a dendrimer-mannose coat at a w/w ratio of 1:1 (1 eq) with respect to coating:mRNA, the transfection efficiency was again increased by ˜10% compared to a non-coated nanoparticle (FIG. 6).

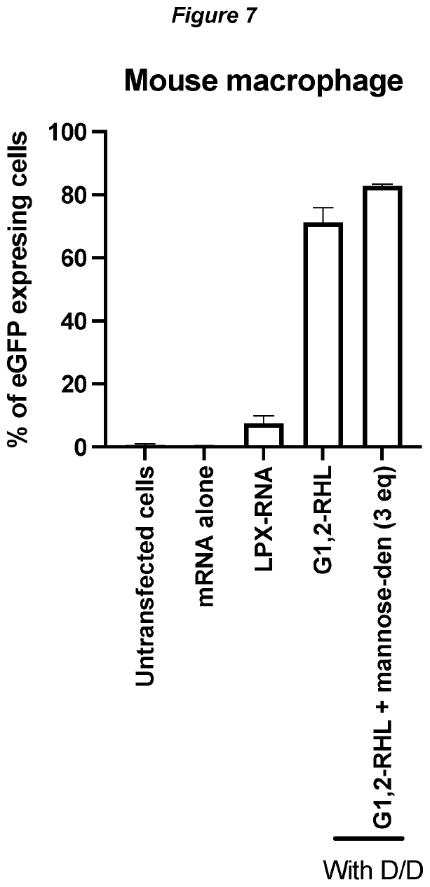

The effect of NP ratio on transfection efficiency was next investigated. Using a nanoparticle comprising GSCG1,2-RHL with an NP=0.6:1 and a lipid:mRNA w/w ratio of 10:1 transfection efficiencies of upwards of ˜70% can be achieved (FIG. 7). This data again further demonstrates the versatility of these delivery systems, offering a wide working range of NP ratio and lipid:mRNA w/w ratio.

As with the other tested nanoparticle formulations, it is possible to further increase the transfection efficiency of GSCG1,2-RHL/lipid nanoparticles with an NP=0.6:1 and a lipid:mRNA w/w ratio of 10:1 by coating the nanoparticle with a mannose-conjugated dendrimer, in this case at a w/w ratio of 3:1 (3 eq) with respect to coating:mRNA (FIG. 7).

Together, the experiments performed above with GSCG1,2-RHL demonstrate that efficient transfection of macrophages can be achieved using these dendrimer/lipid nanoparticles, and that improved transfection can be achieved by optimising the NP ratio (dendrimer:mRNA) and/or the w/w ratio (Lipid:mRNA). Further, the increased transfection efficiencies observed when coating the same nanoparticles with mannose-dendrimers suggests that targeted delivery of nanoparticles to CD206-expressing macrophages may be achieved.

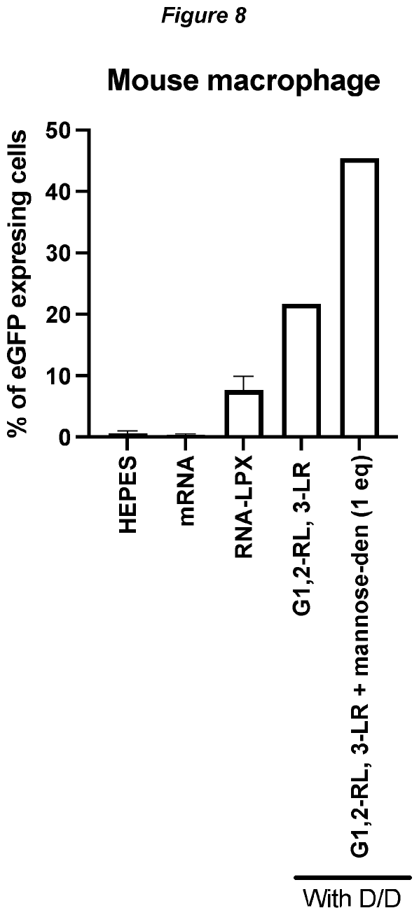

To further investigate the general applicability of the mannose-coating technique for improving delivery of nucleic acid cargo to macrophages, a comparison of nanoparticle comprising GSCG1,2-RL, 3-LR/lipid with and without a coat was conducted. As can be seen in FIG. 8, GSCG1,2-RL, 3-LR/lipid display a moderate transfection efficiency (˜20%) of macrophages in vitro. The transfection efficiency can be increased by ˜30%, to ˜50% efficiency, by including coating the nanoparticles with (Ac-EEEE)2KGSGGSGGSC[(S-S)-α-D-Thiomannose], at a w/w ratio of 3:1 (3 eq) with respect to coating:mRNA. These data confirm that the mannose-conjugated dendrimers can be used to improve transfection efficiency of macrophages using peptide dendrimer/lipid nanoparticles.

### Example 4—Peptide Motifs can be Used to Increase Muscle-Cell Transfection

As shown in Example 3, peptide dendrimer/lipid nanoparticles can be targeted to macrophages by including a targeting motif in the nanoparticle (e.g. a mannose-conjugated dendrimer). To further investigate the ability of these new nucleic acid delivery systems to be targeted to specific cell types, the inventors next produced various peptide dendrimers including a muscle-cell targeting peptide motif (ASSLNIA (SEQ ID NO:1)) to determine if nucleic acid delivery and translation could be increased in muscle cells.

Two variants (NTX2 and NTX3) of the peptide dendrimer GSCG1,2-RL, 3-LR ((LR)8(KRL)4(KRL)2KGSC-NH2) were made which include the ASSLNIA (SEQ ID NO:1) muscle targeting peptide. Transfection of muscle cells with GSCG1,2-RL, 3-LR, NTX2 ((LR)8(KRL)4(KRL)2KGSCGAASSLNIA(Acp)-NH2) or NTX3 ((LR)8(KRL)4(KRL)2KGSCHHHHHHGAASSLNIA(Acp)-NH2) was performed using eGFP mRNA and transfection efficiency was assayed using either flow cytometry or the transgene expression assay. Transfection with eGFP mRNA allows for determination of the levels of mRNA internalised and translated protein from the transfected mRNA. Nanocarriers with cell targeting domain (i.e muscle targeting domain in this case) are expected to yield higher transfection efficacy as compared to the nanocarriers without a targeting domain.

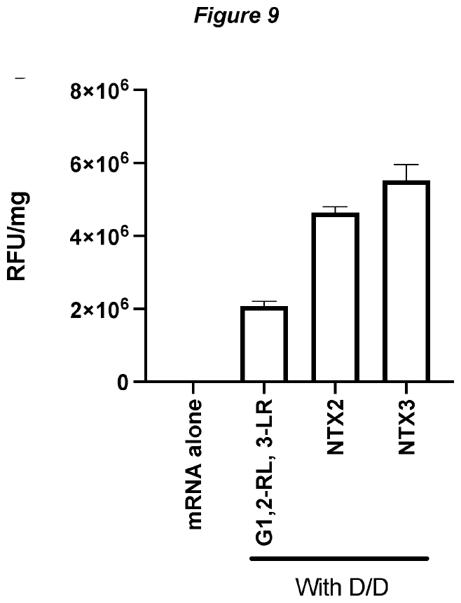

Both NTX2 and NTX3 transfect muscle cells with eGFP mRNA with higher efficiency compared to GSCG1,2-RL, 3-LR, with a two-fold increase in eGFP expression when transfected using NTX2 nanoparticles and a three-fold increase in expression when transfected using NTX3 nanoparticles (FIG. 9). NTX3 contains an extra 6 histidine in the core sequence which may act as a proton sponge to facilitate endosomal escape. This may explain why NTX3 mediates higher transfection efficacy than NTX2.

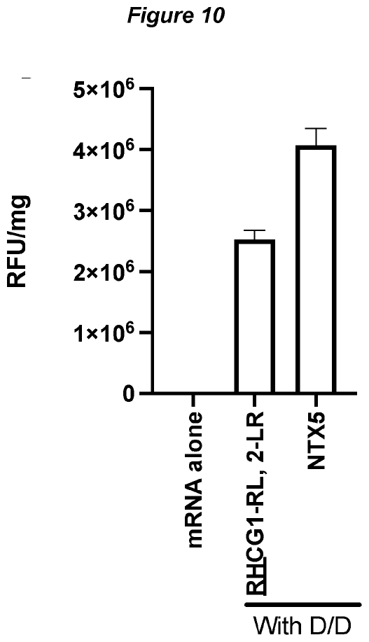

Similar results were obtained when RHCG1-RL, 2-LR ((LR)4(KRL)2KRHC-NH2) dendrimer/lipid nanoparticles were compared to a similar peptide dendrimer which includes the ASSLNIA (SEQ ID NO:1) peptide motif (NTX5, (LR)4(KRL)2KRHCGAASSLNIA-(Acp)-NH2). As can be seen in FIG. 10, including the ASSLNIA muscle targeting motif increased mRNA delivery to muscle cells (FIG. 10) and also increase eGFP expression by ˜two-fold. Together, FIGS. 9 and 10 suggest that including the muscle targeting motif ASSLNIA (SEQ ID NO:1) in a peptide dendrimer increases mRNA delivery to muscle cells and increased transgene expression in the muscle cells.

### Example 5—Peptide Dendrimers can Enhance Tissue-Specific mRNA Expression

The inventors wanted to investigate whether tissue-specific nucleic acid cargo delivery could be improved by including peptide dendrimers in the lipid based nanoparticles. Using a DOTMA/DOPE lipid delivery system (w/w=23:1 to mRNA), luciferase mRNA is efficiently delivered to the lung and spleen, as shown by bioluminescence in FIG. 11. Delivery of mRNA cargo to lung and spleen can be further improved by including either GSCG1-LRLR (NP=0.6:1) or GSCG1,2-RHL (NP=0.6:1).

The above examples emphasise that peptide dendrimer/lipid nanoparticles are a very versatile tool for delivering nucleic acid cargos to various target cells and tissues, including myeloid cells such as macrophages, and in particular M2-phenotype macrophages, muscle cells, lung and spleen.

### Example 6—In Vivo Tissue Distribution

The inventors investigated the tissue distribution of nucleic acid expression achieved by the nanoparticles of the invention.

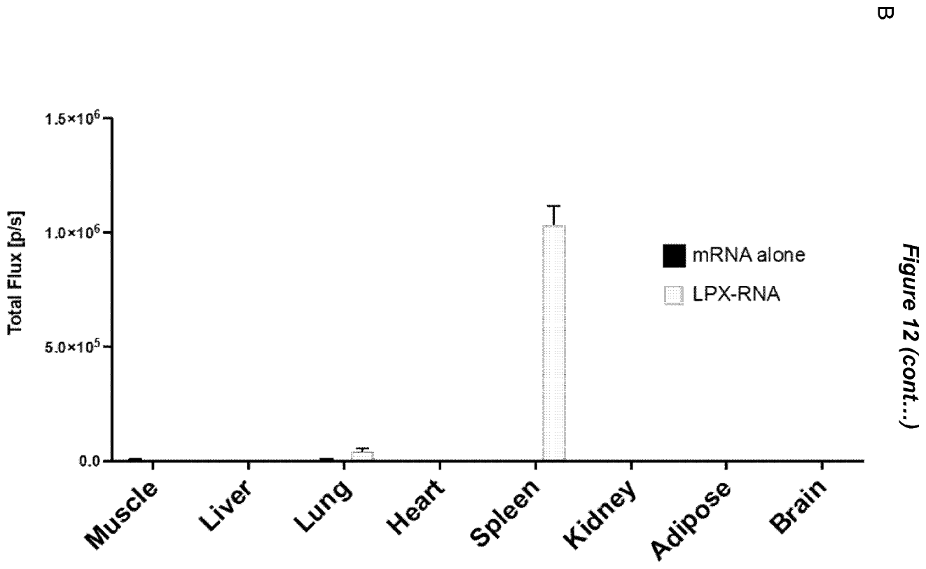

All animal procedures conducted in our studies complied with UK laws and were inclusive of ethics approval. For the data underlying FIG. 12, female BALB/C mice and CD-1 aged 6-8 weeks left to habituate for 1-week upon their arrival. All mice were weighed, and the formulations were delivered intravenously (100 μl) via the tail vein using 30 G insulin syringes (BD biosciences).

### Bioluminescence Imaging (BLI)

All mice were imaged 6 hours post injection. BLI was performed using an IVIS Lumina II (Perkin Elmer) imaging system. Mice were administered D-luciferin (30 mg/mL, XenoLight, Perkin Elmer) at a dose of 150 mg/kg. Mice were anaesthetised 6 mins after receiving D-luciferin in a chamber with 5% isoflurane and then placed on a heated imaging platform while being maintained on 2.5% isoflurane. For the data underlying FIG. 12, mice were imaged 10 min post administration of D-luciferin with an exposure time set to ensure the signal acquired was within an effective detection range (open filter, binning 8, f-stop 1). Bioluminescence signal was quantified by measuring photon flux (photons/s) in the defined region of interest (ROI) using the Living Image software (Perkin Elmer). Following in vivo whole-body imaging, the mice were euthanised, cardiac blood taken, and the tissues extracted for ex vivo imaging.

Each tissue was placed into individual wells of a 24-well imaging plate (black sided, Eppendorf) containing 0.3 mg/mL D-luciferin in PBS). The imaging plate was placed in the centre of imaging platform (Lumina II system) and signal was measured using the acquisition settings detailed above. The results are presented in FIG. 12.

### Example 7—Nanoparticle Size, PDI and Surface Charge Characterization

### Dynamic Light Scattering

The hydrodynamic size was measured using the dynamic light scattering (DLS) technique. DLS is a very sensitive, non-invasive method to measure size and size distribution of nanoparticles in a liquid. The Brownian motion of nanoparticles in suspension resulted in laser light to be scattered at different intensities. Analysing these intensity fluctuations allows us to calculate the velocity of the Brownian motion. The size of the nanoparticles can be determined by using the Stokes-Einstein relationship. With the latest technology, it can measure nanoparticles smaller than 1 nm.

Particle size can also be measured also by:
Nanoparticle tracking analysis
Atomic force microscopy
Electron microscopy
Disc centrifugation
Tunable Resistive Pulse Sensing
Particle Scattering Diffusometry

### Polydispersity Index (PDI)

PDI is used to estimate the average uniformity of a particle solution, The method of cumulants is a standard technique for analysing DLS data on sample polydispersity. PDI is a number calculated from a 2 parameter fit to the correlation data (the cumulants analysis). The cumulants analysis is used to evaluate the autocorrelation function generated by a DLS experiment. The calculation is defined in ISO 13321 and ISO 22412. PDI values greater than 0.7 indicate that the sample has a very broad size distribution and is not suitable for the DLS technique. The calculations for these parameters are defined in the ISO standard document 13321:1996 E and ISO 22412:2008.

### Zeta Potential

Zeta potential is a measurement of the magnitude of the electrostatic or charge repulsion/attraction between particles. This can be determined by analysing particle mobility and charge (Zeta potential) using the Electrophoretic Light Scattering (ELS) technique.

The nanoparticle size, PDI and surface charge characteristics for a nanoparticle of the invention comprising a second generation dendrimer alone is shown in Table 2, alongside the characteristics for a nanoparticle of the invention comprising a second generation dendrimer alongside a further PGA dendrimer that is derivatized with mannose:

TABLE 2

| | Size | | Zeta Potential |
|---|---|---|---|
| Dendrimer(s) | (nm) | PDI | (mV) |
| G1,2-RHL | 149.5 | 0.1776 | 28.23 |
| G1, 2-RHL; G1-EEEE | 175.4 | 0.09906 | −18.63 |
| with mannose | | | |

The size, polydispersity index (PDI) and zeta potentials
of the nanocarriers, mRNA with G1, 2-RHL (N:P 0.6:1) and
DOTMA: DOPE (w/w 10:1 to mRNA), with or without the
G1-EEEE with mannose ((Ac-EEEE)2KGSGGSGGSC[(S-S)-α-D-
Thiomannose]) coating. 3 equivalents of the G1-EEEE with
mannose to mRNA was added to coat the nanocarrier.

### Example 8—In Vivo Delivery of mRNA with Nanoparticles with or without Mannose Coating

The inventors investigated the distribution of nucleic acid in vivo by the nanoparticles of the invention, with or without the coating of mannose-G1-EEEE dendrimer.

### Method

Six week old, female C57Bl/6 mice were supplied from Envigo UK. The mice were acclimatized in the animal facility for at least 7 days prior to use. MC38 cells were implanted into the left flanks at 1×10⁷ cell per mouse. Tumours were measured by callipers as soon as palpable three times per week. The mice were randomised into 4 groups of n=3 when the tumours reached 0.15 cm3 (calliper measurements) and were dosed IV at 2.25 mg/kg or 3 mg/kg.

Four hours post dosing of the formulations, the animals were culled, and tissues were harvested. Terminal blood was collected in EDTA tubes and treated with Red Blood Cell Lysing Buffer HYBRI-MAX (Merck) prior to antibody staining. Spleens were dissociated by pushing through a 70 uM cell strainer using a syringe plunge. The cell suspension was then treated with Red Blood Cell Lysing Buffer HYBRI-MAX (Merck) prior to antibody staining. Lung and tumour samples were cut into small pieces and were enzymatically digested using the Lung and Tumor Dissociation Kit (mouse) and the gentleMACS™ Octo Dissociator with Heaters (Miltenyi) following the manufacturer's instructions.

The final cell suspensions were blocked with mouse Fc block prior to antibody staining. Cells were incubated with the antibody master mix at 4 C for 20 min and then they were fixed using 4% PFA for 30 min RT. They were analysed using the Attune NxT flow cytometer (Thermo Fisher) within 24 hours of fixing and data was analysed using the FlowJo_v10.8.1 software. Compensation was adjusted using the Attune compensation beads.

### Results

TABLE 3
in vivo mRNA delivery to myeloid cells in tumour.

| | % of live cells |
|---|---|
| Cell type | with Alexafluor488 |
| CD45+ CD11b+ (Myeloid cells) | 36.8 ± 11.9 |
| CD45+ CD11b+ F4/80+ (Macrophages) | 50.4 ± 11.8 |

Percentage of myeloid cells targeted by Alexafluor488 tagged mRNA with GSCG1,2-RL, 3-LR (N:P = 0.16:1) and DOTMA:DOPE (w/w 10:1, lipid to mRNA)(at a dose of 3 mg/kg, mRNA to body weight).
Cells were isolated for flow cytometry analysis 4 hours post-injection of the formulations.
N = 3, SEM is shown.

### Conclusion

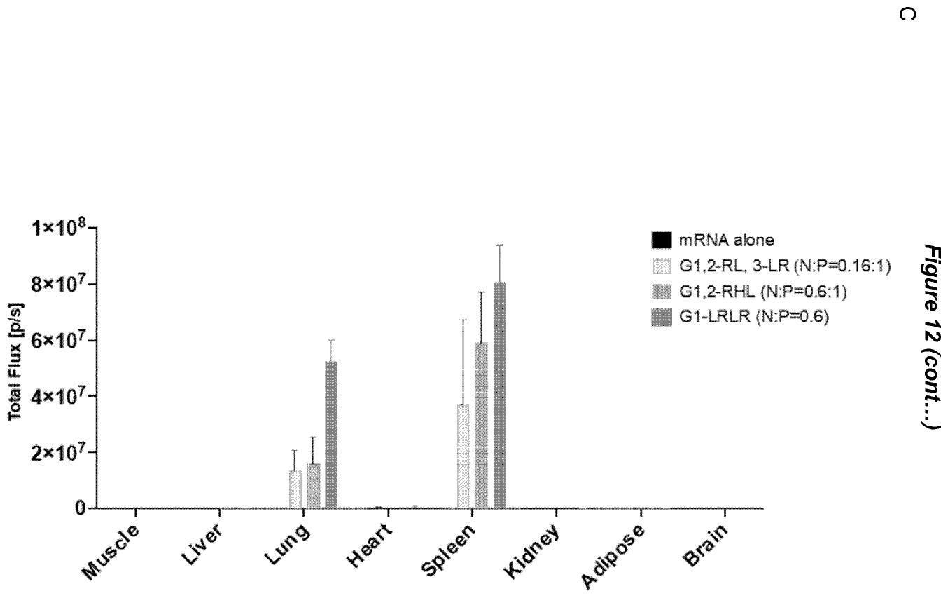

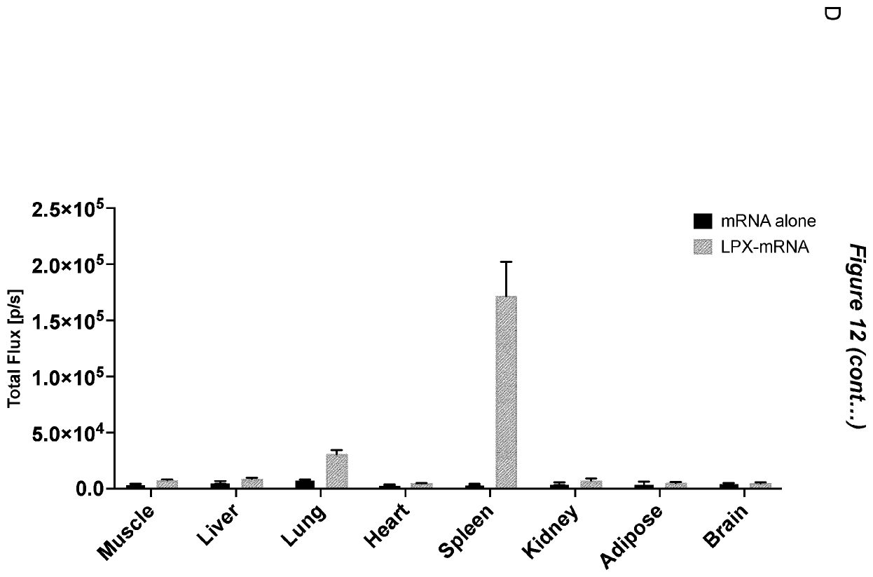

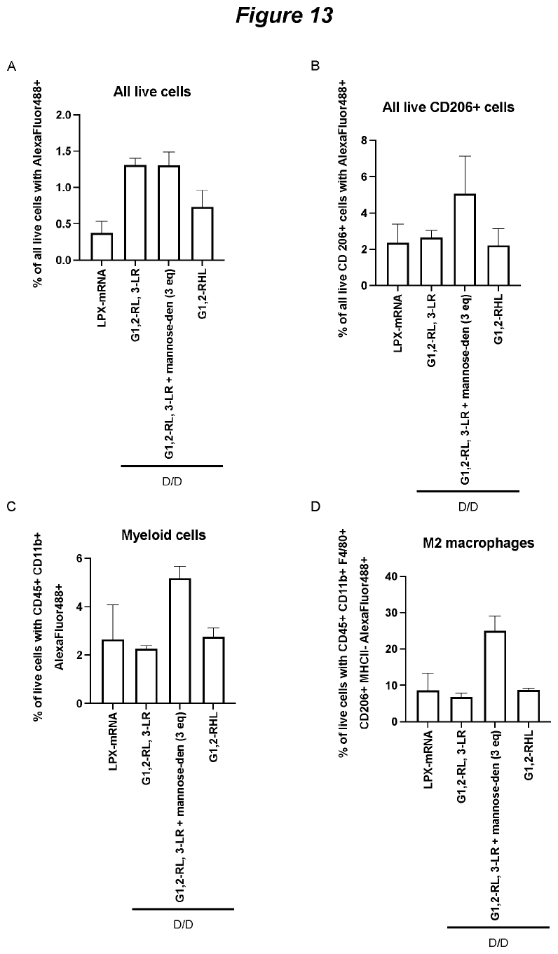

As shown in FIG. 13, nanocarriers coated with mannose-G1-EEEE mediated higher uptake in the lung cells compared to non-coated nanocarrier and LPX-mRNA alone. Without bound by theory, this may be due to the coating of the dendritic PGA resulted in the interaction with serum components which may direct the nanocarrier to the lung tissue more readily.

All the CD206+ cells took up the nanocarriers coated with mannose-G1-EEEE more effectively than the non-coated nanocarrier and LPX-mRNA alone. This may suggest that the mannose targeting is specific to the CD206+ expressing cells, including M2 macrophages.

Table 3 indicates that the mRNA with G1,2-RL, 3-LR (N:P 0.16:1) and DOTMA:DOPE (w/w 10:1 to mRNA) mediated efficient uptake in myeloid cells such as macrophage, neutrophils and dendritic cells within the tumour.

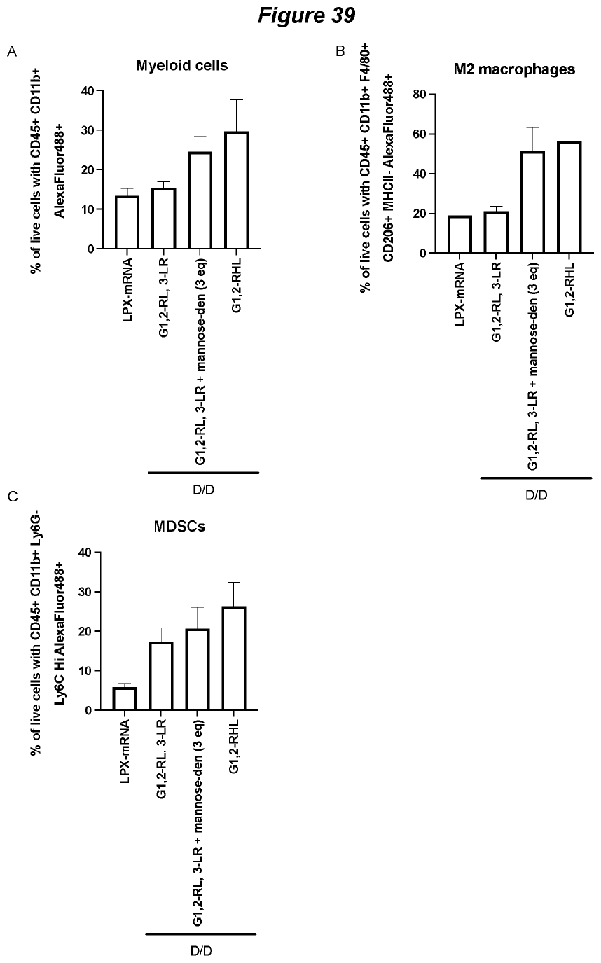

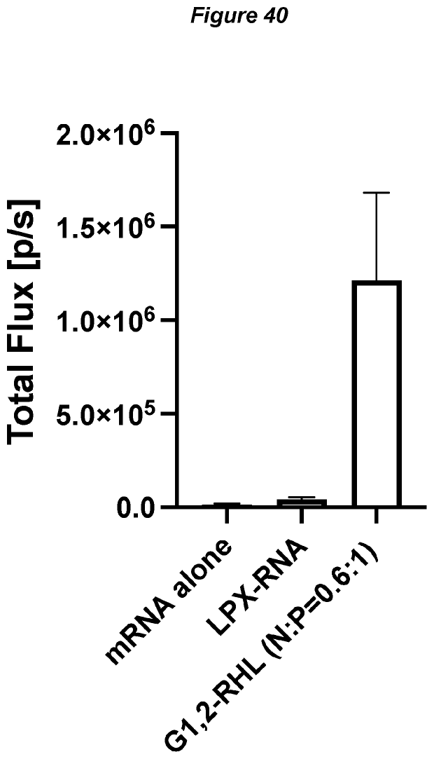

As shown in FIG. 39, the CD206+ M2 macrophage cells took up the nanocarriers coated with mannose-G1-EEEE more effectively than the non-coated nanocarrier and LPX-mRNA alone. This suggests that the mannose targeting is specific to the CD206+ expressing cells, including M2 macrophages.

The above examples emphasise that peptide dendrimer/lipid nanoparticles are a very effective in delivering nucleic acids to myeloid cells in tissues, in some case non-myeloid cells (e.g. non-immune cells in the lung). The cell specificity can be further improved by adding a targeting motif to the nanocarrier (e.g. mannose for CD206 targeting).

### Example 9—Comparison of Third Generation Dendrimer Transfection Efficiency of mRNA and DNA Compared to Commercially Available Transfection Reagents

In vitro transfection efficiency for mRNA and DNA delivery was evaluated using certain dendrimers from Table 1 (shown in Example 1, above) and discussed in the following examples:

Nucleic acid delivery systems based on cationic lipids are one of the most studied and efficient non-viral vector platforms described to date, and the rational design and development of peptidic vectors with natural amino acids are particularly attractive for therapeutic applications due to the non-toxic nature of the amino acids. The inventors have developed a structural framework for nucleic acid delivery, using peptide dendrimers. The structural framework involves layers of peptide (or dipeptide) motifs, bound to lysine residues. The inventors have found that the distribution of cationic amino acid residues (Lys or Arg) in each generation (layer) gave peptide dendrimers transfecting more efficiently than dendrimers with charges localized solely on the surface (Kwok et al, 2013). Using a solid phase peptide dendrimer synthetic procedure, the inventors can precisely manipulate the position of every amino acid residue incorporated within the dendritic scaffold. This allows greater control of the structure and function of the dendrimer, which was normally not possible with previously studied systems such as polymers or other dendrimers where modifications were mainly made on the surface of the molecule. The peptide dendrimer/lipid vector showed high transfection efficiency, good reproducibility of results and low toxicity.

The inventors compared the in vitro transfection efficiency of these new peptide dendrimer systems to other known transfection reagents. HeLa cells were transfected with mRNA encoding luciferase using either a composition comprising GSCG1,2-RL, 3-LR (NP=0.16:1 to mRNA) and DOTMA/DOPE (w/w 10:1 to mRNA) or commercially available Lipofectamine™ 2000. As shown in FIG. 14, top left panel, compositions comprising GSCG1,2-RL, 3-LR (NP=0.16:1 to mRNA) and DOTMA/DOPE increase transfection efficiency in vitro by around an order of magnitude compared to the commercially available transfection reagent Lipofectamine™ 2000. Similarly, HeLa cells transfected with mRNA encoding eGFP using GSCG1,2-RL, 3-LR (NP=0.16:1 to mRNA) and DOTMA/DOPE increased eGFP expression by approximately 4× compared to mRNA transfected using a DLin-MC3-DMA: Cholesterol: DSPC: DMG-PEG lipid nanoparticle delivery system (FIG. 14, top right panel).

To further validate the peptide dendrimer system, C2C12 cells were transfected for 24 hours with either 1) mRNA alone; 2) GSCG1,2-RL, 3-LR (NP=8:1 to mRNA) and DOTMA/DOPE (at a w/w=10:1 to mRNA) with mRNA; 3) DOTMA/DOPE (at a w/w=10:1 to mRNA) with mRNA; 4) polyethylenimine with mRNA; and 5) Lipofectamine 2000 with mRNA. As shown in FIG. 14, bottom panel, peptide dendrimer formulations significantly improve delivery of mRNA to C2C12 cells compared to commercially available lipid-based transfection reagents.

Using a third generation peptide dendrimer, GSCG1,2,3-RL, the inventors also compared the transfection efficiency of DNA using this dendrimer to that of commercially available transfection reagents. HeLA cells (FIG. 15, top panels) or Nuero2A cells (FIG. 15, bottom panels) were transfected with DNA using 1) GSCG1,2,3-RL and DOTMA/DOPE; 2) DOTMA/DOPE alone; 3) Polyethylenimine; or 4) Lipofectamine 2000. In both serum free conditions (FIG. 15, left panels) and serum conditions (FIG. 15, right panels), the dendrimer GSCG1,2,3-RL outperformed all of the tested commercial transfection reagents. The third generation dendrimers transfected 2-600 times better than some of the widely used commercial reagents such as Polyethylenimine (PEI), Lipofectin (also known as DOTMA/DOPE) and Lipofectamine 2000 in HeLa and Neuro2A cells (FIG. 15).

Overall, the G3 dendrimers tested transfect mRNA and DNA efficiently in HeLa cells, and at least mRNA in C2C12 cells and DNA in Neuro2A cells.

### Example 10—Comparison of Generation 1, 2 and 3 Dendrimers on Transfection Efficiency

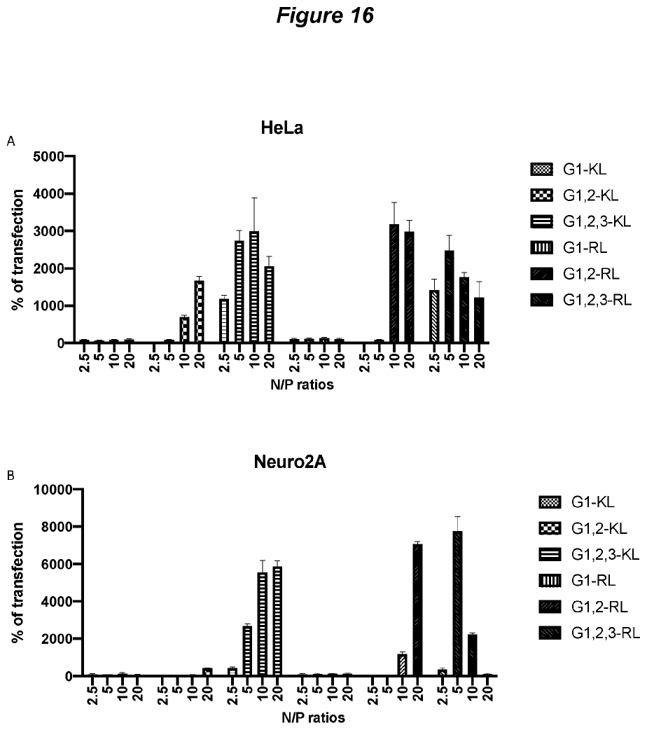

The number of generations from G1 to G2 to G3 based on the KL repeating unit in delivery efficiency was explored. The KL unit was also replaced with the RL repeating unit to compare the effect of protonated basic group with different pKa. Interestingly it was observed that there is a relationship between generations and transfection in the cells (FIG. 16). The generation dependence on transfection reveals that first generation dendrimers GSCG1-KL or GSCG1-RL do not transfect HeLa or Neuro2A cells with lysine or arginine as charged residues. This may be due to the fact that G1 peptides are not physically large enough to fully encapsulate plasmid DNA sufficiently to form a stable nanoparticle, as shown by the complex stability assays (data not shown).

For second generation however the arginine containing GSCG1,2-RL shows a higher transfection efficiency (at an N/P ratio of 10:1 or 20:1 with respect to mRNA in HeLa cells and at an NIP ratio of 20:1 with respect to mRNA in Neuro2A cells) in comparison to GSCG1,2-KL (FIG. 16). Such transfection superiority of GSCG1,2-RL to GSCG1,2-KL is in line with the ability of GSCG1,2-RL to form more stable transfection complexes than GSCG1,2-KL with DNA (data not shown).

### Example 11—Comparison of Single Dendrimer Systems to Hybrid Dendrimer Systems

Hybrid dendrimer systems were investigated to determine if transfection efficiency could be improved further compared to single dendrimer systems.

### Materials and Methods

Cell lines, transfection reagents and mRNA. HeLa cells were maintained in RPMI medium with 10% (v/v) FCS and 1% (v/v) P/S in a humidified atmosphere in 5% CO2 and 37° C. The eGFP mRNA was purchased from Trilink (CleanCap® EGFP mRNA (5 moU)-(L-7201)). DOTMA:DOPE, 1:1 (w/w) were obtained from Invitrogen (Lipofectin™ Transfection Reagent) (Fisher—18292037) or Encapsula NanoSciences LLC.

Nanoparticle formulation procedure: To formulate the hybrid dendrimer nanoparticle, peptide dendrimer 1 was mixed with peptide dendrimer 2 in a molar ratio with respect to N contributed, and the final NIP ratio was usually 8 overall. For example, 10 mg/ml peptide dendrimer 2 solution (1.886 μl, 18.9 μg, 7.50×10-6 mmoles dendrimer, 5.25×10-5 mmoles N, N/P 5.33) in sterile water (2.90 μl) and 200 mM HEPES buffer (0.814 μl) was added to tube A which contained peptide dendrimer 1 (10 mg/ml peptide dendrimer 1 solution (0.904 μl, 9.0 μg, 2.33×10-7 mmoles dendrimer, 2.56×10-6 mmoles N, NIP=2.67). The tube was shaken gently then spun down for 10-15 seconds using a mini centrifuge to ensure all liquid is at the bottom of the tube. 16.25 μl of the mRNA (200.0 μg/ml, 3.25 μg, 9.85×106 mmoles Phosphate of mRNA in 25 mM HEPES buffer) was added to the tube to allow the complexes to form at an N/P=8 by rapidly pipetting up and down 10-15 times. This was allowed to sit for 2-5 minutes. 796.2 μg/ml DOTMA/DOPE liposome solution (42.2 μl, 32.5 μg) was mixed into the tube by rapidly pipetting up and down 10-15 times.

Transfection procedure: 24 hours before transfection, HeLa cells were seeded in 96well plates in order to reach 70% confluence. mRNA transfection complexes were formed by mixing mRNA with the dendrimers in 25 mM HEPES buffer, then with DOTMA:DOPE in 25 mM HEPES buffer at 25° C. All transfection mixes were formulated with a final dendrimer:mRNA NP ratio of 8:1. DOTMA:DOPE was added to the mRNA complexes at a w/w=10:1. The transfection complexes were then overlaid to the cells in full growth medium. The cells were harvested for reporter gene assay 24 hour post transfection.

Transgene expression assay: The cells were washed twice with PBS and incubated with 50 ul of 1× M-PER lysis buffer (Thermo 11874111). Plates were protected from light and gently agitated at RT for 15 minutes to aid cell lysis. 40 ul of lysate from each well was transferred to all black 96 well plates for quantification of eGFP relative fluorescence unit (RFU), absorbance at 535 nm, using Molecular devices SpectraMax iD5.

Protein content determination: The protein content of each cell lysate was determined by mixing the lysate (25 μL) with Pierce™ BCA Protein Assay Kit (200 ul, Thermo Scientific). After 30 minutes of 37° C. incubation in at dark, absorbance was measured at 562 nm with Molecular devices SpectraMax iD5 and converted to protein concentration using a BSA standard curve. RFU per mg of protein represented eGFP expression. The values displayed in the transfection figures are represented after normalization against a control transfection experiment with DOTMA/DOPE and are shown as percentages.

Size, zeta potential and polydispersity index (PDI) measurement: The hydrodynamic size was measured using the dynamic light scattering (DLS) technique using the Zetasizer Advance Series—Pro (Malvern Panalytical Ltd, Malvern, UK) according to the manufacturer's instructions. DLS is a very sensitive, non-invasive method to measure size and size distribution of nanoparticles in a liquid. The Brownian motion of nanoparticles in suspension resulted in laser light to be scattered at different intensities. Analysing these intensity fluctuations allows us to calculate the velocity of the Brownian motion. The size of the nanoparticles can be determined by using the Stokes-Einstein relationship. With the latest technology, it can measure nanoparticles smaller than 1 nm.

The data obtained with DLS measurements can also be used to calculate the PDI of particle in solution. PDI is used to estimate the average uniformity of a particle solution. The method of cumulants is a standard technique for analysing DLS data on sample polydispersity. PDI is a number calculated from a 2 parameter fit to the correlation data (the cumulants analysis). The cumulants analysis is used to evaluate the autocorrelation function generated by a DLS experiment. The calculation is defined in ISO 13321 and ISO 22412. PDI values greater than 0.7 indicate that the sample has a very broad size distribution and is not suitable for the DLS technique. The calculations for these parameters are defined in the ISO standard document 13321:1996 E and ISO 22412:2008.

To measure the size and PDI, the samples were diluted 16× in 25 mM HEPES buffer (5 μl sample+75 μl buffer). Parameters: reference material: polystyrene latex, dispersant: water, 25° C. The PDI measurements were measured in nanoparticles comprising a dendrimer:nucleic acid NP ratio of 8:1.

Zeta potential is a measurement of the magnitude of the electrostatic or charge repulsion/attraction between particles. This can be determined by analysing particle mobility and charge (Zeta potential) using the Electrophoretic Light Scattering (ELS) technique. To measure the zeta potential, the samples were diluted 150× in 25 mM HEPES buffer (10 μl sample+690 μl buffer) and added to a clean DTS1070 cell. Parameters: reference material: polystyrene latex, dispersant: water, 25° C. The measurements of the zeta potential were carried out using the Zetasizer Advance Series—Pro (Malvern Panalytical Ltd, Malvern, UK) according to the manufacturer's instructions.

### Results

The PDIs of nanoparticles comprising mRNA, lipid and single peptide dendrimers was investigated. The polydispersity index (PDI) can be used to measure the ability of particular compositions comprising specific peptide dendrimers to form monodisperse nanoparticle populations. A higher PDI is associated with a decrease in the formation of a monodisperse population of nanoparticles. As can be seen in Table 5, nanoparticles comprising, for example, RHCG1-R, RHCG1-RLR or G1-LRLR have a PDI greater than 0.35, showing that these dendrimers cannot form sufficiently monodisperse mRNA nanoparticles at an NP ratio of 8:1. This inability to form a monodisperse population indicates that the dendrimer-mRNA complexes in nanoparticles with a PDI>0.35 are relatively unstable compared to those dendrimer-mRNA complexes in nanoparticles with a PDI less than or equal to 0.35.

TABLE 5
Polydispersity index (PDI) of peptide
dendrimer, lipid and mRNA complexes.

| | Polydispersity |
|---|---|
| Name | Index (PDI) |
| RHCG1-R | 0.566 |
| RHCG1,2-R | 0.252 |
| RHCG1,2,3-R | 0.396 |
| RHCG1-LR | 0.577 |
| RHCG1-RL, 2-LR | 0.109 |
| RHCG1,2-RL, 3-LR | 0.363 |
| AG1,2-RL, 3-LR | 0.184 |
| RFWG1,2-RL, 3-LR | 0.401 |
| RYMG1,2-RL, 3-LR | 0.257 |
| YMG1,2-RL, 3-LR | 0.213 |
| LinearG1,2-RL, 3-LR | 0.289 |
| RHCG1-RLR | 0.766 |
| RHCG1,2-RLR | 0.214 |
| RHCG1,2,3-RLR | 0.299 |
| GSCG1-LRLR | 0.581 |
| GSCG1,2-LRLR | 0.189 |
| GSCG1,2,3-LRLR | 0.287 |
| RHCG1-RLR, 2-RL, 3-R | 0.429 |
| RHCG1-RLR, 2-R, 3-LR | 0.439 |
| RHCG1-RLRL, 2-R, 3-LR | 0.37 |
| GSCG1-KL, 2-KH, 3-HK | 0.126 |
| GSCG1-RE, 2-RL, 3-LR | 0.158 |
| GSCG1-RF, 2-RM, 3-WR | 0.277 |
| GSCG1-RX, 2-RB, 3-BR | 0.403 |
| GSCG1-RQ, 2-RT, 3-YR | 0.409 |
| GSCG1-RW, 2-RQ, 3-FR | 0.452 |
| GSCG1-RY, 2-RT, 3-QR | 0.178 |
| GSCG1-RC, 2-RC, 3-MR | 0.19 |
| GSCG1-LL, 2-RR | 0.087 |
| gscG1,2-rl, 3-lr (D-form branching lysines) | 0.246 |
| gscG1,2-rl, 3-lr | 0.251 |
| GSCG1-Rl, 2-rL, 3-lR | 0.241 |
| GSCG1,2-RL, 3-LR (Dab branching residues) | 0.286 |
| GSCG1,2,3-RL | 0.509 |
| GSCG1-LR, 2,3-RL | 0.417 |
| gscG1,2,3-rl | 0.21 |
| GSCG1,2-RL, 3-LR | 0.261 |
| GSCG1,2-LR, 3-RL | 0.242 |
| GSCG1,2,3-LR | 0.272 |

The dendrimers were at an N:P ratio of 8:1 and the lipids, DOTMA/DOPE, were at a w/w ratio of 10:1 to mRNA.
The NH₂ groups present at the C terminus of the core sequences are a result of the peptide synthesis method.
Acp or X = 6-aminohexanoic acid, B = beta-alanine, Dab = 2,4-Diaminobutyric acid.
Lowercase letters refer to D-form amino acids, whereas uppercase letters refers to L-form amino acids.
l is the D-form leucine.
There is no D or L form for glycine.

With the above in mind, it was investigated whether including a dendrimer with a PDI>0.35 in a composition comprising a stable dendrimer-mRNA complex (i.e. a PDI less than or equal to 0.35) could increase the transfection efficiency by increasing the rate of mRNA dissociation following entry into cells.

A first comparison was made between HeLa cells transfected using either GSCG1,2-RL, 3-LR alone or a mix of GSCG1,2-RL, 3-LR and GSCG1-LRLR at a ratio of 1:2. The inclusion of the G1 dendrimer, GSCG1-LRLR, in a composition comprising the G2 dendrimer GSCG1,2-RL, 3-LR significantly increases the transfection efficiency of mRNA compared to the single-dendrimer composition comprising GSCG1,2-RL, 3-LR alone (FIG. 17A). The transfection efficiency was increased by ˜100% in the hybrid dendrimer composition compared to the single-dendrimer composition.

A second G1 dendrimer was investigate for use in a hybrid dendrimer system. A comparison was made between transfection mixes comprising either GSCG1,2-RL, 3-LR alone or GSCG1,2-RL, 3-LR and RHCG1-R at ratio of either 2:1 or 1:2. A 2:1 ratio of GSCG1,2-RL, 3-LR:RHCG1-R led to an increase in transfection efficiency of ˜100% (FIG. 17B). A further increase in transfection efficiency was obtained by altering the G2:G1 ratio to 1:2, with a transfection efficiency of ˜300% compared to the G2 dendrimer mix alone.

Next, RHCG1-RL, 2-LR-containing dendrimer transfection mixes were investigated with either RHCG1,2-R, RHCG1-RLR, or GSCG1-RLR (FIG. 17C-E, respectively). Using a 1:1 G2:G1 ratio can be seen to lead to a modest increase in transfection efficiency of between ˜20-50% compared to the G2 dendrimer alone (FIGS. 17D and E, compare first and second bars). While, in accordance with the previously discussed results, using a 1:2 ratio further increases the transfection efficiency by ˜60%-100% (FIGS. 17D and E, compare first and third bars).

Considering the results presented in FIGS. 17A-B and 17D-E, it can be seen that addition of a G1 dendrimer to form a hybrid dendrimer system with either a G2 or G3 dendrimer improves mRNA transfection compared to mRNA complexes formed with a single dendrimer alone. For example, FIG. 17A indicates that combining a G1 dendrimer, GSCG1-RLRL, with a G3 dendrimer, GSCG1,2-RL, 3-LR, improves transfection by 200%. FIG. 17B, D-E also indicates that addition of a G1 dendrimer to a G2 dendrimer can enhance transfection up to 300%.

Surprisingly, it was also seen that hybrid dendrimer mixes comprising two G2 dendrimers, RHCG1-RL, 2-LR and RHCG1,2-R can also increase mRNA transfection efficiency (FIG. 17C). While it is still unclear what leads to this increase in transfection efficiency, one hypothesis is that the inclusion of the relatively less stable RHCG1,2-R in the nanoparticle increases the overall instability of the nanoparticle facilitating mRNA release once the nanoparticle has entered the cell.

As can be seen in Table 5, the PDI of RHCG1,2-R nanoparticles is relatively high compared to that of RHCG1-RL, 2-LR (0.252 vs 0.109). While a PDI of 0.252 would indicate an acceptable monodisperse population is formed in nanoparticles comprising on RHCG1,2-R, it is possible that the increased relative instability compared to the very stable nanoparticle formed using RHCG1-RL, 2-LR aids in the dissociation of mRNA from a nanoparticle comprising both dendrimers. This is supported by the results of PDI assays of the mixed dendrimer composition (Table 6, discussed further below) which demonstrates the PDI of a nanoparticle comprising a 1:1 ratio of RHCG1-RL,2-LR and RHCG1,2-R is higher than a nanoparticle comprising RHCG1-RL,2-LR alone (0.155 vs 0.109, respectively).

### Example 12—PDI Assays of Mixed Dendrimer Systems

While improved transfection efficiency is critical to the development of improved nucleic acid therapies, it is also of great importance that any new formulations have pharmaceutically acceptable properties. For example, it is important that a new formulation will have a sufficiently monodisperse population of nanoparticles so that any in vivo results will be predictable. Therefore, the PDI of the above “hybrid” nanoparticles was analysed. Table 6 demonstrates that the majority of the tested nanoparticles had a sufficiently monodisperse population of nanoparticles which would be suitable for use in a pharmaceutical composition—i.e. all bar one of the tested combinations has a PDI of less than 0.35.

The PDI of nanoparticles comprising a mixed dendrimer population is tuneable by altering the ratio of dendrimers in the nanoparticle. For example, the PDI of the dendrimer combination RHCG1-RL,2-LR and RHCG1-R at a 1:2 ratio vs a 2:1 ratio is 0.521 and 0.108, respectively. Similarly, the PDI of the dendrimer combination RHCG1-RL,2-LR and RHCG1-RLR at a 1:1 ratio vs a 1:2 ratio is 0.0819 and 0.248, respectively. Finally, the PDI of the dendrimer combination RHC_G1-RL,2-LR and GSCG1-LRLR at a 1:1 ratio vs a 1:2 ratio is 0.141 and 0.289 respectively. Taken together, these results indicate that by increasing the proportion of the dendrimer with a higher PDI when used alone increases the PDI of the mixed dendrimer nanoparticles.

TABLE 6
The polydispersity index (PDI) and hydrodynamic
sizes of the mRNA complexes.

| | Number of | |
|---|---|---|
| Name | generation | PDI |
| GSCG1,2-RL, 3-LR:GSCG1-LRLR | 3 & 1 | 0.094 |
| (1:2) | | |
| RHCG1-RL, 2-LR:RHCG1-R (1:2) | 2 & 1 | 0.521 |
| RHCG1-RL, 2-LR:RHCG1-R (2:1) | 2 & 1 | 0.108 |
| RHCG1-RL, 2-LR:RHCG1,2-R (1:1) | 2 & 2 | 0.155 |
| RHCG1-RL, 2-LR:RHCG1-RLR (1:1) | 2 & 1 | 0.0819 |
| RHCG1-RL, 2-LR:RHCG1-RLR (1:2) | 2 & 1 | 0.248 |
| RHCG1-RL, 2-LR:GSCG1-LRLR | 2 & 1 | 0.141 |
| (1:1) | | |
| RHCG1-RL, 2-LR:GSCG1-LRLR | 2 & 1 | 0.289 |
| (1:2) | | |

The N/P ratio of the complexes was 8.
The complexes were formulated with the molar ratio with respect to the N contributed from each dendrimer (i.e. the ratio in bracket represents the molar ratio with respect to the N of each dendrimer).
DOTMA:DOPE was added to the mRNA complexes at an w/w = 10:1.

It can be seen that increasing the proportion of the dendrimer with a greater PDI in a nanoparticle comprising two peptide dendrimers generally increases the transfection efficiency of the nanoparticle. However, as the proportion of the dendrimer with a higher PDI is increased so too is the PDI. With this observation, it is possible that the ratio of peptide dendrimers in a nanoparticle can be optimised to give the highest transfection efficiencies whilst keeping the PDI within acceptable boundaries (e.g. less than or equal to 0.35).

### Example 13—Exemplary Dendrimers for Use in Mixed Peptide Dendrimer/Lipid Nanoparticles

TABLE 7
Exemplary first generation dendrimers for
use in mixed dendrimer nanoparticles.

| Name | Structure | PDI |
|---|---|---|
| RHCG1-R | (R)2KRHC-NH2 | 0.566 |
| RHCG1-LR | (LR)2KRHC-NH2 | 0.577 |
| RHCG1-RLR | (RLR)2KRHC-NH2 | 0.766 |
| GSCG1-LRLR | (LRLR)2KGSC-NH2 | 0.581 |

TABLE 8
Exemplary second and third generation dendrimers
for use in mixed dendrimer nanoparticles.

| Name | Structure | PDI |
|---|---|---|
| RHCG1,2-R | (R)4(KR)2KRHC-NH2 | 0.252 |
| RHCG1-RL, 2-LR | (LR)4(KRL)2KRHC-NH2 | 0.109 |
| AG1,2-RL, 3-LR | (LR)8(KRL)4(KRL)2KA-NH2 | 0.184 |
| RYMG1,2-RL, 3-LR | (LR)8(KRL)4(KRL)2KRYM-NH2 | 0.257 |
| YMG1,2-RL, 3-LR | (LR)8(KRL)4(KRL)2KYM-NH2 | 0.213 |
| LinearG1,2-RL, 3-LR | (LR)8(KRL)4(KRL)2KGSCRHCRFWRYM- | 0.289 |
| | NH2 | |
| RHCG1,2-RLR | (RLR)4(KRLR)2KRHC-NH2 | 0.214 |
| RHCG1,2,3-RLR | (RLR)8(KRLR)4(KRLR)2KRHC-NH2 | 0.299 |
| GSCG1,2-LRLR | (LRLR)4(KLRLR)2KGSC-NH2 | 0.189 |
| GSCG1,2,3-LRLR | (LRLR)8(KLRLR)4(KLRLR)2KGSC-NH2 | 0.287 |
| GSCG1-KL, 2-KH, 3-HK | (HK)8(KKH)4(KKL)2KGSC-NH2 | 0.126 |
| GSCG1-RE, 2-RL, 3-LR | (LR)8(KRL)4(KRE)2KGSC-NH2 | 0.158 |
| GSCG1-RF, 2-RM, 3- | (WR)8(KRM)4(KRF)2KGSC-NH2 | 0.277 |
| WR | | |
| GSCG1-RY, 2-RT, 3-QR | (QR)8(KRT)4(KRY)2KGSC-NH2 | 0.178 |
| GSCG1-RC, 2-RC, 3- | (MR)8(KRC)4(KRC)2KGSC-NH2 | 0.19 |
| MR | | |
| GSCG1-LL, 2-RR | (RR)4(KLL)2KGSC-NH2 | 0.087 |
| gscG1,2-rl, 3-lr (D-form | (lr)8(krl)4(krl)2kgsc-NH2 | 0.246 |
| branching lysines) | | |
| gscG1,2-rl, 3-lr | (lr)8(Krl)4(Krl)2Kgsc-NH2 | 0.251 |
| GSCG1-Rl, 2-rL, 3-lR | (lR)8(KrL)4(kRl)2KGSC-NH2 | 0.241 |
| GSCG1, 2-RL, 3-LR | (LR)8(DabRL)4(DabRL)2DabGSC-NH2 | 0.286 |
| (Dab branching | | |
| residues) | | |
| gscG1,2,3-rl | (rl)₈(krl)₄(krl)₂kgsc-NH₂ | 0.21 |
| GSCG1,2-RL, G3-LR | (LR)₈(KRL)₄(KRL)₂KGSC-NH₂ | 0.261 |
| GSCG1,2-LR, G3-RL | (RL)₈(KLR)₄(KLR)₂KGSC-NH₂ | 0.242 |
| GSCG1,2,3-LR | (LR)₈(KLR)₄(KLR)₂KGSC-NH₂ | 0.272 |

### Example 14—Determining Transfection Efficiency

### Transfection of C2c12 Cells:

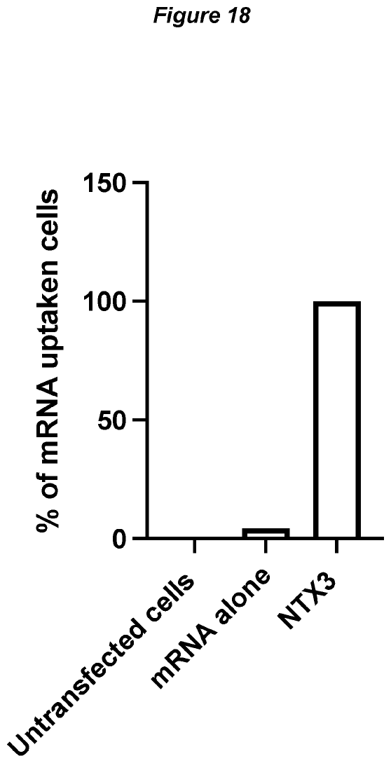

C2c12 cells were maintained in DMEM medium with 10% (v/v) FCS and 1% (v/v) L-glutamine in a humidified atmosphere in 5% CO2 and 37° C. The Alexa Fluor 488 tagged mRNA, expressing an eGFP, was purchased from RiboPro. 24 hours before transfection, C2c12 cells were seeded in 96 well plates in order to reach 70% confluence. mRNA transfection complexes were formed by mixing mRNA with the dendrimers in 25 mM HEPES buffer, then with DOTMA:DOPE (1:1 w/w) in 25 mM HEPES buffer at 25° C. The transfection complexes (“NTX3”) were then overlaid to the cells in full growth medium. The cells were harvested for FACS analysis 4 hours after transfection. Cells medium and PBS used to wash each well, was collected from each treatment before the addition of 5 uM EDTA in PBS. After 10 minutes of incubation at 37° C., the cells were gently detached from the plates and pelleted down at 400 g for 8 minutes, 4° C. Cells were stained with LIVE/DEAD fixable aqua fluorescent reactive dye (Invitrogen) according to manufacturer's instructions. ArC beads were stained with 3 uL neat Live/Dead Aqua for compensation. Following staining, the cells were pelleted down at 400 g for 8 minutes, 4° C., and resuspended in 500 uL/sample PBS wash before repeat of centrifugation. Negative ArC beads were added to the compensation control. GFP compensation beads were dispensed into separate tube as the GFP compensation control. All samples, including beads, were fixed for 15 minutes at room temperature with 4% paraformaldehyde in PBS. Following this, the samples were once again spun down at 400 g for 8 minutes, 4° C. and resuspended in FACS buffer (PBS+2 mM EDTA+0.5% w/v BSA). Samples were stored at 4° C. and flow analysis was carried out within 24 hours of fixation. Data were collected using a BD LSRFortessa I analyser running FACSDIVA software (Beckton Dickinson). Collected data were analysed using FlowJo 10.0 software. FIG. 18 shows that approximately 100% of C2c12 muscle cells were successfully transfected.

### Transfection of J774 Cells:

A day before transfection, J774 cells cultured in a 10 cm diameter dish were washed with PBS, scraped using a cell scraper and collected into a 50 ml Falcon tube. The cells were spun at 400 g for 5 minutes and resuspended in 10 ml of the J774 complete medium (DMEM with 10% heat inactivated FBS and 2 mM GlutaMAX supplement) for counting. The cells were seeded into a 24-well plate format. The cells were transferred to the incubator overnight. For transfection the medium overlaying the J774s cells was replaced with 480 ul/well of the fresh J774 complete medium. Cells were transfected with 120 ul/well 25 mM HEPES or formulation (various concentrations of encapsulated Alexa Fluor 488-eGFP mRNA) for 4 hours in the incubator (i.e. the total volume was 600 uL during transfection). After 4 hours the J774 cells were washed with PBS and harvested using 5 mM EDTA incubated with the cells for 15 minutes at 37 degrees Celsius and using a cell scraper. The J774 cells were then spun down. The supernatant was disposed and the cells were resuspended in 100 ul/sample LIVE/DEAD fixable red diluted in PBS (1:250) and left in the fridge for 30 minutes. The cells were then spun down 400 g for 5 minutes and washed with PBS. The cells were spun down once again and resuspended in 100 ul/sample 4% PFA (diluted in PBS) and fixed for 10 minutes at room temperature. Following fixation, the cells were spun down at 500 g for 5 minutes and washed with PBS. The cells were once again spun down and resuspended in 400 ul/sample FACS buffer (PBS with 0.5% w/v BSA & 2 mM EDTA) and passed through 70 um filters prior to flow analysis. Data were collected using a BD LSRFortessa I analyser running FACSDIVA software (Beckton Dickinson). Collected data were analysed using FlowJo 10.0 software.

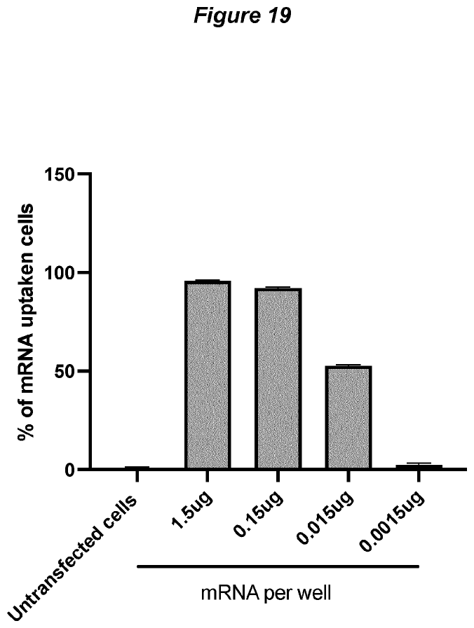

FIG. 19 shows that approximately 100% of J774 macrophages were successfully transfected when 1.5 ug of nucleic acid was applied using the compositions of the invention. Approximately 50% of J774 macrophages were successfully transfected when 0.015 ug of nucleic acid was applied using the compositions of the invention.

### Transfection of Jurkat Cells:

On the day of transfection, Jurkat cells were harvested and spun down at 300 g for 5 minutes and resuspended in 10 ml of the Jurkat complete medium (DMEM with 10% heat inactivated FBS and 2 mM GlutaMAX supplement) for cell counting. The cells were diluted down to 4.17E+5 cells/ml and 480 ul/well cells (i.e. 2E+5 cells/well) were seeded into a 24-well format. The cells were left for 30 minutes prior to transfection. The cells were transfected with 120 ul/well 25 mM HEPES or formulation (various concentrations of encapsulated Alexa Fluor 488-eGFP mRNA) for 4 hours in the incubator (i.e. the total volume was 600 uL during transfection). After 4 hrs transfection, the cells were collected into 1.5 ml Eppendorf tubes and spun down at 300 g for 5 minutes. The cells were then washed 1× with 750 ul/sample PBS and were then spun down. The supernatant was disposed and the cells were resuspended in 100 ul/sample LIVE/DEAD fixable red diluted in PBS (1:250) and left in the fridge for 30 minutes. The cells were then spun down 400 g for 5 minutes and washed with PBS. The cells were spun down once again and resuspended in 100 ul/sample 4% PFA (diluted in PBS) and fixed for 10 minutes at room temperature. Following fixation, the cells were spun down at 500 g for 5 minutes and washed with PBS. The cells were once again spun down and resuspended in 400 ul/sample FACS buffer (PBS with 0.5% w/v BSA & 2 mM EDTA) and passed through 70 um filters prior to flow analysis. Data were collected using a BD LSRFortessa I analyser running FACSDIVA software (Beckton Dickinson). Collected data were analysed using FlowJo 10.0 software.

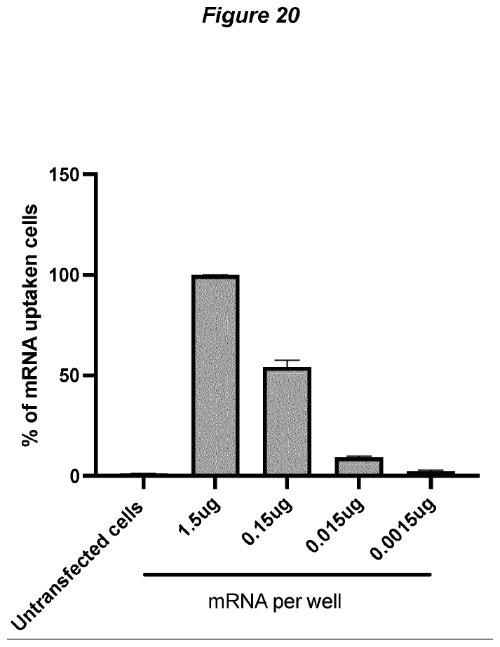

FIG. 20 shows that approximately 100% of Jurkat T cells were successfully transfected when 1.5 ug of nucleic acid was applied using the compositions of the invention. Approximately 50% of Jurkat T cells were successfully transfected when 0.15 ug of nucleic acid was applied using the compositions of the invention.

### Transfection of HeLa Cells:

HeLa cells were seeded in 96 well plates in order to reach 70% confluence. mRNA transfection complexes were formed by mixing mRNA with the dendrimers in 25 mM HEPES buffer, then with DOTMA:DOPE in 25 mM HEPES buffer at 25° C. mRNA expresses eGFP and was Alexa Fluor 488 tagged. The transfection complexes were overlaid on the cells in full growth medium. The cells were harvested 2 hours after transfection, as follows. Cells were washed and of incubated at 37° C. for 10 minutes. Then, the cells were gently detached from the plates and pelleted down. Cells were stained with LIVE/DEAD fixable aqua fluorescent reactive dye (Invitrogen) according to manufacturer's instructions. ArC beads were stained with 3 uL neat Live/Dead Aqua for compensation. Following staining, the cells were pelleted down at 4° C., and resuspended in 500 uL/sample PBS wash before repeat of centrifugation. Negative ArC beads were added to the compensation control. GFP compensation beads were dispensed into separate tube as the GFP compensation control. All samples, including beads, were fixed for 15 minutes at room temperature with 4% paraformaldehyde in PBS. Following this, the samples were once again spun down at 4° C. and then resuspended in FACS buffer (PBS+2 mM EDTA+0.5% w/v BSA). Samples were stored at 4° C. and flow analysis was carried out within 24 hours of fixation. Data were collected using a BD LSRFortessa I analyser running FACSDIVA software (Beckton Dickinson). Collected data were analysed using FlowJo 10.0 software.

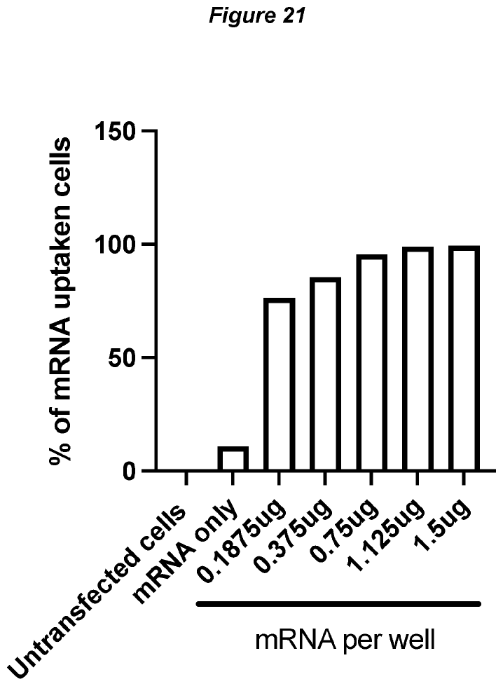

FIG. 21 shows that approximately 100% of HeLa cells were successfully transfected when 1.5 ug or 1.125 ug of nucleic acid was applied using the compositions of the invention. Approximately 75% of HeLa cells were successfully transfected when 0.1875 ug of nucleic acid was applied using the compositions of the invention.

### Example 15—Lipid Mixtures (i)

To formulate nanocarriers with dendrimers and mRNA with different lipids, individual lipids dissolved in solvent were mixed at the desired molar ratios and combined with dendrimer and mRNA.

To form an exemplary nanoparticle composition, GSCG1,2-RHL ((RHL)₄(KRHL)₂KGSC-NH₂) and EGFP mRNA were prepared and mixed in an aqueous buffer. The liposome mixtures disclosed below were then added.

DODAP:DOTAP:DOPE 1:1:1. DODAP (1,2-dioleoyloxy-3-(Dimethylamino)propane), DOTAP (1,2-Dioleoyl-3-trimethylammonium-propane) and DOPE (1,2-Dioleoyl-sn-glycero-3-phosphoethanolamine) were combined at a 1:1:1 molar ratio in an ethanol solvent and mixed with the solution of peptide and mRNA, giving a turbid solution. DLS measurement (27× dilution in 25 mM HEPES buffer): 115.9 nm; PDI 0.11.

DODAP:DOTMA:DOPE 1:1:1. DODAP, DOTMA (1 N-[1-(2,3-dioleyloxy)propyl]-n,n,n-trimethylammonium chloride) and DOPE were combined at a 1:1:1 molar ratio in an ethanol solvent and mixed with the solution of peptide and mRNA, giving a turbid solution. DLS measurement (27× dilution in 25 mM HEPES buffer): 116.9 nm, PDI 0.10.

DODAP:DOTAP:DOPE 1:1:2. DODAP, DOTAP and DOPE were combined at a 1:1:2 molar ratio in an ethanol solvent and mixed with the solution of peptide and mRNA, giving a turbid solution. DLS measurement (27× dilution in 25 mM HEPES buffer): 127.3 nm, PDI 0.09.

DODAP:DOTMA:DOPE 1:1:2. DODAP, DOTMA and DOPE were combined at a 1:1:2 molar ratio in an ethanol solvent and mixed with the solution of peptide and mRNA, giving a turbid solution. DLS measurement (27× dilution in 25 mM HEPES buffer): 136.9 nm, PDI 0.08.

DODAP:DOTMA:DOPE 2:1:1. DODAP, DOTMA and DOPE were combined at a 2:1:1 molar ratio in an ethanol solvent and mixed with the solution of peptide and mRNA, giving a turbid solution. DLS measurement (27× dilution in 25 mM HEPES buffer): 139.5 nm, PDI 0.07.

DODAP:DOTAP:DOPE 2:1:1. DODAP, DOTAP and DOPE were combined at a 2:1:1 molar ratio in an ethanol solvent and mixed with the solution of peptide and mRNA, giving a turbid solution. DLS measurement (27× dilution in 25 mM HEPES buffer): 160.3 nm, PDI 0.06.

DODAP:DOTAP 1:1. DODAP and DOTAP were combined at a 1:1 molar ratio in an ethanol solvent and mixed with the solution of peptide and mRNA, giving a turbid solution. DLS measurement (27× dilution in 25 mM HEPES buffer): 124.6 nm, PDI 0.12.

DODAP:DORI:DOPE 1:1:1. DODAP, DORI (N-(2-hydroxyethyl)-N,N-dimethyl-2,3-bis(oleoyloxy)propan-1-aminium bromide) and DOPE were combined at a 1:1:1 molar ratio in an ethanol solvent and mixed with the solution of peptide and mRNA, giving a turbid solution. DLS measurement (27× dilution in 25 mM HEPES buffer): 148.9 nm, PDI 0.06.

DODAP:DORI:DOPE 1:1:2. DODAP, DORI and DOPE were combined at a 1:1:1 molar ratio in an ethanol solvent and mixed with the solution of peptide and mRNA, giving a turbid solution. DLS measurement (27× dilution in 25 mM HEPES buffer): 160.3 nm, PDI 0.06.

DOPE has CAS Number: 4004-05-1 and formula: C41H7SNOsP); DOTAP has CAS Number:132172-61-3 and formula: C₄₂H₈₀NO₄Cl); DODAP has CAS number: 127512-29-2 and formula: C₄₁H₇₇NO₄. DORI has CAS number: 153312-59-5 and formula: C₄₃H₈₂BrNO₅. DOTMA has CAS number: 104162-48-3 and formula: C₄₂H₈₄NO₂Cl. DOTMA can also be named 1,2-di-O-octadecenyl-3-trimethylammonium propane (chloride salt), (CAS number: 104872-42-6).

The transfection efficacy of nanocarriers comprising each lipid mixture was assessed on human cancer cell lines (HeLa and A549).

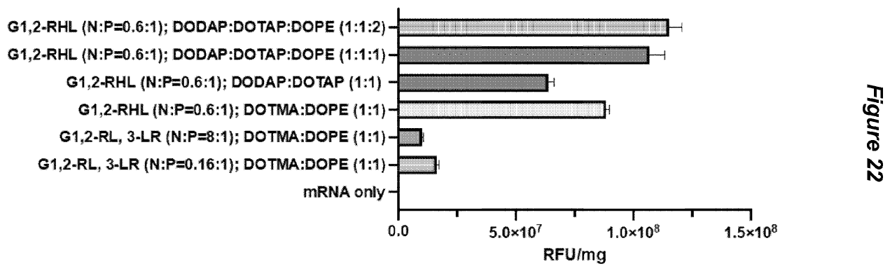

HeLa cells were transfected with nanocarriers formulated with different lipid components, using methods described herein. The nanocarriers used were comprising either (1) GSCG1,2-RL, 3-LR (N:P=0.16:1 to mRNA) with DOTMA:DOPE (with 1:1 in terms of the mole of DOTMA to the mole of DOPE), the overall lipid amount was w/w=10:1 to mRNA; (2) GSCG1,2-RL, 3-LR (N:P=8:1 to mRNA) with DOTMA:DOPE (with 1:1 in terms of the mole of DOTMA to the mole of DOPE), the overall lipid amount was w/w=10:1 to mRNA; (3) GSCG1,2-RHL (N:P=0.6:1 to mRNA) with DOTMA:DOPE (with 1:1 in terms of the mole of DOTMA to the mole of DOPE), the overall lipid amount was w/w=10:1 to mRNA; (4) GSCG1,2-RHL (N:P=0.6:1 to mRNA) with DODAP:DOTAP (with 1:1 in terms of the mole of DODAP to the mole of DOTAP), the overall lipid amount was w/w=10:1 to mRNA; (5) GSCG1,2-RHL (N:P=0.6:1 to mRNA) with DODAP:DOTAP:DOPE (with 1:1:1 in terms of the mole of DODAP to the mole of DOTAP to the mole of DOPE), the overall lipid amount was w/w=10:1 to mRNA and (6) GSCG1,2-RHL (N:P=0.6:1 to mRNA) with DODAP:DOTAP:DOPE (with 1:1:2 in terms of the mole of DODAP to the mole of DOTAP to the mole of DOPE), the overall lipid amount was w/w=10:1 to mRNA. (7) GSCG1,2-RHL (N:P=0.6:1 to mRNA) with DODAP:DOTMA:DOPE (with 1:1:1 in terms of the mole of DODAP to the mole of DOTMA to the mole of DOPE), the overall lipid amount was w/w=10:1 to mRNA; and (8) GSCG1,2-RHL (N:P=0.6:1 to mRNA) with DODAP:DOTMA:DOPE (with 1:1:2 in terms of the mole of DODAP to the mole of DOTMA to the mole of DOPE), the overall lipid amount was w/w=10:1 to mRNA. The mRNA used was mRNA expressing eGFP, and the cells were harvested 24 hours post-transfection to analyse fluorescent proteins expression by a plate reader. Control: Cells transfected with mRNA alone. FIGS. 22 and 23 show the increased transduction efficiencies that were achieved by three lipid systems within a nanoparticle comprising a representative second generation peptide dendrimer, in transfecting HeLa cells.

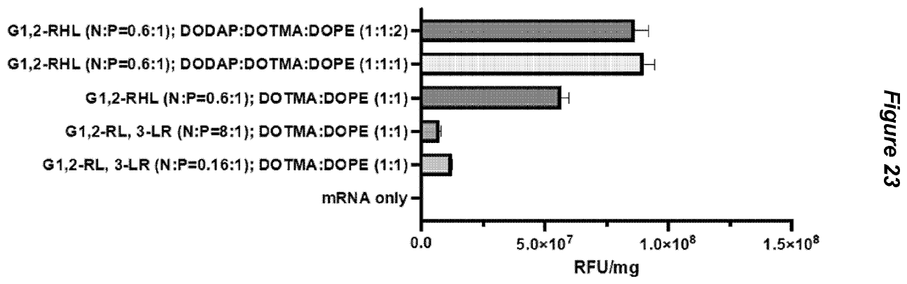

A549 cells were transfected with nanocarriers formulated with different lipid components, using methods described herein. The nanocarriers used were comprising either (1) GSCG1,2-RL, 3-LR (N:P=0.16:1 to mRNA) with DOTMA:DOPE (with 1:1 in terms of the mole of DOTMA to the mole of DOPE), the overall lipid amount was w/w=10:1 to mRNA; (2) GSCG1,2-RL, 3-LR (N:P=8:1 to mRNA) with DOTMA:DOPE (with 1:1 in terms of the mole of DOTMA to the mole of DOPE), the overall lipid amount was w/w=10:1 to mRNA; (3) GSCG1,2-RHL (N:P=0.6:1 to mRNA) with DOTMA:DOPE (with 1:1 in terms of the mole of DOTMA to the mole of DOPE), the overall lipid amount was w/w=10:1 to mRNA; (4) GSCG1,2-RHL (N:P=0.6:1 to mRNA) with DODAP:DOTAP:DOPE (with 2:1:1 in terms of the mole of DODAP to the mole of DOTAP to the mole of DOPE), the overall lipid amount was w/w=10:1 to mRNA; and (5) GSCG1,2-RHL (N:P=0.6:1 to mRNA) with DODAP:DORI:DOPE (with 1:1:1 in terms of the mole of DODAP to the mole of DORI to the mole of DOPE), the overall lipid amount was w/w=10:1 to mRNA; (6) GSCG1,2-RHL (N:P=0.6:1 to mRNA) with DODAP:DORI:DOPE (with 1:1:2 in terms of the mole of DODAP to the mole of DORI to the mole of DOPE), the overall lipid amount was w/w=10:1 to mRNA; (7) GSCG1,2-RHL (N:P=0.6:1 to mRNA) with DODAP:DOTMA:DOPE (with 2:1:1 in terms of the mole of DODAP to the mole of DORI to the mole of DOPE), the overall lipid amount was w/w=10:1 to mRNA; The mRNA used was mRNA expressing eGFP, and the cells were harvested 24 hours post-transfection to analyse fluorescent proteins expression by a plate reader. Formulation (4), (5), (6) and (7) contained only 67% of the eGFP mRNA content compared to formulation (1), (2) and (3). Control: Cells transfected with mRNA alone. FIG. 24 shows the increased transduction efficiencies that were achieved by three lipid systems within a nanoparticle comprising a representative second generation peptide dendrimer, in transfecting A549 cells.

TABLE 4
The size and PDI of the GSCG1,2-RHL (N:P =
0.6:1 to mRNA) nanocarriers with different lipid components.

| Formulations | Size (nm) | PDI |
|---|---|---|
| GSCG1,2-RHL, | 115.9 | 0.11 |
| DODAP:DOTAP:DOPE (1:1:1) | | |
| GSCG1,2-RHL, | 116.9 | 0.1 |
| DODAP:DOTMA:DOPE (1:1:1) | | |
| GSCG1,2-RHL, | 127.3 | 0.09 |
| DODAP:DOTAP:DOPE (1:1:2) | | |
| GSCG1,2-RHL, | 136.9 | 0.08 |
| DODAP:DOTMA:DOPE (1:1:2) | | |
| GSCG1,2-RHL, | 147.5 | 0.1 |
| DODAP:DOTAP:DOPE (2:1:1) | | |
| GSCG1,2-RHL, | 148.9 | 0.06 |
| DODAP:DORI:DOPE (1:1:1) | | |
| GSCG1,2-RHL, | 160.3 | 0.06 |
| DODAP:DORI:DOPE (1:1:2) | | |
| GSCG1,2-RHL, | 139.5 | 0.07 |
| DODAP:DOTMA:DOPE (2:1:1) | | |
| GSCG1,2-RHL, DODAP:DOTAP | 124.6 | 0.12 |
| (1:1) | | |
| GSCG1,2-RHL, | 119.5 | 0.088 |
| DODAP:DOTMA:DOPE:DMG- | | |
| PEG (1:1:1:0.046) | | |
| GSCG1,2-RHL, | 115.3 | 0.115 |
| DODAP:DOTAP:DOPE:DMG- | | |
| PEG (1:1:1:0.046) | | |

The ratio in the bracket indicates the mole ratio between the lipids.
The total amount of the lipid to mRNA was w/w = 10:1.
(The bottom rows, DODAP:DOTMA:DOPE:DMG-PEG and DODAP:DOTAP:DOPE:DMG-PEG are exemplified below at Example 26).

### Example 16—Comparing Linear and Dendritic PGA

Human T cells were transfected with naked mRNA expressing eGFP (enhanced GFP), or with nanocarriers comprising mRNA expressing eGFP, using methods described herein. The nanocarriers comprised GSCG1,2-RHL (N:P=0.6:1 to mRNA) with DOTMA/DOPE (w/w=10:1 to mRNA) and the mRNA.

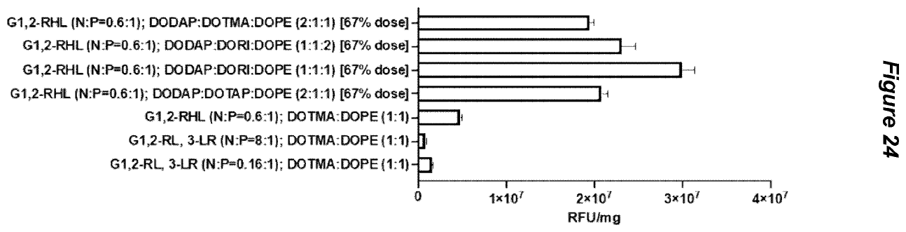

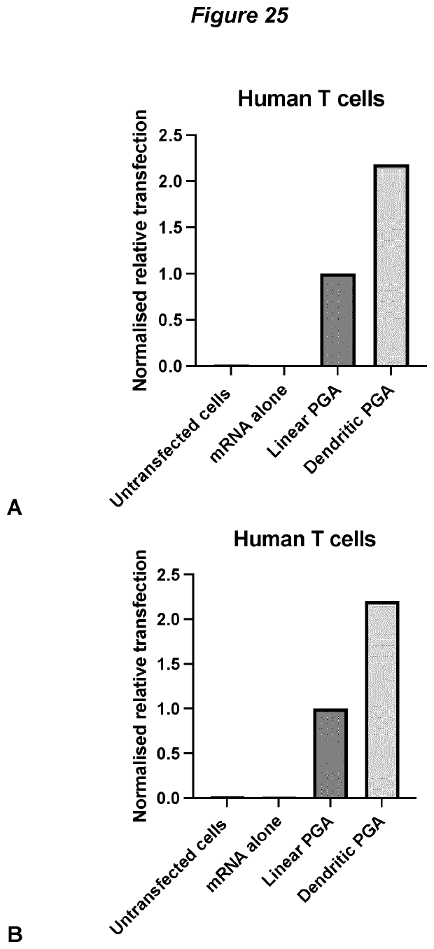

In a first experiment, the nanocarriers were coated with the same number of molecules of either linear PGA or dendritic PGA (FIG. 25A). The linear PGA was the linear PGA with 100 glutamic acids; the dendritic PGA was GSEGSEGSEC(OH)G1-(Ac)ESGESGESG.

TABLE 9

| | | Mol ratio | | Charge ratio |
|---|---|---|---|---|
| | Coating | (Coating to | | (Coating E to |
| Coating material | (mmoles) | mRNA P) | Charge (mmoles) | mRNA P) |
| Linear PGA with 100 | 3.38E−07 | 0.041 | 3.38E−05 | 4.14 |
| glutamic acids | | | | |
| GSEGSEGSEC(OH)G1- | 3.38E−07 | 0.041 | 3.05E−06 | 0.372 |
| (Ac)ESGESGESG | | | | |

Exemplary calculation of coating nanoparticles with linear PGA molecules or
the same number of dendritic PGA molecules (see FIG. 25A).

TABLE 10
Hydrodynamic size, polydispersity index (PDI) and zeta potential
of nanoparticles coated with linear PGA molecules or the same
number of dendritic PGA molecules (see FIG. 25A).

| Ratio | Size (nm) | PDI | Zeta potential (mV) |
|---|---|---|---|
| Linear PGA coated particles | 180 | 0.12 | −59 |
| Dendritic PGA coated particles | 184 | 0.16 | −32 |

In a second experiment, the nanocarriers were coated with equivalent molar charge of either linear PGA or dendritic PGA (FIG. 25B). The linear PGA was the linear PGA with 100 glutamic acids; the dendritic PGA was G1-EEEE.

TABLE 11
Exemplary calculation of coating nanoparticles with
linear PGA molecules or the equivalent molar charge
of dendritic PGA molecules (see FIG. 25B).

| | | Mol ratio | | Charge ratio |
|---|---|---|---|---|
| | Coating | (Coating to | Charge | (Coating E |
| Coating material | (mmoles) | mRNA P) | (mmoles) | to mRNA P) |
| Linear PGA with | 3.47E−07 | 0.042 | 3.47E−05 | 4.25 |
| 100 glutamic acids | | | | |
| G1-EEEE | 4.35E−06 | 0.532 | 3.48E−05 | 4.25 |

TABLE 12
Hydrodynamic size, polydispersity index (PDI) and zeta potential
of nanoparticles coated with linear PGA molecules or the equivalent
molar charge of dendritic PGA molecules (see FIG. 25B).

| | | | Zeta potential |
|---|---|---|---|
| Ratio | Size (nm) | PDI | (nmV) |
| Linear PGA coated particles | 175 | 0.14 | −46 |
| Dendritic PGA coated particles | 187 | 0.10 | −22 |

The expression of the eGFP protein within the cells was quantified by flow cytometry. FIGS. 25A and 25B shows expression levels normalised to the level achieved by the formulation coated with linear PGA (“normalised relative transfection”). Negative control cells were either not transfected or transfected only with mRNA.

### Example 17—Muscle Targeting Nanocarriers

The nanoparticles of the invention were coated with a dendrimer containing a muscle targeting peptide to improve targeting and transfection of muscle cells, even differentiated muscle cells.

Nanocarrier comprising GSCG1,2-RHL (N:P=0.6:1 to mRNA) with DOTMA/DOPE (w/w=10:1 to mRNA) and mRNA was firstly prepared as described. The nanoparticles were then coated with a dendrimer with a muscle targeting domain, the muscle-G1-EEEE (Ac-EEEE)2KGSCGAASSLNIA-(Acp)-NH2), at different mRNA:coating mole ratios, between 0.5:1 to 5:1, preferably around 2.74:1. As a control, the nanoparticles were coated with a dendrimer without a muscle targeting domain, the G1-EEEE, at different mRNA:coating mole ratios, between 0.5:1 to 5:1, preferably around 2.74:1.

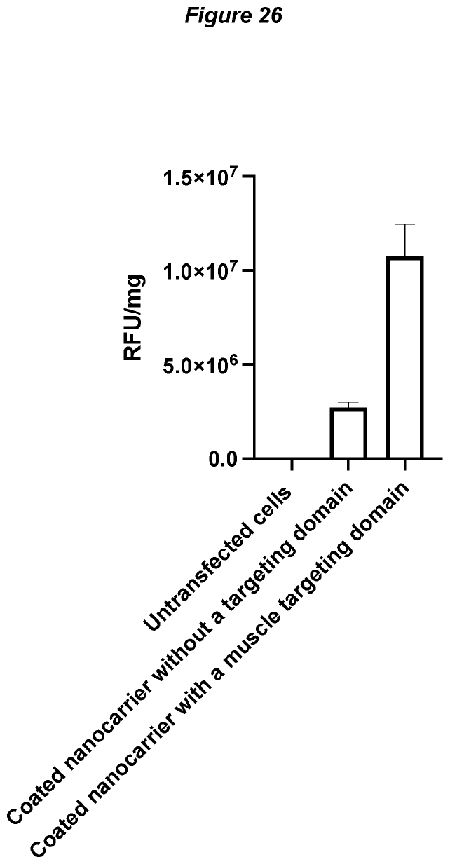

In a first experiment, mouse muscle cells (C2c12 cells) were transfected with formulations comprising mRNA expressing eGFP. The formulations used were comprising GSCG1,2-RHL (N:P=0.6:1 to mRNA) with DOTMA/DOPE (w/w=10:1 to mRNA) and mRNA, and coated with either the same equivalent of mole of dendritic PGA with or without the muscle targeting domain. The cells were harvested 24 hours post-transfection. Control: Cells were not transfected with mRNA. The dendrimer with the muscle targeting domain was (Ac-EEEE)2KGSCGAASSLNIA-(Acp)-NH2 (named “muscle-G1-EEEE”. ASSLNIA, SEQ ID NO:1, is the muscle targeting motif). The dendrimer without the muscle targeting domain was (Ac-EEEE)2KGSGGSGGSC-NH2 (“GSGGSGGSCG1-EEEE”). FIG. 26 shows the dramatic increase in eGFP expression in muscle cells transfected with nanocarriers comprising the muscle targeting domain.

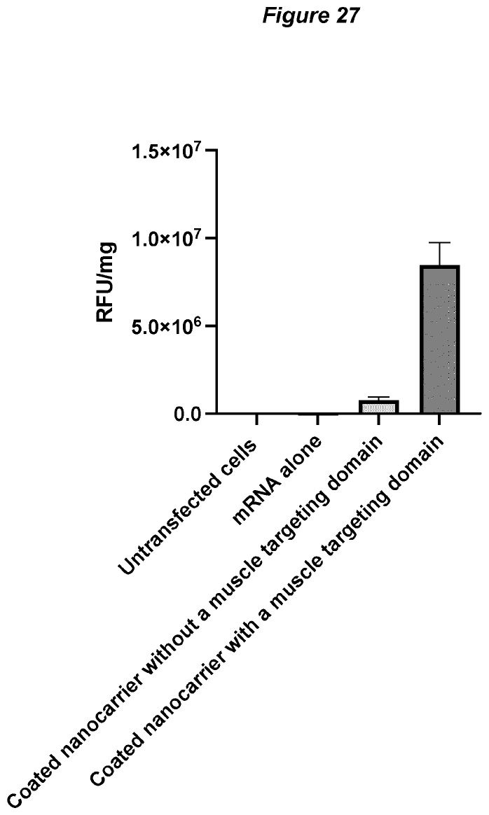

In a second experiment, differentiated mouse muscle cells (C2c12 cells) were transfected with formulations comprising mRNA expressing eGFP. The muscle cells were differentiated as follows: C2c12 cells were seeded and allowed to grow to confluence in a growth medium. Once the cells were confluent, the medium on the cells was replaced with a differentiation medium for 2 days to allow the cells to develop myotube characteristics. The differentiation medium was replaced with the growth medium 30 mins before transfection. The cells were transfected for 24 hours with nanocarriers indicated in FIG. 27 and harvested to assay the eGFP reporter gene expression using a plate reader. The mRNA used expressed eGFP. The growth medium contained high glucose DMEM (Gibco), 10% FBS (Gibco), 2 mM L-glutamine (Gibco). The differentiated medium contained high glucose DMEM (Gibco), 2% horse serum (Gibco), 2 mM L-glutamine (Gibco), 1 uM insulin.

As in the first experiment, the formulations used were comprising GSCG1,2-RHL (N:P=0.6:1 to mRNA) with DOTMA/DOPE (w/w=10:1 to mRNA) and mRNA, and coated with either the same equivalent of mole of dendritic PGA with or without the muscle targeting domain. The cells were harvested 24 hours post-transfection. Control: Cells were not transfected with mRNA or transfected with mRNA alone. The dendrimer with the muscle targeting domain was muscle-G1-EEEE. The dendrimer without the muscle targeting domain was GSGGSGGSCG1-EEEE. FIG. 27 shows the dramatic increase in eGFP expression in differentiated muscle cells transfected with nanocarriers comprising the muscle targeting domain.

Nanoparticles can also be coated with the muscle-G1-EEEE different mole ratio of the dendrimer such as 2.72:1 and 2.06:1 (dendrimer to mRNA) and showed improved mRNA transfection on differentiated C2c12 cells compared to the nanoparticles coated with dendrimer without the targeting domain. muscle-G1-EEEE coated at 2.72:1 had a PDI of 0.14 and a zeta potential of ˜44.0 mV, size 172 nm. muscle-G1-EEEE coated at 2.06:1 had a PDI of 0.15 and a zeta potential of ˜38.7 mV, size 176 nm. GSGGSGGSCG1-EEEE coated at 2.72:1 had a PDI of 0.13 and a zeta potential of ˜35.4 mV, size 169 nm. GSGGSGGSCG1-EEEE coated at 2.06:1 had a PDI of 0.12 a zeta potential of ˜33.2 mV, size 166 nm. The uncoated particle had a PDI of 0.21, zeta potential of +37.1 and size 190 nm.

### Example 18—Tumour Targeting Nanocarriers

A549 is a cancer cell that highly expresses integrin (Guo et al, 2009). A549 cells were transfected with nanocarriers coated with dendritic PGA with or without an integrin targeting domain ACDCRGDCFCG (SEQ ID NO:5) and comprising an mRNA expressing eGFP. The nanocarriers also comprise GSCG1,2-RHL (N:P=0.6:1 to mRNA) and DOTMA/DOPE (w/w=10:1 to mRNA). Molar equivalent of dendritic PGA (with or without the integrin targeting domain) was used. The cells were harvested 24 hours post-transfection. Control: Cells were not transfected with mRNA or transfected with mRNA only. The dendrimer with the integrin targeting domain was (Ac-EEEE)2KGSGGSGGSACDCRGDCFCG-NH2 (Disulfide bridges:C1-C4, C2-C3). The dendrimer without the integrin targeting domain was (Ac-EEEE)2KGSGGSGGSC-NH2 (“GSGGSGGSCG1-EEEE”).

Methods: Firstly, an initial solution of GSCG1,2-RHL and DOTMA:DOPE nanoparticles in 25 mM HEPES buffer (150 ug/mL mRNA) was prepared, as follows: To a sterile polypropylene tube Peptide (RHL)4(KRHL)2KGSC-NH2 (4912 g/mol inc. TFA counter ions, 10 N per peptide) at 10 mg/ml in water (4.240 uL, 42.4 ug, 8.632×10⁻⁶ mmoles, 8.632×10⁻⁵ mmoles N), sterile water (37.34 uL) and 200 mM HEPES buffer (5.94 uL) were added and mixed. To a second tube, CleanCap® EGFP mRNA (5 moU) at 1 mg/mL in 1 mM Sodium Citrate pH 6.4 (46.44 uL, 46.44 ug, 1.407×10⁻⁴ mmoles P), sterile water (48.37 uL) and 200 mM HEPES buffer (13.54 uL) were added and mixed. To a third tube, DOTMA:DOPE liposome (1:1 molar ratio) at 25 mM (17.68 mg/mL) in water (16.27 uL, 287.7 ug, 3.287×10⁻⁴ mmoles N), sterile water (109.2 uL) and 200 mM HEPES buffer (19.34 uL) were added and mixed. 43.20 uL of the peptide mixture (7.847×10⁻⁶ mmoles, 7.847×10⁻⁵ mmoles N, 0.60 equivalents wrt. mRNA P) was transferred to a fresh polypropylene tube. 100.8 uL of the mRNA mixture (1.309×10⁻⁴ mmoles P) was added into the tube containing peptide, rapidly mixing up and down with a pipette. The solution was allowed to incubate for 2 minutes. 144.0 uL of the Liposome mixture (3.057×10⁻⁴ mmoles N) was added into the tube containing peptide and mRNA, rapidly mixing up and down with a pipette, giving a turbid solution. DLS measurement (40× dilution in 25 mM HEPES buffer): 184 nm, Pdl 0.13). Zeta potential measurement (Diluted 3× in 25 mM HEPEs, and then a further 60× dilution in water): +33.5 mV.

### Coating Material Stock Solutions:

Int-GSGGSGGSCG1-EEEE (Ac-EEEE)2KGSGGSGGSACDCRGDCFCG (disulfide bridge between C1 and C4, and disulfide bridge between C2 and C3, 3081 g/mol, 8 negative charges per peptide). 2.00 mg was dissolved in 133.3 uL 50 mM Ammonium carbonate solution, followed by 266.7 uL 25 mM HEPES buffer to give a final concentration of 5.0 mg/mL. Tube was centrifuged at 11,000 g for 3 minutes and aliquoted, before storing at −80° C.

GSGGSGGSCG1-EEEE ((Ac-EEEE)2KGSGGSGGSC-NH2, 1912 g/mol, 8 negative charges per peptide). 10 mg was dissolved in 156.2 uL 50 mM Ammonium carbonate solution, followed by 468.8 uL 25 mM HEPES buffer to give a final concentration of 16.0 mg/mL. Tube was centrifuged at 11,000 g for 3 minutes and aliquoted, before storing at −80° C.

### Coated Nanoparticles:

The base nanoparticle described above was diluted two-fold in 25 mM HEPES to an mRNA concentration of 75 μg/mL. To 134.0 uL (10.05 ug mRNA, 3.045×10⁻⁵ mmoles P) of these nanoparticles, 67.0 uL of coating material dissolved in 25 mM HEPES was added, rapidly mixing up and down with a pipette. The final mRNA concentration of the solution was 50 μg/mL. A breakdown of the coatings is provided in the table below:

TABLE 13

| | | | | Charge | | | |
|---|---|---|---|---|---|---|---|
| | | Mol ratio | | ratio | Z-average | | Zeta |
| Coating | Coating | (Coating | Charge | (Coating E | diameter | | Potential |
| material | (mmoles) | to mRNA P) | (mmoles) | to mRNA P) | (nm) | Pdl | (mV) |
| Int- | 6.263E−05 | 2.06 | 5.010E−04 | 16.45 | 191 | 0.20 | −31.9 |
| GSGGSGGSC | | | | | | | |
| G1-EEEE | | | | | | | |
| GSGGSGGSC | 6.262E−05 | 2.06 | 5.009E−04 | 16.45 | 165 | 0.16 | −29.3 |
| G1-EEEE | | | | | | | |

An example of the coating calculation and the size, zeta potential and PDl of the
resulting nanoparticles. The size (nm), zeta potential (mV) and PDl of the uncoated
nanocarrier were 190 nm, +37.1 mV and 0.21 respectively.

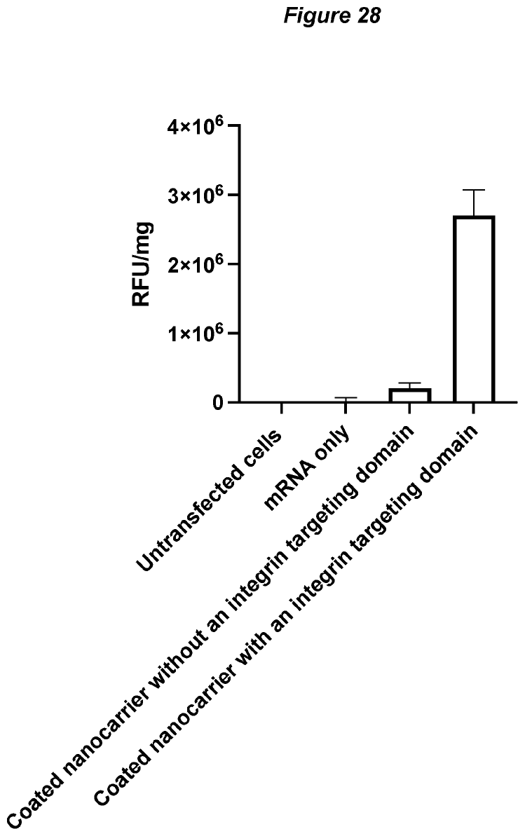

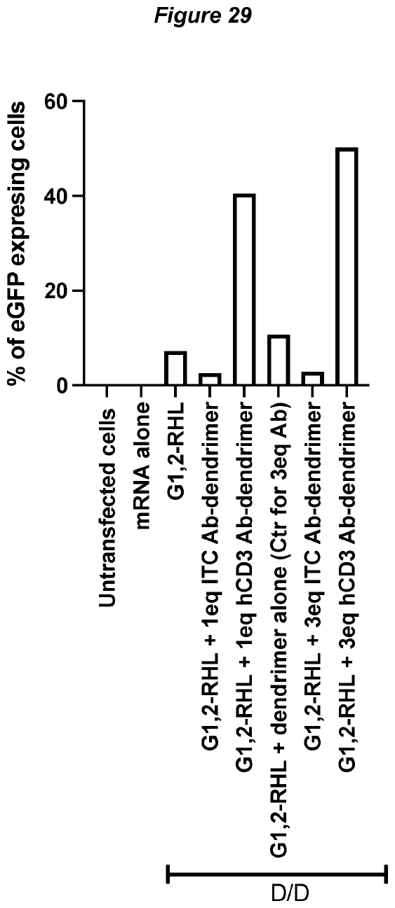

FIG. 28 shows the dramatic increase in eGFP expression in A549 cells transfected with nanocarriers comprising the tumour targeting integrin binding domain.

### Example 19—Nanocarriers Targeted Via an Antibody-Conjugated Dendrimers

GSEGSEGSEC(OH)G1-(Ac)ESGESGESG ([(Ac-ESGESGESG)2K]GSEGSEGSEC, 2793 g/mol, 9 negative charges per peptide) at 10 mg/mL in water (180 ug, 6.446×10⁻⁵ mmoles, 5.801×10⁻⁴ mmoles COOH) was added to a 500 uL polypropylene tube. N-(3-Dimethylaminopropyl)-N′-ethylcarbodiimide hydrochloride (EDC·HCl, 191.7 g/mol) at 2 mg/mL in water (6.80 uL, 13.59 ug, 7.090×10⁻⁵ mmoles) was added and solution allowed to incubate for 2 minutes at room temperature. N-Hydroxysulfosuccinimide sodium salt (Sulfo-NHS, 217.1 g/mol) at 2 mg/mL in water (8.47 uL, 16.94 ug, 7.799×10⁻⁵ mmoles) was added and the solution allowed to incubate for 15 minutes at room temperature. InVivoMAb anti-human CD3 (BioXCell BE0001-2, 150 kDa) at 7.19 mg/mL in PBS pH 7.0 (134.5 uL, 967 ug, 6.446×10⁻⁶ mmoles) was added and the solution allowed to incubate for 4 h at room temperature. 182.2 uL of PBS was added, and the 350 μL of solution was dialysed (MWCO 50 kDa) in 500 mL PBS overnight at 4° C. The resulting conjugate had a theoretical concentration of 3.373 mg/mL in PBS (0.529 mg/mL GSEGSEGSEC(OH)G1-(Ac)ESGESGESG peptide, 2.844 mg/mL antibody).

An initial solution of lipid nanoparticles, comprising mRNA, are prepared before coating them with the antibody-conjugated dendrimer, e.g. at a 2:1 volumetric ratio by mixing. Increasing the coating materials reducing the zeta potential, indicating coating of the nanoparticles.

### Example 20—T Cell Targeting Nanocarriers Comprising CD3-Binding Antibody

Nanocarriers were coated with human CD3 targeting antibody improve human T cells targeting mRNA delivery. Jurkat cells were untransfected, or were transfected with the following compositions:

(1) eGFP expressing mRNA alone; (2) GSCG1,2-RHL (N:P=0.6 to mRNA) and DOTMA/DOPE (w/w=10 to mRNA) with mRNA (control for (4) and (7)); (3) GSCG1,2-RHL (N:P=0.6 to mRNA) and DOTMA/DOPE (w/w=10 to mRNA) with mRNA, coated with 1 equivalent of isotype antibody control (ITC) conjugated dendrimer (control for (4)); (4) GSCG1,2-RHL (N:P=0.6 to mRNA) and DOTMA/DOPE (w/w=10 to mRNA) with mRNA, coated with 1 equivalent of anti-CD3 antibody conjugated dendrimer; (5) GSCG1,2-RHL (N:P=0.6 to mRNA) and DOTMA/DOPE (w/w=10 to mRNA) with mRNA, coated with dendrimer alone (control for (7)); (6) GSCG1,2-RHL (N:P=0.6 to mRNA) and DOTMA/DOPE (w/w=10 to mRNA) with mRNA, coated with 3 equivalent of an isotype antibody control (ITC) conjugated dendrimer (control for (7)); (7) GSCG1,2-RHL (N:P=0.6 to mRNA) and DOTMA/DOPE (w/w=10 to mRNA) with mRNA, coated with 3 equivalent of anti-CD3 antibody conjugated dendrimer. D/D denotes DOTMA:DOPE (w/w=10:1 to mRNA).

The T cells were transfected for 2 hours using 4.5 ug mRNA in 24 well plates. eGFP expression was analysed by cytometry 24 hours post-transfection of the formulations. Isotype control (ITC): InVivoMAb mouse IgG2a isotype control (BioXCell BE0085, C1.18.4 Clone).

### Example 21—Delivery of Multiple Nucleic Acids in a Single Formulation to Cells, In Vitro

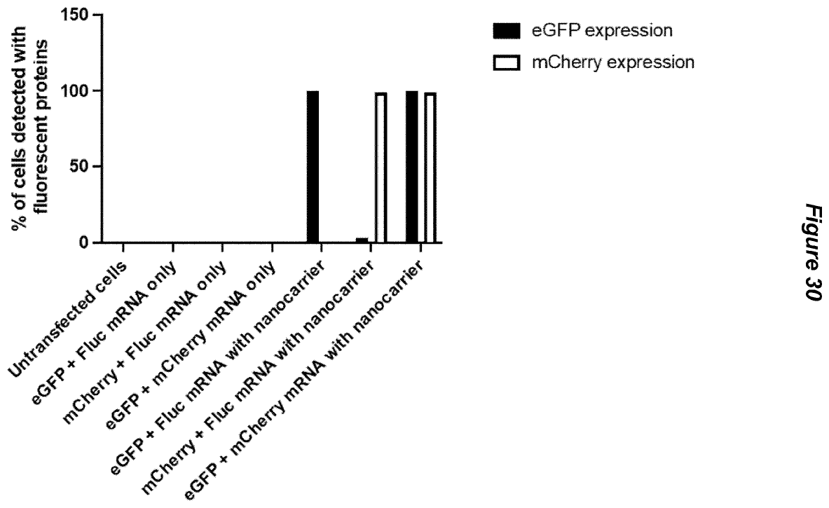

HeLa cells were transfected with formulations comprising two nucleic acids; selected from a first mRNA that expresses eGFP, a second mRNA that expresses mCherry, and a third mRNA that expresses luciferase. The nanocarriers also comprised GSCG1,2-RHL (N:P=0.6:1 to mRNA) with DOTMA/DOPE (w/w=10:1 to mRNA). The mRNA molecules were mRNA expressing eGFP and mRNA expressing mCherry or mRNA expressing luciferase (Fluc). The cells were harvested 24 hours post-transfection to analyse fluorescent proteins expression my flow cytometry. The eGFP+Fluc mRNA only or with nanocarrier consisted of 1:1 w/w eGFP mRNA to Fluc mRNA; The mCherry+Fluc mRNA only or with nanocarrier consisted of 1:1 w/w mCherry mRNA to Fluc mRNA; The eGFP+mCherry mRNA only or with nanocarrier consisted of 1:1 w/w eGFP mRNA to mCherry mRNA. FIG. 30 shows that the nanocarriers can encapsulate and deliver more than one mRNA for functional protein expression.

TABLE 14
The size and PDI of the GSCG1,2-RHL
(N:P = 0.6:1) nanocarriers with 2 mRNAs.
All nanocarriers also contained DOTMA:DOPE.

| | Hydrodynamic size | Polydispersity index |
|---|---|---|
| Nanocarrier | (nm) | (PDI) |
| GSCG1,2-RHL with | 162.9 | 0.1478 |
| eGFP and mCherry | | |
| mRNA | | |
| GSCG1,2-RHL with | 163.9 | 0.1336 |
| eGFP and Fluc mRNA | | |
| GSCG1,2-RHL with | 149 | 0.1394 |
| mCherry and Fluc | | |
| mRNA | | |

### Example 22—Delivery of Multiple Nucleic Acids in a Single Formulation to Subjects, In Vivo

The nanocarrier of GSCG1,2-RHL (N:P=0.6:1 to mRNA), DOTMA:DOPE (10:1 to mRNA) with mRNA was prepared as described before. To prepare the nanocarrier of GSCG1,2-RHL (N:P=0.6:1 to mRNA) with CpG, DOTMA:DOPE (10:1 to mRNA & CpG) and mRNA, firstly the mRNA and CpG were mixed at w/w=9:1, mRNA to CpG. This mixture was then added to the GSCG1,2-RHL (N:P=0.6:1), followed with the addition of DOTMA:DOPE (10:1 to mRNA & CpG). The CpG used was the CpG ODN 2006 (CpG 7909) purchased from InvivoGen.

Luciferase expression in mice tissue following intravenous administration of compositions comprising GSCG1,2-RHL (N:P=0.6:1 to mRNA) with DOTMA/DOPE (w/w=10:1 to mRNA), an mRNA expressing luciferase and a CpG molecule, with DOTMA/DOPE (w/w=10:1 to nucleic acids). CpG oligonucleotides (ODNs) are synthetic ODNs that contain unmethylated CpG dinucleotides (CpG motifs). mRNA alone treatment was used as a control. Mice were injected with the compositions and 6 hours later the tissues were harvested to measure luciferase signal in muscle (gastrocnemius), liver, lung, heart, spleen, kidney, adipose tissues (adipose) and brain.

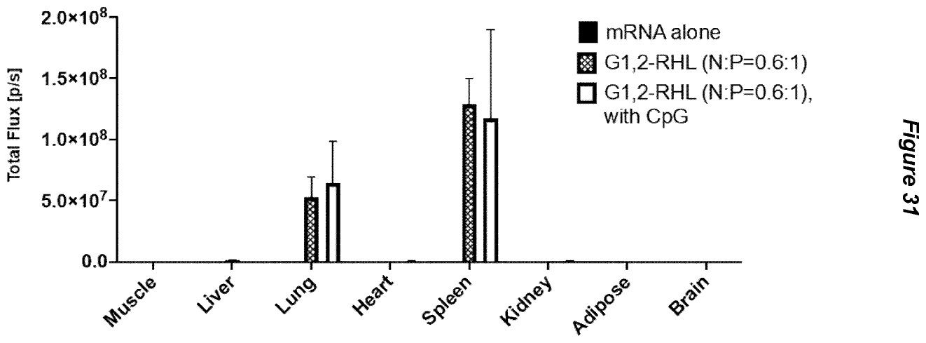

FIG. 31 shows that high levels of luciferase were detected in lung and spleen, 6 hours after administration of nanocarriers comprising luciferase mRNA and CpG (white bars).

TABLE 15

| | | Zeta | |
|---|---|---|---|
| | Hydrodynamic | Potential | |
| Nanocarrier | size (nm) | (mV) | PDI |
| GSCG1, 2-RHL with | 176 | +30.5 | 0.18 |
| CpG | | | |
| GSCG1, 2-RHL | 174 | +48 | 0.17 |

TSize, zeta potential and PDI of the GSCG1,2-RHL
(N: P = 0.6:1) nanocarriers, with DOTMA: DOPE,
coated with GSGGSGGSCG1-EEEE with/without CpG
nanocarriers.

### Example 23—Functional Delivery to Primary Leucocytes Using Nanoparticles Carrying Two Nucleic Acids

Bone marrow cells were isolated from BALB/c mice resuspended in complete DC media (RPMI-1640+10% heat inactivated FBS+1% P/S+40 ng/ml murine GM-CSF from Peprotech) at 2-3E+5 cells/ml and seeded into 10 cm diameter dishes (2-3E+6 cells/dish). 2-3 days after seeding an equal volume of complete DC media was added. A half-media exchange was performed 5-6 days and day 8 post-seeding.

Transfection and staining: Suspension cells (moDCs) cultured for 13 days were spun down and resuspended at a concentration of 5.68E+5 cells/ml in complete DC media and seeded in a 24-well plate format 2.5E+5 cells/well (i.e. 440 ul/well). Cells were then transfected with (1) GSCG1,2-RHL (N:P=0.6:1 to mRNA) with DOTMA/DOPE (w/w=10:1 to mRNA) and mRNA encoding eGFP or (2)) GSCG1,2-RHL (N:P=0.6:1 to mRNA) with DOTMA/DOPE (w/w=10:1 to mRNA), mRNA encoding eGFP and a CpG nucleotides for 2, 4 or 22 hours. Transfection efficiency (% eGFP+) of moDCs was measured 22-24 hours after the start of the transfection using flow cytometry. moDCs were distinguished by surface expression of CD11c and the absence of F4/80 (macrophage marker) expression. D/D refers to DOTMA/DOPE (w/w=10:1 to mRNA).

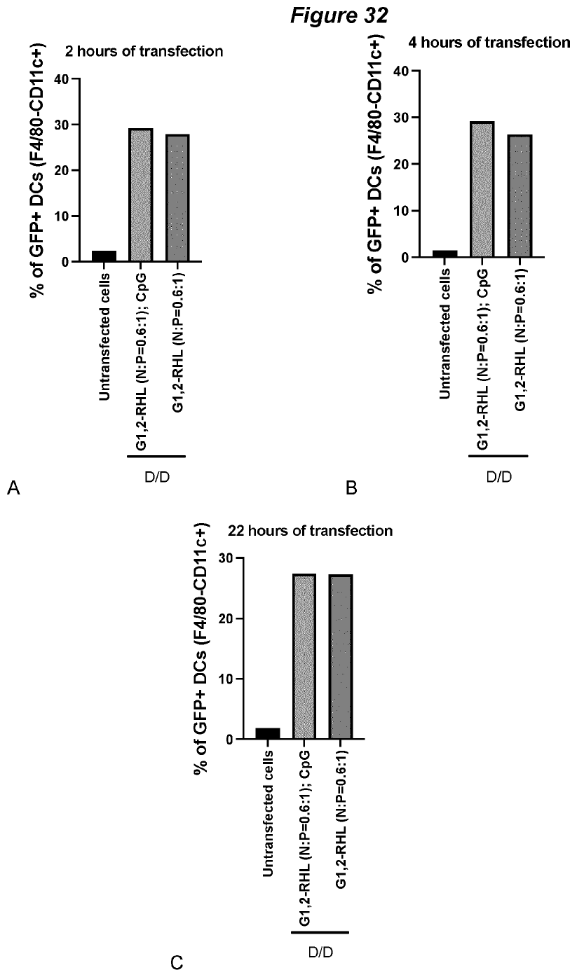

Cells were subsequently spun down and resuspended in 1 ml fresh complete media and reseeded into 24-well plate. The next day cells were harvested and stained with live/dead aqua (1:250 in PBS) for 30 minutes. After PBS washes cells were blocked with 50 ul/sample 2× TruStain human FcX block diluted in FACS buffer (1:20; end concentration 1:40) for 5 minutes. Subsequently cells were stained with a 50 ul/sample 2× cocktail of surface marker antibodies diluted in FACS buffer for 20 minutes. The final concentrations of these antibodies used were: 1:100 CD11c-PerCP-Cy5.5 (Biolegend; 117328), 1:200 CD11b-APC (Biolegend; 101212) and 1:100 F4/80-PE (Biolegend; 123110). After FACS buffer washes, the cells were fixed using 4% PFA for 10 minutes. Cells were washed using PBS and resuspended in FACS buffer for flow cytometry analysis. Arc, eGFP and UltraComp Plus beads were used for compensation. FIG. 32 shows that eGFP expression was achieved at a similar level by all three transfection times.

### Example 24—Functional Repolarization of Primary Macrophages

Nanocarriers of the invention were used to deliver modified IRF5 to polarise M2 macrophages to M1 macrophages. Modified IRF5 is a protein with mutations which functions as the activated version of the wildtype IRF5. The nanocarrier formulation used was comprising GSCG1,2-RHL (N:P=0.6:1 to mRNA) with DOTMA/DOPE (w/w=10:1 to mRNA) and mRNA expressing modified IRF5.

Primary murine bone marrow derived macrophages were polarized to the M2 phenotypes, which were then transfected for 24 hours with the nanocarrier formulation.

M1 characterisation and activation induced by the modified IRF5 mRNA was then confirmed as follows. RNA sequencing was performed to evaluate the underlying gene expression profile. The volcano plot above shows the change in gene expression on the x axis vs the -Log10 P-value on the y axis. P-values were adjusted using the Benjamini-Hochberg method. Fold changes were adjusted using the method for visualisation described by Love et al, 2014.

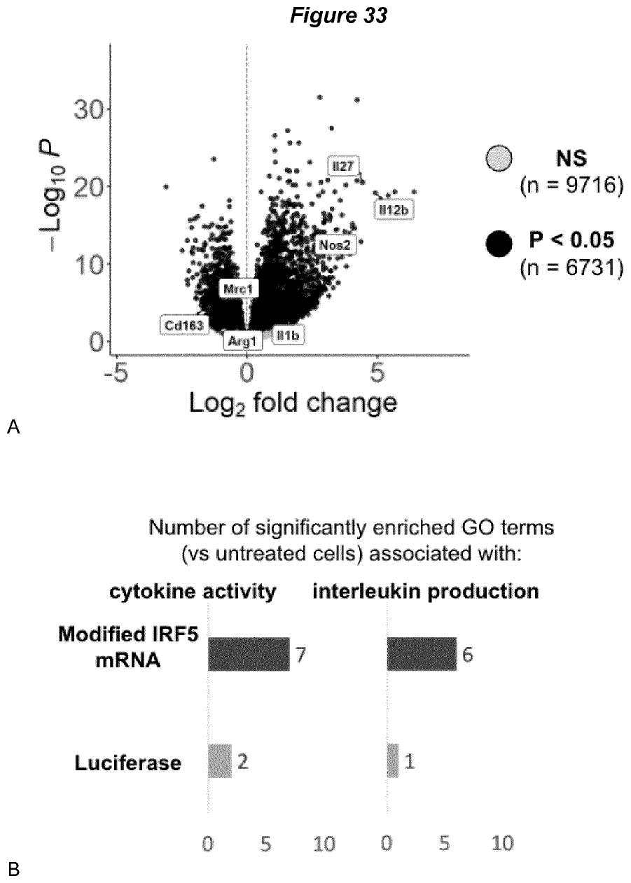

FIG. 33A shows that many genes were significantly differentially expressed (DE) vs the luciferase control. Genes upregulated in activated M1 macrophages (interleukins: II27, II12b, II1b and nitric oxide synthesis gene Nos2) or M2 macrophages (Arg1, Cd163, Mrc1) are labelled, showing significant upregulation M1 genes and downregulation M2 genes. FIG. 33B shows the results of a gene set enrichment analysis (GSEA) that was performed on the RNA sequencing dataset to discover sets of DE genes that were significantly enriched in cells transfected with the modified IRF5 mRNA (FDR <0.05). Cells were highly enriched with genes associated with cell killing, cytokine activity, and interleukin production. GSEA was performed in WebGestalt, with Wald statistic ranked inputs.

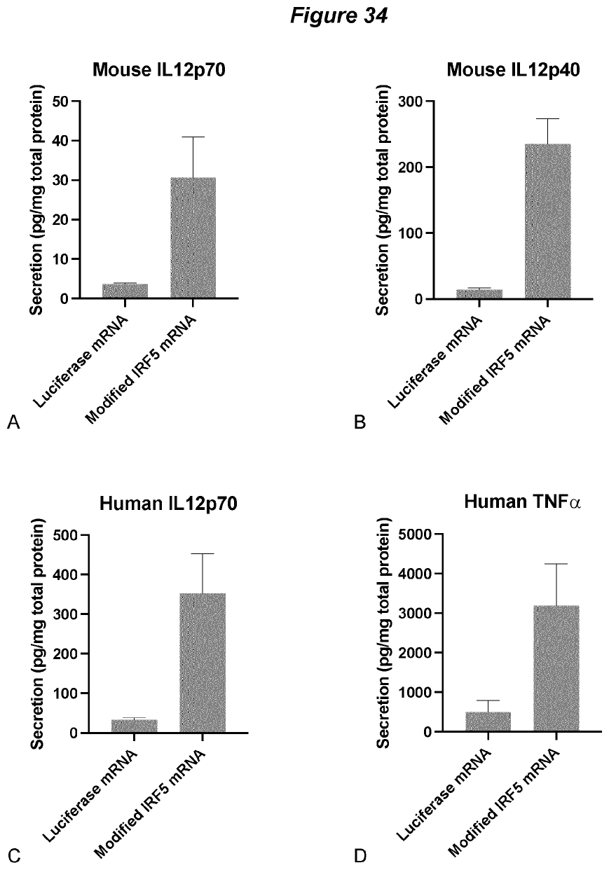

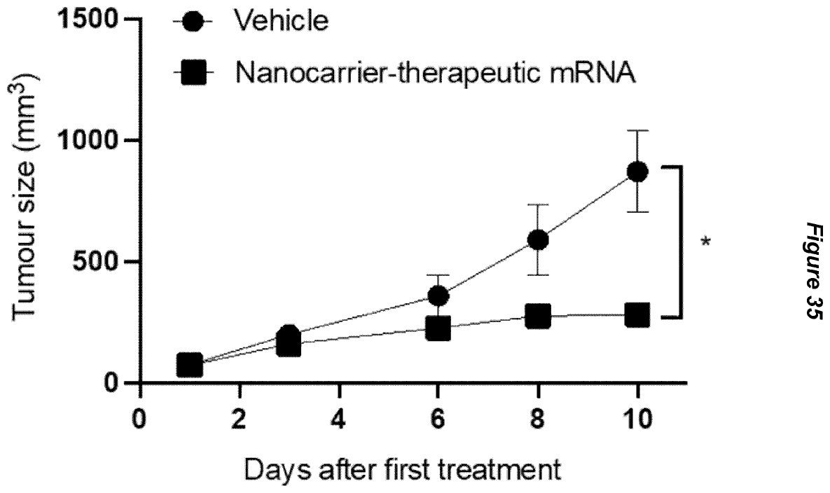

IL12 secretion in primary mouse macrophages transfected with IRF5 using nanocarriers of the invention: Primary murine bone marrow derived macrophages were polarized to the M2 phenotypes. Cells were then transfected for 24 hours with an mRNA expressing a modified version of IRF5 protein which can polarise M2 cells to M1 cells. The secretion of cytokines was measured 24 hours post-transfection. Control: Cells were transfected with an mRNA expressing luciferase (Control mRNA). Modified IRF5 is a protein with mutations which functions as the activated version of the WT IRF5. The formulation used was comprising GSCG1,2-RHL (N:P=0.6:1 to mRNA) with DOTMA/DOPE (w/w=10:1 to mRNA) and mRNA. IL12p70 and IL12p40 secretion was significantly increased with the cells transfected with the modified IRF5 mRNA (see FIGS. 34A and 34B).

IL12 and TNF secretion in primary human macrophages transfected with IRF5 using nanocarriers of the invention: Primary human macrophages were polarized to the M2 phenotypes. Cells were then transfected for 24 hours with an mRNA expressing a modified version of IRF5 protein which can polarise M2 cells to M1 cells. The secretion of cytokines was measured 24 hours post-transfection. Control: Cells were transfected with an mRNA expressing luciferase (Control mRNA). Modified IRF5 is a protein with mutations which functions as the activated version of the WT IRF5. The formulation used was comprising GSCG1,2-RHL (N:P=0.6:1 to mRNA) with DOTMA/DOPE (w/w=10:1 to mRNA) and mRNA. IL12p70 and TNFα secretion was significantly increased with the cells transfected with the modified IRF5 mRNA (see FIGS. 34C and 34D).

Methods for cytokine secretion experiments: Murine (bone marrow derived) and human (peripheral blood derived) monocytes were differentiated by cytokine exposure using a standard 6 day protocol. Transfection was performed 6 days post seeding. Overlaying media was aspirated at around 20-24 hrs post-transfection and replaced with 500 ul/well complete BMDM media (mouse) or ImmunoCult media (human) with cytokines (IL4+M-CSF). 24 hrs after media addition, the media was harvested, spun for 10 minutes and transferred to a series of 96-well plates that were stored in the −80 freezer. Meanwhile the cells were washed with PBS and lysed with 200 ul/well RIPA buffer for 5 minutes on the shaker. Lysates were spun for 10 minutes and supernatants were later analysed for cytokine content. Mouse cytokines were analysed using the LEGENDplex mouse macrophage/microglia panel (Biolegend; 740845). Human cytokines were analysed using the LEGENDplex human macrophage/microglia panel (Biolegend; 740503). Lysates were analysed using the Pierce BCA assay (ThermoFisher; 23225) to determine total protein content of each well.

### Example 25—Cancer Treatment In Vivo

MC38 tumour bearing mice were treated with vehicle control or a nanocarrier of the invention delivering a therapeutic mRNA. The therapeutic mRNA expresses modified (activated) IRF5 protein.

Mice were received doses of the nanocarrier comprising 1.75 mg/kg of the therapeutic mRNA three times per week. The treatment was started 9 days after tumour implantation.

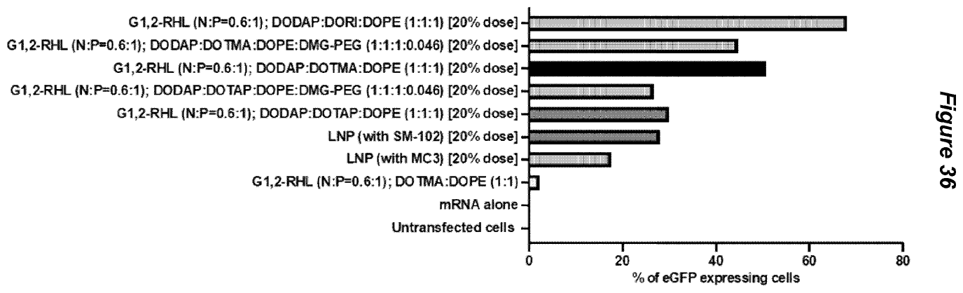

As shown in FIG. 35, significant suppression of tumour growth was observed following the treatment with nanocarrier delivering therapeutic mRNA (p=0.03; 2-way ANOVA).

Median survival of mice treated with mRNA therapeutics (via the nanocarrier of the invention) was significantly extended (time to endpoint, 1.5 cm³). p=0.004; Log-rank (Mantel-Cox) test was performed.

### Example 26—Lipid Mixtures (ii)

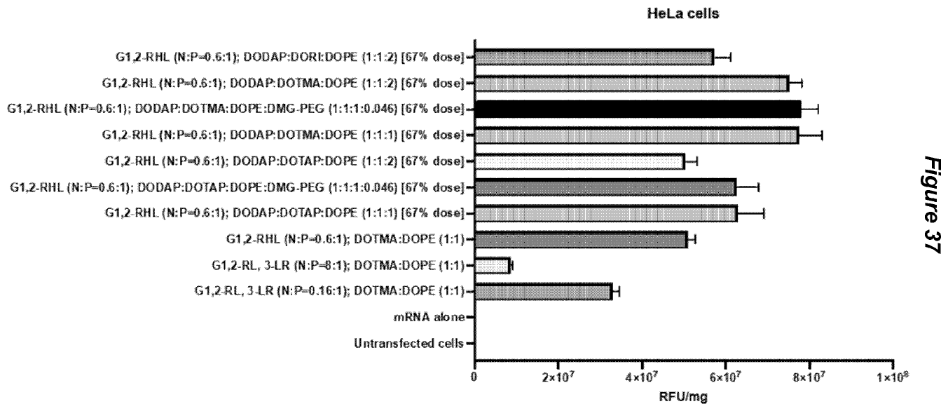

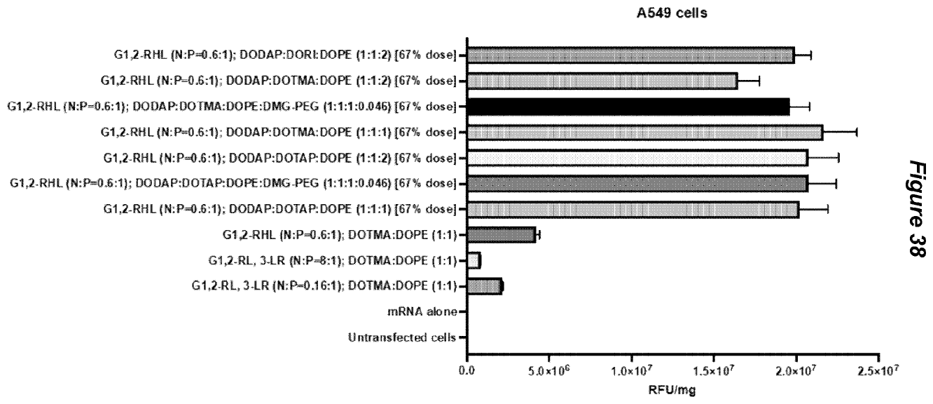

Jurkat, HeLa and A549 cells were transfected with nanocarriers of the invention, formulated with different lipid components (see FIGS. 36, 37 and 38 respectively).

Jurkat cells were transfected with the following formulations:

(1) GSCG1,2-RHL (N:P=0.6:1 to mRNA) with DOTMA:DOPE (with 1:1 in terms of the mole of DOTMA to the mole of DOPE), the overall lipid amount was w/w=10:1 to mRNA; (2) LNP containing the MC3 ionisable lipid (LNP with MC3); (3) LNP containing the SM-102 ionisable lipid (LNP with SM-102); (4) GSCG1,2-RHL (N:P=0.6:1 to mRNA) with DODAP:DOTAP:DOPE (with 1:1:1 in terms of the mole of DODAP to the mole of DOTAP to the mole of DOPE), the overall lipid amount was w/w=10:1 to mRNA; (5) GSCG1,2-RHL (N:P=0.6:1 to mRNA) with DODAP:DOTAP:DOPE:DMG-PEG (with 1:1:1:0.046 in terms of the mole of DODAP to the mole of DOTAP to the mole of DOPE to the mole of DMG-PEG), the overall lipid amount was w/w=10:1 to mRNA; (6) GSCG1,2-RHL (N:P=0.6:1 to mRNA) with DODAP:DOTMA:DOPE (with 1:1:1 in terms of the mole of DODAP to the mole of DOTMA to the mole of DOPE), the overall lipid amount was w/w=10:1 to mRNA; (7) GSCG1,2-RHL (N:P=0.6:1 to mRNA) with DODAP:DOTMA:DOPE:DMG-PEG (with 1:1:1:0.046 in terms of the mole of DODAP to the mole of DOTMA to the mole of DOPE to the mole of DMG-PEG), the overall lipid amount was w/w=10:1 to mRNA; (8) GSCG1,2-RHL (N:P=0.6:1 to mRNA) with DODAP:DORI:DOPE (with 1:1:1 in terms of the mole of DODAP to the mole of DORI to the mole of DOPE), the overall lipid amount was w/w=10:1 to mRNA.

The mRNA used was mRNA expressing eGFP, and the cells were transfected for 1 hour and were harvested 24 hours post-transfection to analyse fluorescent proteins expression by flow cytometry. Formulation (2)-(8) contained only 20% of the eGFP mRNA content (i.e. 0.3 ug mRNA per well) compared to formulation (1) and the mRNA alone control (i.e. 1.5 ug mRNA per well).

Hela and A549 cells were transfected with the following formulations:

(1) GSCG1,2-RL, 3-LR (N:P=0.16:1 to mRNA) with DOTMA:DOPE (with 1:1 in terms of the mole of DOTMA to the mole of DOPE), the overall lipid amount was w/w=10:1 to mRNA; (2) GSCG1,2-RL, 3-LR (N:P=8:1 to mRNA) with DOTMA:DOPE (with 1:1 in terms of the mole of DOTMA to the mole of DOPE), the overall lipid amount was w/w=10:1 to mRNA; (3) GSCG1,2-RHL (N:P=0.6:1 to mRNA) with DOTMA:DOPE (with 1:1 in terms of the mole of DOTMA to the mole of DOPE), the overall lipid amount was w/w=10:1 to mRNA; (4) GSCG1,2-RHL (N:P=0.6:1 to mRNA) with DODAP:DOTAP:DOPE (with 1:1:1 in terms of the mole of DODAP to the mole of DOTAP to the mole of DOPE), the overall lipid amount was w/w=10:1 to mRNA; (5) GSCG1,2-RHL (N:P=0.6:1 to mRNA) with DODAP:DOTAP:DOPE:DMG-PEG (with 1:1:1:0.046 in terms of the mole of DODAP to the mole of DORI to the mole of DOPE to the mole of DME-PEG), the overall lipid amount was w/w=10:1 to mRNA; (6) GSCG1,2-RHL (N:P=0.6:1 to mRNA) with DODAP:DOTAP:DOPE (with 1:1:2 in terms of the mole of DODAP to the mole of DOTAP to the mole of DOPE), the overall lipid amount was w/w=10:1 to mRNA; (7) GSCG1,2-RHL (N:P=0.6:1 to mRNA) with DODAP:DOTMA:DOPE (with 1:1:1 in terms of the mole of DODAP to the mole of DOTMA to the mole of DOPE), the overall lipid amount was w/w=10:1 to mRNA; (8) GSCG1,2-RHL (N:P=0.6:1 to mRNA) with DODAP:DOTMA:DOPE:DMG-PEG (with 1:1:1:0.046 in terms of the mole of DODAP to the mole of DOTMA to the mole of DOPE to the mole of DMG-PEG), the overall lipid amount was w/w=10:1 to mRNA; (9) GSCG1,2-RHL (N:P=0.6:1 to mRNA) with DODAP:DOTMA:DOPE (with 1:1:2 in terms of the mole of DODAP to the mole of DOTMA to the mole of DOPE), the overall lipid amount was w/w=10:1 to mRNA; (10) GSCG1,2-RHL (N:P=0.6:1 to mRNA) with DODAP:DORI:DOPE (with 1:1:2 in terms of the mole of DODAP to the mole of DORI to the mole of DOPE), the overall lipid amount was w/w=10:1 to mRNA;

The mRNA used was mRNA expressing eGFP, and the cells were harvested 24 hours post-transfection to analyse fluorescent proteins expression by a plate reader. Formulation (4)-(10) contained only 67% of the eGFP mRNA content compared to formulation (1)-(3). Control: Cells transfected with mRNA alone or untransfected.

More detailed methods are as follows: MC3 LNP (NP6): Stock solutions of DLin-MC3-DMA, DSPC, Cholesterol and DMG-PEG2000 were combined to give a molar ratio of 50:10:38.5:1.5. This lipid mixture was added to the mRNA solution (21.34 w/w lipids/mRNA) in citrate buffer and mixed with a pipette. The formulation was diluted 2-fold in PBS before removing aliquots for DLS and Zeta potential measurements. The Formulation was diluted further in PBS for transfection. DLS measurement (20× dilution in PBS): 162 nm, Pdl 0.11). Zeta potential measurement (86× dilution in Water): +39.1 mV.

SM102 LNP (NP 5.7): Stock solutions of SM-102, DSPC, Cholesterol and DMG-PEG2000 in ethanol were combined to give a molar ratio of 50:10:38.5:1.5. This lipid mixture was added to the mRNA solution (21.34 w/w lipids/mRNA) and mixed with a pipette. The formulation was diluted 2-fold in PBS before removing aliquots for DLS and Zeta potential measurements. The Formulation was diluted further in PBS for transfection. The Formulation was diluted further in PBS for transfection. DLS measurement (20× dilution in PBS): 160 nm, Pdl 0.12). Zeta potential measurement (86× dilution in Water): +38.0 mV.

DODAP:DOTAP:DOPE:DMG-PEG2000 were combined at a 1:1:1:0.046 molar ratio in an ethanol solvent and mixed with the solution of peptide and mRNA, giving a turbid solution. DLS measurement (27× dilution in 25 mM HEPES buffer): 112.6 nm, Pdl 0.093). Zeta potential measurement (60× dilution in water): 25.2 mV.

DODAP:DOTMA:DOPE:DMG-PEG2000 were combined at a 1:1:1:0.046 molar ratio in an ethanol solvent and mixed with the solution of peptide and mRNA, giving a turbid solution. DLS measurement (27× dilution in 25 mM HEPES buffer): 114.2 nm, Pdl 0.069). Zeta potential measurement (60× dilution in water): 19.1 mV.

### Example 27—IRF5 Expression in BMDM Cells

### BMDM Polarisation

BMDM cells were isolated using the StemCell Tech EasySep Mouse Monocyte Isolation Kit. Resuspended cells in DMEM+1% glutamax+10% heat inactivated FBS+1% Pen/Strep (complete media) were enriched with 50 ng/ml murine M-CSF (1:1000 of 50 ug/ml stock) at 1E+6 cells/mi. 2M cells (i.e. 2 mls) in each well of 6-well plate(s) were seeded and left in 37 degree c. incubator for 4 days. Half media exchange using complete media enriched with 50 ug/ml murine M-CSF and 40 ng/ml murine IL-4 was performed. On the next day (day 5), cells were harvested by washing with PBS and a 10 minute incubation with 5 mM EDTA (diluted in PBS) at 37 degrees C., followed by plate tapping and resuspension using a P1000. Cells were resuspended in complete media containing 50 ug/ml murine M-CSF and 20 ng/ml murine IL-4 (activation media) at 2.4E+5 cells/mi and 2.4E+5 cells (i.e. 1 ml) were seeded into each well of a 12-well plate.

### BMDM Transfection

The media overlaying the polarised BMDM cells was aspirated and exchanged with 960 ul/well activation media. The following formulation was used as the “IRF5 mRNA” delivery composition: GSCG1,2-RHL (N:P=0.6:1 to mRNA) with DOTMA/DOPE (w/w=10:1 to mRNA) and mRNA. Modified IRF5 is a protein with mutations which functions as the activated version of the WT IRF5. The DNA sequence encoding the mRNA, and the amino acid sequence of the modified IRF5 sequence are shown below. 240 ul/well of each test condition was added (test conditions were prepared in 25 mM HEPES solution). Untransfected control consisted of neat 25 mM HEPES and the transfection control was a luciferase payload (L-7202; TriLink Biotechnologies). Between addition of each test condition the plate was shaken back and forth to ensure mixing. The plate was placed into the incubator overnight.

### DNA Sequence of the Modified IRF5 (Open Reading Frame)

(SEQ ID NO: 7)
ATGAACCAGAGCATTCCAGTGGCTCCTACACCTCCTAGAAGAGTGAGAC
TGAAACCTTGGCTGGTGGCTCAGGTGAACTCTTGTCAGTATCCAGGACT
CCAGTGGGTGAACGGAGAAAAGAAGCTGTTTTGCATCCCTTGGAGACAC
GCTACAAGACACGGACCTTCTCAGGACGGAGATAACACCATCTTCAAGG
CTTGGGCTAAGGAGACAGGAAAGTACACAGAGGGAGTGGACGAAGCAGA
TCCAGCTAAGTGGAAGGCTAATCTCAGGTGCGCCCTGAATAAGAGCAGA
GATTTCAGGCTGATCTACGACGGACCTAGAGATATGCCTCCTCAGCCTT
ACAAGATCTACGAAGTGTGCTCTAACGGACCAGCTCCTACAGATTCTCA
GCCACCAGAAGATTATAGCTTTGGAGCCGGAGAAGAAGAGGAGGAAGAA
GAAGAACTGCAGAGAATGCTGCCTAGTCTGTCTCTGACAGAAGATGTCA
AGTGGCCTCCTACACTGCAGCCTCCTACACTGCAGCCTCCAGTGGTGCT
GGGACCTCCAGCTCCAGATCCTTCTCCTCTGGCTCCTCCTCCAGGAAAT
CCAGCAGGATTTAGAGAGCTGCTGTCAGAAGTGCTGGAACCAGGACCTC
TGCCAGCTTCTCTGCCTCCAGCAGGAGAACAGCTGCTGCCAGATCTGCT
GATTTCTCCTCATATGCTGCCTCTGACAGATCTGGAGATCAAGTTCCAG
TACAGGGGAAGACCTCCTAGAGCTCTGACAATCTCTAACCCTCACGGCT
GTAGACTGTTCTACTCTCAGCTGGAAGCTACACAGGAACAGGTGGAACT
GTTCGGACCTATCTCTCTGGAACAGGTGAGATTCCCTTCTCCAGAGGAT
ATCCCTAGCGATAAGCAGAGGTTCTACACAAATCAGCTGCTGGACGTGC
TGGATAGAGGACTGATTCTGCAGCTGCAGGGACAGGATCTGTACGCTAT
TAGGCTCTGCCAGTGTAAGGTGTTTTGGTCAGGACCTTGCGCTAGCGCT
CACGATTCTTGTCCTAATCCTATCCAGAGGGAGGTGAAGACAAAGCTGT
TCTCTCTGGAGCACTTCCTGAACGAACTGATCCTGTTCCAGAAAGGCCA
GACAAATACCCCTCCTCCTTTCGAGATCTTCTTTTGCTTCGGCGAAGAG
TGGCCAGATAGAAAGCCTAGAGAGAAGAAGCTGATTACAGTGCAGGTGG
TGCCAGTGGCAGCTAGACTGCTCCTGGAAATGTTTAGCGGAGAACTGGA
TTGGGACGCAGACGATATCAGACTGCAGATCGACAACCCAGATCTGAAG
GACAGAATGGTGGAGCAGTTCAAGGAGCTGCATCACATTTGGCAGAGTC
AGCAGAGACTGCAGCCAGTGGCTCAGGCTCCTCCAGGAGCAGGACTGGG
AGTGGGACAGGGACCTTGGCCTATGCATCCAGCAGGAATGCAG.

### Protein Sequence of the Modified IRF5

(SEQ ID NO: 8)
MNQSIPVAPTPPRRVRLKPWLVAQVNSCQYPGLQWVNGEKKLFCIPWRH
ATRHGPSQDGDNTIFKAWAKETGKYTEGVDEADPAKWKANLRCALNKSR
DFRLIYDGPRDMPPQPYKIYEVCSNGPAPTDSQPPEDYSFGAGEEEEEE
EELQRMLPSLSLTEDVKWPPTLQPPTLQPPVVLGPPAPDPSPLAPPPGN
PAGFRELLSEVLEPGPLPASLPPAGEQLLPDLLISPHMLPLTDLEIKFQ
YRGRPPRALTISNPHGCRLFYSQLEATQEQVELFGPISLEQVRFPSPED
IPSDKQRFYTNQLLDVLDRGLILQLQGQDLYAIRLCQCKVFWSGPCASA
HDSCPNPIQREVKTKLFSLEHFLNELILFQKGQTNTPPPFEIFFCFGEE
WPDRKPREKKLITVQVVPVAARLLLEMFSGELDWDADDIRLQIDNPDLK
DRMVEQFKELHHIWQSQQRLQPVAQAPPGAGLGVGQGPWPMHPAGMQ.
Underlined aspartic acid residues (D) are the
modified residues.

### Staining and Fixation

After 24 hrs the media overlaying each test condition was transferred to corresponding 1.5 ml Eppendorf tubes (in order to conserve cells that may have died during transfection). Cells were then washed with 1 ml/well PBS which was aspirated. Afterwards 1 ml/well 5 mM EDTA was added and the plate(s) transferred to the 37 degree c. incubator for 10 minutes until the cells started to detach. Using a cell scrapper and P1000 pipette, cells were then detached from the plate and collected into 1.5 ml tubes. Cells were spun down at 400 g for 5 minutes and resuspended in 100 ul/sample fixable Live/Dead Aqua stain (1:250 diluted in PBS). ArC beads were stained with 3 ul neat Live/Dead Aqua for compensation. These cells were stained in the fridge for 30 minutes. Following this, tubes were spun down at 400 g for 5 minutes. The supernatant was discarded and the cells resuspended in 500 ul/sample PBS wash. Negative ArC beads were added to the compensation control. See below for the further steps concerning sample staining depending on the purpose of the flow cytometry assay:

### For Surface Staining (CD11b, CD206 and CD80):

The samples were spun down at 400 g for 5 minutes. The samples were resuspended in 50 ul/sample 2× FcX block diluted in FACS buffer (1:50; end concentration 1:100), then incubated in fridge for 5 minutes. 50 ul/sample 2× surface antigen antibody mixes made up in FACS buffer including corresponding FMO controls were added to the samples. The final concentrations of the antibodies used were: 1:200 Cd11b-APC (Biolegend; 101212), 1:25 CD206-BV711 (Biolegend; 141727) and 1:50 CD80-PE (Biolegend; 104708). For each antibody used 1 drop UltraComp Plus beads and 3 ul of each antibody were added on top (will act as antibody compensation control). The samples were incubated in fridge for 20 minutes. The cells and beads were spun down at 400 g for 5 minutes. The samples were washed with PBS, fixed.

### For Intracellular Staining (IRF5):

The samples were spun down at 400 g for 5 minutes. Samples were fixed and permeabilised using the eBioscience FoxP3/Transcription Factor Staining Buffer Set (00-5523-00) as per manufacturer's instructions. Briefly, samples were fixed overnight in the fridge using the Foxp3 Fixation/Permeabilisation working solution. Subsequently samples were washed with permeabilisation buffer and blocked with 50 ul/sample of 2× FcX block diluted in permeabilisation buffer (1:50; end concentration 1:100) for 15 minutes at room temperature. 50 ul/sample of 2×IRF5-AF555 (ab210656; Abcam) diluted in FACS buffer with a final concentration of 1:50 was then added and the samples left for 45 minutes at room temperature. Alongside the stained cell samples, 1 drop of UltraComp Plus beads were also stained with 3 ul of the IRF5 antibody as a compensation control. Samples were then washed twice with permeabilisation buffer and then resuspended in 400 ul/sample FACS buffer for flow analysis within 24 hrs.

### Results

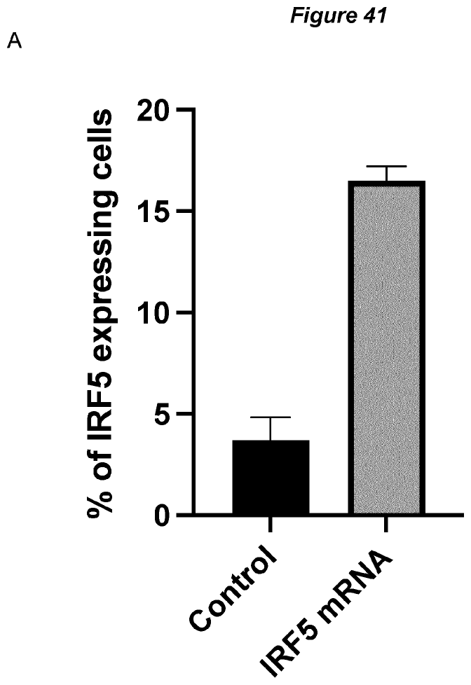

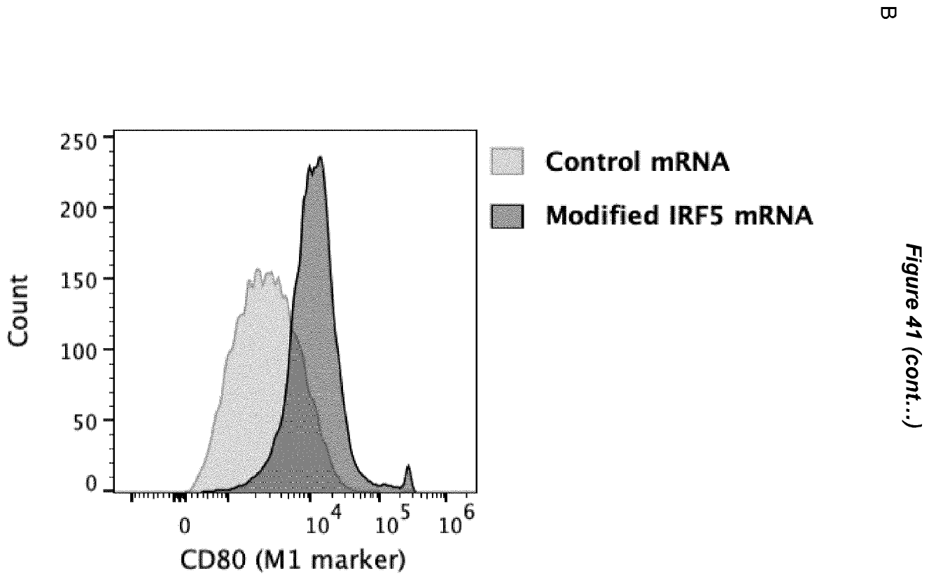

FIG. 41A shows expression of activated IRF5 in over 15% of BMDM cells that were transfected with the composition comprising the IRF5 mRNA, as detected by flow cytometry. FIG. 41B shows that BMDM cells transfected with the composition comprising the IRF5 mRNA exhibit increased expression the M1 marker (CD80), indicating M1 polarisation.

### Example 28—Coating LNPs with Antibody-Conjugated Dendrimer

Antibody conjugation: GSEGSEGSEC(OH)G1-(Ac)ESGESGESG ([(Ac-ESGESGESG)2K]GSEGSEGSEC, 2793 g/mol, 9 negative charges per peptide) was prepared at 10 mg/mL in water. N-(3-Dimethylaminopropyl)-N′-ethylcarbodiimide hydrochloride solution was added and allowed incubate for 2 minutes at room temperature. N-Hydroxysulfosuccinimide sodium salt (Sulfo-NHS, 217.1 g/mol) was added and the solution allowed to incubate for 15 minutes at room temperature. InVivoMAb anti-human CD3 (BioXCell BE0001-2, 150 kDa) at 7.19 mg/mL in PBS pH 7.0 was added and the solution allowed to incubate for 4 h at room temperature prior to dialysis in PBS overnight.

LNP preparation: Lipid stock solution in ethanol: DLin-MC3-DMA at 105.5 mg/mL in EtOH (10.08 uL, 1.064 mg, 1.657×10⁻³ mmoles), DSPC at 7.8 mg/mL in EtOH (33.6 uL, 0.262 mg, 3.317×10-4 mmoles), Cholesterol at 12.8 mg/mL in EtOH (38.6 uL, 0.494 mg, 1.278×10⁻³ mmoles), and DMG-PEG2000 at 18.8 mg/mL in EtOH (6.64 uL, 0.125 mg, 4.971×10⁻⁵ mmoles), and 62.1 uL EtOH were added to a glass vial and mixed. Final molar ratio of lipids was DLin-MC3-DMA:DSPC:Cholesterol:DMG-PEG2000 50:10:38.5:1.5 with an overall lipid concentration of 22.11 mM.

LNP formulation: CleanCap® EGFP mRNA (5 moU) at 1 mg/mL in 1 mM Sodium Citrate pH 6.4 (45.0 uL, 45.0 ug, 1.364×10⁻⁴ mmoles P), sterile water (175.5 uL) and 500 mM Citrate buffer pH 5.0 (4.5 uL) were added and mixed in a polypropylene tube. 75.0 uL of the lipid mixture described above (1.364×10⁻⁴ mmoles DLin-MC3-DMA, NIP ratio 6.07) was added and rapidly mixed up and down with a pipette, giving a turbid solution. The final mRNA concentration was 150 μg/mL. DLS measurement (27× dilution in PBS): 160 nm, PDI 0.10). Zeta potential measurement (120× dilution in water): +36.7 mV.

Coating nanoparticles: The base LNP described above was diluted two-fold in PBS to an mRNA concentration of 75 μg/mL. To 100.0 uL (7.50 ug mRNA, 2.273×10⁻⁵ mmoles P) of these nanoparticles, 50.0 uL of coating material dissolved in PBS was added, rapidly mixing up and down with a pipette. The final mRNA concentration of the solution was 50 μg/mL. A breakdown of the coatings are provided in the table below:

TABLE 16
An example of the coating calculation and the size, zeta potential and PDI of the resulting nanoparticles.
Increasing the coating materials reducing the zeta potential, suggesting coating of the LNP.

| Mass eq | | | | | | Charge | | | Z- | | |
|---|---|---|---|---|---|---|---|---|---|---|---|
| conjugate | Coating | Coating | Peptide | Mol ratio | | ratio | Coating | Ab | average | | Zeta |
| (wrt. | conjugate | peptide | coating | (Peptide | Charge | (Coating E | Ab | coating | diameter | | Potential |
| mRNA) | (ug) | (ug) | (mmoles) | to mRNA P) | (mmoles) | to mRNA P) | (ug) | (mmoles) | (nm) | PDI | (mV) |
| 11.18 | 83.9 | 13.15 | 4.71E−06 | 0.21 | 4.24E−05 | 1.86 | 70.7 | 4.71E−07 | 152 | 0.11 | 17.5 |
| 6.71 | 50.3 | 7.89 | 2.83E−06 | 0.12 | 2.54E−05 | 1.12 | 42.4 | 2.83E−07 | 153 | 0.15 | 26.0 |
| 3.35 | 25.1 | 3.94 | 1.41E−06 | 0.06 | 1.27E−05 | 0.56 | 21.2 | 1.41E−07 | 145 | 0.18 | 32.8 |
| 1.12 | 8.4 | 1.32 | 4.72E−07 | 0.02 | 4.25E−06 | 0.19 | 7.1 | 4.72E−08 | 154 | 0.15 | 32.9 |

### Example 29—Antibody Mediated Targeting of Lymphocytes

Conjugation of the antibody to the peptide: Anti-CD3 antibody was conjugated to a first generation dendritic glutamic acid containing peptide having sequence (Ac-ESGESGESG)2KGSEGSEGSEC-OH (“Ac” represent acetylation of the N-terminus of the peptide dendrimer).

Nanocarrier Formulations: GSCG1,2-RHL, eGFP mRNA and DOTMA/DOPE liposome were mixed in a polypropylene tube. The conjugated antibody-peptide solution was then coated onto the resultant nanoparticles at either 3 weight equivalents of antibody conjugate relative to mRNA (3 eq) or 1 weight equivalents of antibody conjugate relative to mRNA (1 eq).

As a negative control, non-conjugated dendritic glutamic acid containing peptide (Ac-ESGESGESG)2KGSEGSEGSEC-OH was applied as a particle coating. The amount of dendritic glutamic acid containing peptide was equal to the quantity of present in the antibody-conjugated coated particles.

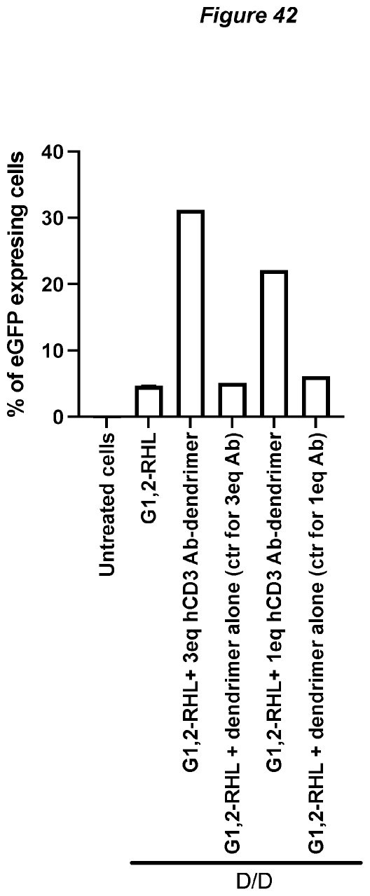

Transfection procedure: 200,000 Jurkat cells (immortalized line of human T lymphocyte cells) were seeded per well in 24 well plates in full growth medium overnight. The cells were then transfected with the nanocarrier formulations. The cells were harvested and fixed. eGFP expression was measured by flow cytometry (BD Fortessa) as described in Example 1. Results are shown in FIG. 42.

### REFERENCES

A number of publications are cited above in order to more fully describe and disclose the invention and the state of the art to which the invention pertains. Full citations for these references are provided below. The entirety of each of these references is incorporated herein.
Benizri et al. Bioconjugated Oligonucleotides: Recent Developments and Therapeutic Applications. Bioconjug Chem. 30(2): 366-383. (2019).
Bonnet et al. Systemic Delivery of DNA or siRNA Mediated by Linear Polyethylenimine (L-PEI) Does Not Induce an Inflammatory Response. Pharmaceutical Res. 25, 2972 (2008).
Braum. Non-viral Vector for Muscle-Mediated Gene Therapy; Chapter 9 Muscle Gene Therapy, Springer Nature Switzerland A G D. Duan, J. R. Mendell (eds.) 157-178 (2019).
Chong Z X, Yeap S K, Ho W Y. Transfection types, methods and strategies: a technical review. PeerJ. 2021 Apr. 21; 9:e11165. doi: 10.7717/peerj.11165. PMID: 33976969; PMCID: PMC8067914.
Srividya Gorantla, Ganesh Gorantla, Ranendra N. Saha & Gautam Singhvi (2021) CD44 receptor-targeted novel drug delivery strategies for rheumatoid arthritis therapy, Expert Opinion on Drug Delivery, 18:11, 1553-1557
Hao Cui, Xinying Zhu, Shuyue Li, Peipei Wang, and Jianping Fang. Liver-Targeted Delivery of Oligonucleotides with N-Acetylgalactosamine Conjugation. ACS Omega 2021, 6 (25), 16259-16265. DOI: 10.1021/acsomega.1c01755
Holland, R., et al. Ligand conjugate SAR and enhanced delivery in NHP. Molecular Therapy Volume 29, Issue 10, 6 Oct. 2021, Pages 2910-2919 (2021).
Huang et al. Delivery of Therapeutics Targeting the mRNA-Binding Protein HuR Using 3DNA Nanocarriers Suppresses Ovarian Tumor Growth. Cancer Research. 76(6), 1549-1559 (2016).
Jasinski et al. The Effect of Size and Shape of RNA Nanoparticles on Biodistribution. Mol Ther. 26(3), 784-792 (2018).
John et al. Human MicroRNA Targets; PLoS Biology, 11(2), 1862-1879 (2004).
Jones B, Buenaventura T, Kanda N, Chabosseau P, Owen B M, Scott R, Goldin R, Angkathunyakul N, Corrêa I R Jr, Bosco D, Johnson P R, Piemonti L, Marchetti P, Shapiro A M J, Cochran B J, Hanyaloglu A C, Inoue A, Tan T, Rutter G A, Tomas A, Bloom S R. Targeting GLP-1 receptor trafficking to improve agonist efficacy. Nat Commun. 2018 Apr. 23; 9(1):1602. doi: 10.1038/s41467-018-03941-2. PMID: 29686402; PMCID: PMC5913239.
Kwok et al. Comparative structural and functional studies of nanoparticle formulations for DNA and siRNA delivery; Nanomedicine: Nanotechnology, Biology and Medicine 7; 210-219 (2011).
Kwok et al. Peptide Dendrimer/Lipid Hybrid Systems Are Efficient DNA Transfection Reagents: Structure-Activity Relationships Highlight the Role of Charge Distribution Across Dendrimer Generations; ACSNano 7; 5 4668-4682 (2013).
Kwok et al. Systematic Comparisons of Formulations of Linear Oligolysine Peptides with siRNA and Plasmid DNA; Chem Biol Drug Des 87: 747-763 (2016).
Kwok et al. Developing small activating RNA as a therapeutic: current challenges and promises Therapeutic delivery 10(3):151-164 (2019)
Lim et al. Engineered Nanodelivery Systems to Improve DNA Vaccine Technologies; Pharmaceutics 12(1) 30 (2020).
Linlang Guo, Fan Zhang, Yingqian Cai, Tengfei Liu. Expression profiling of integrins in lung cancer cells. Pathology—Research and Practice, Volume 205, Issue 12, 2009, pp 847-853, ISSN 0344-0338
Love, M. I., Huber, W. & Anders, S. Moderated estimation of fold change and dispersion for RNA-seq data with DESeq2. Genome Biol 15, 550 (2014). https://doi.org/10.1186/s13059-014-0550-8
Luo et al. Arginine functionalized peptide dendrimers as potential gene delivery vehicles. Biomaterials 33, 4917-4927 (2012).
Jami Mandelin, Marina Cardó-Vila, Wouter H. P. Driessen, et al. Selection and identification of ligand peptides targeting a model of castrate-resistant osteogenic prostate cancer and their receptors. PNAS, 112 (12) 3776-3781. Mar. 11, 2015.
Myers et al. Recombinant Dicer efficiently converts large dsRNAs into siRNAs suitable for gene silencing; Nature Biotechnology 21:324-328 (2003).
Philippidis. Fourth Boy Dies in Clinical Trial of Astellas' AT132. Human Gene Therapy. 32, 19-20 (2021).
Qiu et al. Developing Biodegradable Lipid Nanoparticles for Intracellular mRNA Delivery and Genome Editing. Acc. Chem. Res. 54(21), 4001-4011 (2021).
Ren et al. Structural basis of DOTMA for its high intravenous transfection activity in mouse; Gene Therapy 7, 764-768 (2000).
Saher et al. Novel peptide-dendrimer/lipid/oligonucleotide ternary complexes for efficient cellular uptake and improved splice-switching activity; Eur J Pharmaceutics and Biopharmaceutics 132: 29-40 (2018).
Saher et al. Sugar and Polymer Excipients Enhance Uptake and Splice-Switching Activity of Peptide-Dendrimer/Lipid/Oligonucleotide Formulations. Pharmaceutics. 11(12): 666 (2019)
Sheridan et al. Gene therapy finds its niche. Nat Biotechnol 29 (2), 121-8 (2011).
Sakamaki et al. Maltotriose Conjugated Metal-Organic Frameworks for Selective Targeting and Photodynamic Therapy of Triple Negative Breast Cancer Cells and Tumor Associated Macrophages. Adv Ther (Weinh). 2020 August; 3(8):2000029. doi: 10.1002/adtp.202000029. Epub 2020 Jun. 8. PMID: 33072859; PMCID: PMC7567337.
Senoo H, Yoshikawa K, Morii M, Miura M, Imai K, Mezaki Y. Hepatic stellate cell (vitamin A-storing cell) and its relative—past, present and future. Cell Biol Int. 2010 December; 34(12):1247-72. doi: 10.1042/CB120100321. PMID: 21067523.
Sloas et al. Engineered CAR-Macrophages as adoptive immunotherapies for solid tumors. Front. Immunol. 12:783305. (2021)
Fenny H. F. Tang, Fernanda I. Staquicini, André A. R. Teixeira, Ricardo J. Giordano, et al. A ligand motif enables differential vascular targeting of endothelial junctions between brain and retina. PNAS, 116 (6) 2300-2305. Jan. 22, 2019.
Wang et al. Adeno-associated virus vector as a platform for gene therapy delivery. Nat Rev Drug Discov. 18(5): 358-378 (2019).
Lulu Xue, Ningqiang Gong, Sarah J. Shepherd, Xinhong Xiong, Xueyang Liao, Xuexiang Han, Gan Zhao, Chao Song, Xisha Huang, Hanwen Zhang, Marshall S. Padilla, Jingya Qin, Yi Shi, Mohamad-Gabriel Alameh, Darrin J. Pochan, Karin Wang, Fanxin Long, Drew Weissman, and Michael J. Mitchell. Rational Design of Bisphosphonate Lipid-like Materials for mRNA Delivery to the Bone Microenvironment. Journal of the American Chemical Society 2022 144 (22), 9926-9937. DOI: 10.1021/jacs.2c02706
Artjom Wischnjow, Dikran Sarko, Maria Janzer, Christina Kaufman, Barbro Beijer, Sebastian Brings, Uwe Haberkorn, Gregor Larbig, Armin Kubelbeck, and Walter Mier. Renal Targeting: Peptide-Based Drug Delivery to Proximal Tubule Cells. Bioconjugate Chemistry 2016 27 (4), 1050-1057. DOI: 10.1021/acs.bioconjchem.6b00057.
Vahatupa M, Salonen N, Uusitalo-Jarvinen H, Jarvinen T A H. Selective Targeting and Tissue Penetration to the Retina by a Systemically Administered Vascular Homing Peptide in Oxygen Induced Retinopathy (OIR). Pharmaceutics. 2021 Nov. 15; 13(11):1932. doi: 10.3390/pharmaceutics13111932. PMID: 34834347; PMCID: PMC8618640.
Zahid M, Feldman K S, Garcia-Borrero G, Feinstein T N, Pogodzinski N, Xu X, Yurko R, Czachowski M, Wu Y L, Mason N S, Lo C W. Cardiac Targeting Peptide, a Novel Cardiac Vector: Studies in BioDistribution, Imaging Application, and Mechanism of Transduction. Biomolecules. 2018 Nov. 14; 8(4):147. doi: 10.3390/biom8040147. PMID: 30441852; PMCID: PMC6315548.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A nanoparticle comprising a peptide dendrimer, a nucleic acid and a lipid, wherein the peptide dendrimer comprises at least: a core peptide sequence, a first branching residue and two first peptide motifs,
wherein the nanoparticle is targeted to a target cell or tissue, e.g. a myeloid, lymphoid, muscle, lung cell, a CD206+ cell and/or tumour cell, and/or a myeloid, lymphoid, muscle, lung tissue and/or tumour tissue,
and wherein the nanoparticle is capable of transfecting the target cell in vitro at an efficiency of at least 10%, at least 12.5%, at least 15%, at least 17.5%, at least 20%, at least 25%, at least 30%, at least 40%, at least 45%, at least 50%, at least 55%, at least 60%, at least 65%, at least 70%, at least 75%, at least 80%, at least 85%.

2. The nanoparticle of claim 1, wherein the nanoparticle comprises a myeloid, lymphoid, muscle, lung, CD206+ cell or tumour cell targeting motif; optionally selected from;
a. a muscle targeting motif, optionally comprising an ASSLNIA (SEQ ID NO:1), PYDQLRH (SEQ ID NO:2), or KAMHQMQ (SEQ ID NO:3) peptide motif;
b. an integrin targeting motif, optionally comprising an RGD or ACDCRGDCFCG (SEQ ID NO:5) peptide motif;
c. a lung targeting motif, optionally comprising the peptide sequence CGFECVRQCPERC (SEQ ID NO:4);
d. a mannose receptor targeting motif, optionally a mannose sugar or maltotriose; or
e. an antibody or target-binding fragment thereof, optionally wherein the antibody specifically binds to CD3
f. a lipid linked with a bisphosphonate (BP) group.

3. The nanoparticle of claim 1 or claim 2, wherein the lung cell is selected from an alveolar macrophage, a ciliated cell, epithelial cell, basal cell, secretory cell, club cell, alveolar cell, fibroblast, a CD206+ cell and/or an endothelial cell.

4. The nanoparticle according to any one of claims 2 or 3, wherein the myeloid, lymphoid, muscle, lung, CD206+ cell and/or tumour cell targeting motif is bound to a negatively charged or neutrally charged polymer or lipid.

5. The nanoparticle according to claim 4, wherein the polymer or lipid is selected from polyglutamic acid (PGA), poly(acrylic acid), alginic acid, polyethylene glycol (PEG), or cholesteryl hemisuccinate/1,2-dioleoyl-sn-glycero-3-phosphoethanolamine, a neutrally-charged zwitterionic polymer or lipid, or a glutamic acid containing peptide.

6. The nanoparticle according to claim 4 or claim 5, wherein the polymer is a linear or dendritic PGA.

7. The nanoparticle according to claim 4 or claim 5, wherein the polymer is a dendritic glutamic acid containing peptide.

8. The nanoparticle according to any one of claims 2 to 7, wherein the myeloid, lymphoid, muscle, lung, CD206+ cell or tumour cell targeting motif is covalently bound to the peptide dendrimer, or wherein the nanoparticle further comprises a second peptide comprising the myeloid, lymphoid, muscle, lung or tumour cell targeting motif.

9. The nanoparticle according to claim 8, wherein the myeloid, lymphoid or lung cell targeting motif comprises a mannose sugar, optionally wherein the mannose sugar is covalently bound to the C-terminus and/or N-terminus of the peptide dendrimer and/or the second peptide.

10. The nanoparticle according to claim 9, wherein the mannose sugar is covalently bound to a second peptide comprising polyglutamic acid (PGA).

11. The nanoparticle according to claim 10, wherein the second peptide comprises linear PGA comprising 2 to 10, 2 to 25, 2 to 15 or 2 to 100 glutamic acid residues, or wherein the second peptide comprises branched PGA comprising 2 to 10, 2 to 25, 2 to 15 or 2 to 100 glutamic acid residues in each branch and/or in the core sequence, optionally wherein the second peptide comprises the structure (Ac-EEEE)2KGSGGSGGSC[(S-S)-α-D-Thiomannose].

12. The nanoparticle according to any one of the preceding claims, wherein the lipid:nucleic acid weight/weight ratio is between 0.5:1 and 40:1, between 1:1 and 30:1, or between 2:1 and 25:1, optionally wherein the lipid:nucleic acid weight/weight ratio is at least 10:1 and wherein the nanoparticle is targeted to spleen and lung, or wherein the lipid:nucleic acid weight/weight ratio is about 23:1 and the nanoparticle is targeted to lung and spleen.

13. The nanoparticle according to any one of the preceding claims, wherein the lipid comprises two lipids, three lipids, four lipids, five lipids, or more than five lipids, optionally wherein the lipids comprise DOPE, DOTMA, DODAP, DORI, DMG-PEG and/or DOTAP.

14. The nanoparticle according to claim 13, wherein the lipid comprises DOTMA and DOPE.

15. The nanoparticle according to claim 13 or claim 14, wherein the lipid comprises three lipid components, or four lipid components, wherein each lipid component is selected from the group comprising DOPE, DOTMA, DODAP, DORI, DMG-PEG and DOTAP.

16. The nanoparticle according to claim 13 or claim 15, wherein the lipid comprises DODAP, DOTMA and DOPE; or wherein the lipid comprises DODAP, DOTAP, and DOPE; or wherein the lipid comprises DODAP, DORI, and DOPE; or wherein the lipid comprises DODAP, DOTMA, DOPE and DMG-PEG; or wherein the lipid comprises DODAP, DOTAP, DOPE and DMG-PEG.

17. The nanoparticle according to any one of the preceding claims, wherein the peptide dendrimer further comprises two second branching residues and four second peptide motifs, wherein one of the second branching residues is covalently bound to one of the first peptide motifs and the other second branching residue is covalently bound to the other first peptide motif, and wherein each second branching residue is covalently bound to two second peptide motifs.

18. The nanoparticle according to claim 17, wherein the peptide dendrimer further comprises at least four third branching residues and eight third peptide motifs,
wherein each second peptide motif is respectively covalently bound to one of the third branching residues such that each third branching residue is covalently bound to one second peptide motif, and
wherein each third branching residue is covalently bound to two third peptide motifs.

19. The nanoparticle according to any one of the preceding claims, wherein the peptide dendrimer is selected from Table 1 or Table 1B.

20. The nanoparticle according to any one of the preceding claims, wherein the peptide dendrimer has a structure selected from G1,2-RL, 3-LR; G1,2-R; G1-RL, 2-LR; G1,2-RHL; G1-LRLR; G1,2-RF, 3-HL; G1-R; GSCG1,2-RL, 3-LR; RHCG1,2-R; RHCG1-RL, 2-LR; GSCG1,2-RHL; GSCG1-LRLR; GSCG1,2-RF, 3-HL; or GSCG1-R.

21. The nanoparticle according to any one of the preceding claims, wherein the nucleic acid is:
a. a DNA, optionally an antisense oligonucleotide, a ssDNA, a dsDNA, a plasmid, a cDNA and/or a CpG molecule; or
b. an RNA, optionally an mRNA, a circRNA, an ssRNA, a dsRNA, an sgRNA, a crRNA, a tracrRNA, a lncRNA, an siRNA, an saRNA and/or a self-amplifying RNA.

22. The nanoparticle according to any one of the preceding claims, wherein the nucleic acid encodes a chimeric antigen receptor (CAR) and/or a transcription factor.

23. The nanoparticle according to claim 22, wherein the CAR specifically binds to carcinoembryonic antigen (CEA) or CEA cell Adhersion Molecule 5 (CEACAM5) or CEA Cell Adhesion Molecule 7 (CEACAM7).

24. The nanoparticle according to claim 22, wherein the transcription factor is selected from interferon regulatory factor 5 (IRF5), activated IRF5, inhibitor of nuclear factor kappa B kinase subunit beta (IKK2), CCAAT enhancer binding protein alpha (CEBPA).

25. A nanoparticle according to any one of claims 1 to 24 for use in medicine.

26. A nanoparticle according to any one of claims 1 to 24 for use in a method of treating cancer, an autoimmune disease, an immune cell related disease, a lung disease and/or a myopathy.

27. The nanoparticle for use according to claim 26, wherein the cancer comprises a solid tumour.

28. The nanoparticle for use according to claim 26 or claim 27, wherein the cancer is a lung cancer, such as NSCLC or small cell lung cancer (SCLC), liver cancer, prostate cancer, thyroid cancer, a pancreatic cancer such as pancreatic ductal adenocarcinoma (PDAC), acute myeloid lymphoma (AML), myelodysplastic syndromes (MDS), colorectal cancer such as WT KRAS CRC, a KRAS mutated metastatic tumour, a haematological tumour, an oesophageal cancer, breast cancer, prostate cancer, bladder cancer, a tumour of the GI tract, head and neck squamous cell carcinomas (HNSCC), renal cancer, myelofibrosis, a CD206+ cancer, a melanoma, prostate cancer or anal cancer.

29. The nanoparticle for use according to claim 26, wherein the autoimmune disease is selected from multiple sclerosis (MS), Type I or Type II Diabetes, rheumatoid arthritis, Crohn's diseases, Uveitis, Inflammatory bowel disease or Systemic lupus erythematosus, or wherein the immune related disease is Gaucher's disease, a graft versus host disease, allograft rejection, acute rejection after transplant, chronic rejection after transplant, primary graft dysfunction or chronic granulamatous disease (CGD).

30. The nanoparticle for use according to claim 26, wherein the lung disease is cystic fibrosis, asthma, tuberculosis (TB), acute lung injury (ALI), pulmonary fibrosis, such as idiopathic pulmonary fibrosis, allergic airway disease, chronic obstructive lung disease (COPD), alpha-1 antitrypsin deficiency (AATD), pulmonary arterial hypertension, pulmonary alveolar proteinosis, fibrotic lung disease, chronic lung disease or a respiratory tract infection; or wherein the muscle disease is a muscular dystrophy or a muscle wasting disease.

31. A nanoparticle according to any one of claims 1 to 24 for use as a vaccine.

32. A method of producing a coated nanoparticle that is able to transfect a target cell, the method comprising mixing a solution of peptides or peptide dendrimers with a solution of preformed nanoparticles, to form the coated nanoparticle, wherein:
a) the preformed nanoparticles have a positive surface charge and the peptide dendrimers have a net charge that is negative, or
b) the preformed nanoparticles have a negative surface charge and the peptides or peptide dendrimers have a net charge that is positive, or
c) the surface of the preformed nanoparticles is uncharged and the peptides or peptide dendrimers comprise a hydrophobic region, or
d) the preformed nanoparticles have a positive surface charge and the peptides comprise a more than 2 and less than 100 amino acid residues.

33. The method according to claim 32(a), wherein the peptide dendrimers comprise a PGA, or a glutamic acid containing peptide comprising a glutamic acid rich domain comprising at least 4, at least 6, or at least 8 amino acid residues in total, of which at least 2 are glutamic acid, and wherein at least 20% of the amino acid residues of the glutamic acid rich domain are glutamic acid, or the method according to claim 31(d), wherein the peptides comprise linear PGA comprising more than 2 and less than 100 glutamic acid residues.

34. The method according to claim 32 or 33, wherein the peptide dendrimer comprises a myeloid, lymphoid, muscle, lung, CD206+ cell, tumour cell, bone marrow, pancreas, neuronal tissue, kidney tissue, heart tissue, liver tissue, eye, joint or prostate, or to a stem cell, pancreatic cell, neuronal cell, a kidney cell, a cardiac cell, liver cell, ocular cell, synovial cell or prostate cell targeting motif; optionally selected from;
a. a muscle targeting motif, optionally comprising an ASSLNIA (SEQ ID NO:1), PYDQLRH (SEQ ID NO:2), or KAMHQMQ (SEQ ID NO:3) peptide motif;
b. an integrin targeting motif, optionally comprising an RGD or ACDCRGDCFCG (SEQ ID NO:5) peptide motif;
c. a lung targeting motif, optionally comprising the peptide sequence CGFECVRQCPERC (SEQ ID NO:4);
d. a mannose receptor targeting motif, optionally a mannose sugar or maltotriose;
e. an antibody or target-binding fragment thereof, optionally wherein the antibody specifically binds to CD3;
f. a bone marrow or stem cell targeting motif, optionally comprising an antibody that specifically binds CD34, and/or a lipid comprising a bisphosphonate (BP) group;
g. a pancreas targeting motif, optionally comprising a glucagon-like peptide-1 (GLP-1) homolog such as Exendin-4 (SEQ ID NO:9) or a variant or binding fragment thereof;
h. a kidney targeting motif, optionally comprising (KKEEE)₃K;
i. a neuronal tissue or cell targeting motif, optionally comprising YTIWMPENPRPGTPCDIFTNSRGKRASNGGGG (SEQ ID NO:11) or KSVRTWNEIIPSKGCLRVGGRCHPHVNGGG (SEQ ID NO:12) or a neuronal targeting peptide comprising a Phe-Arg-Trp (FRW) motif;
j. a heart targeting or cardiac cell targeting motif, optionally comprising APWHLSSQYSRT (SEQ ID NO:13);
k. a liver targeting or hepatocyte targeting motif, optionally comprising a asialoglycoprotein receptor binder such as a N-Acetylgalactosamine (Gal-NAc) sugar, or a vitamin A moiety;
l. an eye or ocular cell targeting motif, optionally comprising CARSKNKDC (SEQ ID NO:14);
m. a joint or synovial cell targeting motif, optionally comprising an antibody that specifically binds CD44, and/or a hyaluronan sugar; or
n. a prostate targeting motif, optionally comprising PKRGFQD (SEQ ID NO:15) or SNTRVAP (SEQ ID NO:16).

35. A coated nanoparticle that is able to transfect a target cell, wherein the coated nanoparticle comprises a peptide at its surface, wherein:
a) the nanoparticle is a nanoparticle according to any one of claims 1-24; and/or
b) the peptide is linear PGA comprising more than 2 and less than 100 glutamic acid residues, or the peptide is branched PGA; and/or
c) the peptide is a glutamic acid containing peptide comprising a glutamic acid rich domain comprising at least 4, at least 6, or at least 8 amino acid residues in total, of which at least 2 are glutamic acid, and wherein at least 20% of the amino acid residues of the glutamic acid rich domain are glutamic acid; and/or
d) the nanoparticle is coated with a dendrimer selected from Table 1 or Table 1B; or
e) the nanoparticle is coated with a peptide selected from Table 1A, wherein the peptide is not E100.

36. The coated nanoparticle according to claim 35(b), (c), (d) or (e), wherein the PGA and/or glutamic acid containing peptide comprises a myeloid, lymphoid, muscle, lung, CD206+ cell, tumour cell, neuronal tissue, kidney tissue, heart tissue, neuronal cell, kidney cell, or cardiac cell targeting motif; optionally selected from;
a. a muscle targeting motif, optionally comprising an ASSLNIA (SEQ ID NO:1), PYDQLRH (SEQ ID NO:2), or KAMHQMQ (SEQ ID NO:3) peptide motif;
b. an integrin targeting motif, optionally comprising an RGD or ACDCRGDCFCG (SEQ ID NO:5) peptide motif;
c. a lung targeting motif, optionally comprising the peptide sequence CGFECVRQCPERC (SEQ ID NO:4);
d. a mannose receptor targeting motif, optionally a mannose sugar or maltotriose;
e. an antibody or target-binding fragment thereof, optionally wherein the antibody specifically binds to CD3;
f. a bone marrow or stem cell targeting motif, optionally comprising an antibody that specifically binds CD34, and/or a lipid comprising a bisphosphonate (BP) group;
g. a pancreas targeting motif, optionally comprising a glucagon-like peptide-1 (GLP-1) homolog such as Exendin-4 (SEQ ID NO:9) or a variant or binding fragment thereof;
h. a kidney targeting motif, optionally comprising (KKEEE)₃K;
i. a neuronal tissue or cell targeting motif, optionally comprising YTIWMPENPRPGTPCDIFTNSRGKRASNGGGG (SEQ ID NO:11) or KSVRTWNEIIPSKGCLRVGGRCHPHVNGGG (SEQ ID NO:12) or a neuronal targeting peptide comprising a Phe-Arg-Trp (FRW) motif;
j. a heart targeting or cardiac cell targeting motif, optionally comprising APWHLSSQYSRT (SEQ ID NO:13);
k. a liver targeting or hepatocyte targeting motif, optionally comprising a asialoglycoprotein receptor binder such as a N-Acetylgalactosamine (Gal-NAc) sugar, or a vitamin A moiety;
l. an eye or ocular cell targeting motif, optionally comprising CARSKNKDC (SEQ ID NO:14);
m. a joint or synovial cell targeting motif, optionally comprising an antibody that specifically binds CD44, and/or a hyaluronan sugar; or
n. a prostate targeting motif, optionally comprising PKRGFQD (SEQ ID NO:15) or SNTRVAP (SEQ ID NO:16).

37. A composition comprising a nanoparticle comprising first peptide dendrimer, a second peptide dendrimer, a nucleic acid and a lipid, wherein the first and second peptide dendrimers comprise at least: a core peptide sequence, a first branching residue and two first peptide motifs.

38. The composition of claim 37, wherein the first peptide dendrimer is a first generation peptide dendrimer comprising a core peptide sequence, a first branching residue and two first peptide motifs.

39. The composition according to claim 37 or claim 38, wherein the second peptide dendrimer further comprises at least two second branching residues and four second peptide motifs, wherein one of the second branching residues is covalently bound to one of the first peptide motifs and the other second branching residue is covalently bound to the other first peptide motif, and wherein each second branching residue is covalently bound to two second peptide motifs.

40. The composition according to any one claims 37 to 39, wherein the second peptide dendrimer further comprises at least four third branching residues and eight third peptide motifs, wherein each second peptide motif is respectively covalently bound to one of the third branching residues such that each third branching residue is covalently bound to one second peptide motif, and wherein each third branching residue is covalently bound to two third peptide motifs.

41. The composition according to any one of claims 37 to 40, wherein the branching residues of the first and second peptide dendrimers are independently selected from lysine, 2,4-diaminobutyric acid, ornithine, or diaminopropionic acid.

42. The composition according to any one of claims 37 to 41, wherein the peptide motifs of the first and second peptide dendrimers independently consist of a single amino acid, dipeptide, tripeptide or tetrapeptide motifs.

43. The composition according to any one of claims 37 to 42, wherein the first and second peptide dendrimer peptide motifs independently comprise an amino acid with a basic side chain, a non-polar side chain, an acidic side chain, and/or an uncharged polar side chain.

44. The composition according to any one of claims 37 to 43, wherein the first and second peptide dendrimer peptide motifs independently comprise a leucine (L), an arginine (R) and/or a histidine (H) residue.

45. The composition according to any one of claims 37 to 44, wherein the core sequence of the first and/or second peptide dendrimer comprises at least two amino acids.

46. The composition according to any one of claims 37 to 45, wherein the core sequence of the first and/or second peptide dendrimer comprises up to 30 amino acids.

47. The composition according to any one of claims 37 to 46, wherein the first and/or second peptide dendrimer core peptide sequence comprises the peptide sequence RHC, GSA, or GSC.

48. The composition according to any one of claims 37 to 47, wherein the core sequence of the first and/or second peptide dendrimer comprises an ionisable amino acid such as histidine.

49. The composition according to any one of claims 37 to 48, wherein the nanoparticle has a polydispersity index of less than 0.6, 0.55, 0.5, 0.45, 0.4, 0.39, 0.38, 0.37, 0.36, 0.35, 0.34, 0.33, 0.32, 0.31, or 0.30.

50. The composition according to any one of claims 37 to 49, wherein the first peptide dendrimer, when used in a first reference nanoparticle consisting of the first peptide dendrimer, a nucleic acid and a lipid, the first reference nanoparticle has a PDI of between 0.05 and 0.9, between 0.06 and 0.8, between 0.07 and 0.6, between 0.08 and 0.5, between 0.09 and 0.4, between 0.1 and 0.3, between 0.12 and 0.2, or between 0.13 and 0.15 greater than the PDI of a second reference nanoparticle consisting of the second peptide a nucleic acid and a lipid.

51. The composition according to any one of claims 37 to 50, wherein the molar ratio with respect to the nitrogen contributed by each of the first peptide dendrimer and the second peptide dendrimer is between 10:1 and 1:10, 5:1 and 1:5, 1:4 and 4:1, 1:3 and 3:1, or 1:2 and 2:1.

52. The composition according to any one of claims 37 to 51, wherein the molar ratio with respect to the nitrogen contributed by each of the first peptide dendrimer and the second peptide dendrimer is 1:4, 1:3, 1:2, 1:1, 2:1, 3:1, or 4:1.

53. The composition according to any one of claims 37 to 52, wherein the first peptide dendrimer comprises a structure set forth in Table 7 and/or the second peptide dendrimer comprises a structure set forth in Table 8.

54. The composition according to any one of claims 37 to 53, wherein the first peptide dendrimer comprises a structure G1-LRLR; G1-R; G1,2-R; G1-RLR; GSCG1-LRLR; RHCG1-R; RHCG1,2-R; or RHCG1-RLR; and/or the second peptide dendrimer comprises the structure G1,2-RL, 3-LR; G1-RL, 2-LR; GSCG1,2-RL, 3-LR; or RHCG1-RL, 2-LR.

55. The composition according to any one of claims 37 to 54, wherein the nucleic acid is selected from;
a. a DNA, optionally an antisense oligonucleotide, a ssDNA, a dsDNA, a plasmid, a CpG molecule and/or a cDNA; or
b. an RNA, optionally an mRNA, a circRNA, a ssRNA, a dsRNA, an sgRNA, a crRNA, a tracrRNA, a lncRNA, an siRNA, an saRNA and/or a self-amplifying RNA.

56. The composition according to any one of claims 37 to 55, wherein the nucleic acid encodes a protein or peptide, optionally wherein the protein or peptide comprises a Chimeric Antigen Receptor (CAR), a transcription factor, an antigen, a hormone, a receptor, a chimeric antigen receptor, a transcription factor and/or a cytokine such as IL-2, IL-7, IL-12, IL-15, IL-21 and/or interferon.

57. The composition according to any one of claims 37 to 56, wherein delivery of the nucleic acid to a tissue or cell is increased by at least 10%, 15%, 20%, 25%, 30%, 35%, 40%, 45%, 50%, 55%, 60%, 65%, 75%, 85%, 90%, 95%, or 100% compared to delivery of the same nucleic acid to the same tissue or cell type using a composition comprising a lipid, nucleic acid and only either the first or second peptide dendrimer.

58. A composition according to any one of claims 37 to 57 for use in medicine.

59. A composition according to any one of claims 37 to 57 for use as a vaccine.

60. A composition according to any one of claims 37 to 57 for use in a method of treating cancer, an autoimmune disease and/or a lung disease.

61. The composition for use according to claim 60, where the cancer is a solid tumour, a lung cancer, such as NSCLC or small cell lung cancer (SCLC), liver cancer, prostate cancer, thyroid cancer, a pancreatic cancer such as pancreatic ductal adenocarcinoma (PDAC), acute myeloid lymphoma (AML), myelodysplastic syndromes (MDS), colorectal cancer such as WT KRAS CRC, a KRAS mutated metastatic tumour, a haematological tumour, an oesophageal cancer, breast cancer, prostate cancer, bladder cancer, a tumour of the GI tract, head and neck squamous cell carcinomas (HNSCC), renal cancer, a CD206+ cancer, a melanoma, prostate cancer or anal cancer.

62. The composition for use according to claim 60, where the autoimmune disease is selected from multiple sclerosis (MS), Type I or Type II Diabetes, rheumatoid arthritis, Crohn's diseases, Uveitis, or Inflammatory bowel disease or Systemic lupus erythematosus, or wherein the immune related disease is Gaucher's disease, a graft versus host disease, allograft rejection, acute rejection after transplant, chronic rejection after transplant, primary graft dysfunction or chronic granulamatous disease (CGD), or wherein the lung disease is cystic fibrosis, asthma, tuberculosis (TB), acute lung injury (ALI), pulmonary fibrosis, such as idiopathic pulmonary fibrosis, allergic airway disease, chronic obstructive lung disease (COPD), alpha-1 antitrypsin deficiency (AATD), pulmonary arterial hypertension, pulmonary alveolar proteinosis, fibrotic lung disease, chronic lung disease or a respiratory tract infection; or wherein the muscle disease is a muscular dystrophy or a muscle wasting disease.

63. A nanoparticle comprising a peptide dendrimer, a nucleic acid and a lipid, wherein the peptide dendrimer comprises at least: a core peptide sequence, a first branching residue and two first peptide motifs,
wherein the nanoparticle is targeted to bone marrow, pancreas, neuronal tissue, kidney tissue, heart tissue, liver tissue, eye, joint or prostate, or to a stem cell, pancreatic cell, neuronal cell, a kidney cell, a cardiac cell, liver cell, ocular cell, synovial cell or prostate cell.

64. The nanoparticle according to claim 63, wherein the nanoparticle comprises a targeting motif selected from:
a. a bone marrow or stem cell targeting motif, optionally comprising an antibody that specifically binds CD34, and/or a lipid comprising a bisphosphonate (BP) group;
b. a pancreas targeting motif, optionally comprising a glucagon-like peptide-1 (GLP-1) homolog such as Exendin-4 (SEQ ID NO:9) or a variant or binding fragment thereof;
c. a kidney targeting motif, optionally comprising (KKEEE)₃K;
d. a neuronal tissue or cell targeting motif, optionally comprising YTIWMPENPRPGTPCDIFTNSRGKRASNGGGG (SEQ ID NO:11) or KSVRTWNEIIPSKGCLRVGGRCHPHVNGGG (SEQ ID NO:12) or a neuronal targeting peptide comprising a Phe-Arg-Trp (FRW) motif;
e. a heart targeting or cardiac cell targeting motif, optionally comprising APWHLSSQYSRT (SEQ ID NO:13);
f a liver targeting or hepatocyte targeting motif, optionally comprising a asialoglycoprotein receptor binder such as a N-Acetylgalactosamine (Gal-NAc) sugar, or a vitamin A moiety;
g an eye or ocular cell targeting motif, optionally comprising CARSKNKDC (SEQ ID NO:14);
h a joint or synovial cell targeting motif, optionally comprising an antibody that specifically binds CD44, and/or a hyaluronan sugar; or
i a prostate targeting motif, optionally comprising PKRGFQD (SEQ ID NO:15) or SNTRVAP (SEQ ID NO:16).

65. The nanoparticle according to claim 63 or claim 64, wherein the nanoparticle comprises a cell targeting motif that targets the stem cell, pancreatic cell, neuronal cell, a kidney cell, a cardiac cell, liver cell, ocular cell, synovial cell or prostate cell.

66. The nanoparticle according to any one of claims 63 to 65, wherein the nanoparticle is capable of transfecting the targeted stem cell, pancreatic cell, neuronal cell, a kidney cell, a cardiac cell, liver cell, ocular cell, synovial cell or prostate cell in vitro at an efficiency of at least 10%, at least 12.5%, at least 15%, at least 17.5%, at least 20%, at least 25%, at least 30%, at least 40%, at least 45%, at least 50%, at least 55%, at least 60%, at least 65%, at least 70%, at least 75%, at least 80%, at least 85%.

67. The nanoparticle according to any one of claims 63 to 66, wherein the bone marrow, pancreas, neuronal tissue, kidney tissue, heart tissue, liver tissue, eye, joint or prostate, stem cell, pancreatic cell, neuronal cell, a kidney cell, a cardiac cell, liver cell, ocular cell, synovial cell or prostate cell targeting motif is bound to a negatively charged or neutrally charged polymer or lipid.

68. The nanoparticle according to claim 67, wherein the polymer or lipid is selected from polyglutamic acid (PGA), poly(acrylic acid), alginic acid, polyethylene glycol (PEG), or cholesteryl hemisuccinate/1,2-dioleoyl-sn-glycero-3-phosphoethanolamine, a neutrally-charged zwitterionic polymer or lipid, or a glutamic acid containing peptide.

69. The nanoparticle according to claim 67 or claim 68, wherein the polymer is a linear or dendritic PGA.

70. The nanoparticle according to claim 67 or claim 68, wherein the polymer is a dendritic glutamic acid containing peptide.

71. The nanoparticle according to any one of claims 63 to 70, wherein the bone marrow, pancreas, neuronal tissue, kidney tissue, heart tissue, liver tissue, eye, joint or prostate, stem cell, pancreatic cell, neuronal cell, a kidney cell, a cardiac cell, liver cell, ocular cell, synovial cell or prostate cell targeting motif is covalently bound to the peptide dendrimer, or wherein the nanoparticle further comprises a second peptide comprising the bone marrow, pancreas, neuronal tissue, kidney tissue, heart tissue, liver tissue, eye, joint or prostate, stem cell, pancreatic cell, neuronal cell, a kidney cell, a cardiac cell, liver cell, ocular cell, synovial cell or prostate cell targeting motif.

72. The nanoparticle according to claim 71, wherein the second peptide comprises linear PGA comprising 2 to 10, 2 to 25, 2 to 15 or 2 to 100 glutamic acid residues, or wherein the second peptide comprises branched PGA comprising 2 to 10, 2 to 25, 2 to 15 or 2 to 100 glutamic acid residues in each branch and/or in the core sequence, optionally wherein the second peptide comprises the structure (Ac-EEEE)2KGSGGSGGSC[(S-S)-α-D-Thiomannose].

73. The nanoparticle according to any one of claims 63 to 72, wherein the lipid:nucleic acid weight/weight ratio is between 0.5:1 and 40:1, between 1:1 and 30:1, or between 2:1 and 25:1, optionally wherein the lipid:nucleic acid weight/weight ratio is at least 10:1 and wherein the nanoparticle is targeted to spleen and lung, or wherein the lipid:nucleic acid weight/weight ratio is about 23:1 and the nanoparticle is targeted to lung and spleen.

74. The nanoparticle according to any one of claims 63 to 73, wherein the lipid comprises two lipids, three lipids, four lipids, five lipids, or more than five lipids, optionally wherein the lipids comprise DOPE, DOTMA, DODAP, DORI, DMG-PEG and/or DOTAP.

75. The nanoparticle according to claim 74, wherein the lipid comprises DOTMA and DOPE.

76. The nanoparticle according to claim 74 or claim 75, wherein the lipid comprises three lipid components, or four lipid components, wherein each lipid component is selected from the group comprising DOPE, DOTMA, DODAP, DORI, DMG-PEG and DOTAP.

77. The nanoparticle according to claim 75 or claim 76, wherein the lipid comprises DODAP, DOTMA and DOPE; or wherein the lipid comprises DODAP, DOTAP, and DOPE; or wherein the lipid comprises DODAP, DORI, and DOPE; or wherein the lipid comprises DODAP, DOTMA, DOPE and DMG-PEG; or wherein the lipid comprises DODAP, DOTAP, DOPE and DMG-PEG.

78. The nanoparticle according to any one of claims 63 to 77, wherein the peptide dendrimer further comprises two second branching residues and four second peptide motifs, wherein one of the second branching residues is covalently bound to one of the first peptide motifs and the other second branching residue is covalently bound to the other first peptide motif, and wherein each second branching residue is covalently bound to two second peptide motifs.

79. The nanoparticle according to claim 78, wherein the peptide dendrimer further comprises at least four third branching residues and eight third peptide motifs,
wherein each second peptide motif is respectively covalently bound to one of the third branching residues such that each third branching residue is covalently bound to one second peptide motif, and wherein each third branching residue is covalently bound to two third peptide motifs.

80. The nanoparticle according to any one of the preceding claims, wherein the peptide dendrimer is selected from Table 1 or Table 1B, or wherein the nanoparticle is coated with a peptide selected from Table 1A.

81. The nanoparticle according to any one of claims 63 to 80, wherein the peptide dendrimer has a structure selected from G1,2-RL, 3-LR; G1,2-R; G1-RL, 2-LR; G1,2-RHL; G1-LRLR; G1,2-RF, 3-HL; G1-R; GSCG1,2-RL, 3-LR; RHCG1,2-R; RHCG1-RL, 2-LR; GSCG1,2-RHL; GSCG1-LRLR; GSCG1,2-RF, 3-HL; or GSCG1-R.

82. The nanoparticle according to any one of claims 63 to 81, wherein the nucleic acid is:
a. a DNA, optionally an antisense oligonucleotide, a ssDNA, a dsDNA, a plasmid, a cDNA and/or a CpG molecule; or
b. an RNA, optionally an mRNA, a circRNA, an ssRNA, a dsRNA, an sgRNA, a crRNA, a tracrRNA, a lncRNA, an siRNA, an saRNA and/or a self-amplifying RNA.

83. The nanoparticle according to any one of claims 63 to 82, wherein the nucleic acid encodes a chimeric antigen receptor (CAR) and/or a transcription factor.

84. The nanoparticle according to claim 83, wherein the CAR specifically binds to carcinoembryonic antigen (CEA) or CEA cell Adhersion Molecule 5 (CEACAM5) or CEA Cell Adhesion Molecule 7 (CEACAM7).

85. The nanoparticle according to claim 83, wherein the transcription factor is selected from interferon regulatory factor 5 (IRF5), activated IRF5, inhibitor of nuclear factor kappa B kinase subunit beta (IKK2), CCAAT enhancer binding protein alpha (CEBPA).

86. A nanoparticle according to any one of claims 63 to 85 for use in medicine.

87. A nanoparticle according to any one of claims 63 to 85 for use as a vaccine.

88. A nanoparticle according to any one of claims 63 to 85 for use in a method of treating cancer, an autoimmune disease, an immune cell related disease, a lung disease and/or a myopathy.

89. The nanoparticle for use according to claim 88, wherein the cancer comprises a solid tumour.

90. The nanoparticle for use according to claim 88 or claim 89, wherein the cancer is a lung cancer, such as NSCLC or small cell lung cancer (SCLC), liver cancer, prostate cancer, thyroid cancer, a pancreatic cancer such as pancreatic ductal adenocarcinoma (PDAC), acute myeloid lymphoma (AML), myelodysplastic syndromes (MDS), colorectal cancer such as WT KRAS CRC, a KRAS mutated metastatic tumour, a haematological tumour, an oesophageal cancer, breast cancer, prostate cancer, bladder cancer, a tumour of the GI tract, head and neck squamous cell carcinomas (HNSCC), renal cancer, myelofibrosis, a CD206+ cancer, a melanoma, prostate cancer or anal cancer.

91. The nanoparticle for use according to claim 88, wherein the autoimmune disease is selected from multiple sclerosis (MS), Type I or Type II Diabetes, rheumatoid arthritis, Crohn's diseases, Uveitis, or Inflammatory bowel disease, or wherein the immune related disease is Gaucher's disease, a graft versus host disease, allograft rejection, acute rejection after transplant, chronic rejection after transplant, primary graft dysfunction or chronic granulamatous disease (CGD).

92. The nanoparticle for use according to claim 88, wherein the lung disease is cystic fibrosis, asthma, tuberculosis (TB), acute lung injury (ALI), pulmonary fibrosis, such as idiopathic pulmonary fibrosis, allergic airway disease, chronic obstructive lung disease (COPD), a Ipha-1 antitrypsin deficiency (AATD), pulmonary arterial hypertension, pulmonary alveolar proteinosis, fibrotic lung disease, chronic lung disease or a respiratory tract infection; or wherein the muscle disease is a muscular dystrophy or a muscle wasting disease.
